(19) United States
(12) Patent Application Publication
(10) Pub. No.: US 2011/0268744 A1
(43) Pub. Date: Nov. 3, 2011

(54) Method of Diagnosis of Infection by Mycobacteria and Reagents Therefor

(76) Inventors: Ian Garthwaite, Brookvale NSW (AU); Robyn Lindner, Five Dock NSW (AU); Susanne Pedersen, North Ryde NSW (AU)

(21) Appl. No.: 12/994,714

(22) PCT Filed: May 26, 2009
(86) PCT No.: PCT/AU2009/000656

§ 371 Date: May 25, 2011

(30) Foreign Application Priority Data
May 26, 2008 (AU) 2008902611

(51) Int. Cl. A61K 39/395; G01N 33/573; A61P 31/06; C12N 1/21; C07K 17/00; A61P 31/04; C12N 5/10; C07K 16/40

(52) U.S. Cl. 424/158.1; 435/338; 435/7.1; 530/389.5; 435/252.33; 435/7.6; 435/7.92; 435/7.94; 530/391.1

(57) ABSTRACT

The present invention provides isolated M. tuberculosis protein that is a putative Ketol-acid reductoisomerase (KARI; SEQ ID NO: 1) and immunogenic peptide fragments thereof, and antibodies produced against the full-length protein and immunogenic peptide fragments for the diagnosis of tuberculosis and/or infection by one or more mycobacteria of the M. tuberculosis complex in humans, for example using an antigen-based sandwich ELISA format. The present invention also provides multianalyte assays in which the KARI-based diagnostic assays of the present invention are multiplexed with the detection of one or more immunogenic epitopes from one or more other proteins of said mycobacteria e.g., anyone of SEQ IDS NOs: 2, 14, 21, 28-29, 36, or 44, including any combinations thereof.

### RELATED APPLICATION DATA

This application claims priority from Australian Patent Application No. 2008902611 filed May 26, 2008, the contents of which are incorporated herein in their entirety.

### FIELD OF THE INVENTION

The present invention relates to novel diagnostic, prognostic and therapeutic reagents for infection of an animal subject such as a human by M. tuberculosis, and conditions associated with such infections, such as, for example, tuberculosis. More particularly, the present to invention provides the first enabling disclosure of the expression in an infected subject of a Ketol-acid reductoisomerase (KARI) protein of M. tuberculosis (SEQ ID NO: 1) and immunogenic epitopes thereof suitable for the preparation of immune-logical reagents, such as, for example, antigenic proteins/peptides and/or antibodies, for the diagnosis, prognosis and therapy of infection, and vaccine development.

### BACKGROUND OF THE INVENTION

### Description of the Related Art

Tuberculosis is a chronic, infectious disease that is generally caused by infection with Mycobacterium tuberculosis or by one or more organisms of the Mycobacterium tuberculosis complex. As used herein, the term “Mycobacterium tuberculosis complex” means one or more organisms selected from the group consisting of M. tuberculosis, M. bovis, M. africanum, M. canetti and M. microti. The skilled artisan is also aware that the M. tuberculosis complex is distinct from the so-called M. avium complex including M. avium and M. intracellulaire which are causative agents of the unrelated disease known as paratuberculosis e.g., in agricultural animals.

Tuberculosis is a major disease in developing countries, as well as an increasing problem in developed areas of the world, with about eight million new cases and three million deaths each year. Although the infection may be asymptomatic for a considerable period of time, the disease is most commonly manifested as an acute inflammation of the lungs, resulting in fever and a productive cough. If left untreated, M. tuberculosis infection may progress beyond the primary infection site in the lungs to any organ in the body and generally results in serious complications and death.

The problems of the rapidly growing global incidence of tuberculosis and microbial resistance have been often described by many workers in the health care industry and are well known to skilled artisans in that field. In particular there is a growing recognition that new diagnostics, drugs and vaccines are urgently needed.

The immune-logical mechanisms by which M. tuberculosis maintains and multiplies within the host are poorly understood. Consequently, any new information regarding the immune-logical relationship between tuberculosis and the host could clearly be used in many different ways to improve diagnosis, therapy and treatment of that disease.

The incidence of tuberculosis is especially common in late-staging AIDS patients, a majority of whom suffer from it. In fact, HIV infection is a most important risk factor for the development of active tuberculosis in purified protein derivative (PPD)-tuberculin-positive subjects, and the risk of acquisition of tuberculosis infection in HIV-infected immune-suppressed individuals may be markedly enhanced compared to those individuals that are not HIV-infected. It is also likely that co-infections with HIV-1, and M. tuberculosis mediate a shortened HIV symptom-free period and shortened survival time in subjects, possibly by triggering increased viral replication and virus load that results in depletion of CD4+ T-cells and immune deficiency or immune suppression (Corbett et al 2003; Ho, Mem. Inst. Oswaldo Cruz, 91, 385-387, 1996).

The sequencing of the Mycobacterium tuberculosis genome has facilitated an enormous research effort to identify potential M. tuberculosis proteins that theoretically may be expressed by the organism. However, sequence data alone are insufficient to conclude that any particular protein is expressed in vivo by the organism, let alone during infection of a human or other animal subject. Nor does the elucidation of open reading frames in the genome of M. tuberculosis indicate that any particular protein encoded or actually expressed by the bacterium comprises any immune-dominant B-cell epitopes or T-cell epitopes that are required for the preparation of diagnostic, prognostic and therapeutic immune-logical reagents. For example, to conclude that a particular protein of M. tuberculosis or a peptide fragment derived there from has efficacy as a diagnostic reagent in an immune-assay format, or is suitable for use in a vaccine preparation, it is necessary to show that the protein is expressed during infectious cycle of the bacterium, and that the host organism mounts an immune response to the protein, and/or to a peptide fragment that comprises a B cell epitope or T-cell epitope (e.g., CD8⁺-restricted CTL epitope).

The ability to grow M. tuberculosis in culture has provided a convenient model to identify expressed tuberculosis proteins in vitro. However, the culture environment is markedly different to the environment of a human macrophage, lung, or extrapulmonary site where M. tuberculosis is found in vivo. Recent evidence indicates that the protein expression profile of intracellular parasites, such as, for example, M. tuberculosis, varies markedly depending on environmental cues, such that the expression profile of the organism in vitro may not accurately reflect the expression profile of the organism in situ.

Infection with M. tuberculosis bacilli, or reactivation of a latent infection, induces a host response comprising the recruitment of monocytes and macrophages to the site of infection. As more immune cells accumulate a nodule of granulomata forms comprising immune cells and host tissue that have been destroyed by the cytotoxic products of macrophages. As the disease progresses, macrophage enzymes cause the hydrolysis of protein, lipid and nucleic acids resulting in liquefaction of surrounding tissue and granuloma formation. Eventually the lesion ruptures and the bacilli are released into the surrounding lung, blood or lymph system.

During this infection cycle, the bacilli are exposed to four distinct host environments, being alveoli macrophage, caseous granuloma, extracellular lung and extrapulmonary sites, such as, for example the kidneys or peritoneal cavities, lymph, bone, or spine.

It is thought that bacilli can replicate to varying degrees in all these environments, however, little is known about the environmental conditions at each site. All four host environments are distinct, suggesting that the expression profile of M. tuberculosis in each environment will be different.

Accordingly, the identification of M. tuberculosis proteins from logarithmic phase cultures does not necessarily suggest which proteins are expressed or highly immunogenic in each environment in vivo. Similarly, the identification of M. tuberculosis proteins in a macrophage grown in vitro will not necessarily emulate the protein expression profile of M. tuberculosis in caseous granuloma, highly aerated lung, or at an extrapulmonary site having a low oxygen content.

Furthermore, M. tuberculosis infection within the host can be seen as a dynamic event where the host immune system is continually trying to encapsulate and destroy bacilli through destruction of infected macrophages. Consequently, the M. tuberculosis bacilli progress through cycles of intracellular growth, destruction (where both intracellular and secreted bacterial proteins are exposed and destroyed), and rapid extracellular multiplication. Host and pathogen interaction is a result of many factors, which can not be replicated in vitro.

Accordingly, until the present invention, it was not clear which M. tuberculosis proteins were the most highly expressed and/or highly immune-logically active or immunogenic proteins of M. tuberculosis in any particular environment in vivo.

There clearly remains a need for rapid and cost-effective diagnostic and prognostic reagents for determining infection by M. tuberculosis and/or disease conditions associated therewith.

Conventional techniques of molecular biology, microbiology, proteomics, virology, recombining DNA technology, peptide synthesis in solution, solid phase peptide synthesis, and immune-logy described, for example, in the following texts:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Immobilized Cells and Enzymes: A Practical Approach (1986) IRL Press, Oxford, whole of text;
6. Perbal, B., A Practical Guide to Molecular Cloning (1984);
7. Methods In Enzymology (S. Colowick and N. Kaplan, eds., Academic Press, Inc.), whole of series;
8. J. F. Ramalho Ortigão, “The Chemistry of Peptide Synthesis” In: Knowledge database of Access to Virtual Laboratory website (Interactiva, Germany);
9. Sakakibara, D., Teichman, J., Lien, E. Land Fenichel, R. L. (1976). Biochem. Biophys. Res. Commun. 73 336-342
10. Merrifield, R. B. (1963). J. Am. Chem. Soc. 85, 2149-2154.
11. Barany, G. and Merrifield, R. B. (1979) in The Peptides (Gross, E. and Meienhofer, J. eds.), vol. 2, pp. 1-284, Academic Press, New York.
12. Wunsch, E., ed. (1974) Synthese von Peptiden in Houben-Weyls Metoden der Organischen Chemie (Müller, E., ed.), vol. 15, 4th edn., Parts 1 and 2, Thieme, Stuttgart.
13. Bodanszky, M. (1984) Principles of Peptide Synthesis, Springer-Verlag, Heidelberg.
14. Bodanszky, M. & Bodanszky, A. (1984) The Practice of Peptide Synthesis, Springer-Verlag, Heidelberg.
15. Bodanszky, M. (1985) Int. J. Peptide Protein Res. 25, 449-474.
16. Handbook of Diagnostic testal Immune-logy, Vols. I-IV (D. M. Weir and C. C. Blackwell, eds., 1986, Blackwell Scientific Publications).
17. Wilkins M. R., Williams K. L., Appel R. D. and Hochstrasser (Eds) 1997 Proteome Research: New Frontiers in Functional Genomics Springer, Berlin.

### SUMMARY OF INVENTION

### 1. Introduction

In work leading up to the present invention, the inventors sought to elucidate the range of proteins expressed by M. tuberculosis complex organism(s) in a range of in vivo environments, to thereby identify highly expressed and/or highly immunogenic proteins of M. tuberculosis and other organism(s) of the M. tuberculosis complex.

The inventors used a proteomics approach to identify M. tuberculosis complex proteins expressed in vivo and present in the body fluids of a cohort of diseased patients, including sputum, pleural fluid, plasma and serum. An M. tuberculosis complex protein was identified in vivo by 2-dimensional electrophoresis of immune-globulin-containing samples, in particular IgG, obtained previously from a cohort of patients diagnosed with tuberculosis e.g., patients infected with M. tuberculosis or other organism of the M. tuberculosis complex. A peptide fragment was identified, and the amino acid sequences of peptide fragments were determined by mass spectrometry of tryptic fragments, and shown to align to the amino acid sequence of the Ketol-Acid Reducto Isomerase (KARI) protein (SEQ ID NO: 1). In particular, a matched peptide aligned to a region of the KARI protein sequence.

In one example, the inventors have made more than ten (10) distinct preparations of antibodies that bind to recombinant full-length KARI protein and to peptide regions of the full-length KARI protein, for the development of antigen-based diagnostic and prognostic assays, including a polyclonal antibody preparation designated “Ch34/35” prepared in chickens against full length recombinant KARI protein (SEQ ID NO: 1) conjugated to a hexahistidine tag, three monoclonal antibodies designated “Mo1283F”, “Mo2B1”, and Mo1F6″ against the full-length recombinant KARI protein (SEQ ID NO: 1) conjugated to a hexahistidine tag, two monoclonal antibodies designated “Mo4F7 and “Mo4C10” prepared against residues 40-56 of SEQ ID NO: 1, a monoclonal antibody designated “Mo2D6” prepared against residues 290-300 of SEQ ID NO: 1, and two monoclonal antibodies prepared against residues 298-310 of SEQ ID NO: 1. As will be known to the skilled artisan, antibodies can also be prepared against other immunogenic peptide fragments of the full-length KARI protein without undue experimentation.

As exemplified herein, antibodies raised against recombinant protein and peptides bind to recombinant KARI protein and to endogenous KARI protein in clinical samples from subjects diagnosed previously by culture and smear tests to have tuberculosis e.g., patients infected with M. tuberculosis or other organism of the M. tuberculosis complex. The antibodies also detect endogenous KARI protein expressed by clinical and laboratory strains of M. tuberculosis, and to have low cross-reactivity with other microorganisms including Bacillus subtilis, Escherichia coli, or Pseudomonas aeruginosa. Antibodies prepared against KARI are also capable of detecting low levels of KARI protein in sandwich ELISA and a point-of-need assay e.g., as described in U.S. Pat. No. 7,205,159 and European Pat. No. 1461615 incorporated herein by reference.

Antibodies are also obtained with a view to selecting high-affinity antibodies capable of detecting M. tuberculosis KARI at sub-picogram/ml levels in patient body fluids, such as sputum, saliva, pleural fluid, serum, plasma, etc.

Similarly, antibodies are obtained with a view to selecting high-affinity antibodies capable of detecting KARI from one or more other organisms of the M. tuberculosis complex by virtue of the high sequence identity between KARI protein expressed by M. tuberculosis and a KARI protein expressed by one or more organisms selected from the group consisting of M. bovis, M. africanum, M. canetti and M. microti and the conservation of B-cell epitopes there between permitting cross-reactivity between one or more of said organisms.

These findings have provided the means for producing novel antigen-based diagnostics for the diagnosis of tuberculosis e.g., by virtue of the detection of M. tuberculosis or other M. tuberculosis complex organism in a subject, and novel antigen-based prognostic indicators for the progression of infection or a disease state associated therewith. Preferably, one or more antibodies that bind to the KARI protein or a B-cell epitope thereof are useful for the early diagnosis of infection or disease. It will also be apparent to the skilled person that such diagnostic and prognostic tests may be used in conjunction with therapeutic treatments for tuberculosis or an infection associated therewith e.g., to determine efficacy of therapeutic intervention.

In a further example described herein, multianalyte assays, e.g., using one or more antibodies that binds to KARI protein and one or more antibodies that bind to one or more other proteins of M. tuberculosis or other organism of the M. tuberculosis complex, e.g., a protein selected from the group consisting of BSX protein, S9 protein, Rv1265 protein, EF-Tu protein, P5CR protein, TetR-like protein, glutamine synthetase protein and combinations thereof, provide high sensitivity and specificity.

In a further example, such antigen-based diagnostic and prognostic assays are combined with standard culture tests for the diagnosis of tuberculosis e.g., by virtue of detecting the presence of M. tuberculosis or other organisms of the M. tuberculosis complex in clinical samples e.g., to confirm an initial diagnosis and/or to indicate the specific pathogen involved. In one example, a culture test confirms the presence of M. tuberculosis in a clinical sample as opposed to another mycobacteria pathogen. In another example, a culture test confirms the strain of M. tuberculosis or other mycobacteria pathogen present in a clinical specimen obtained from a subject.

Notwithstanding that smear tests are less accurate than culture tests, it is to be understood that such testing may be readily combined with antigen-based assays of the present invention without undue experimentation, as may any other art-recognized means for diagnosing tuberculosis or determining or detecting the presence of a causative agent of tuberculosis, e.g., M. tuberculosis or other organism of the M. tuberculosis complex, in a specimen from a subject suspected of being infected, or at risk of being infected.

In a further example, antibodies that bind to the amino acid sequence set forth in SEQ ID NO: 1 or a variant thereof from an organism of the M. tuberculosis complex and that are present in subjects suffering form extrapulmonary tuberculosis provide additional means for diagnosing an active or past infection by mycobacteria of the M. tuberculosis complex. To assay for the presence of such antibodies in subjects, recombinant KARI protein comprising the sequence set forth in SEQ ID NO: 1 or a variant thereof or an immunogenic peptide derived from the full-length protein sequence is employed in antibody-based assays as a detection reagent for identifying the presence of antibodies in an antibody-containing sample obtained from the subject e.g., blood, serum, a fraction of serum, etc. As with antigen-based assays, the detection of anti-KARI antibodies of the M. tuberculosis complex in a subject is readily combined with detection of antibodies against other immunogenic proteins of the M. tuberculosis complex e.g., by additionally detecting antibodies against one or more proteins of M. tuberculosis or other causative agent of tuberculosis, or one or more B cell epitopes thereof, wherein said protein(s) is(are) selected from the group consisting of BSX protein, S9 protein, Rv1265 protein, EF-Tu protein, P5CR protein, TetR-like protein, glutamine synthetase protein and combinations thereof, by using immunogenic peptides derived from the full-length protein sequences of such immunogenic proteins as detector reagents for screening an antibody-containing sample. Such multi-analyte antibody-based assays provide high sensitivity and specificity.

Single-analyte and multi-analyte antibody-based diagnostic and prognostic assays are also readily combined with any other art-recognized means e.g., a culture test, for determining or diagnosing tuberculosis e.g., by detecting the presence of M. tuberculosis or other organism of the M. tuberculosis complex in a specimen from a subject suspected of being infected, or at risk of being infected, e.g., to confirm an initial diagnosis and/or to indicate the specific pathogen involved. In one example, a culture test confirms the presence of M. tuberculosis in a clinical sample as opposed to another mycobacteria that is not a pathogen causative of tuberculosis. In another example, a culture test confirms the strain of M. tuberculosis or other mycobacterial agent of tuberculosis present in a clinical specimen obtained from a subject.

Preferred antibody-based tests provide for the early detection of infection or disease and/or for monitoring the efficacy of therapeutic regimens when used in conjunction with therapeutic treatments for tuberculosis or an infection associated therewith.

### 2. Specific Examples

The scope of the invention will be apparent from the claims as filed with the application that follow the examples. The claims as filed with the application are hereby incorporated into the description. The scope of the invention will also be apparent from the following description of specific examples.

In one example, the present invention provides an isolated or recombinant immunogenic KARI protein of Mycobacterium tuberculosis or an immunogenic KARI peptide or immunogenic KARI protein fragment or epitope thereof.

Preferably, the isolated or recombinant immunogenic KARI protein of M. tuberculosis comprises the amino acid sequence set forth in SEQ ID NO: 1 or comprises an amino acid sequence that is at least about 95% identical to SEQ ID NO: 1, including a homologous sequence from an organism of the M. tuberculosis complex.

Preferably, the immunogenic KARI peptide is a synthetic peptide. Preferably the KARI peptide, fragment or epitope comprises at least about 5 consecutive amino acid residues of the sequence set forth in SEQ ID NO: 1, more preferably at least about 10 consecutive amino acid residues of the sequence set forth in SEQ ID NO: 1, even more preferably at least about 15 consecutive amino acid residues of the sequence set forth in SEQ ID NO: 1, and still more preferably at least about 5 consecutive amino acid residues of the sequence set forth in SEQ ID NO: 1 fused to about 1-5 additional amino acid residues at the N-terminus and/or the C-terminus. This clearly includes any synthetic peptide, fragment or epitope of a KARI protein isolated from or derived from any organism of the M. tuberculosis complex.

In another example, the immunogenic KARI peptide is a synthetic peptide comprising or consisting of amino acid residues selected individually or collectively from the group consisting of:

a) residues 1-23 of SEQ ID NO: 1;
b) residues 40-71 of SEQ ID NO: 1, and preferably residues 57-71 of SEQ ID NO: 1;
c) residues 97-111 of SEQ ID NO: 1;
d) residues 169-199 of SEQ ID NO: 1;
e) residues 265-279 of SEQ ID NO: 1;
f) residues 290-300 of SEQ ID NO: 1, preferably residues 298-300 of SEQ ID NO: 1; and
g) residues 313-333 of SEQ ID NO: 1.

In yet another example, a synthetic peptide consisting of or comprising residues 40-71 of SEQ ID NO: 1, or residues 57-71 of SEQ ID NO: 1 or residues 290-300 of SEQ ID NO: 1 or residues 298-300 of SEQ ID NO: 1 is provided e.g., for producing antibodies suitable for immunoassays or as a positive control peptide for immunoassays such as to demonstrate antibody binding.

It is clearly within the scope of the present invention for the isolated or recombinant immunogenic KARI protein of Mycobacterium tuberculosis or other organism of the M. tuberculosis complex, or an immunogenic KARI peptide or immunogenic KARI fragment or epitope thereof, to comprise one or more labels or detectable moieties e.g., to facilitate detection or isolation or immobilization. Preferred labels include, for example, biotin, glutathione-S-transferase (GST), FLAG epitope, hexa-histidine, β-galactosidase, horseradish peroxidase, streptavidin or gold.

The present invention also provides a fusion protein comprising one or more immunogenic KARI peptides, fragments or epitopes according to any example hereof. For example, the N-terminal and C-terminal portions of KARI protein can be fused. The skilled artisan will be aware that it is preferred to include an internal linking residue e.g., cysteine in such compositions of matter. Alternatively, a preferred fusion protein comprises a linker separating an immunogenic KARI peptide from one or more other peptide moieties, such as, for example, a single amino acid residue (e.g., glycine, cysteine, lysine), a peptide linker (e.g., a non-immunogenic peptide such as a poly-lysine or poly-glycine), poly-carbon linker comprising up to about 6 or 8 or 10 or 12 carbon residues, or a chemical linker. Such linkers may facilitate antibody production or vaccine formulation e.g., by permitting linkage to a lipid or hapten, or to permit cross-linking or binding to a ligand. The expression of proteins as fusions may also enhance their solubility.

Preferred fusion proteins will comprise the KARI protein, peptide, fragment or epitope fused to a carrier protein, detectable label or reporter molecule e.g., glutathione-S-transferase (GST), FLAG epitope, hexa-histidine, β-galactosidase, thioredoxin (TRX) (La Vallie et al., Bio/Technology 11, 187-193, 1993), maltose binding protein (MBP), Escherichia coli NusA protein (Fayard, E. M. S., Thesis, University of Oklahoma, USA, 1999; Harrison, inNovations 11, 4-7, 2000), E. coli BFR (Harrison, inNovations 11, 4-7, 2000) or E. coli GrpE (Harrison, inNovations 11, 4-7, 2000).

The present invention also provides an isolated protein aggregate comprising one or more immunogenic KARI peptides, fragments or epitopes according to any example hereof. Preferred protein aggregates will comprise the protein, peptide, fragment or epitope complexed to an immune-globulin e.g., IgA, IgM or IgG, such as, for example as a circulating immune complex (CIC). Exemplary protein aggregates may be derived, for example, from an antibody-containing biological sample of a subject.

The present invention also encompasses the use of the isolated or recombinant immunogenic KARI protein of Mycobacterium tuberculosis or other organism of the M. tuberculosis complex, an immunogenic KARI peptide or immunogenic KARI fragment or epitope thereof according to any example hereof, or a combination or mixture of said proteins, peptides or epitopes or fragments, for detecting a past or present infection or latent infection by M. tuberculosis in a subject, wherein said infection is determined by the binding of antibodies in a sample obtained from the subject to said isolated or recombinant immunogenic KARI protein or an immunogenic KARI peptide or immunogenic KARI fragment or epitope.

The present invention also encompasses the use of the isolated or recombinant immunogenic KARI protein of Mycobacterium tuberculosis or other organism of the M. tuberculosis complex, an immunogenic KARI peptide or immunogenic KARI fragment or epitope thereof according to any example hereof, or a combination or mixture of said proteins, peptides or epitopes or fragments, for eliciting the production of antibodies that bind to M. tuberculosis KARI protein.

The present invention also encompasses the use of the isolated or recombinant immunogenic KARI protein of Mycobacterium tuberculosis or other organism of the M. tuberculosis complex, an immunogenic KARI peptide or immunogenic KARI fragment or epitope thereof according to any example hereof, or a combination or mixture of said proteins, peptides or epitopes or fragments, in the preparation of a medicament for immunizing a subject against infection by M. tuberculosis.

The present invention also provides a pharmaceutical composition comprising the isolated or recombinant immunogenic KARI protein of Mycobacterium tuberculosis or other organism of the M. tuberculosis complex, an immunogenic KARI peptide or immunogenic KARI fragment or epitope thereof according to any example hereof, or a combination or mixture of said proteins, peptides or epitopes or fragments, in combination with a pharmaceutically acceptable diluent, e.g., an adjuvant.

Nucleic acid encoding the KARI protein of SEQ ID NO: 1, or any variant thereof within the scope of the present invention, is encoded by the ilvC gene of M. tuberculosis or a homolog, analog or other sequence variant thereof. The present invention also provides an isolated nucleic acid encoding the isolated or recombinant immunogenic KARI protein of Mycobacterium tuberculosis or an immunogenic KARI peptide or immunogenic KARI fragment or epitope thereof according to any example hereof or encoding a combination or mixture of said peptides or epitopes or fragments e.g., as a fusion protein, such as for the preparation of nucleic acid based vaccines or for otherwise expressing the immunogenic polypeptide, protein, peptide, fragment or epitope. For example the isolated nucleic acid comprises a nucleotide sequence encoding the amino acid sequence set forth in SEQ ID NO: 1 or a homolog thereof from an organism of the M. tuberculosis complex.

The present invention also provides a cell expressing the isolated or recombinant immunogenic KARI protein of Mycobacterium tuberculosis or other organism of the M. tuberculosis complex, or expressing an immunogenic KARI peptide or immunogenic KARI fragment or epitope thereof according to any example hereof, or expressing a combination or mixture of said proteins, peptides or epitopes or fragments. The cell may preferably consist of an antigen-presenting cell (APC) that expresses the isolated or recombinant immunogenic KARI protein or an immunogenic KARI peptide or immunogenic KARI fragment or epitope thereof e.g., on its surface.

The present invention also provides an isolated ligand, e.g., a small molecule, peptide, antibody, or immune reactive fragment of an antibody, that binds specifically to the isolated or recombinant immunogenic KARI protein of Mycobacterium tuberculosis or other organism of the M. tuberculosis complex, an immunogenic KARI peptide or immunogenic KARI fragment or epitope thereof according to any example hereof, or a combination or mixture of said proteins, peptides or epitopes or fragments, or to a fusion protein or protein aggregate comprising said immunogenic KARI protein, peptide, fragment or epitope. Preferred ligands are peptides or antibodies. Preferred antibodies include, for example, a monoclonal or polyclonal antibody preparation. This extends to any isolated antibody-producing cell or antibody-producing cell population, e.g., a hybridoma or plasmacytoma producing antibodies that bind to a KARI protein or immunogenic fragment of a KARI protein or other immunogenic peptide comprising a sequence derived from the sequence of a KARI protein.

For example, the present invention provides an isolated ligand consisting of or comprising an isolated antibody that binds to an immunogenic KARI protein or an immunogenic KARI peptide or immunogenic KARI fragment or epitope thereof. In one example, the antibody is a polyclonal antibody e.g., derived by immunization of chickens such as an antibody having a binding characteristic, e.g., specificity, of the polyclonal antibody preparation designated herein as Ch34 or Ch35 or a pool there between designated Ch34/35. In another example, the antibody is a monoclonal antibody e.g., a monoclonal antibody prepared by immunization with a full-length KARI protein of SEQ ID NO: 1, or by immunization with residues 40-56 of SEQ ID NO: 1 with an optional C-terminal cysteine residue added, or by immunization with residues 290-300 of SEQ ID NO: 1 with optional C-terminal cysteine residue added, or by immunization with residues 298-310 of SEQ ID NO: 1 with optional C-terminal cysteine residue added e.g., a monoclonal antibody having a binding characteristic, e.g., specificity, of an antibody selected individually or collectively from the group consisting of Mo1283F, Mo1E7, Mo2C7, Mo3A2, Mo2B1, Mo4F7, Mo3C3, Mo1C10, Mo4C10, Mo1F6, Mo2D6, Mo3H3 and Mo4D11 and mixtures thereof, and preferably, a monoclonal antibody having a binding characteristic, e.g., specificity, of an antibody selected individually or collectively from the group consisting of Mo1283F, Mo2B1, Mo4F7, Mo4C10, Mo1F6, Mo2D6, Mo3H3 and Mo4D11 and mixtures thereof.

In a further example, the present invention provides an isolated ligand consisting of or comprising an isolated monoclonal antibody that binds to an immunogenic KARI protein or an immunogenic KARI peptide or immunogenic KARI fragment or epitope thereof, wherein said monoclonal antibody has a binding characteristic, e.g., specificity, of an antibody selected individually or collectively from the group consisting of Mo1283F, Mo2B1, Mo1F6 and Mo2D6 and mixtures thereof.

In yet a further example, the present invention provides a pair of antibodies selected from the group consisting of:

(a) a polyclonal antibody having a binding characteristic, e.g., specificity, of the polyclonal antibody preparation designated herein as Ch34/35 and a monoclonal antibody has a binding characteristic, e.g., specificity, of an antibody selected from Mo1823F and Mo2B1 and mixtures thereof; and
(b) a pair of monoclonal antibodies selected individually or collectively from monoclonal antibodies having a binding characteristic, e.g., specificity, of antibodies selected from the group consisting of Mo1283F, Mo2B1, Mo1F6 and Mo2D6.

In a further example, the present invention provides an antibody produced by the hybridoma 2B1C11 deposited with ATCC on May 21, 2009 as described according to any example hereof. The present invention clearly extends to an isolated hybridoma as described according to any example hereof, and any panels of hybridomas producing such antibodies subject to at least one such hybridoma expressing antibodies that bind to KARI protein.

In a further example, the present invention provides a panel of antibodies comprising at least one antibody that binds to a KARI protein as described according to any example hereof, especially M. tuberculosis KARI protein e.g., in combination with one or more antibodies as described according to any example hereof that bind to one or more other M. tuberculosis antigens such as antibody combinations that bind to BSX and/or S9 and/or Ef-Tu, and/or Rv1265.

The present invention also provides for the use of the isolated ligand according to any example hereof, especially any peptide ligand, antibody or an immune-reactive fragment thereof in medicine.

The present invention also provides for the use of the isolated ligand according to any example hereof or a combination of said ligands, especially any peptide ligand, antibody or an immune-reactive fragment thereof for detecting a past or present (i.e., active) infection or a latent infection by an organism of the M. tuberculosis complex e.g., M. tuberculosis in a subject, wherein said infection is determined by the binding of the ligand to KARI protein or an immunogenic fragment or epitope thereof present in a biological sample obtained from the subject.

The present invention also provides for the use of the isolated ligand according to any example hereof or a combination of said ligands, especially any peptide ligand, antibody or an immune-reactive fragment thereof for identifying a bacterium of the M. tuberculosis complex or cells infected by a bacterium of the M. tuberculosis complex or for sorting or counting of said bacterium or said cells. This example clearly encompasses the identification of a plurality of bacteria of the M. tuberculosis complex e.g., M. tuberculosis and/or M. bovis and/or M. africanum and/or M. canetti and/or M. microti.

The isolated ligand according to any example hereof, especially any peptide ligand, antibody or an immune-reactive fragment thereof or combination thereof, is also useful in therapeutic, diagnostic and research applications for detecting a past or present infection, or a latent infection, by one or more Mycobacteria of the M. tuberculosis complex as determined by the binding of the ligand to a KARI protein or immunogenic fragment or epitope of the present invention according to any examples hereof in a biological sample from a subject (i.e., an antigen-based immune-assay).

Other applications of the subject ligands include the purification and study of the diagnostic/prognostic KARI protein, identification of cells infected with one or more Mycobacteria of the M. tuberculosis complex e.g., M. tuberculosis and/or M. bovis and/or M. africanum and/or M. canetti and/or M. microti, or for sorting or counting of such cells.

The ligands are also useful in therapy including prophylaxis, diagnosis, or prognosis of tuberculosis, and the use of such ligands for the manufacture of a medicament for use in treatment of tuberculosis. For example, specific humanized antibodies or other ligands are produced that bind and neutralize a KARI protein of the present invention, especially in vivo. The humanized antibodies or other ligands are used as in the preparation of a medicament for treating TB-specific disease or infection by one or more Mycobacteria of the M. tuberculosis complex in a human subject, such as, for example, in the treatment of an active or chronic infection by M. tuberculosis and/or M. bovis and/or M. africanum and/or M. canetti and/or M. microti.

The present invention also provides a composition comprising the isolated ligand according to any example hereof or comprising a combination of said ligands, especially any peptide ligand, antibody or an immune-reactive fragment thereof, and a pharmaceutically acceptable carrier, diluent or excipient.

The present invention also provides a method of diagnosing tuberculosis or an infection by one or more Mycobacteria of the M. tuberculosis complex in a subject comprising detecting in a biological sample from said subject antibodies that bind to an immunogenic KARI protein or an immunogenic KARI peptide or immunogenic KARI fragment or epitope thereof, the presence of said antibodies in the sample is indicative of infection. The infection may be a past or active infection, or a latent infection; however this assay format is particularly useful for detecting active infection and/or recent infection.

For example, the method may be an immune-assay, e.g., comprising contacting a biological sample derived from the subject with the isolated or recombinant immunogenic KARI protein or an immunogenic KARI peptide or immunogenic KARI fragment or epitope thereof according to any example hereof or a combination or mixture of said peptides or epitopes or fragments for a time and under conditions sufficient for an antigen-antibody complex to form and then detecting the formation of an antigen-antibody complex. The sample is an antibody-containing sample e.g., a sample that comprises blood or serum or plasma or an immune-globulin fraction obtained from the subject. The sample may contain circulating antibodies in the form of complexes with KARI antigenic fragments. Generally, the antigen-antibody complex will be detected in such assay formats using antibodies capable of binding to the patient's immune-globulin e.g., anti-human Ig antibodies.

In the assay of the present invention, the detection of KARI protein, optionally with one or more other M. tuberculosis proteins is indicative of active TB. Optionally, such test results are confirmed by testing for one or more other TB-specific analytes e.g., a protein marker described herein. It is also within the scope of the present invention for the inventive assay to confirm smear-test data or culture test data for TB.

The immune-assay may be a two-site assay or sandwich assay employing multiple antibodies e.g., a capture antibody and a detector antibody. In one example, an antibody having the specificity of the mouse-derived monoclonal antibody Mo1283F, or the antibody Mo1283F per se, is employed as a capture antibody and an antibody having the specificity of the chicken-derived polyclonal antibody Ch34/35, or the Ch34/35 antibody preparation per se, is employed as a detector antibody. In another example, an antibody having the specificity of the mouse-derived monoclonal antibody Mo2B1 (or simply “2B1”), or the antibody Mo2B1 per se, is employed as a capture antibody and an antibody having the specificity of the chicken-derived antibody Ch34/35, or the Ch34/35 antibody preparation per se, is employed as a detector antibody. In another example, an antibody having the specificity of the mouse-derived monoclonal antibody Mo1F6 (or simply “1F6”), or the antibody Mo1F6 per se, is employed as a capture antibody and an antibody having the specificity of the mouse-derived monoclonal antibody Mo2B1, or the antibody Mo2B1 per se, is employed as a detector antibody. In another example, an antibody having the specificity of the mouse-derived monoclonal antibody Mo2D6 (or simply “2D6”), or the antibody Mo2D6 per se, is employed as a capture antibody and an antibody having the specificity of the mouse-derived monoclonal antibody Mo2B1, or the antibody Mo2B1 per se, is employed as a detector antibody. In another example, an antibody having the specificity of the chicken-derived antibody Ch34/35, or the Ch34/35 antibody preparation per se, is employed as a capture antibody and an antibody having the specificity of the monoclonal antibody Mo2B1, or the antibody Mo2B1 per se, is employed as a detector antibody. In another example, an antibody having the specificity of the monoclonal antibody 2B1, or the antibody Mo2B1 per se, is employed as a capture antibody and an antibody having the specificity of the monoclonal antibody 1F6, or the antibody Mo1F6 per se, is employed as a detector antibody. In another example, an antibody having the specificity of the monoclonal antibody 2B1, or the antibody Mo2B1 per se, is employed as a capture antibody and an antibody having the specificity of the monoclonal antibody 2D6, or the antibody Mo2D6 per se, is employed as a detector antibody. In another example, an antibody having the specificity of the monoclonal antibody 2D6, or the antibody Mo2D6 per se, is employed as a capture antibody and an antibody having the specificity of the monoclonal antibody 1F6, or the antibody Mo1F6 per se, is employed as a detector antibody. In another example, an antibody having the specificity of the monoclonal antibody 1F6, or the antibody Mo1F6 per se, is employed as a capture antibody and an antibody having the specificity of the monoclonal antibody 2D6, or the antibody Mo2D6 per se, is employed as a detector antibody. It is to be understood that these examples extend to uses of antibody fragments and equivalent antibodies to those exemplified herein, which the data indicate clearly may be produced without undue experimentation or requiring the exercise of inventive effort. Other orientations and antibody combinations are not excluded.

In a preferred example, the subject is suspected of suffering from tuberculosis or an infection by one or more Mycobacteria of the M. tuberculosis complex and/or the subject is at risk of developing tuberculosis or at risk of being infected by said one or more Mycobacteria e.g., M. tuberculosis and/or M. bovis and/or M. africanum and/or M. canetti and/or M. microti.

A subject suspected of suffering from tuberculosis or an infection by one or more Mycobacteria of the M. tuberculosis complex displays one or more symptoms of tuberculosis or such infection, such as, for example, fever, productive cough, haemoptysis (blood in the sputum), chest pain, night sweats, weight loss, malaise, cavitations and/or calcification of the nodes of the lungs. A subject suspected of suffering from tuberculosis or such infection may have been exposed to one or a plurality of bacteria of the M. tuberculosis complex e.g., M. tuberculosis and/or M. bovis and/or M. africanum and/or M. canetti and/or M. microti. e.g. by virtue of having come into contact with a person suffering from tuberculosis.

A subject at risk of developing tuberculosis is a subject that is exposed to a condition or suffers from a condition that increases the risk of developing tuberculosis or being infected by one or more bacteria of the M. tuberculosis complex such subjects include a subject who has come into contact with a person suffering from tuberculosis, a subject that has traveled in a country or region in which tuberculosis is common and/or the causative agent(s) prevalent (e.g. South Africa), a subject that works in a hospital or nursing facility, a subject infected with HIV-1 or HIV-2, a subject that uses corticosteroids, an immune-compromised or immune-suppressed subject, a subject that suffers from silicosis or a subject suffering from a latent infection by one or more Mycobacteria of the M. tuberculosis complex, e.g., M. tuberculosis and/or M. bovis and/or M. africanum and/or M. canetti and/or M. microti.

It is within the scope of the present invention to include a multi-analyte test in this assay format, wherein multiple antigenic epitopes derived from other proteins expressed by one or more Mycobacteria of the M. tuberculosis complex are used to confirm a diagnosis obtained using KARI or peptide derived there from. For example, the other M. tuberculosis complex-derived protein is selected from the group consisting of BSX protein (UnitProtKB/TrEMBL Accession No. A5TZK2; SEQ ID NO: 2), ribosomal protein S9 (UnitProtKB/Swiss-Prot Accession No. A5U8B8; SEQ ID NO: 14), protein Rv1265 (UniProtKB/Swiss-Prot Accession No. P64789; SEQ ID NO: 21), elongation factor-Tu (EF-Tu) protein (UniProtKB/Swiss-Prot Accession No. A5U071; SEQ ID NO: 28-29), P5CR protein (UniProtKB/Swiss-Prot Accession No. Q11141; SEQ ID NO: 36), TetR-like protein (UnitProtKB/TrEMBL Accession No. A1QW92; SEQ ID NO: 44) glutamine synthase (GS) protein (UnitProtKB/TrEMBL Accession No. O33342), an immunogenic peptide derived from said BSX protein, an immunogenic peptide derived from said S9, an immunogenic peptide derived from said Rv1265, an immunogenic peptide derived from said EF-Tu protein, an immunogenic peptide derived from said P5CR protein, an immunogenic peptide derived from said TetR-like protein and an immunogenic peptide derived from GS protein, and combinations thereof. It is to be understood in this context that the stated proteins include homologs of the exemplified proteins exemplified by way of the Sequence Listing, wherein said homologs are expressed by one or a plurality of bacteria of the M. tuberculosis complex e.g., M. tuberculosis and/or M. bovis and/or M. africanum and/or M. canetti and/or M. microti.

In one example, an immune-assay employing antibodies against KARI protein is performed simultaneously or sequentially with a smear test for TB and/or a culture test for TB and/or an immune-assay to detect another TB protein e.g., BSX and/or Rv1265 and/or Ef-Tu and/or S9 protein such as for the purpose of reducing positive combination of testing for KARI protein and testing for S9 protein reduces false positive detection, or otherwise enhancing assay specificity and/or sensitivity, wherein a negative result for one or two or three or four proteins other than KARI protein indicates or confirms a negative smear result and/or is indicative of the absence of active TB in a subject. In one example the combination of testing for KARI protein and testing for BSX protein reduces false positive detection, wherein a negative result for BSX, or BSX and KARI proteins, indicates or confirms a negative smear result and/or is indicative of the absence of active TB in a subject. In another example, the combination of testing for KARI protein and testing for Rv1265 protein reduces false positive detection wherein a negative result for Rv1265, or Rv1265 and KARI proteins, indicates or confirms a negative smear result and/or is indicative of the absence of active TB in a subject. In another example, the combination of testing for KARI protein and testing for S9 protein and testing for BSX protein reduces false positive detection wherein a negative result for BSX and S9 proteins, or BSX and S9 and KARI proteins, indicates or confirms a negative smear result and/or is indicative of the absence of active TB in a subject.

The skilled artisan will be aware that UniProtKB/Swiss-Prot is a curated protein sequence database of the Swiss Institute of Bioinformatics providing data on protein function, domain structure, post-translational modifications, and variants; and that UniProtKB/TrEMBL is a computer-annotated supplement of Swiss-Prot that contains translations of EMBL nucleotide sequence entries not yet integrated in Swiss-Prot. Access to UniProtKB/Swiss-Prot and UniProtKB/TrEMBL data can be obtained readily e.g., via the ExPASy (Expert Protein Analysis System) proteomics server of the Swiss Institute of Bioinformatics.

For example, the patient sample may be contacted with KARI protein or immunogenic KARI peptide or fragment or epitope and with a M. tuberculosis BSX protein (e.g., UnitProtKB/TrEMBL Accession No. A5TZK2; SEQ ID NO: 2) or immunogenic peptide derived there from, e.g., a peptide derived from a BSX protein, or comprising a sequence selected from the group consisting of SEQ ID Nos: 3-13 and mixtures/combinations thereof. Immunogenic M. tuberculosis BSX and peptide derivatives for detecting tuberculosis or infection by M. tuberculosis are also described in detail in the instant applicant's International Patent Application No. PCT/AU2005/001254 filed Aug. 19, 2005 (WO 2006/01792) the disclosure of which is incorporated herein in its entirety.

Alternatively, or in addition, the patient sample may be contacted with KARI protein or immunogenic KARI peptide or fragment or epitope and with M. tuberculosis ribosomal protein S9 (e.g., UniProtKB/Swiss-Prot Accession No. A5U8B8; SEQ ID NO: 14), or immunogenic peptide derived there from, e.g., a peptide derived from S9 protein, or comprising a sequence selected from the group consisting of SEQ ID Nos: 15-20 and mixtures/combinations thereof. Immunogenic M. tuberculosis S9 and peptide derivatives for detecting tuberculosis or infection by M. tuberculosis are also described in detail in the instant applicant's International Patent Application No. PCT/AU2007/000093 filed on Jan. 31, 2007 (WO 2007/087679) the disclosure of which is incorporated herein in its entirety.

Alternatively, or in addition, the patient sample may be contacted with KARI protein or immunogenic KARI peptide or fragment or epitope and with M. tuberculosis protein Rv1265 (e.g., UniProtKB/Swiss-Prot Accession No. P64789; SEQ ID NO: 21) or immunogenic peptide derived there from, e.g., a peptide derived from Rv1265 protein, or comprising a sequence selected from the group consisting of SEQ ID Nos: 22-27 and mixtures/combinations thereof. Immunogenic M. tuberculosis Rv1265 protein and peptide derivatives for detecting tuberculosis or infection by M. tuberculosis are also described in detail in the instant applicant's International Patent Application No. PCT/AU2007/000662 filed on May 16, 2007 (WO 2007/131291) the disclosure of which is incorporated herein in its entirety.

Alternatively, or in addition, the patient sample may be contacted with KARI protein or immunogenic KARI peptide or fragment or epitope and with M. tuberculosis elongation factor-Tu (EF-Tu) protein (e.g., UniProtKB/Swiss-Prot Accession No. A5U071; SEQ ID NO: 28-29) or immunogenic peptide derived there from, e.g., a peptide derived from EF-Tu protein, or comprising a sequence selected from the group consisting of SEQ ID Nos: 30-35 and mixtures/combinations thereof. Immunogenic M. tuberculosis EF-Tu protein and peptide derivatives for detecting tuberculosis or infection by M. tuberculosis are also described in detail in the instant applicant's International Patent Application No. PCT/AU2007/000810 filed on Jun. 8, 2007 (WO 2007/140545) the disclosure of which is incorporated herein in its entirety.

Alternatively, or in addition, the patient sample may be contacted with KARI protein or immunogenic KARI peptide or fragment or epitope and with M. tuberculosis pyrroline-5-carboxylate reductase (P5CR) protein (e.g., UniProtKB/Swiss-Prot Accession No. Q11141; SEQ ID NO: 36) or immunogenic peptide derived there from, e.g., a peptide derived from P5CR protein, or comprising a sequence selected from the group consisting of SEQ ID Nos: 37-43 and mixtures/combinations thereof. Immunogenic M. tuberculosis P5CR protein and peptide derivatives for detecting tuberculosis or infection by M. tuberculosis are also described in detail in the instant applicant's International Patent Application No. PCT/AU2007/000664 filed on May 16, 2007 (WO 2007/131293) the disclosure of which is incorporated herein in its entirety.

Alternatively, or in addition, the patient sample may be contacted with KARI protein or immunogenic KARI peptide or fragment or epitope and with M. tuberculosis putative regulatory protein of the TetR-like protein family (TetR-like protein) (e.g., UnitProtKB/TrEMBL Accession No. A1QW92; SEQ ID NO: 44) or immunogenic peptide derived there from, e.g., a peptide derived from TetR-like protein, or comprising a sequence selected from the group consisting of SEQ ID Nos: 45-56 and mixtures/combinations thereof. Immunogenic M. tuberculosis TetR-like protein and peptide derivatives for detecting tuberculosis or infection by M. tuberculosis are also described in detail in the instant applicant's International Patent Application No. PCT/AU2007/000663 filed May 16, 2007 (WO 2007/131292) the disclosure of which is incorporated herein in its entirety.

Alternatively, or in addition, the patient sample may be contacted with KARI protein or immunogenic KARI peptide or fragment or epitope and with a M. tuberculosis glutamine synthetase (GS) protein (e.g., UnitProtKB/TrEMBL Accession No. 033342), or immunogenic peptide derived there from e.g., a peptide derived from a surface-exposed region of a GS protein, or comprising a sequence selected from the group set forth in SEQ ID Nos: 57-60 and mixtures/combinations thereof. Immunogenic M. tuberculosis GS and peptide derivatives for detecting tuberculosis or infection by M. tuberculosis are also described in detail in the instant applicant's International Patent Application No. PCT/AU2005/000930 filed Jun. 24, 2005 (WO 2006/000045) the disclosure of which is incorporated herein in its entirety.

Further specific examples of a multi-analyte test in this assay format comprise the use of antigenic epitopes derived from M. tuberculosis KARI protein and/or M. tuberculosis BSX protein and/or M. tuberculosis ribosomal protein S9 and/or M. tuberculosis protein Rv1265, or alternatively, the use of antigenic epitopes derived from M. tuberculosis KARI protein and/or M. tuberculosis BSX protein and/or M. tuberculosis protein Rv1265.

Assays for one or more secondary analytes e.g., antibodies that bind to BSX and/or glutamine synthetase, are conveniently performed in the same manner as for detecting antibodies that bind to KARI protein in serum or plasma or other body fluid. The assays may be performed simultaneously or at different times, and using the same or different patient samples. The assays may also be performed in the same reaction vessel; provided that different detection systems are used to detect the different antibodies, e.g., anti-human Ig labelled using different reporter molecules such as different coloured dyes, fluorophores, radionucleotides or enzymes.

It is also within the scope of the present invention to further perform one or more art-recognized tests for determining infection by one or more mycobacteria pathogens e.g., M. tuberculosis, M. avium, M. intracellulaire, such as for the purposes of confirming an initial or preliminary diagnosis obtained using the method of the present invention and/or to determine the specific infectious agent in a clinical sample. Exemplary surrogate tests for use with the antibody-based assays of the present invention include culture tests and/or smear tests, however other antibody-based tests than those specifically described are clearly encompassed by the present invention, the only requirement being that one or more antibodies that bind to a mycobacteria KARI protein and/or one or more immunogenic fragments thereof is/are detected.

As used herein, the term “infection” shall be understood to mean invasion and/or colonisation by a microorganism and/or multiplication of a micro-organism, in particular, a bacterium or a virus, in the respiratory tract of a subject. Such an infection may be unapparent or result in local cellular injury. The infection may be localised, subclinical and temporary or alternatively may spread by extension to become an acute or chronic clinical infection. The infection may also be a past infection wherein residual KARI antigen, or alternatively, reactive host antibodies that bind to isolated KARI protein or peptides, remain in the host. The infection may also be a latent infection, in which the microorganism is present in a subject, however the subject does not exhibit symptoms of disease associated with the organism. Preferably, the infection is a pulmonary or extra-pulmonary infection by M. tuberculosis, and more preferably an extra-pulmonary infection. By “pulmonary” infection is meant an infection of the airway of the lung, such as, for example, an infection of the lung tissue, bronchi, bronchioles, respiratory bronchioles, alveolar ducts, alveolar sacs, or alveoli. By “extra-pulmonary” is meant outside the lung, encompassing, for example, kidneys, lymph, urinary tract, bone, skin, spinal fluid, intestine, peritoneal, pleural and pericardial cavities.

The antibodies of the present invention are also useful in the diagnosis of tuberculosis or infection by one or more Mycobacteria of the M. tuberculosis complex. For example, the present invention also provides a method of diagnosing tuberculosis or infection by M. tuberculosis in a subject comprising detecting in a biological sample from said subject an immunogenic KARI protein or an immunogenic KARI peptide or immunogenic KARI fragment or epitope thereof, wherein the presence of said protein or immunogenic fragment or epitope in the sample is indicative of disease, disease progression or infection. In a related example, the presence of said protein or immunogenic fragment or epitope in the sample is indicative of infection.

Preferably, the subject is suspected of suffering from tuberculosis or an infection by one or more Mycobacteria of the M. tuberculosis complex and/or the subject is at risk of developing tuberculosis or being infected.

For example, the method may be an immune-assay, e.g., comprising contacting a biological sample derived from the subject with an antibody that binds to the endogenous KARI protein of Mycobacterium tuberculosis or an immunogenic KARI peptide or immunogenic KARI fragment or epitope thereof according to any example hereof or a combination or mixture of said peptides or epitopes or fragments for a time and under conditions sufficient for an antigen-antibody complex to form and then detecting the formation of an antigen-antibody complex. Preferred samples according to this example are those samples in which M. tuberculosis or peptide fragments from bacterial debris are likely to be found, or immune-globulin-containing fraction, e.g., an extract from brain, breast, ovary, lung, colon, pancreas, testes, liver, muscle, bone or mixtures thereof; body fluid(s) such as sputum, serum, plasma, whole blood, saliva, urine, pleural fluid or mixtures thereof or derivatives thereof e.g., sputum, serum, plasma, whole blood, saliva, urine, pleural fluid, etc. The sample may contain circulating antibodies complexed with KARI antigenic fragments.

It is within the scope of the present invention to include a multi-analyte test in this assay format, wherein multiple antibodies are used to confirm a diagnosis obtained using antibodies that bind to the KARI protein or epitope. For example, the patient sample may be contacted with antibodies that bind to KARI protein or immunogenic KARI peptide or fragment or epitope, and with antibodies that bind another M. tuberculosis protein e.g., a protein selected from the group consisting of M. tuberculosis BSX protein (UnitProtKB/TrEMBL Accession No. A5TZK2; SEQ ID NO: 2), M. tuberculosis ribosomal protein S9 (UniProtKB/Swiss-Prot Accession No. A5U8B8; SEQ ID NO: 14), M. tuberculosis protein Rv1265 (UniProtKB/Swiss-Prot Accession No. P64789; SEQ ID NO: 21), M. tuberculosis elongation factor-Tu (EF-Tu) protein (UniProtKB/Swiss-Prot Accession No. A5U071; SEQ ID NO: 28-29), M. tuberculosis P5CR protein (UniProtKB/Swiss-Prot Accession No. Q11141; SEQ ID NO: 36), M. tuberculosis TetR-like protein (UnitProtKB/TrEMBL Accession No. A1QW92; SEQ ID NO: 44) M. tuberculosis glutamine synthase (GS) protein (UnitProtKB/TrEMBL Accession No. 033342), an immunogenic peptide derived from said BSX protein, an immunogenic peptide derived from said S9, an immunogenic peptide derived from said Rv1265, an immunogenic peptide derived from said EF-Tu protein, an immunogenic peptide derived from said P5CR protein, an immunogenic peptide derived from said TetR-like protein and an immunogenic peptide derived from GS protein, and combinations thereof.

For example, the patient sample may be contacted with antibodies that bind to KARI protein or immunogenic KARI peptide or fragment or epitope and with antibodies that bind to M. tuberculosis BSX protein (e.g., UnitProtKB/TrEMBL Accession No. A5TZK2; SEQ ID NO: 2) and/or antibodies that bind to an immunogenic peptide derived from BSX protein, e.g., antibodies that bind to a peptide comprising a sequence selected from the group consisting of SEQ ID Nos: 3-13. Exemplary antibodies are described herein, and in the applicant's International Patent Application No. PCT/AU2005/001254 filed Aug. 19, 2005 (WO 2006/01792) the disclosure of which is incorporated herein in its entirety.

Alternatively, or in addition, the patient sample may be contacted with antibodies that bind to a KARI protein or immunogenic KARI peptide or fragment or epitope and with antibodies that bind to M. tuberculosis ribosomal protein S9 (e.g., UniProtKB/Swiss-Prot Accession No. A5U8B8; SEQ ID NO: 14), and/or antibodies that bind to an immunogenic peptide derived from S9 protein, e.g., antibodies that bind to a peptide comprising a sequence selected from the group consisting of SEQ ID Nos: 15-20. Exemplary antibodies are described herein, and in the applicant's International Patent Application No. PCT/AU2007/000093 filed on Jan. 31, 2007 (WO 2007/087679) the disclosure of which is incorporated herein in its entirety.

Alternatively, or in addition, the patient sample may be contacted with antibodies that bind to a KARI protein or immunogenic KARI peptide or fragment or epitope and with antibodies that bind to M. tuberculosis protein Rv1265 (e.g., UniProtKB/Swiss-Prot Accession No. P64789; SEQ ID NO: 21) and/or antibodies that bind to an immunogenic peptide derived from Rv1265 protein e.g., antibodies that bind to a peptide comprising a sequence selected from the group consisting of SEQ ID Nos: 22-27. Exemplary antibodies are described herein, and the applicant's International Patent Application No. PCT/AU2007/000662 filed on May 16, 2007 (WO 2007/131291) the disclosure of which is incorporated herein in its entirety.

Alternatively, or in addition, the patient sample may be contacted with antibodies that bind to a KARI protein or immunogenic KARI peptide or fragment or epitope and with antibodies that bind to M. tuberculosis protein EF-Tu (e.g., UniProtKB/Swiss-Prot Accession No. A5U071; SEQ ID NOs: 28-29) and/or antibodies that bind to an immunogenic peptide derived from EF-Tu protein e.g., antibodies that bind to a peptide comprising a sequence selected from the group consisting of SEQ ID Nos: 30-35. Exemplary antibodies are described herein, and the applicant's International Patent Application No. PCT/AU2007/000810 filed on Jun. 8, 2007 (WO 2007/140545) the disclosure of which is incorporated herein in its entirety.

Alternatively, or in addition, the patient sample may be contacted with antibodies that bind to a KARI protein or immunogenic KARI peptide or fragment or epitope and with antibodies that bind to M. tuberculosis protein P5CR (e.g., UniProtKB/Swiss-Prot Accession No. Q11141; SEQ ID NOs: 36) and/or antibodies that bind to an immunogenic peptide derived from P5CR protein e.g., antibodies that bind to a peptide comprising a sequence selected from the group consisting of SEQ ID Nos: 37-43. Exemplary antibodies are described herein, and the applicant's International Patent Application No. PCT/AU2007/000664 filed on May 16, 2007 (WO 2007/131293) the disclosure of which is incorporated herein in its entirety.

Alternatively, or in addition, the patient sample may be contacted with antibodies that bind to a KARI protein or immunogenic KARI peptide or fragment or epitope and with antibodies that bind to M. tuberculosis TetR-like protein (e.g., UnitProtKB/TrEMBL Accession No. A1QW92; SEQ ID NO: 44) and/or antibodies that bind to an immunogenic peptide derived from TetR-like protein e.g., antibodies that bind to a peptide comprising a sequence selected from the group consisting of SEQ ID Nos: 45-56. Exemplary antibodies are described herein, and the applicant's International Patent Application No. PCT/AU2007/000663 filed May 16, 2007 (WO 2007/131292) the disclosure of which is incorporated herein in its entirety.

Alternatively, or in addition, the patient sample may be contacted with antibodies that bind to a KARI protein or immunogenic KARI peptide or fragment or epitope and with antibodies that bind to M. tuberculosis GS protein (e.g., UnitProtKB/TrEMBL Accession No. O33342) and/or antibodies that bind to an immunogenic peptide derived from GS protein e.g., antibodies that bind to a peptide comprising a sequence selected from the group consisting of SEQ ID Nos: 57-60. Exemplary antibodies are described herein, and the applicant's International Patent Application No. PCT/AU2005/000930 filed Jun. 24, 2005 (WO 2006/000045) the disclosure of which is incorporated herein in its entirety.

Further specific examples of a multi-analyte test in this assay format comprise the use of antibodies that bind to M. tuberculosis KARI protein or immunogenic fragments or epitopes thereof and/or M. tuberculosis BSX protein or immunogenic fragments or epitopes thereof and/or M. tuberculosis ribosomal protein S9 or immunogenic fragments or epitopes thereof and/or M. tuberculosis protein Rv1265 or immunogenic fragments or epitopes thereof, or alternatively, the use of antibodies that bind to M. tuberculosis KARI protein or immunogenic fragments or epitopes thereof and/or M. tuberculosis BSX protein or immunogenic fragments or epitopes thereof and/or M. tuberculosis protein Rv1265 or immunogenic fragments or epitopes thereof.

Assays for one or more secondary analytes e.g., BSX and/or glutamine synthetase and/or S9, are conveniently performed in the same manner as for detecting KARI protein in the sample. The assays may be performed simultaneously or at different times, and using the same or different patient samples. The assays may also be performed in the same reaction vessel, provided that different detection systems are used to detect the bound antibodies, e.g., secondary antibodies that bind to the anti-KARI antibodies and antibodies that bind to the secondary analyte(s).

As with antibody-based assays, antigen-based assay systems can comprise an immune-assay e.g., contacting a biological sample derived from the subject with one or more isolated ligands according to any example hereof, especially any peptide ligand, antibody or an immune-reactive fragment thereof capable of binding to a KARI protein or an immunogenic fragment or epitope thereof, and detecting the formation of a complex e.g., an antigen-antibody complex. In a particularly preferred example, the ligand is an antibody, preferably a polyclonal or monoclonal antibody or antibody fragment that binds specifically to the isolated or recombinant immunogenic KARI protein of Mycobacterium tuberculosis or an immunogenic KARI peptide or immunogenic KARI fragment or epitope thereof according to any example hereof or to a combination or mixture of said peptides or epitopes or fragments or to a fusion protein or protein aggregate comprising said immunogenic KARI protein, peptide, fragment or epitope. Whilst useful for subjects who are not immune-compromised, e.g., HIV-negative subjects, the assay is also particularly useful for detecting TB in a subject that is immune compromised or immune deficient, e.g., a subject that is infected with human immune-deficiency virus (i.e., “HIV+”). The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof; and (iii) samples derived from body fluid(s) selected from the group consisting of sputum, serum, plasma, whole blood, saliva, urine, pleural fluid and mixtures thereof.

The present invention also provides a method for determining the response of a subject having tuberculosis or an infection by one or more Mycobacteria of the M. tuberculosis complex to treatment with a therapeutic compound for said tuberculosis or infection, said method comprising detecting a KARI protein or an immunogenic fragment or epitope thereof in a biological sample from said subject, wherein a level of the protein or fragment or epitope that is enhanced, or not decreased or decreasing, compared to the level of that protein or fragment or epitope detectable in a normal or healthy subject indicates that the subject is not responding to said treatment or has not been rendered free of disease or infection. For example, the method can comprise an immune-assay e.g., contacting a biological sample derived from the subject with one or more antibodies capable of binding to a KARI protein or an immunogenic fragment or epitope thereof, and detecting the formation of an antigen-antibody complex. In a particularly preferred example, an antibody is an isolated or recombinant antibody or immune reactive fragment of an antibody that binds specifically to the isolated or recombinant immunogenic KARI protein of Mycobacterium tuberculosis or an immunogenic KARI peptide or immunogenic KARI fragment or epitope thereof according to any example hereof or to a combination or mixture of said peptides or epitopes or fragments or to a fusion protein or protein aggregate comprising said immunogenic KARI protein, peptide, fragment or epitope. Whilst useful for subjects who are not immune-compromised, e.g., HIV-negative subjects, the diagnostic assay of the present invention is also particularly useful for detecting TB in a subject that is immune compromised or immune deficient, e.g., a subject that is HIV+. The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof; and (iii) samples derived from body fluid(s) selected from the group consisting of sputum, serum, plasma, whole blood, saliva, urine, pleural fluid and mixtures thereof.

The present invention also provides a method for determining the response of a subject having tuberculosis or an infection by one or more mycobacteria of the M. tuberculosis complex to treatment with a therapeutic compound for said tuberculosis or infection, said method comprising detecting a KARI protein or an immunogenic fragment or epitope thereof in a biological sample from said subject, wherein a level of the protein or fragment or epitope that is lower than the level of the protein or fragment or epitope detectable in a subject suffering from tuberculosis or said infection indicates that the subject is responding to said treatment or has been rendered free of disease or infection. For example, the method can comprise an immune-assay e.g., contacting a biological sample derived from the subject with one or more antibodies capable of binding to a KARI protein or an immunogenic fragment or epitope thereof, and detecting the formation of an antigen-antibody complex. In a particularly preferred example, an antibody is an isolated or recombinant antibody or immune reactive fragment of an antibody that binds specifically to the isolated or recombinant immunogenic KARI protein of Mycobacterium tuberculosis or an immunogenic KARI peptide or immunogenic KARI fragment or epitope thereof according to any example hereof or to a fusion protein or protein aggregate comprising said immunogenic KARI protein, peptide, fragment or epitope. Whilst useful for subjects who are not immune-compromised, e.g., HIV-negative subjects, the diagnostic assay of the present invention is also particularly useful for detecting TB in a subject that is immune compromised or immune deficient, e.g., a subject that is HIV+. The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof; and (iii) samples derived from body fluid(s) selected from the group consisting of sputum, serum, plasma, whole blood, saliva, urine, pleural fluid and mixtures thereof.

The present invention also provides a method of monitoring disease progression, responsiveness to therapy or infection status by one or more mycobacteria of the M. tuberculosis complex in a subject comprising determining the level of a KARI protein or an immunogenic fragment or epitope thereof in a biological sample from said subject at different times, wherein a change in the level of the KARI protein, fragment or epitope indicates a change in disease progression, responsiveness to therapy or infection status of the subject. In a preferred example, the method further comprises administering a compound for the treatment of tuberculosis or infection by M. tuberculosis when the level of KARI protein, fragment or epitope increases over time. For example, the method can comprise an immune-assay e.g., contacting a biological sample derived from the subject with one or more antibodies capable of binding to a KARI protein or an immunogenic fragment or epitope thereof, and detecting the formation of an antigen-antibody complex. In a particularly preferred example, an antibody is an isolated or recombinant antibody or immune reactive fragment of an antibody that binds specifically to the isolated or recombinant immunogenic KARI protein of Mycobacterium tuberculosis or an immunogenic KARI peptide or immunogenic KARI fragment or epitope thereof according to any example hereof or to a combination or mixture of said peptides or epitopes or fragments or to a fusion protein or protein aggregate comprising said immunogenic KARI protein, peptide, fragment or epitope. Whilst useful for subjects who are not immune-compromised, e.g., HIV-negative subjects, the diagnostic assay of the present invention is particularly useful for detecting TB in a subject that is immune compromised or immune deficient, e.g., a subject that is HIV+. The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof; and (iii) samples derived from body fluid(s) selected from the group consisting of sputum, serum, plasma, whole blood, saliva, urine, pleural fluid and mixtures thereof.

In a particularly preferred example, circulating immune complexes (CICs) are detected in an antigen-based assay platform or antibody-based assay platform. For antigen-based assay platforms, the detection of CICs may provide a signal amplification over the detection of isolated antigen in circulation, by virtue of detecting the immune-globulin moiety of the CIC. In accordance with this example, a capture reagent e.g., a capture antibody is used to capture the KARI antigen (KARI polypeptide or an immune reactive fragment or epitope thereof) complexed with the subject's immune-globulin, in addition to isolated antigen in the subject's circulation. Anti-Ig antibodies, optionally conjugated to a detectable label, are used to specifically bind the captured CIC thereby detecting CIC patient samples. Within the scope of this invention, the anti-Ig antibody binds preferentially to IgM, IgA or IgG in the sample. In a particularly preferred example, the anti-Ig antibody binds to human Ig, e.g., human IgA, human IgG or human IgM. The anti-Ig antibody may be conjugated to any standard detectable label known in the art. This is particularly useful for detecting infection by a pathogenic agent, e.g., a bacterium or virus, or for the diagnosis of any disease or disorder associated with CICs. Accordingly, the diagnostic methods described according to any example herein are amenable to a modification wherein the sample derived from the subject comprises one or more circulating immune complexes comprising immune-globulin (Ig) bound to KARI protein of Mycobacterium tuberculosis or one or more immunogenic KARI peptides, fragments or epitopes thereof and wherein detecting the formation of an antigen-antibody complex comprises contacting an anti-Ig antibody with an immune-globulin moiety of the circulating immune complex(es) for a time and under conditions sufficient for a complex to form and then detecting the bound anti-Ig antibody.

Antigen-based multi-analyte tests for monitoring disease progression and/or efficacy of treatment are clearly within the scope of the present invention, and these are performed essentially as described herein above for diagnosis of infection albeit using samples from patients that are known to be infected e.g., by virtue of having been previously diagnosed using one or more of the preceding antigen-based assay formats. As with other multi-analyte tests, multiple antibodies of different specificities are employed in the context of monitoring disease progression and/or efficacy of treatment for infection, e.g., selected from the group consisting of antibodies that bind to M. tuberculosis BSX protein (UnitProtKB/TrEMBL Accession No. A5TZK2; SEQ ID NO: 2) and/or M. tuberculosis ribosomal protein S9 (UniProtKB/Swiss-Prot Accession No. A5U8B8; SEQ ID NO: 14) and/or M. tuberculosis protein Rv1265 (UniProtKB/Swiss-Prot Accession No. P64789; SEQ ID NO: 21) and/or M. tuberculosis elongation factor-Tu (EF-Tu) protein (UniProtKB/Swiss-Prot Accession No. A5U071; SEQ ID NO: 28-29) and/or M. tuberculosis P5CR protein (UniProtKB/Swiss-Prot Accession No. Q11141; SEQ ID NO: 36) and/or M. tuberculosis TetR-like protein (UnitProtKB/TrEMBL Accession No. A1QW92; SEQ ID NO: 44) and/or M. tuberculosis glutamine synthase (GS) protein (UnitProtKB/TrEMBL Accession No. O33342) and/or an immunogenic peptide derived from said BSX protein and/or an immunogenic peptide derived from said S9 and/or an immunogenic peptide derived from said Rv1265 and/or an immunogenic peptide derived from said EF-Tu protein and/or an immunogenic peptide derived from said P5CR protein and/or an immunogenic peptide derived from said TetR-like protein and/or an immunogenic peptide derived from GS protein, and combinations thereof. or any combination of said antibodies, are used to confirm a diagnosis obtained using antibodies raised against KARI and/or antibodies raised against a KARI peptide, thereby enhancing specificity and/or selectivity. Again, the use of cross-reactive antibodies that bind to homologs of any one or more mycobacteria of the M. tuberculosis complex are useful in performing the invention.

The antigen-antibody complexes formed are then detected using antibodies capable of binding to each protein analyte, or in the case of CIC detections, antibodies capable of binding to human immune-globulins. The assays may be performed simultaneously or at different times, and using the same or different patient samples. The assays may also be performed in the same reaction vessel, provided that different detection systems are used to detect the different antigens or CICs comprising the different antigens, e.g., anti-human Ig labelled using different reporter molecules such as different coloured dyes, fluorophores, radionucleotides, enzymes, or colloidal gold particles; or differentially-labelled anti-KARI antibodies, anti-BSX antibodies, and anti-GS antibodies. As with other immune-assays described herein, the secondary antibody is optionally conjugated to a suitable detectable label e.g., horseradish peroxidase (HRP) or β-galactosidase or β-glucosidase, colloidal gold particles, amongst others. Standard methods for employing such labels in the detection of the complexes formed will be apparent to the skilled artisan.

It is also within the scope of the present invention to further perform one or more art-recognized tests for determining infection by one or more mycobacteria of the M. tuberculosis complex or other pathogens e.g., M. avium, M. intracellulaire, such as for the purposes of confirming an initial or preliminary diagnosis obtained using the method of the present invention and/or to determine the specific infectious agent in a clinical sample. Exemplary surrogate tests for use with the antigen-based assays of the present invention include culture tests and/or smear tests, however other antigen-based tests than those specifically described are clearly encompassed by the present invention, the only requirement being that a mycobacteria KARI protein and/or one or more immunogenic fragments thereof is/are detected. Such examples are described in detail in the examples preceding the claims.

The present invention also provides a method of treatment of tuberculosis or infection by one or more mycobacteria of the M. tuberculosis complex comprising:

(i) performing a diagnostic method according to any example hereof thereby detecting the presence of one or more mycobacteria of the M. tuberculosis complex in a biological sample from a subject; and
(ii) administering a therapeutically effective amount of a pharmaceutical composition to reduce the number of pathogenic bacilli in the lung, blood or lymph system of the subject.

The present invention also provides a method of treatment of tuberculosis or infection by one or more mycobacteria of the M. tuberculosis complex comprising:

(i) performing a diagnostic method according to any example hereof thereby detecting the presence of one or more mycobacteria of the M. tuberculosis complex in a biological sample from a subject being treated with a first pharmaceutical composition; and
(ii) administering a therapeutically effective amount of a second pharmaceutical composition to reduce the number of pathogenic bacilli in the lung, blood or lymph system of the subject.

The present invention also provides a method of treatment of tuberculosis in a subject comprising performing a diagnostic method or prognostic method as described herein. In one example, the present invention provides a method of prophylaxis comprising:

(i) detecting the presence of one or more mycobacteria of the M. tuberculosis complex infection in a biological sample from a subject; and
(ii) administering a therapeutically effective amount of a pharmaceutical composition to reduce the number of pathogenic bacilli in the lung, blood or lymph system of the subject.

More particularly, an immunogenic KARI protein or one or more immunogenic KARI peptides, fragments or epitopes thereof induce(s) the specific production of a high titer antibody when administered to an animal subject.

Accordingly, the invention also provides a method of eliciting the production of antibody against one or more mycobacteria of the M. tuberculosis complex comprising administering an immunogenic KARI protein or one or more immunogenic KARI peptides or immunogenic KARI fragments or epitopes thereof to said subject for a time and under conditions sufficient to elicit the production of antibodies, such as, for example, neutralizing antibodies that bind to M. tuberculosis.

The present invention clearly contemplates the use of an immunogenic KARI protein or one or more immunogenic KARI peptides or immunogenic KARI fragments or epitopes thereof in the preparation of a therapeutic or prophylactic subunit vaccine against one or more mycobacteria of the M. tuberculosis complex in a human or other animal subject.

Accordingly, this invention also provides a vaccine comprising an immunogenic KARI protein or one or more immunogenic KARI peptides or immunogenic KARI fragments or epitopes thereof in combination with a pharmaceutically acceptable diluent. Preferably, the protein or peptide(s) or fragment(s) or epitope(s) thereof is(are) formulated with a suitable adjuvant.

Alternatively, the peptide or derivative or variant is formulated as a cellular vaccine via the administration of an autologous or allogeneic antigen presenting cell (APC) or a dendritic cell that has been treated in vitro so as to present the peptide on its surface.

Nucleic acid-based vaccines that comprise nucleic acid, such as, for example, DNA or RNA, encoding an immunogenic KARI protein or one or more immunogenic KARI peptides or immunogenic KARI fragments or epitopes thereof cloned into a suitable vector (e.g. vaccinia, canary pox, adenovirus, or other eukaryotic virus vector) are also contemplated. Preferably, DNA encoding an immunogenic KARI protein or an immunogenic KARI peptide or immunogenic KARI fragment or epitope thereof is formulated into a DNA vaccine, such as, for example, in combination with the existing Calmette-Guerin (BCG) or an immune adjuvant such as vaccinia virus, Freund's adjuvant or another immune stimulant.

The present invention further provides for the use of an immunogenic KARI protein or one or more immunogenic KARI peptides or one or more immunogenic KARI fragments or one or more epitopes thereof in the preparation of a composition for the prophylactic or therapeutic treatment or diagnosis of tuberculosis or infection by one or more mycobacteria of the M. tuberculosis complex in a subject, such as, for example, a subject infected with HIV-1 and/or HIV-2 or a subject a risk of developing tuberculosis or being infected by M. tuberculosis, including the therapeutic treatment of a latent infection in a human subject.

In an alternative example, the present invention provides for the use of an immunogenic KARI protein or one or more immunogenic KARI peptides or one or more immunogenic KARI fragments or one or more epitopes thereof in the preparation of a composition for the prophylactic or therapeutic treatment or diagnosis of tuberculosis or infection by one or more mycobacteria of the M. tuberculosis complex in a subject wherein the subject has been subjected previously to antiviral therapy against HIV-1 and/or HIV-2.

The present invention also provides a kit for detecting one or more mycobacteria of the M. tuberculosis complex in a biological sample, said kit comprising:

(i) one or more isolated antibodies or immune reactive fragments thereof that bind specifically to the isolated or recombinant immunogenic KARI protein of one or more mycobacteria of the M. tuberculosis complex or an immunogenic KARI peptide or immunogenic KARI fragment or epitope thereof according to any example hereof or to a combination or mixture of said peptides or epitopes or fragments or to a fusion protein or protein aggregate comprising said immunogenic KARI protein, peptide, fragment or epitope; and
(ii) means for detecting the formation of an antigen-antibody complex, optionally packaged with instructions for use.

The present invention also provides a kit for detecting one or more mycobacteria of the M. tuberculosis complex in a biological sample, said kit comprising:

(i) isolated or recombinant immunogenic KARI protein of one or more mycobacteria or the M. tuberculosis complex or an immunogenic KARI peptide or immunogenic KARI fragment or epitope thereof according to any example hereof or a combination or mixture of said peptides or epitopes or fragments; and
(ii) means for detecting the formation of an antigen-antibody complex, optionally packaged with instructions for use.

The assays described herein are amenable to any assay format, and particularly to solid phase ELISA, flow through immune-assay formats, lateral flow formats, capillary formats, and for the purification or isolation of immunogenic proteins, peptides, fragments (e.g., using a solid matrix conjugated to antibody, protein G or protein A).

Accordingly, the present invention also provides a solid matrix having adsorbed thereto an isolated or recombinant KARI protein or an immunogenic KARI peptide or immunogenic KARI fragment or epitope thereof according to any one example described herein or a fusion protein or protein aggregate comprising said immunogenic KARI protein, peptide, fragment or epitope. For example, the solid matrix may comprise a membrane, e.g., nylon or nitrocellulose. Alternatively, the solid matrix may comprise a polystyrene or polycarbonate microwell plate or part thereof (e.g., one or more wells of a microtiter plate), a dipstick, a glass support, or a chromatography resin.

In an alternative example, the invention also provides a solid matrix having adsorbed thereto an antibody that binds to an isolated or recombinant KARI protein or an immunogenic KARI peptide or immunogenic KARI fragment or epitope thereof according to any example hereof or to a combination or mixture of said peptides or epitopes or fragments or to a fusion protein or protein aggregate comprising said immunogenic KARI protein, peptide, fragment or epitope. For example, the solid matrix may comprise a membrane, e.g., nylon or nitrocellulose. Alternatively, the solid matrix may comprise a polystyrene or polycarbonate microwell plate or part thereof (e.g., one or more wells of a microtiter plate), a dipstick, a glass support, or a chromatography resin.

It is clearly within the scope of the present invention for such solid matrices to comprise additional antigens and/or antibodies as required to perform an assay described herein, especially for multianalyte tests employing multiple antigens or multiple antibodies.

### 3. Definitions

As used herein, the term “Ketol-acid reductoisomerase” or “KARI” will be taken to mean M. tuberculosis protein composition comprising or having at least about 80% identity to SEQ ID NO: 1 or substantially the same sequence as set forth in SEQ ID NO: 1 of the present application and/or comprising or having a sequence that is at least about 80% identical to the sequence encoded by an ilvC gene of a Mycobacterium tuberculosis, said composition being suitable for the purposes of producing immunogenic peptides or preparing antibodies that cross react with one or more Mycobacteria of the M. tuberculosis complex or clinical matrix from subjects infected with said one or more Mycobacteria and not requiring any other functionality e.g., a role in protein translation. Until the present invention, the M. tuberculosis protein set forth in SEQ ID NO: 1 was not shown to be expressed in vivo, or to be immunogenic or immune-logically non-cross-reactive with other organisms, and information in relation to the KARI protein was derived from a bioinformatic analysis of open reading frames in the M. tuberculosis genome that encodes a polypeptide of SEQ ID NO: 1.

As used herein the term “derived from” shall be taken to indicate that a specified integer may be obtained from a particular source albeit not necessarily directly from that source.

Unless the context requires otherwise or specifically stated to the contrary, integers, steps, or elements of the invention recited herein as singular integers, steps or elements clearly encompass both singular and plural forms of the recited integers, steps or elements.

The examples of the invention described herein with respect to any single example and, in particular, with respect to any protein or a use thereof in the diagnosis, prognosis or therapy of M. tuberculosis shall be taken to apply mutatis mutandis to any other example of the invention described herein.

The diagnostic examples described here for individual subjects clearly apply mutatis mutandis to the epidemiology of a population, racial group or sub-group or to the diagnosis or prognosis of individuals having a particular MHC restriction. All such variations of the invention are readily derived by the skilled artisan based upon the subject matter described herein.

A reference herein to the detection or identification of M. tuberculosis and/or a reference to the diagnosis, prognosis or monitoring of tuberculosis or infection by M. tuberculosis clearly extends to the detection of any one or more organisms of the M. tuberculosis complex but not to the diagnosis of paratuberculosis and/or one or more organisms of the M. avium complex, unless the context requires otherwise. For example, as described herein the invention encompasses the use of antibodies that cross-react with M. tuberculosis KARI and fragments and one or more of M. avium and M. intracellulaire as a generic screen for mycobacteria, coupled to the use of one or more surrogate assays for detecting tuberculosis and/or for detecting one or more mycobacteria of the M. tuberculosis complex (but not coupled to any surrogate assay for diagnosing paratuberculosis and/or detecting one or more mycobacteria of the M. avium complex).

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein. Functionally equivalent products, compositions and methods are clearly within the scope of the invention, as described herein.

The present invention is performed without undue experimentation using, unless otherwise indicated, conventional techniques of molecular biology, microbiology, proteomics, virology, recombining DNA technology, peptide synthesis in solution, solid phase peptide synthesis, and immune-logy. Texts 1-17 infra teaching such conventional techniques are incorporated herein in their entirety by way of reference.

### BRIEF DESCRIPTION OF THE DRAWINGS

FIG. 1 is a graphical representation showing amplified sandwich ELISA standard curves for detection of M. tuberculosis ketol-acid reductoisomerase (KARI). Standard curves were generated using the optimised ELISA conditions for detection of KARI in buffer as described in the examples. The concentration of recombinant KARI protein (pg/ml) is indicated on the X-axis [ilvC] in logarithmic scale, and the mean OD is shown on the Y-axis. The capture and detector antibodies (Mo1283F and Ch34/35, respectively) were used at 5 and 2.5 μg/mL respectively. A 4-parameter logistic equation was used to fit a standard curve to the data for representative ELISA (n=2) (#1217; LOD=1690 pg/mL).

FIG. 2 is a graphical representation showing KARI protein expression (relative to total cellular protein) in one laboratory strain (H37Rv) and two clinical strains (CSU93 and HN878) of M. tuberculosis, as determined by sandwich ELISA. Whole cell lysates (WCL) from M. tuberculosis strain H37Rv (left), M. tuberculosis strains CSU93 (middle) and HN878 (right) were analysed by sandwich ELISA. The concentration of endogenous protein was calculated by interpolation from the standard curve and was corrected for the spiking level. Data were obtained from replicate experiments for which each sample was analyzed in duplicate. The level of endogenous protein (expressed as pg/μg total cell protein) was plotted as mean±SD for each of the three culture strains. M. tuberculosis strains were obtained courtesy of Colorado State University.

FIG. 3 is a graphical representation showing KARI protein expression (relative to total cell protein) in M. tuberculosis, M. intracellulaire and M. avium, as determined by sandwich ELISA. Whole cell lysates from M. tuberculosis strain H37Rv (left), and from M. avium (middle) and M. intracellulaire (right) were assayed in duplicate in two independent experiments. The concentration of endogenous protein was calculated by interpolation from the standard curve and was corrected for dilution factor. The level of endogenous protein expressed as pg/μg total cellular protein was plotted as mean±SD for each of the three Mycobacteria tested.

FIG. 4 is a graphical representation showing KARI protein expression in filtrates obtained from whole cell lysates of M. tuberculosis, M. intracellulaire and M. avium, as determined by sandwich ELISA. Filtrates obtained from whole cell lysates of M. tuberculosis strain H37Rv (left), M. avium (middle) and M. intracellulaire (right) were assayed in duplicate. The concentration of endogenous protein was calculated by interpolation from the standard curve and was corrected for dilution factor (if any). The level of endogenous protein expressed as pg/μL filtrate was plotted as mean±SD for each of the three Mycobacteria.

FIG. 5 is a graphical representation of sandwich ELISA results showing lack of significant cross-reactivity of antibodies against M. tuberculosis KARI protein with 0.1 μg/ml (columns 2, 4, 6) or 100 μg/ml (columns 1, 3, 5) of whole cell lysate from the non-mycobacteria pathogens Escherichia coli (columns 1 and 2), Bacillus subtilis (columns 3 and 4), and Pseudomonas aeruginosa (columns 5 and 6). Whole cell lysates were assayed in duplicate in 2 separate experiments. As a control, purified recombinant KARI protein was present at 0 ng/ml (column 7), 0.12 ng/ml (column 8), 0.49 ng/ml (column 9), 1.95 ng/ml (column 10), 7.8 ng/ml (column 11), 31.3 ng/ml (column 12), or 125 ng/ml (column 13), prepared by serial dilution of recombinant protein in blocking buffer. The mean OD+SD are plotted for the samples and controls.

FIG. 6 is a graphical representation showing the expression of KARI protein in clinical sputa obtained from patients categorized according to their TB smear test results, TB culture test results and HIV status. The series of histograms to the left of the figure show mean OD values for ELISA assays of KARI protein present in calibration standards comprising serial dilutions of M. tuberculosis strain H37Rv whole cell lysates: 60 μg/ml column 1; 20 μg/ml column 2; 6.67 μg/ml column 3; 2.22 μg/ml column 4; 0.74 μg/ml column 5; 0.25 μg/ml column 6; 0.08 μg/ml column 7; 0 μg/ml column 8. The series of histograms to the left of the figure show mean OD values for ELISA assays of KARI protein present in patient samples prepared as described in the accompanying examples (Method 3: 4.5 mL sputum-C1, 17×150 μL replacement amplification ELISA). “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of KARI protein cross-reactivity in smear positive/culture positive samples independent of HIV status of the subject.

FIG. 7 is a graphical representation showing the expression in clinical sputa obtained from patients categorized according to their TB smear test results, TB culture test results and HIV status expressed as pg KARI protein/ml sample volume. Data shown in FIG. 6 were converted to pg antigen based on KARI protein calibration values therein which permitted interpolation of ug/mL KARI protein for whole cell extracts of M. tuberculosis H37Rv into pg/mL rKARI protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of KARI protein cross-reactivity in smear positive/culture positive samples independent of HIV status of the subject. LOD for assay=˜900 pg/mL.

FIG. 8 is a graphical representation showing the effect of undiluted sputa in masking and/or quenching detection of endogenous M. tuberculosis KARI protein in the amplified sandwich ELISA assay described herein, and recovery of lost signal by dilution of the sputa. Sufficient whole cell lysate of M. tuberculosis H37Rv to provide 1.2 ng/ml KARI protein was spiked into undiluted blocking solution or sputum and incubated for 16 hours prior to assay or assayed directly. The same samples were also diluted serially in blocking solution prior to assay, wherein dilutions ranged from 1:3 (v/v) block:“neat” sample to 1:27 (v/v) block:“neat” sample as indicated on the x-axis. For assays, an ELISA plate was coated overnight with capture antibody Mo1283F. Following washing to remove unbound antibody, 50 μl aliquots of each undiluted sample (“neat”), or a 1:3 (v/v) dilution (“1 in 3”), a 1:9 (v/v) dilution (“1 in 9”), or a 1:27 (v/v) dilution (“1 in 27”) were added to the wells of the antibody-coated ELISA plates. Following incubation for 1 hour and washing to remove unbound antigen, antibody Ch34/35 was contacted with the bound antigen-body complexes. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show that there is significant attenuation/suppression of signal by sputum, irrespective of whether or not samples are assayed immediately or incubated for a prolonged period prior to assay, as indicated by the non-detectable signal in the first two columns of the “neat” samples compared to signal in blocking buffer. Up to about 50-75% of signal is recovered by dilution of samples in blocking buffer, and this recovery may occur even though the samples have been incubated in sputum for 16 hr prior to assay, suggesting that a significant contribution to signal loss in sputum is due to masking of the signal and quenching of signal by sputum.

FIG. 9 is a graphical representation showing the effect of undiluted sputa in masking and/or quenching detection of recombinant M. tuberculosis KARI protein in the amplified sandwich ELISA assay described herein, and recovery of lost signal by dilution of the sputa. KARI protein was spiked into undiluted blocking solution or sputum at a final concentration of 10 ng/ml, and samples were incubated for 16 hours prior to assay or assayed directly. The same samples were also diluted serially in blocking solution prior to assay, wherein dilutions ranged from 1:3 (v/v) block:“neat” sample to 1:27 (v/v) block:“neat” sample as indicated on the x-axis. For assays, an ELISA plate was coated overnight with capture antibody Mo1283F. Following washing to remove unbound antibody, 50 μl aliquots of each undiluted sample (“neat”), or a 1:3 (v/v) dilution (“1 in 3”), a 1:9 (v/v) dilution (“1 in 9”), or a 1:27 (v/v) dilution (“1 in 27”) were added to the wells of the antibody-coated ELISA plates. Following incubation for 1 hour and washing to remove unbound antigen, antibody Ch34/35 was contacted with the bound antigen-body complexes. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show that there is significant attenuation/suppression of signal by sputum, irrespective of whether or not samples are assayed immediately or incubated for a prolonged period prior to assay, as indicated by the non-detectable signal in the first two columns of the “neat” samples compared to signal in blocking buffer. Signal is recovered by dilution of samples in blocking buffer, even though the samples were incubated in sputum for prolonged periods, suggesting that a significant contribution to signal loss in sputum is due to masking of the signal and quenching of signal by sputum.

FIG. 10 is a graphical representation of screening results of clinical smear-positive sputum samples using KARI as target antigen and antibody Mo1283F as a capture antibody and Ch34/35 as a detector antibody. Samples from left to right were as follows: (i) a positive control sample comprising a serial dilution of M. tuberculosis H37Rv whole cell lysate at the protein concentrations indicated (μg protein/10; (ii) two smear-positive samples (MPC306 and MPC315) each assayed to a 1:1 (v/v) dilution, or a 1:3 (v/v) dilution, or a 1:9 (v/v) dilution in blocking buffer i.e., at the dilution factor indicated on the x-axis; (iii) a negative control (BD_{—}1) assayed at a 1:1 (v/v) dilution, or a 1:3 (v/v) dilution, or a 1:9 (v/v) dilution in blocking buffer i.e., at the dilution factor indicated on the x-axis; and (iv) a further positive control comprising the sample BD_{—}1 spiked with 30 μg/ml recombinant KARI protein and then diluted serially to a 1:1 (v/v) dilution, or a 1:3 (v/v) dilution, or a 1:9 (v/v) dilution in blocking buffer i.e., at the dilution factor indicated on the x-axis. ELISA signals are indicated on the y-axis. Data show background signal for negative controls, and detectable signals above background for the two smear-positive samples.

FIG. 11 is a graphical representation of screening results of clinical smear-positive sputum samples using KARL as target antigen and antibody Mo1283F as a capture antibody and Ch34/35 as a detector antibody, in a continuation of the experiment for which data are shown in FIG. 10. Samples from left to right were as follows: (i) a positive control sample comprising a serial dilution of M. tuberculosis H37Rv whole cell lysate at the protein concentrations indicated (μg protein/ml); (ii) two smear-positive samples (MPC305 and MPC316) each assayed to a 1:1 (v/v) dilution, or a 1:3 (v/v) dilution, or a 1:9 (v/v) dilution in blocking buffer i.e., at the dilution factor indicated on the x-axis; (iii) a smear-negative sample (MPC313) assayed to a 1:1 (v/v) dilution, or a 1:3 (v/v) dilution, or a 1:9 (v/v) dilution in blocking buffer i.e., at the dilution factor indicated on the x-axis; and (iv) a negative control sample (BD_{—}1) assayed at a 1:1 (v/v) dilution, or a 1:3 (v/v) dilution, or a 1:9 (v/v) dilution in blocking buffer i.e., at the dilution factor indicated on the x-axis. ELISA signals are indicated on the y-axis. Data show background signal for negative controls, and detectable signals above background for the two smear-positive samples and one smear-negative sample.

FIG. 12 is a graphical representation of screening results of clinical smear-positive sputum samples using KARI as target antigen and antibody Mo1283F as a capture antibody and Ch34/35 as a detector antibody, in a continuation of the experiment for which data are shown in FIG. 10. Samples from left to right were as follows: (i) a positive control sample comprising a serial dilution of M. tuberculosis H37Rv whole cell lysate at the protein concentrations indicated (μg protein/ml); and (ii) two smear-positive samples (MPC309 and MPC307) each assayed to a 1:1 (v/v) dilution, or a 10:1 (v/v) dilution in blocking buffer i.e., at the dilution factor indicated on the x-axis; and (iii) a smear-negative sample (MPC311) assayed at a 1:1 (v/v) dilution, or a 3:1 (v/v) dilution, or a 9:1 (v/v) dilution in blocking buffer i.e., at the dilution factor indicated on the x-axis. ELISA signals are indicated on the y-axis. Data show background signal for negative controls, and detectable signals above background for the two smear-positive samples and one smear-negative sample.

FIG. 13 provides photographic representations showing detection of recombinant KARI protein and endogenous M. tuberculosis KARI protein by western blot analysis using polyclonal Ch34/35 antibody (left panel) and monoclonal Mo1283F antibody (right panel). Recombinant KARI protein (rilvC, 10 ng), and 10 μg whole cell lysate (WCL) from cultured M. tuberculosis strains H37Rv, CSU93 or HN878 were resolved by SDS-PAGE, transferred to nitrocellulose membrane and probed with primary Ch34/35 polyclonal antibody followed by a secondary anti-chicken antibody (left panel headed “Chicken 34/35”), or with monoclonal Mo1283F antibody followed by a secondary anti-mouse antibody (left panel headed “Mouse 1283F”). Replica blots were also probed with the Ch34/35 antibody in the presence of 1 μg/ml unlabelled recombinant KARI protein (middle panel headed “Chicken 34/35”), or alternatively, with secondary antibody alone (right panel headed “Chicken 34/35”, and right panel headed “Mouse 1283F”). Boxed areas indicate the KARI protein bands. Molecular weights of proteins are indicated on the left of each group of panels. Data show the ability of both antibodies to bind to the recombinant KARI protein in all three strains tested, and the ability of the polyclonal antibody to detect endogenous protein in Western blots at the concentration tested. Data also show that antibody binding is abrogated by excess unlabelled protein.

FIG. 14 is a photographic representation of the detection of recombinant KARI protein and endogenous KARI protein by western blot analysis using different antibody preparations. Molecular weight marker proteins (lane 1), 1 ng recombinant KARI protein (lane 2), and 5 mg whole cell lysate (WCL) of cultured M. tuberculosis H37Rv (lane 3) were resolved by SDS-PAGE. Proteins were transferred to nitrocellulose membrane and probed using the primary antibodies indicated above each panel. Data show detectable binding to recombinant and endogenous KARI protein using Ch35 antibody preparation, a pooled Ch34/35 antibody preparation, monoclonal antibody 2B1 and monoclonal antibody 3A2, and detectable binding to endogenous KARI protein using monoclonal antibody preparations Mo1E7 and Mo2C7.

FIG. 15 is a graphical representation showing cross-reactivity of KARI protein in different M. tuberculosis strains as determined by amplified sandwich ELISA. ELISA plates were coated overnight with capture antibody Mo2B1 produced by plasmacytoma 2B1C11. Following washing to remove unbound antibody, 50 μl aliquots of serial dilutions of whole cell lysates of M. tuberculosis strain H37Rv obtained from Colorado University or in-house at Tyrian Diagnostics, and cell lysates of M. tuberculosis strains HN878 and CDC1551, were added to the wells of the antibody-coated ELISA plates. Following incubation for 1 hour and washing to remove unbound antigen, antibody Ch34/35 was contacted with the bound antigen-body complexes. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show that there is significant cross-reactivity between the strains tested using this antibody combination.

FIG. 16 is a graphical representation showing KARI protein in the cytosolic fraction of cell lysates from different M. tuberculosis strains as determined by amplified sandwich ELISA. ELISA plates were coated overnight with capture antibody Mo2B1 produced by plasmacytoma 2B1C11. Following washing to remove unbound antibody, 50 μl aliquots of serial dilutions of cytosolic protein from M. tuberculosis strains H37Rv, HN878 and CDC1551 were added to the wells of the antibody-coated ELISA plates. Following incubation for 1 hour and washing to remove unbound antigen, antibody Ch34/35 was contacted with the bound antigen-body complexes. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show that there is detectable KARI protein in the cytosol of all three strains tested using this antibody combination, with higher levels in H37Rv and lower levels in CDC1551.

FIG. 17 is a graphical representation showing KARI protein in the cell membrane fraction of cell lysates from different M. tuberculosis strains as determined by amplified sandwich ELISA. ELISA plates were coated overnight with capture antibody Mo2B1 produced by plasmacytoma 2B1C11. Following washing to remove unbound antibody, 50 μl aliquots of serial dilutions of solubilised cell membrane protein from M. tuberculosis strains H37Rv, HN878 and CDC1551 were added to the wells of the antibody-coated ELISA plates. Following incubation for 1 hour and washing to remove unbound antigen, antibody Ch34/35 was contacted with the bound antigen-body complexes. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show that there is detectable KARI protein in the cell membrane of all three strains tested using this antibody combination, with higher levels in H37Rv and lower levels in CDC1551 and HN878.

FIG. 18 is a graphical representation showing KARI protein in the cell wall fraction of cell lysates from different M. tuberculosis strains as determined by amplified sandwich ELISA. ELISA plates were coated overnight with capture antibody Mo2B1 produced by plasmacytoma 2B1C11. Following washing to remove unbound antibody, 50 μl aliquots of serial dilutions of solubilised cell wall protein from M. tuberculosis strains H37Rv, HN878 and CDC1551 were added to the wells of the antibody-coated ELISA plates. Following incubation for 1 hour and washing to remove unbound antigen, antibody Ch34/35 was contacted with the bound antigen-body complexes. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show that there is detectable KARI protein in the cell walls of all three strains tested using this antibody combination, with higher levels in H37Rv and lower levels in CDC1551.

FIG. 19 is a graphical representation showing relative ELISA signals for M. tuberculosis strain H37Rv whole cell lysate (right curve) and recombinant KARI protein (left curve) as determined by amplified sandwich ELISA. ELISA plates were coated overnight with capture antibody Mo2B1 produced by plasmacytoma 2B1C11. Following washing to remove unbound antibody, 50 μl aliquots of serial dilutions of whole cell lysate from M. tuberculosis strain H37Rv in-house at Tyrian Diagnostics, and 50 μl aliquots of serial dilutions of recombinant KARI protein were added to the wells of the antibody-coated ELISA plates. Following incubation for 1 hour and washing to remove unbound antigen, antibody Ch34/35 was contacted with the bound antigen-body complexes. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show relative titration values for endogenous and recombinant KARI protein. Data also demonstrate that endogenous protein is clearly detectable, notwithstanding that there may be masking and/or quenching of endogenous protein in whole cell lysates.

FIG. 20 is a graphical representation showing lack of detectable cross-reactivity between endogenous M. tuberculosis KARI protein and proteins in cell lysates of Escherichia coli, Pseudomonas aeruginosa, Bacillus subtilis, and Saccharomyces cerevisiae. ELISA plates were coated overnight with capture antibody Mo2B1 produced by plasmacytoma 2B1C11. Following washing to remove unbound antibody, 50 μl aliquots of serial dilutions of whole cell lysates from M. tuberculosis strain H37Rv, Escherichia coli, Pseudomonas aeruginosa, Bacillus subtilis, and Saccharomyces cerevisiae were added to the wells of the antibody-coated ELISA plates. Following incubation for 1 hour and washing to remove unbound antigen, antibody Ch34/35 was contacted with the bound antigen-body complexes. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show detectable signal generated to M. tuberculosis KARI protein, and absence of significant signal to proteins in whole cell lysates of the other organisms demonstrating specificity of the assay using this antibody pair.

FIG. 21 is a graphical representation showing lack of detectable cross-reactivity between endogenous M. tuberculosis KARI protein and proteins in cell lysates of Escherichia coli, Pseudomonas aeruginosa, Bacillus subtilis, and Saccharomyces cerevisiae, and weak cross-reactivity between M. tuberculosis KARI protein and M. intracellulaire and M. avium KARI proteins. ELISA plates were coated overnight with capture antibody Mo2B1 produced by plasmacytoma 2B1C11. Following washing to remove unbound antibody, 50 μl aliquots of serial dilutions of whole cell lysates from M. tuberculosis strain H37Rv (M.tb lysate), Escherichia coli (E. coli), Pseudomonas aeruginosa (Pseud), Bacillus subtilise B. sub), Saccharomyces cerevisiae (Yeast), M. intracellulaire (Intrce. Lys) and M. avium (Avium Lys) were added to the wells of the antibody-coated ELISA plates. Following incubation for 1 hour and washing to remove unbound antigen, antibody Ch34/35 was contacted with the bound antigen-body complexes. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show detectable signal generated to M. tuberculosis KARI protein, with much weaker signal generation to M. intracellulaire and M. avium proteins (approximately 100-fold less sensitive), and the absence of significant signal to proteins in whole cell lysates of the other organisms demonstrating specificity of the assay using this antibody pair.

FIG. 22 has been intentionally omitted.

FIGS. 23a-e provide graphical representations showing screening results of clinical smear-positive sputum samples using KARI as target antigen and antibody Mo2B1 as a capture antibody and Ch34/35 as a detector antibody. In FIGS. 23a-e, the left series of columns provide standard calibration for KARI protein from a serial dilution of M. tuberculosis H37Rv whole cell lysate at the protein concentrations indicated (μg protein/ml), and a blank negative control comprising blocking buffer; and the right-most columns provide signals for a negative control sputa, or the same negative control sputa comprising 10 μg/ml recombinant KARI protein, and then diluted serially to a 1:1 (v/v) dilution, or a 1:3 (v/v) dilution in blocking buffer. Samples in each figure included smear-negative and smear-positive samples as follows:

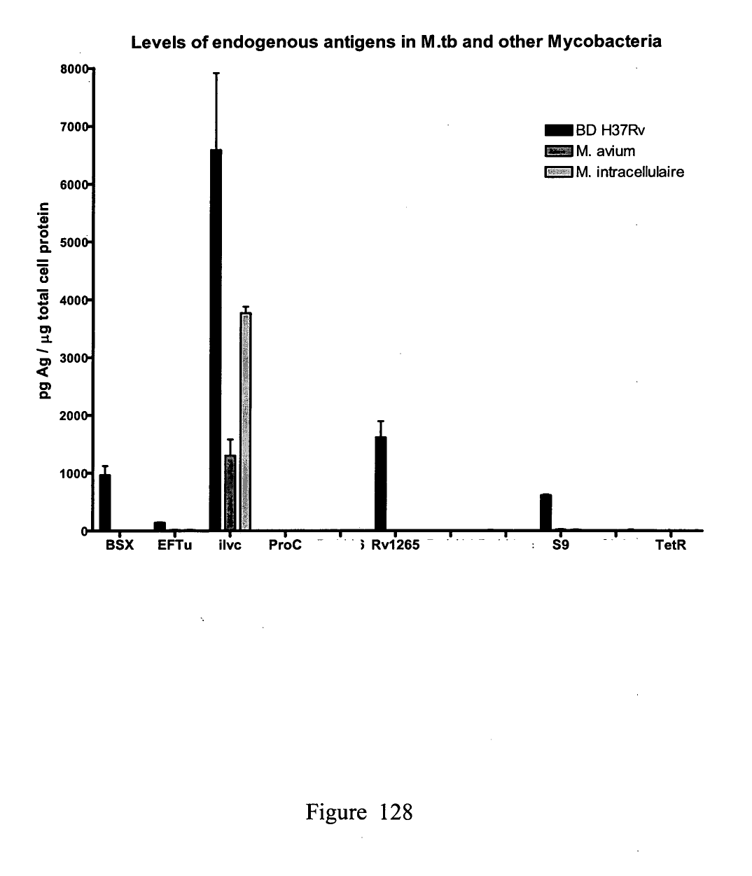

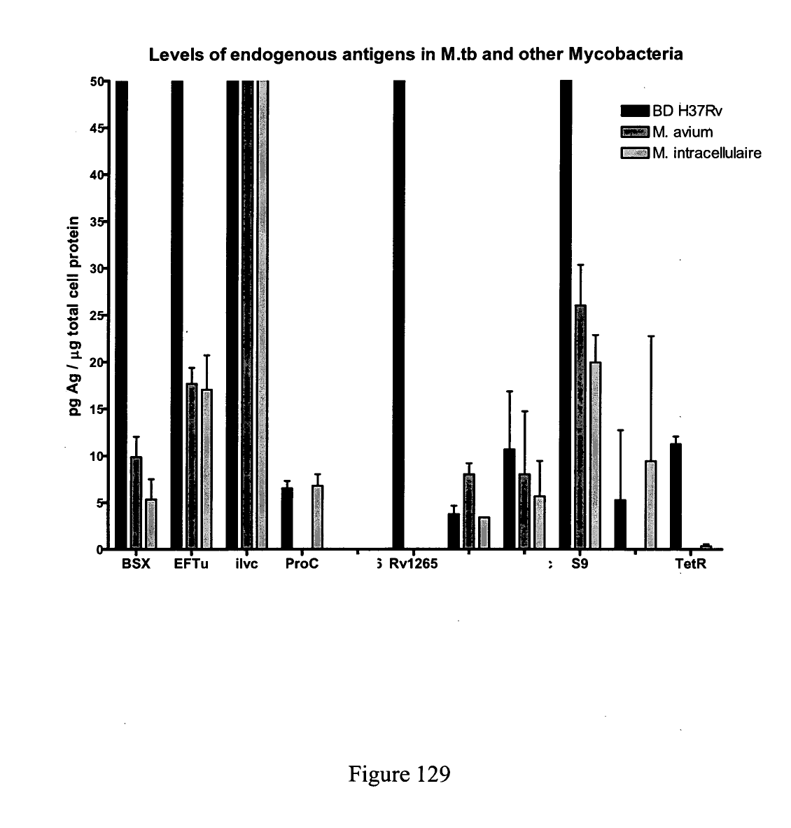

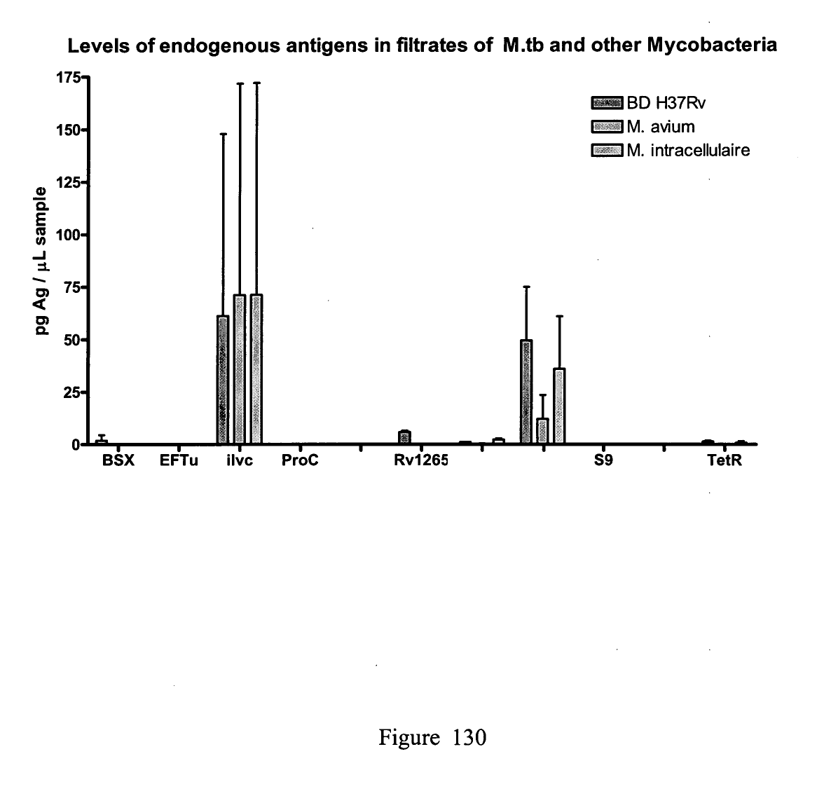

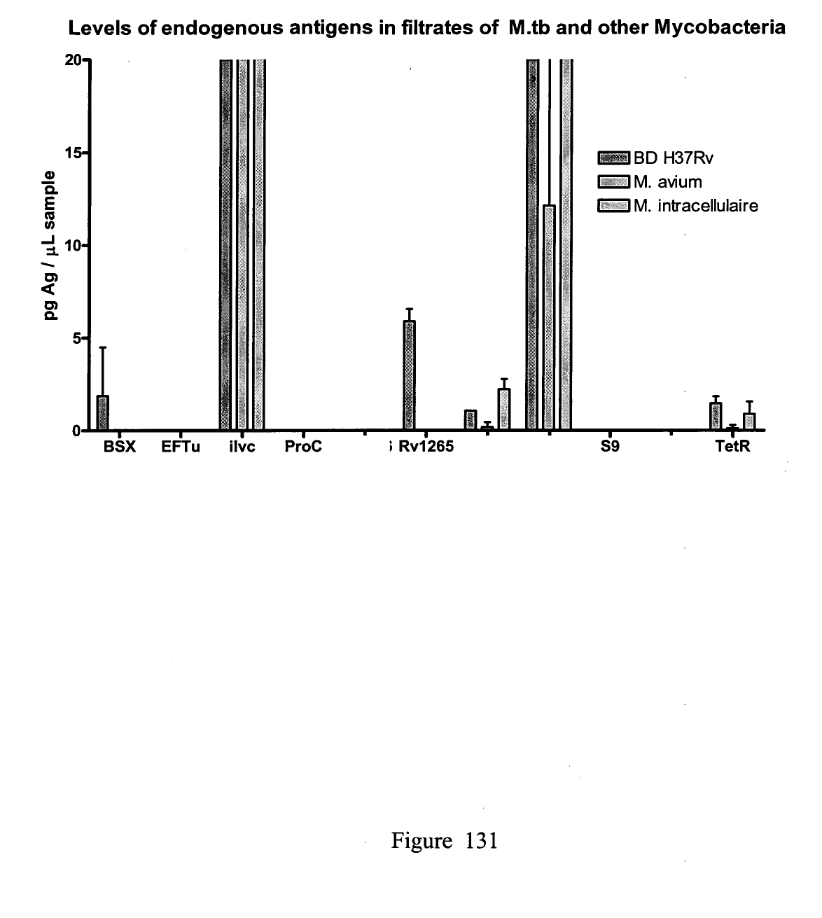

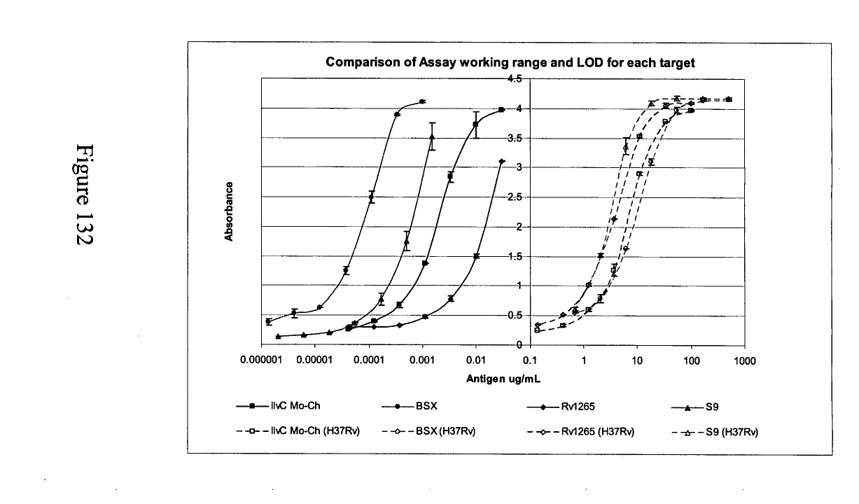

Smear-negative in FIG. 23a: BD1229; BD1288; MPC364; BD1287; BD1187; MPC363;
Smear-positive in FIG. 23a: MPC360; MPC374; MPC366; MPC357; MPC356;
Smear-negative in FIG. 23b: CGS123; MPC375; Phru0905; CGS119; and MPC388;
Smear-positive in FIG. 23b: MPC381; MPC380; MPC379; MPC378;
Smear-negative in FIG. 23c: BD505; MPC339;
Smear-positive in FIG. 23c: MPC370; MPC365; MPC335; MPC372; MPC342; MPC377; MPC324; MPC367; MPC359; MPC368;
Smear-negative in FIG. 23d: All samples listed
Smear-positive in FIG. 23d: No samples listed
Smear-negative in FIG. 23e: 3-D; 3-E; 3-H; 3-J; 3-L; and
Smear-positive in FIG. 23e: 3-A; 3-B; 3-C; 3-E; 3-F; 3-G; 3-I; 3-K.
Amplified ELISA assays were performed on the samples at the dilutions indicated, as described in the preceding figure legends, using monoclonal antibody Mo2B1 as a capture antibody and polyclonal Ch34/35 serum as detector antibody. Sample codes and smear values are indicated on the x-axes. ELISA signals are indicated on the y-axes as the means±SD (n=2). Data show background signal for negative controls, and detectable signals above background for the positive controls comprising whole cell lysates or recombinant KARI protein. For clinical samples, data show detectable signals significantly above background for all smear-positive samples, and signals below background for a majority of smear-negative samples. Some smear-positive samples also provided a signal above background e.g., MPC364, MPC363, MPC375, MPC388, and MPC339, however all of these false-positive detections could be resolved by surrogate assay using one or more antigen-based assays employing antibodies against Rv1265 and/or BSX and/or S9 proteins (data not shown).

FIG. 24 provides a graphical representation showing detection of endogenous M. tuberculosis KARI protein using an amplified sandwich ELISA employing monoclonal antibody Mo1F6 as a capture antibody and biotinylated monoclonal antibody Mo2B1 (2B1-Bi) as a detector antibody. Amplified ELISA was performed essentially as described herein in two separate experiments. Data indicate that the assay detects KARI protein.

FIG. 25 provides a graphical representation of amplified sandwich ELISA using monoclonal antibodies raised against peptides comprising regions of KARI protein (Mo4F7, Mo3C3, Mo4C10, Mo1C10) or recombinant KARI protein (Mo2B1) to bind endogenous KARI protein in M. tuberculosis whole cell lysates. Two batches of each monoclonal antibody were tested. Data show strongest binding to endogenous KARI protein by monoclonal antibody Mo2B1 prepared against full-length recombinant protein, and detectable binding by monoclonal antibodies Mo4F7 and Mo4C10 produced against a synthetic peptide comprising residues 40-56 of SEQ ID NO: 1.

FIG. 26 provides graphical representations showing antibody titrations for the monoclonal antibodies Mo1A4, Mo1H2, Mo2D6, Mo2E5, Mo2G2, Mo3H3, Mo4C3, Mo4D2, and Mo4D 11. Antibodies were titrated at the dilutions indicated on the x-axis, wherein at each antibody dilution tested the signal generated relates to the following antibodies from left to right: 1A4, 1H2, 2D6, 2E5, 2G2, 3H3, 4C3, 4D2, and 4D11. Data indicate the highest titer for antibodies 2D6, 3H3 and 4D11 in the order 2D63>>H3>4D11.

FIG. 27 provides graphical representations showing the ability of monoclonal antibodies Mo1A4, Mo1H2, Mo2D6, Mo2E5, Mo2G2, Mo3H3, Mo4C3, Mo4D2, and Mo4D11 to detect recombinant KARI protein. Antibodies were titrated against equal volumes of the concentrations of recombinant KARI protein indicated on the x-axis, wherein at each concentration tested the signal generated relates to the following antibodies from left to right: 1A4, 1H2, 2D6, 2E5, 2G2, 3H3, 4C3, 4D2, and 4D11. Data indicate that antibodies 2D6, 3H3 and 4D11 detect KARI protein in the microgram-to-nanogram concentration range.

FIG. 28 provides a graphical representation showing detection of recombinant KARI protein using an amplified sandwich ELISA employing monoclonal antibody Mo1F6 or Mo2D6 as a capture antibody and biotinylated monoclonal antibody Mo2B1 (2B1-Bi) as a detector antibody. Amplified ELISA was performed essentially as described herein in two separate experiments for each antibody pair. Data indicate that both assays detect KARI protein.

FIG. 29 provides photographic representations (above) and a graphical representation (below) showing a standard curve of endogenous M. tuberculosis KARI protein in whole cell lysates of strain H37Rv in a point-of-need assay format (DiagnostIQ™, Tyrian Diagnostics, Australia). For each assay, 500 μL of M. tuberculosis strain H37Rv whole cell lysates (WCL) comprising protein in the range of 7.5-120 μg/mL was tested. Endogenous KARI was captured using chicken anti-KARI polyclonal antibody pool designated Ch34/35, and detected using gold-conjugated monoclonal antibody Mo2B1. Each assay point was performed in duplicate. Data indicate that the ELISA assay is reducible to a point-of-need format.

FIG. 30 is a graphical representation showing a comparison of the concentration of recombinant BSX detected using a chicken anti-BSX polyclonal antibody preincubated with recombinant BSX (solid diamonds); a chicken anti-BSX antibody without preincubation (grey squares); a rabbit anti-BSX polyclonal antibody (solid triangles) and a mouse anti-BSX monoclonal antibody (solid squares). The concentration of the recombinant protein is indicated on the X-axis and the optical density indicated on the Y-axis.

FIG. 31 is a graphical representation showing the detection of recombinant BSX using a sandwich ELISA in which monoclonal antibody Mo403B was used as a capture reagent and polyclonal antibody C44 was used as a detection reagent. Titrating amounts of recombinant BSX from 50 ng/ml down to 0.39 ng/ml were screened. Concentrations of detection and capture reagents are indicated. The concentration of BSX is shown on the X-axis and the mean OD is shown on the Y-axis.

FIG. 32 is a graphical representation showing the detection of BSX in sputa of TB and control subjects using a Sandwich ELISA. The optical density is indicated on the Y-axis and the sample type and number is indicated on the X-axis.

FIG. 33 is a graphical representation showing the detection of recombinant BSX using an amplified sandwich ELISA in which monoclonal antibody Mo403B was used as a capture reagent detection reagent (as indicated) and polyclonal antibody C44 was used as a detection reagent or capture reagent (as indicated). Titrating amounts of recombinant BSX from 50 ng/ml down to 0.39 ng/ml were screened. Concentrations of detection and capture reagents are indicated. The concentration of BSX is shown on the X-axis and the mean OD is shown on the Y-axis.

FIG. 34 is a graphical representation showing the detection of recombinant BSX using an amplified ELISA in which C44 is used as a capture reagent. Purified chicken anti-BSX pAb C44 was immobilised onto an ELISA plate as a Capture antibody at a concentration of 20 μg/ml using 50 μl per well. Titrating amounts of recombinant BSX from 10 ng/ml down to 0.078 ng/ml were then screened by sequential addition of purified Rabbit anti-BSX (Peptide 28) pAb at a concentration of 5 μg/ml, and then a Goat anti-Rabbit IgG at a dilution of either 1:30,000 (v/v) or 1:60,000 (v/v), as a second Detector. Donkey anti-Goat IgG HRP at a dilution of 1:5,000 (v/v) and TMB were used for signal detection.

FIG. 35 is a graphical representation showing the measurement of detection limits of standard sandwich ELISA versus biotin based Amplification System. Purified Rabbit anti-BSX pAb R16 was immobilised onto an ELISA plate at a concentration of 20 μg/ml. Titrating amounts of recombinant BSX were added at a concentration of 50 ng/ml down to 0.39 ng/ml for 1 hr unless specified otherwise (i.e. 2 hr). Antigen detection was performed using either a standard sandwich system where Chicken anti-BSX pAb C44 was added at a concentration of 5 μg/ml followed by Sheep anti-Chicken IgG HRP conjugate at a dilution of 1:5000 (v/v), or an amplifying system where Chicken anti-BSX was first added at 5 μg/ml followed by Donkey anti-Chicken IgG biotin conjugate at various dilutions as specified above, and finally streptavidin-HRP at a 1:5000 (v/v) dilution. Background (i.e. signal without BSX present) has been subtracted from the above curves.

FIG. 36 is a graphical representation showing detection of titrating amounts of recombinant BSX using a Biotin-based amplified ELISA. Purified Rabbit anti-BSX (anti-Peptide 28) pAb R16 was immobilised onto an ELISA plate as a capture antibody at a concentration of either 20 or 40 μg/ml. Titrating amounts of recombinant BSX from 10 ng/ml down to 4.9 pg/ml were then screened by sequential addition of purified chicken anti-BSX pAb C44 at a concentration of 5 μg/ml, and then a Donkey anti-Chicken IgG biotin conjugate at a dilution of 1:20,000 (v/v) as a second detector. Streptavidin HRP conjugate at a dilution of 1:5000 (v/v) and TMB were used for signal detection. Background OD intensity was obtained for both of the Rabbit anti-BSX capture concentrations where the recombinant BSX was not added.

FIG. 37 is a graphical representation showing screening of sputum for endogenous BSX using sandwich ELISA with a Biotin Amplification System. Sputum samples (50 μl+50 μl blocking buffer) from South African TB patients and control patients with non-TB respiratory disease from South Africa (prefix ‘M’) and Australia (prefix ‘CGS’) respectively were screened by sandwich ELISA for the presence of BSX antigen. Purified Rabbit anti-BSX (peptide 28) pAb was immobilised onto the ELISA plate as a Capture antibody at a concentration of 20 μg/ml. Purified Chicken anti-BSX pAb, C44, at a concentration of 5 μg/ml, was used as the Detector antibody. Biotinylated Donkey anti-Chicken IgG at a dilution of 1:20000 (v/v) was used as a second detector. Streptavidin HRP at a dilution of 1:5000 (v/v) and TMB were used for signal detection. Sputum from control patient CGS25 was spiked with 5 ng/ml and 1 ng/ml recombinant BSX as a positive control.

FIG. 38 is a graphical representation showing the effect of multiple sample loads on detection of BSX protein by Amplified Sandwich ELISA. Rabbit anti-BSX pAb R16 was immobilised onto an ELISA plate as the capture antibody at a concentration of 20 μg/ml using 50 μl per well. Sputum samples from TB patients and non-TB respiratory disease control patients were diluted at a 1:1 (v/v) ratio with blocker solution. The positive control is recombinant BSX at 1 ng/ml spiked in CGS23 sputum sample. Sputum samples were either (i) incubated for 1 hr as per a standard ELISA; (ii) incubated for 2 hr; or (iii) incubated for 2 hr, removed and fresh sputum added for an additional 1 hr of incubation. Endogenous BSX was detected using purified Chicken anti-BSX pAb C44 at 5 μg/ml, followed by Donkey anti-Chicken IgG biotin conjugate at a dilution of 1:20,000 (v/v) and finally with streptavidin HRP conjugate at 1:5000 (v/v) dilution.

FIG. 39 is a graphical representation showing standard and amplified sandwich ELISA standard curves for detection of M. tuberculosis BSX protein. Standard curves were generated using optimised ELISA conditions for detection of BSX in buffer as described in the examples. The concentration of recombinant BSX protein (pg/ml) is indicated on the X-axis in logarithmic scale, and the mean OD is shown on the Y-axis. The capture and detector antibodies (Mo639F and Ch12/13 respectively) were used at 2 μg/mL and 5 μg/mL respectively. The data were plotted as an X-Y graph for mean OD+SD (n=3) against log pg/mL of rBSX, and a curve fit used to determine the LOD for the assays (#733 standard and amplified ELISA, LOD=552 and 89 pg/mL respectively).

FIG. 40 is a graphical representation showing BSX protein expression (relative to total cellular protein) in one laboratory strain (H37Rv) and two clinical strains (CSU93 and HN878) of M. tuberculosis, as determined by sandwich ELISA. Whole cell lysates (WCL) from M. tuberculosis strain H37Rv (left), M. tuberculosis strains CSU93 (middle) and HN878 (right) were analysed by sandwich ELISA. The concentration of endogenous protein was calculated by interpolation from the standard curve and was corrected for the spiking level. Data were obtained from replicate experiments for which each sample was analyzed in duplicate. The level of endogenous protein (expressed as pg/μg total cell protein) was plotted as mean±SD for each of the three culture strains. M. tuberculosis strains were obtained courtesy of Colorado State University.

FIG. 41a is a graphical representation showing BSX protein expression (relative to total cell protein) in M. tuberculosis, M. intracellulaire and M. avium, as determined by sandwich ELISA. Whole cell lysates from M. tuberculosis strain H37Rv (left), and from M. avium (middle) and M. intracellulaire (right) were assayed in duplicate in two independent experiments. The concentration of endogenous protein was calculated by interpolation from the standard curve and was corrected for dilution factor. The level of endogenous protein expressed as pg/μg total cellular protein was plotted as mean±SD for each of the three Mycobacteria tested.

FIG. 41b is a graphical representation showing BSX protein expression in filtrates obtained from whole cell lysates of M. tuberculosis, M. intracellulaire and M. avium, as determined by sandwich ELISA. Filtrates obtained from whole cell lysates of M. tuberculosis strain H37Rv (left), M. avium (middle) and M. intracellulaire (right) were assayed in duplicate. The concentration of endogenous protein was calculated by interpolation from the standard curve and was corrected for dilution factor (if any). The level of endogenous protein expressed as pg/μL filtrate was plotted as mean±SD for each of the three Mycobacteria.

FIG. 42 is a graphical representation of sandwich ELISA results showing lack of significant cross-reactivity of antibodies against M. tuberculosis BSX protein with 0.1 μg/ml (columns 2, 4, 6) or 100 μg/ml (columns 1, 3, 5) of whole cell lysate from the non-mycobacteria pathogens Escherichia coli (columns 1 and 2), Bacillus subtilis (columns 3 and 4), and Pseudomonas aeruginosa (columns 5 and 6). Whole cell lysates were assayed in duplicate in 2 separate experiments. As a control, purified recombinant BSX protein was present at 0 pg/ml (column 7) and 3 ng/ml (column 8), prepared by serial dilution of recombinant protein in blocking buffer. The mean OD+SD are plotted for the samples and controls.

FIG. 43 is a graphical representation showing detection of M. tuberculosis BSX protein in sputa from clinical samples by immune-magnetic bead assay. A total of 1.2×10⁷ magnetic beads, coated with 1.8 μg of anti-BSX Ch8 pAb, were incubated overnight with 500 μL of either TB positive or negative sputum (treatment Sputum-M1) after sample pre-treatment: 10 mM EDTA and 1× protease inhibitor cocktail were added to each sample, followed by 1 hr reduction with 10 mM DTT on ice, 1 hr alkylation with 30 mM IAA on ice, 30 min incubation with 0.25% SDS at room temperature with rotation. The samples were diluted 1:10 (v/v) in sample diluting buffer (BNTT—1% BSA, 100 mM NaCl, 10 mM Tris and 0.05% Tween 20). Recombinant BSX protein (100 pg) was added to 500 μL of a Mitha control pool subjected to the same pre-treatment process, to form a positive control for the assay. Bound endogenous antigen was detected using 1 mL of 5 μg/mL anti-BSX antibody (Mo639F) diluted in BNTT as a detector antibody, followed by 100 μL of anti-mouse HRP-conjugated antibody diluted 1:5000 (v/v) in conjugate diluent buffer [0.1% (w/v) casein, 0.1% (v/v) Tween 20]. Data are expressed as OD450-620 and plotted for the samples and standards alike.

FIG. 44 is a graphical representation showing the expression of BSX protein in clinical sputa obtained from patients categorized according to their TB smear test results, TB culture test results and HIV status. Sputa were from each of 4 TB-positive and TB-negative subjects collected in Cameroon and processed as described in the examples (Method 3:9.0 mL sputum-C1, 4×150 μL replacement amplification ELISA). Briefly, sputum-C1 was size-fractionated to remove contaminants less than 100 kDa molecular weight, equilibrated to 50 mM Tris, pH 7.8, 5 mM MgCl₂, concentrated and analyzed as 4×150 uL aliquots by replacement amplification ELISA. The series of histograms to the left of the figure show mean OD values for ELISA assays of BSX protein present in calibration standards comprising serial dilutions of M. tuberculosis strain H37Rv whole cell lysates: 60 μg/ml column 1; 20 μg/ml column 2; 6.67 μg/ml column 3; 2.22 μg/ml column 4; 0.74 μg/ml column 5; 0.25 μg/ml column 6; 0.08 μg/ml column 7; 0 μg/ml column 8. The series of histograms to the left of the figure show mean OD values for ELISA assays of BSX protein present in patient samples prepared as described in the examples. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of BSX protein cross-reactivity in smear positive/culture positive samples.

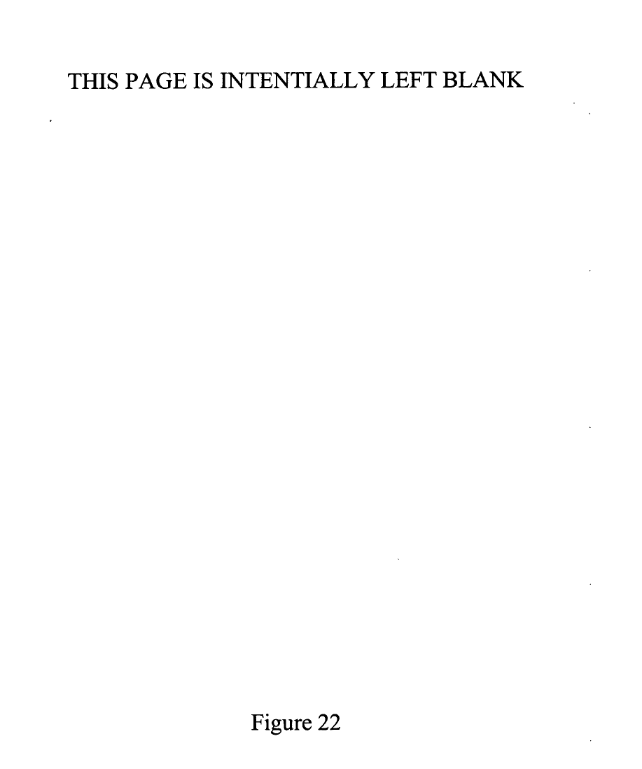

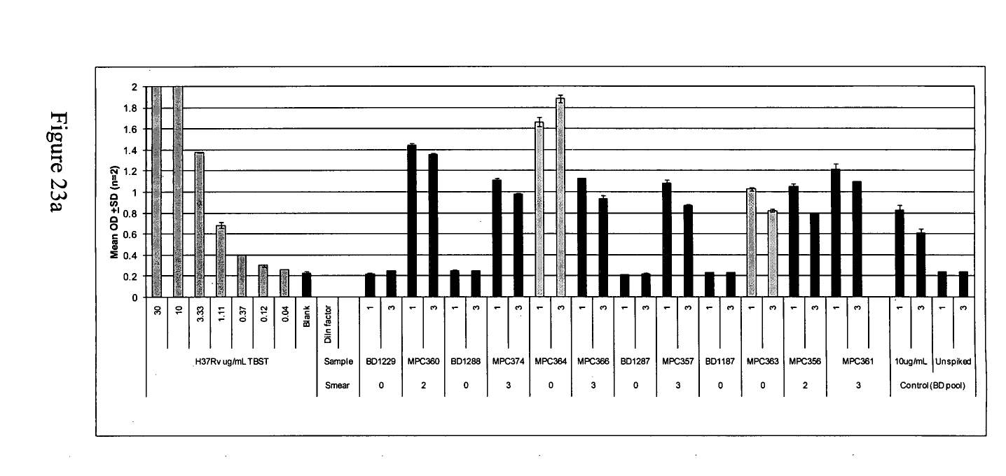

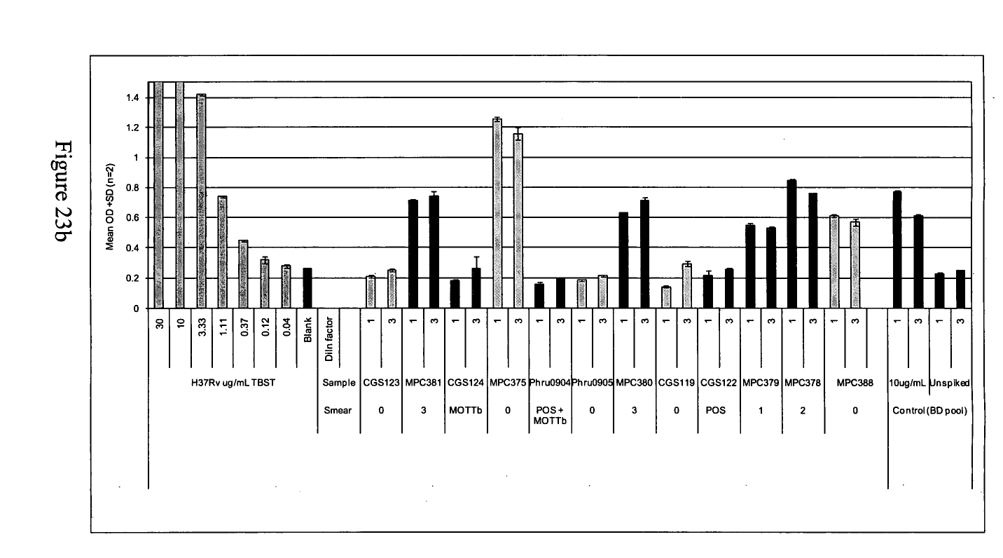

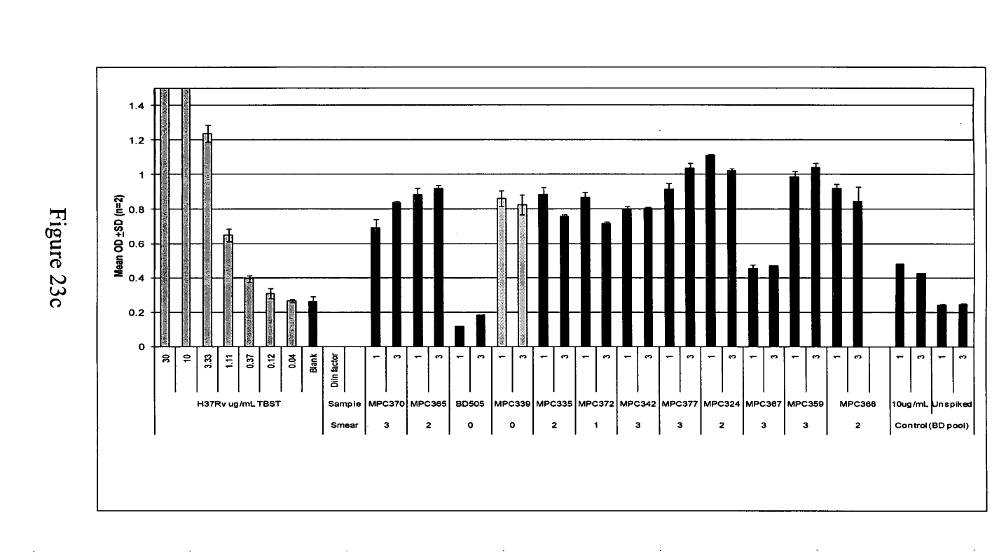

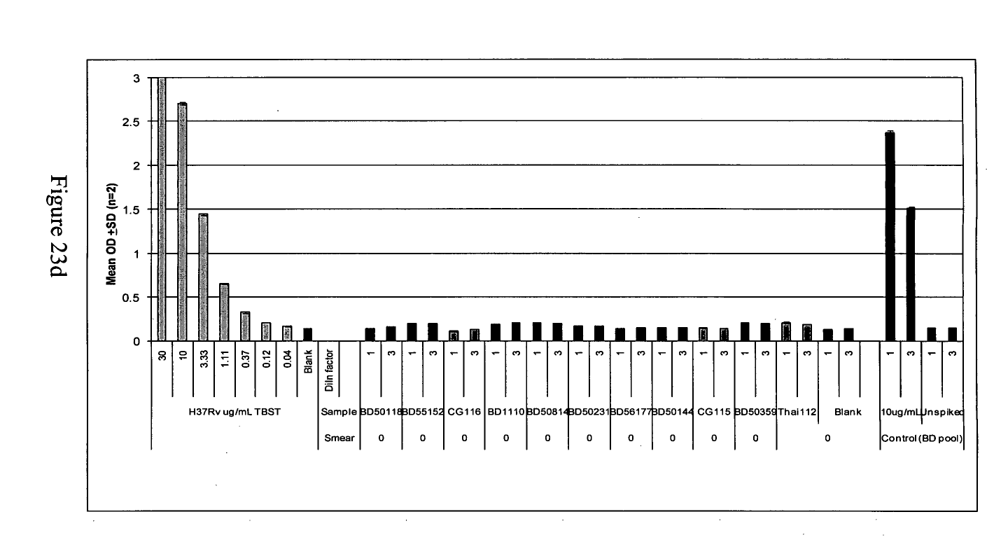

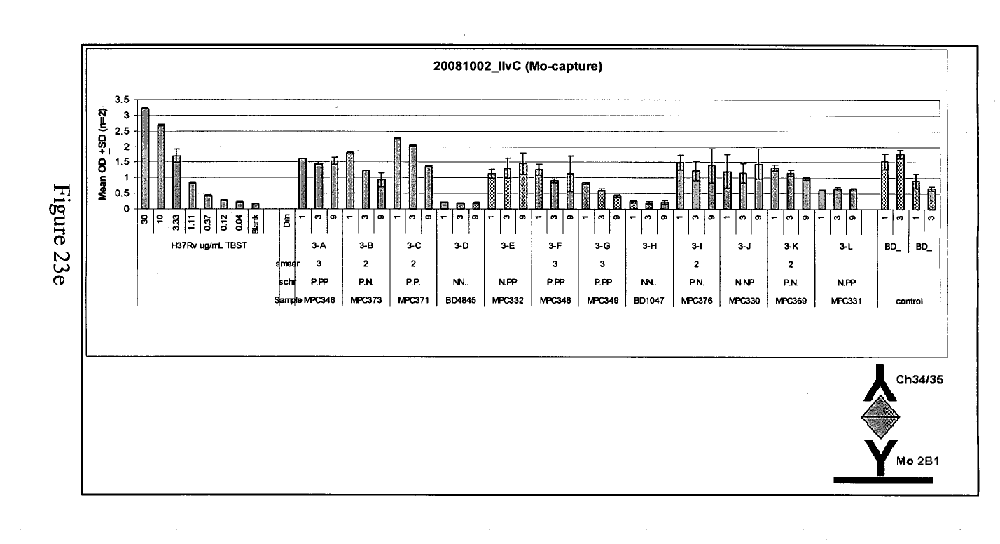

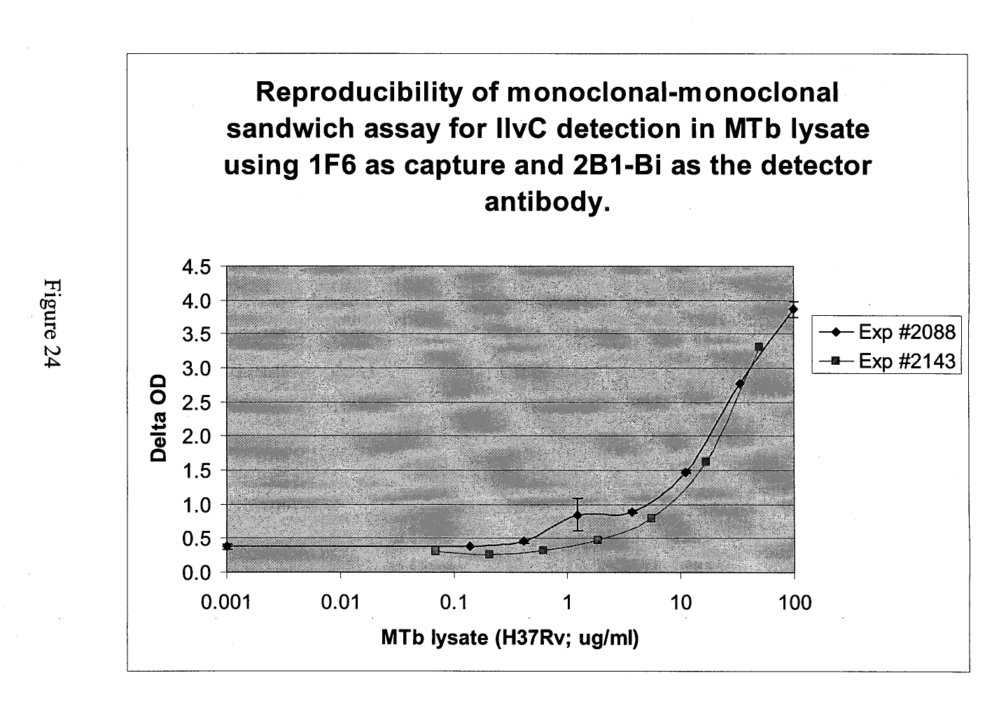

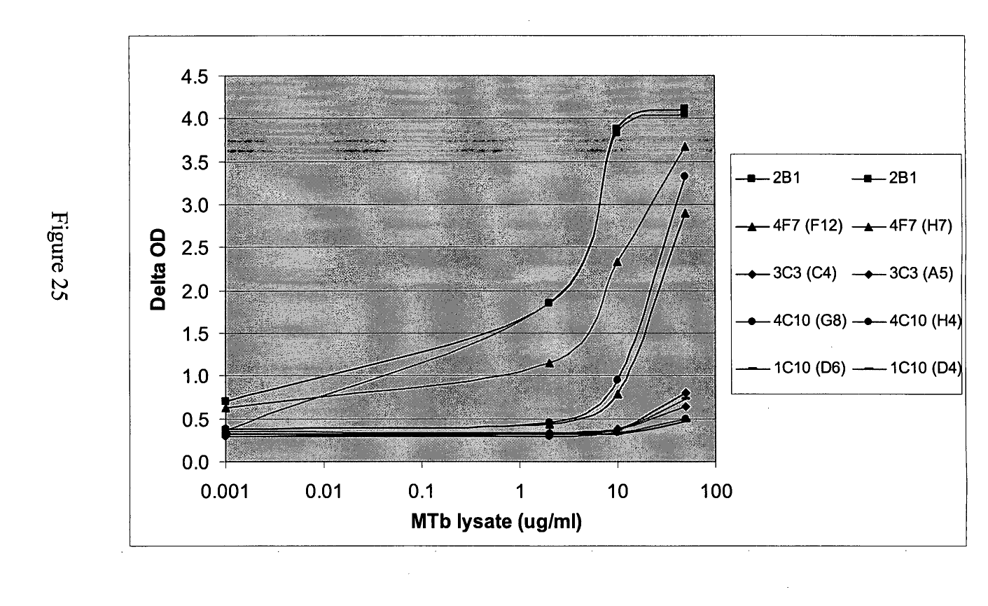

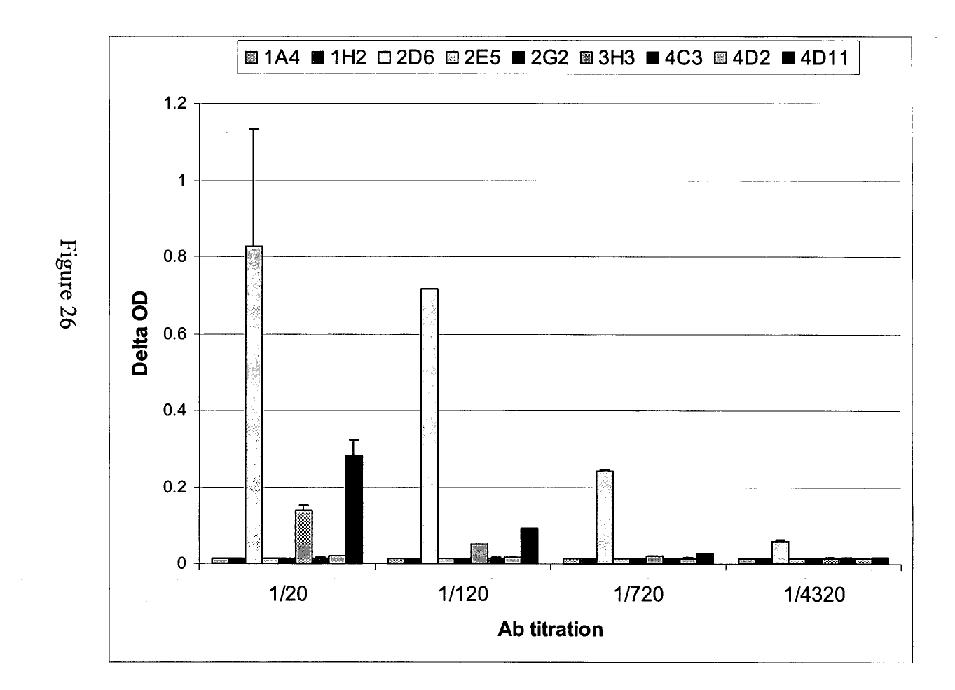

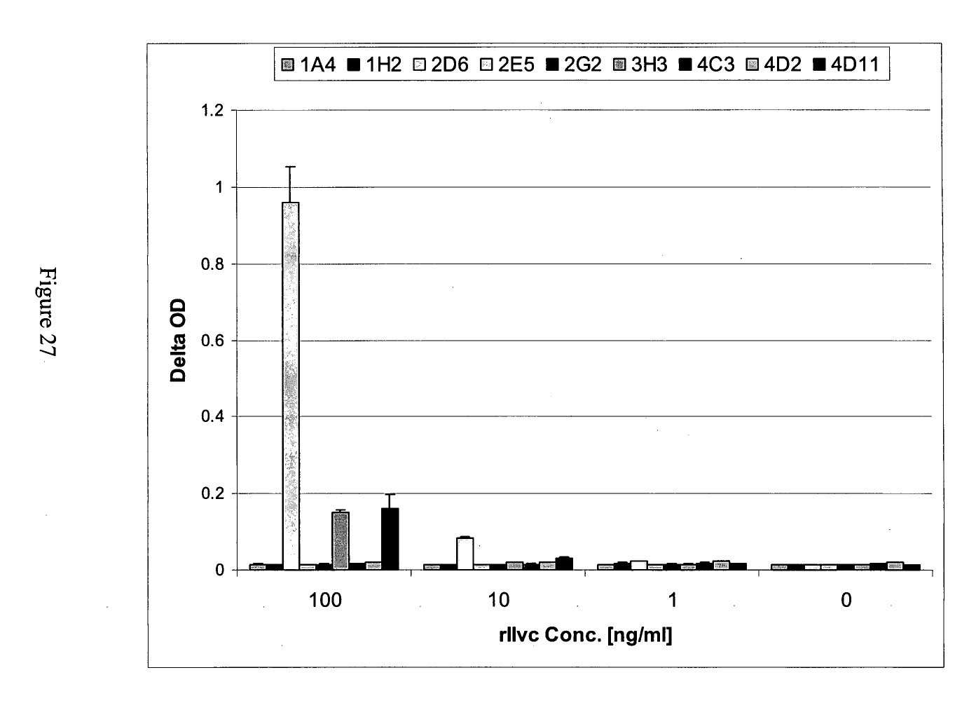

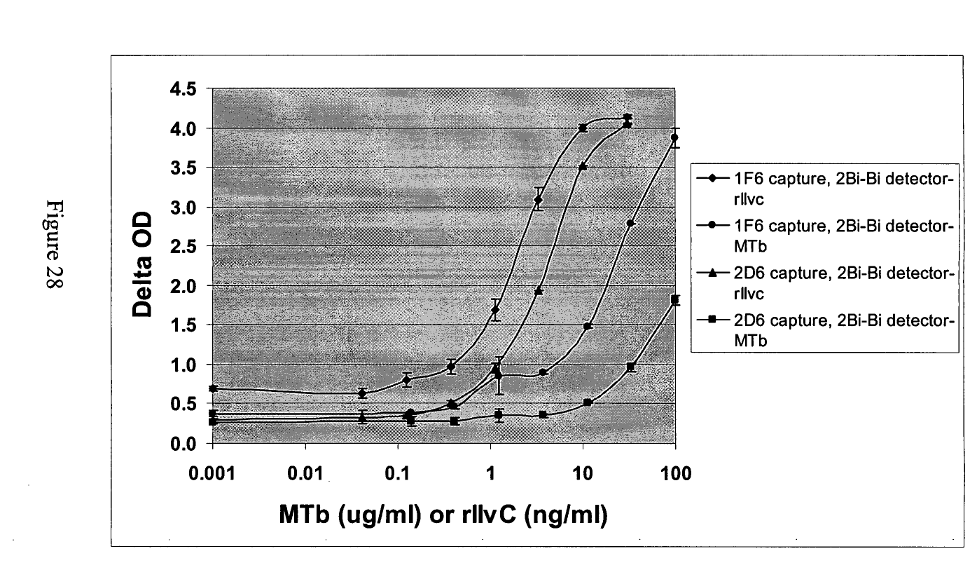

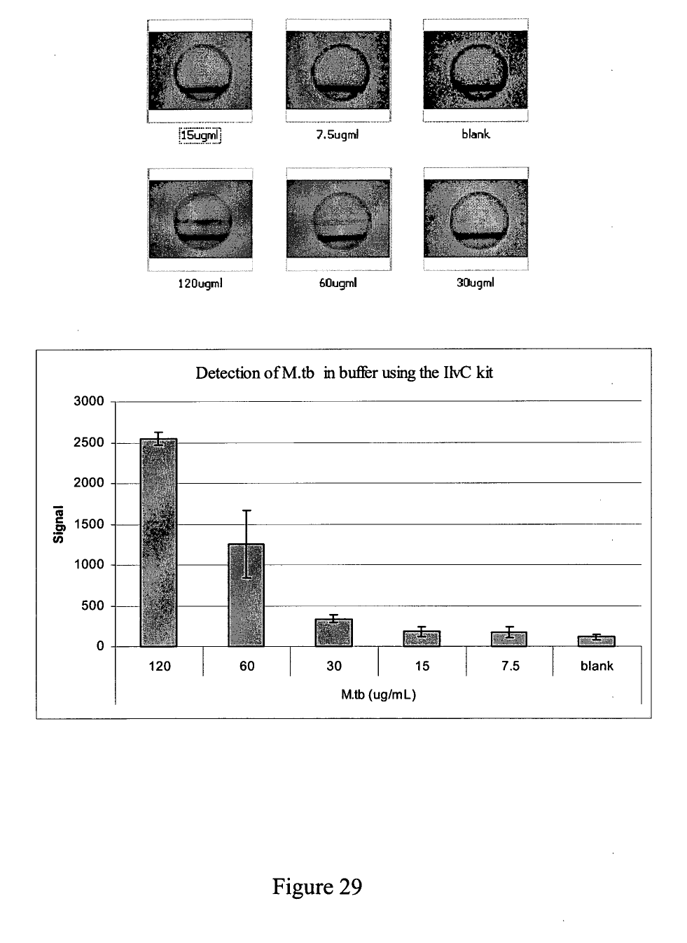

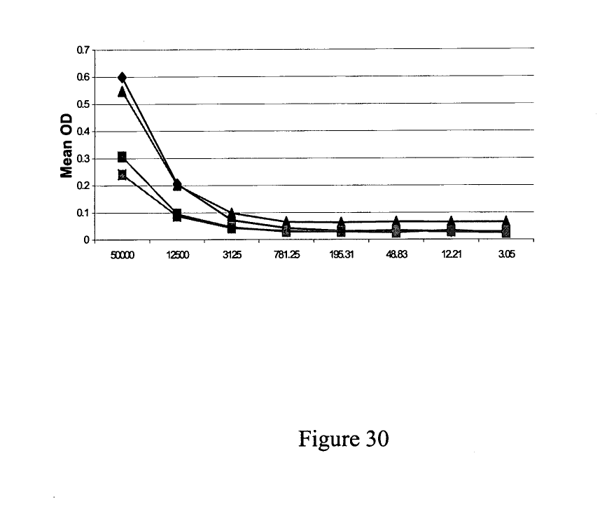

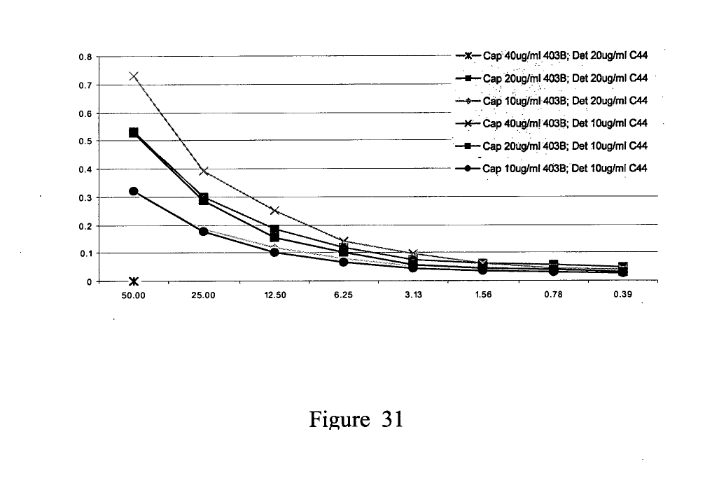

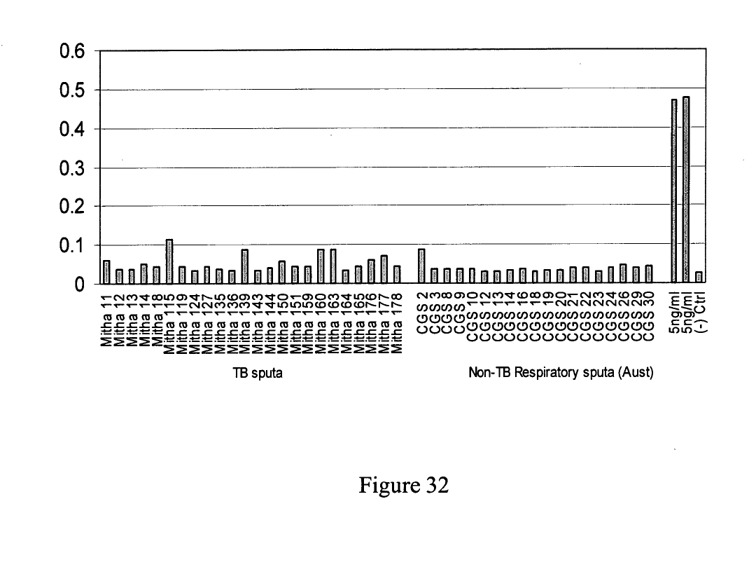

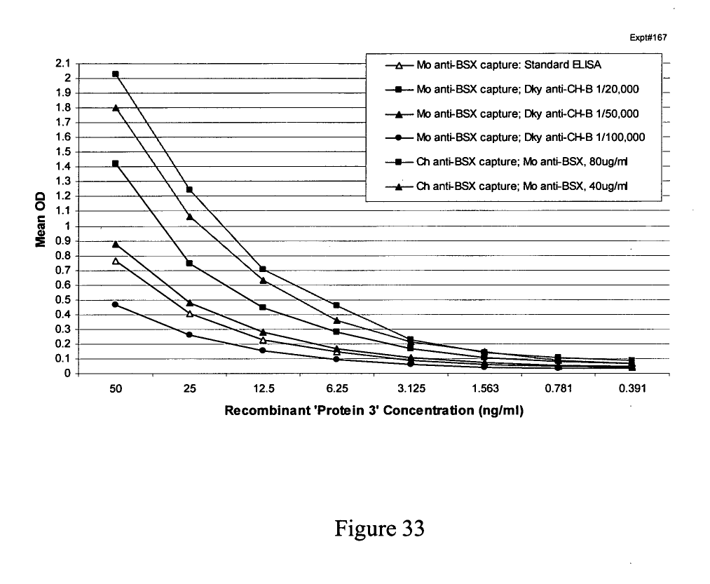

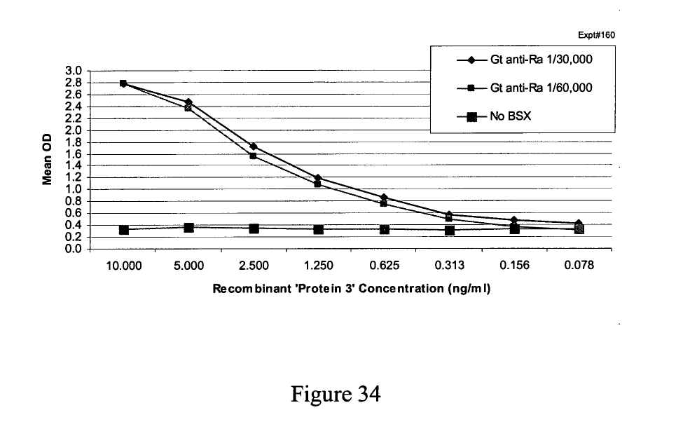

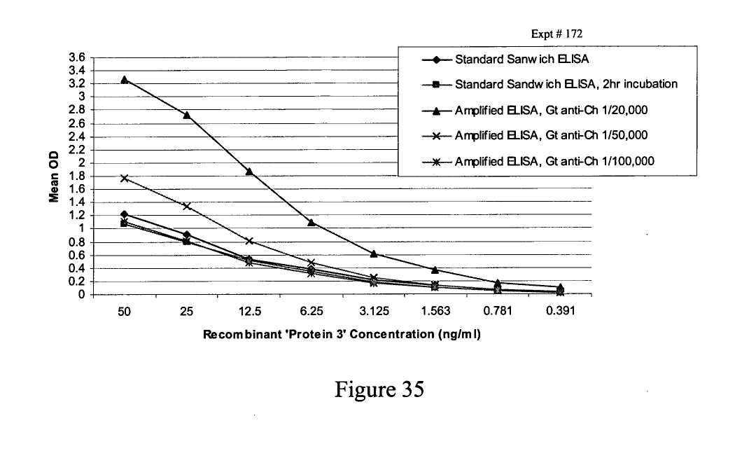

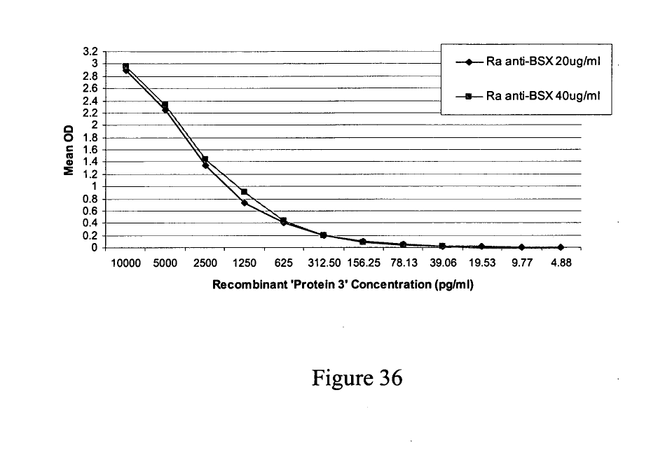

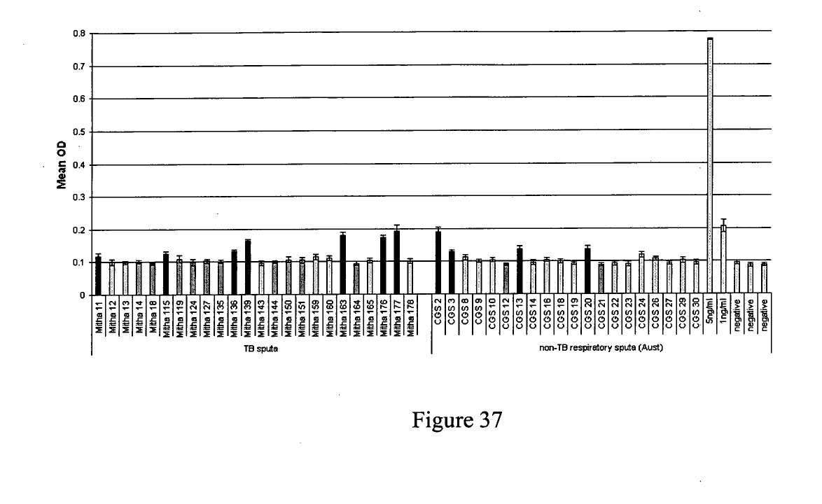

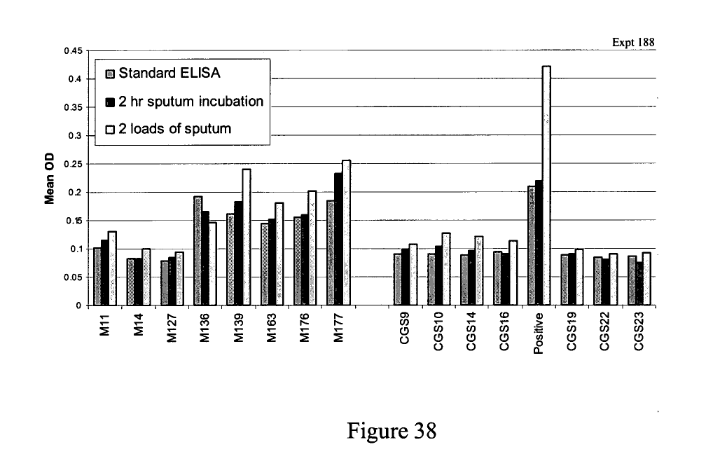

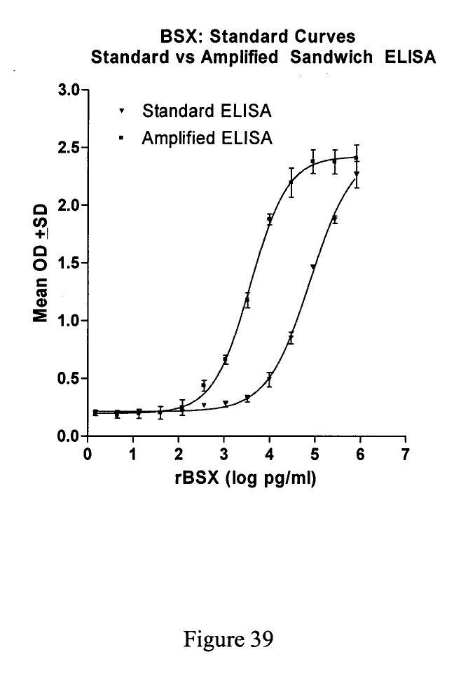

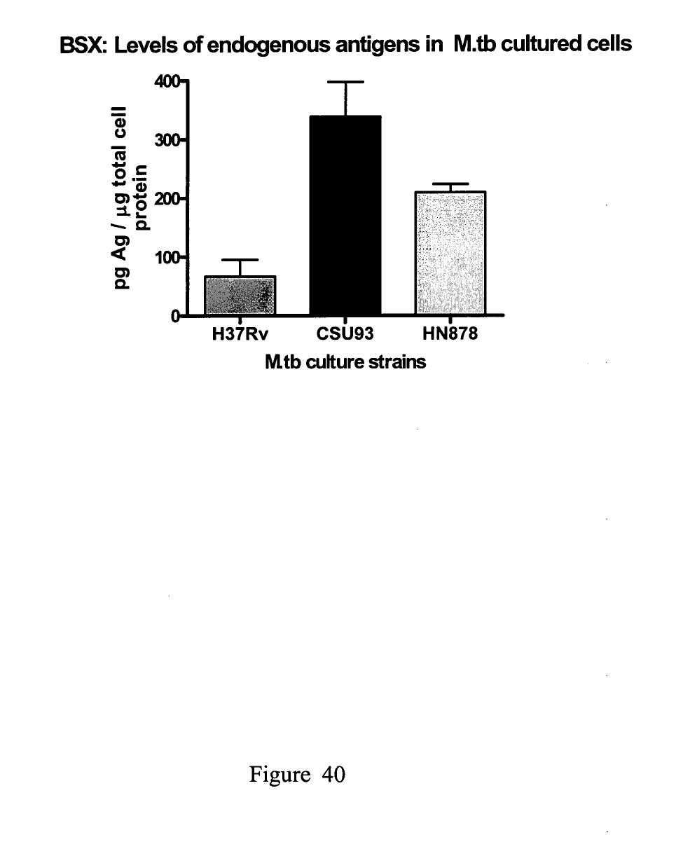

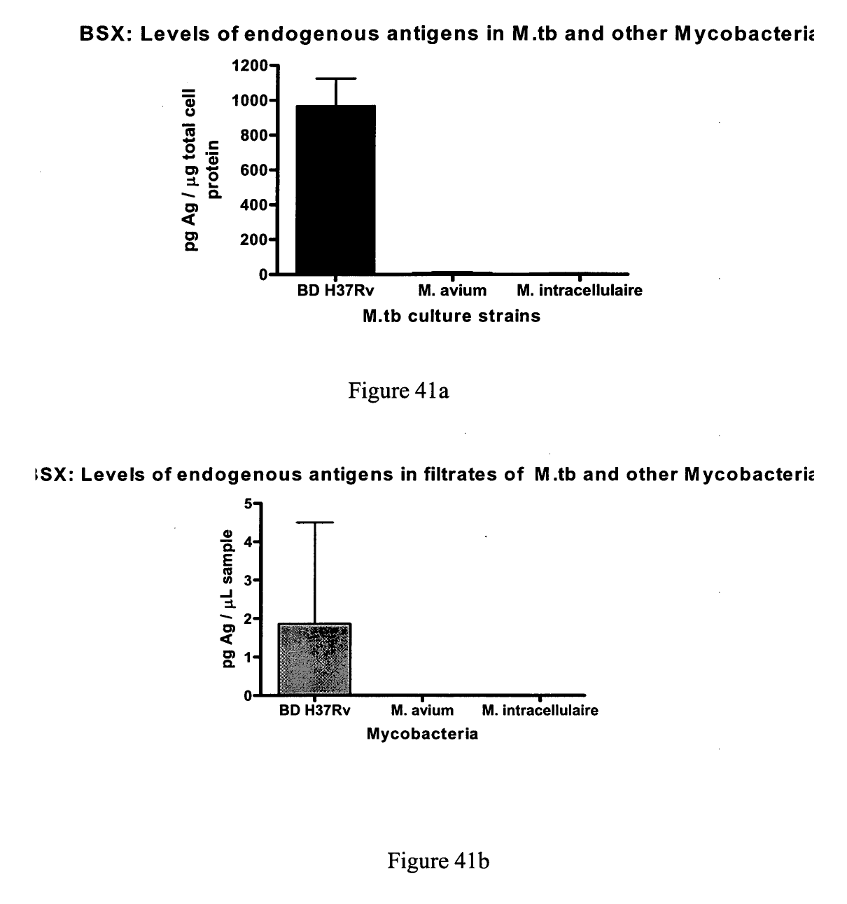

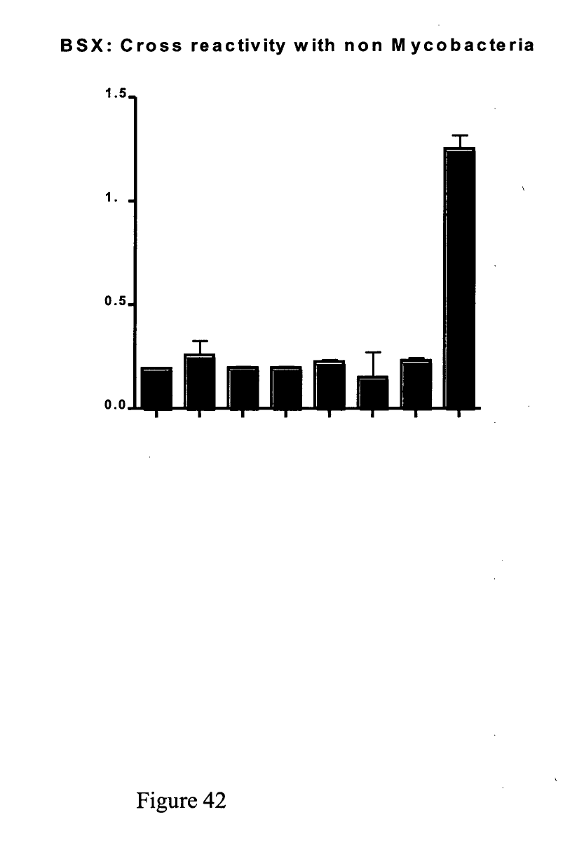

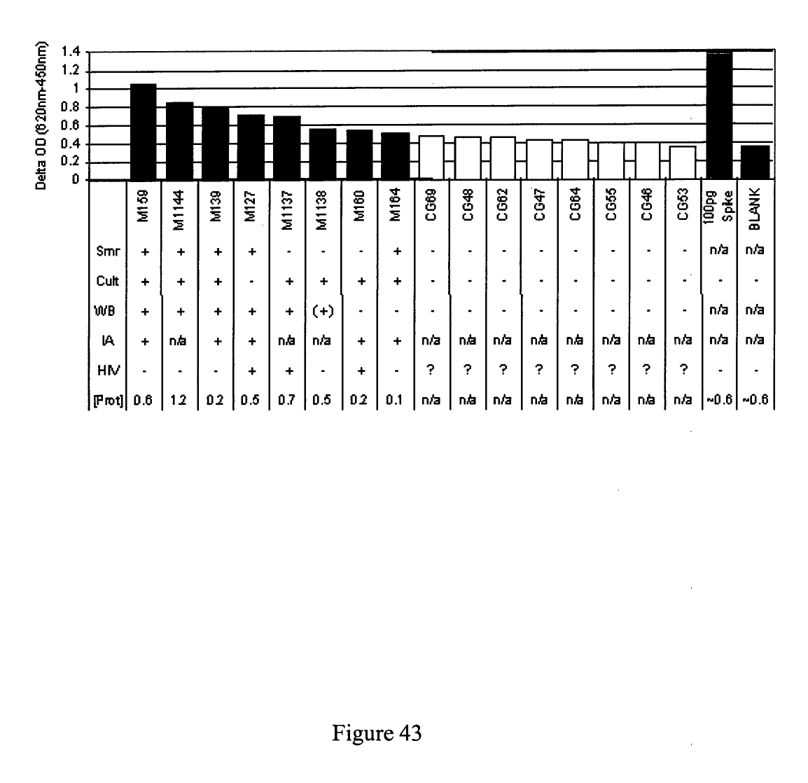

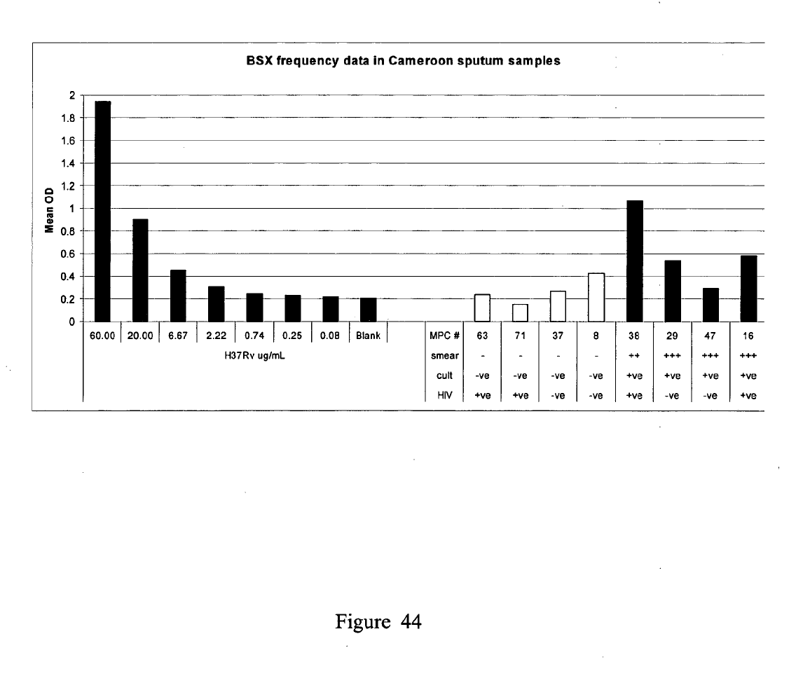

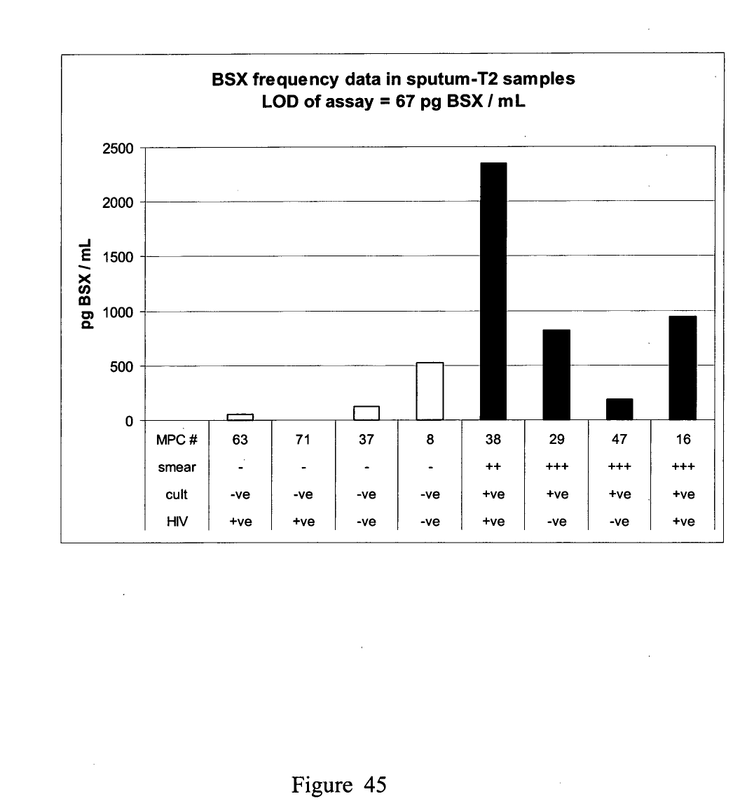

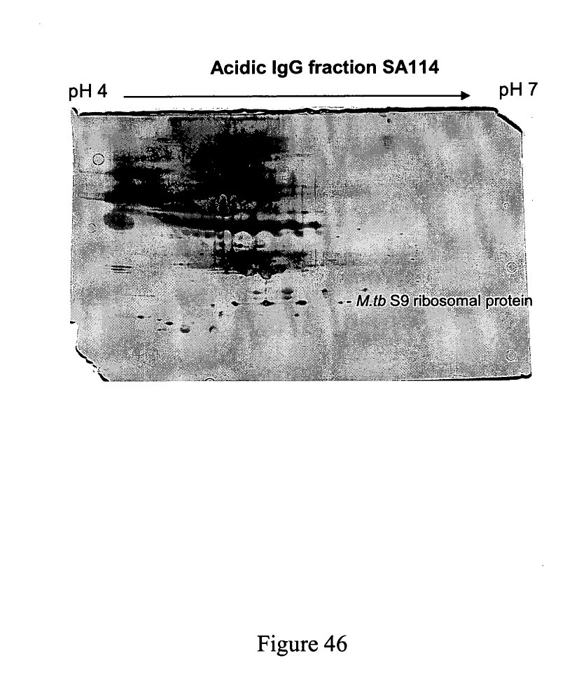

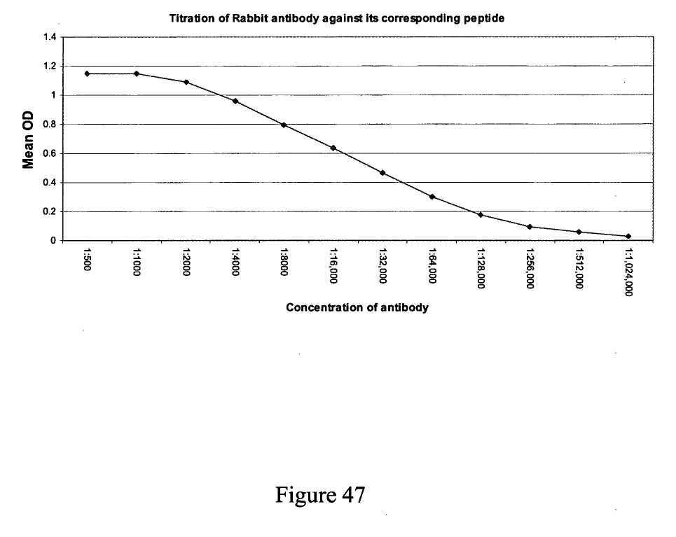

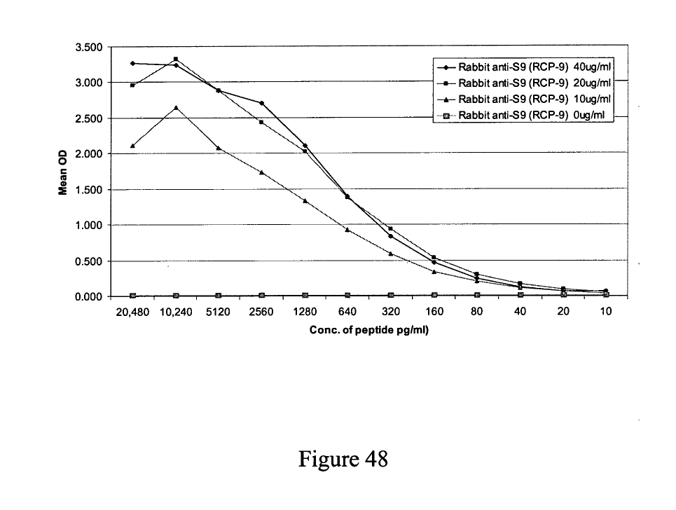

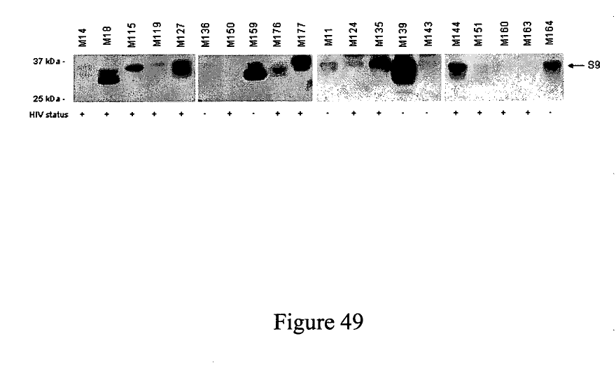

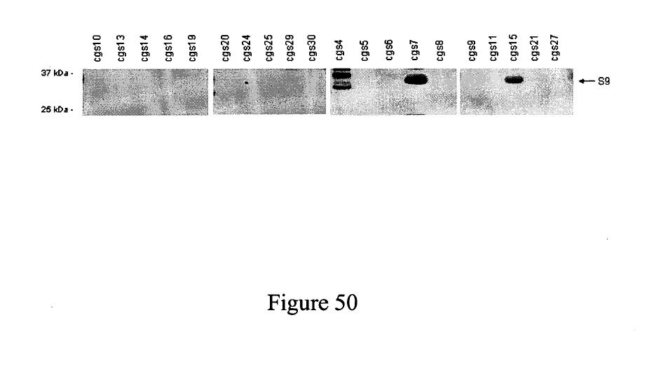

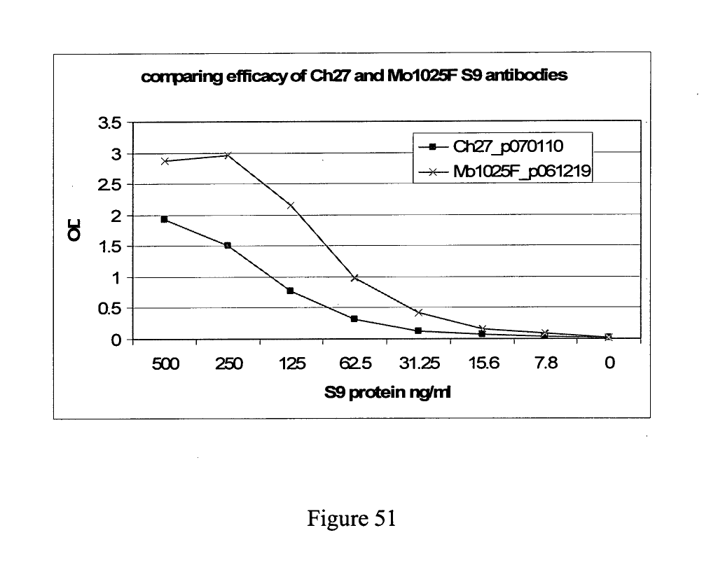

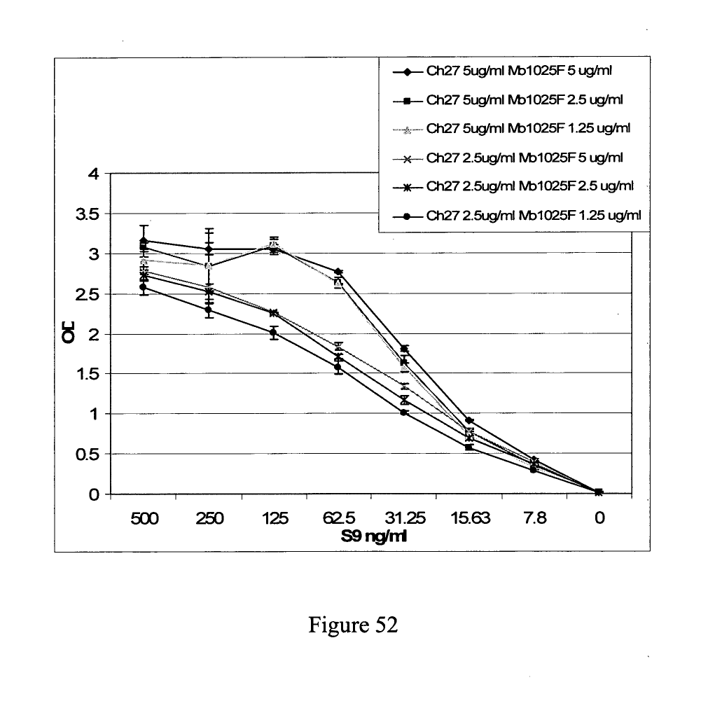

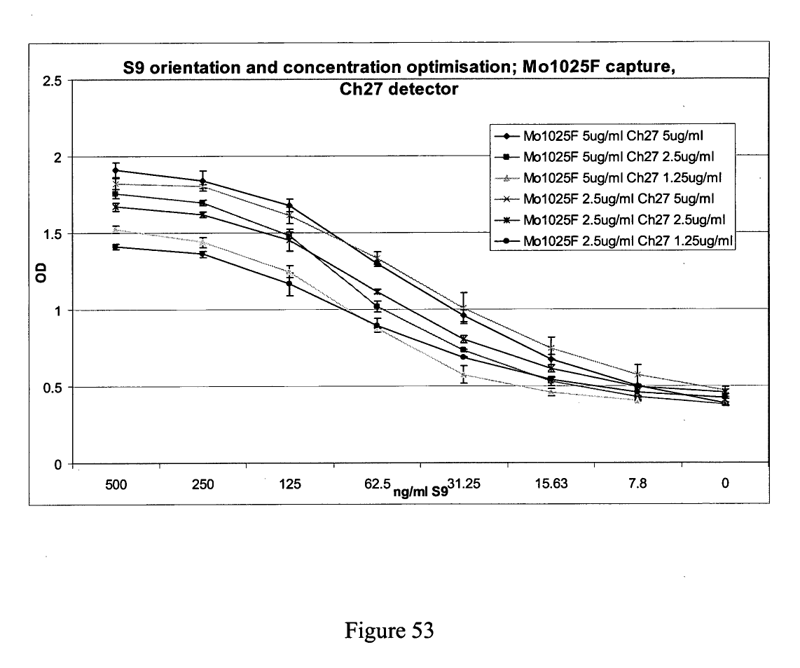

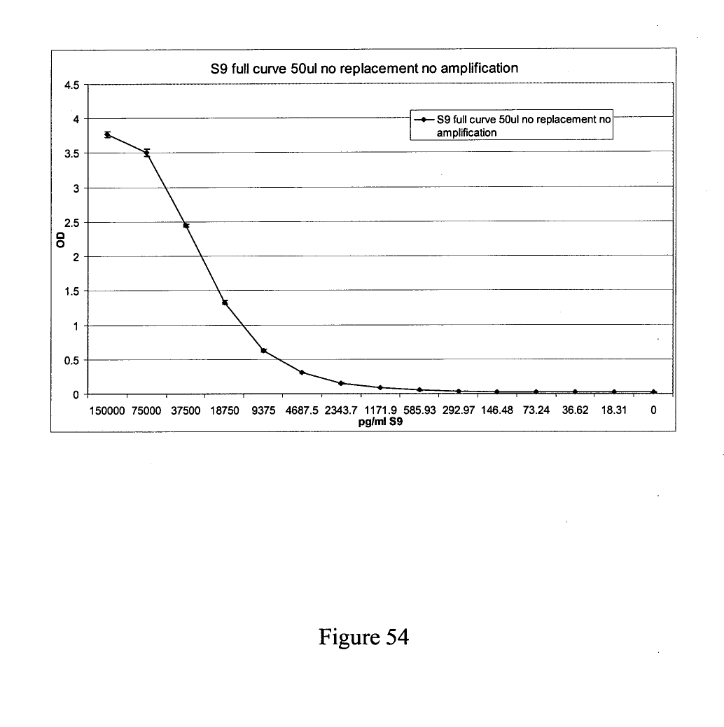

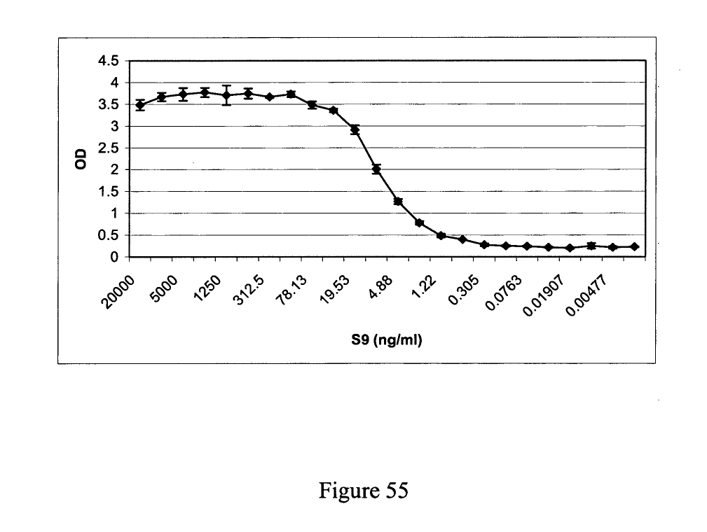

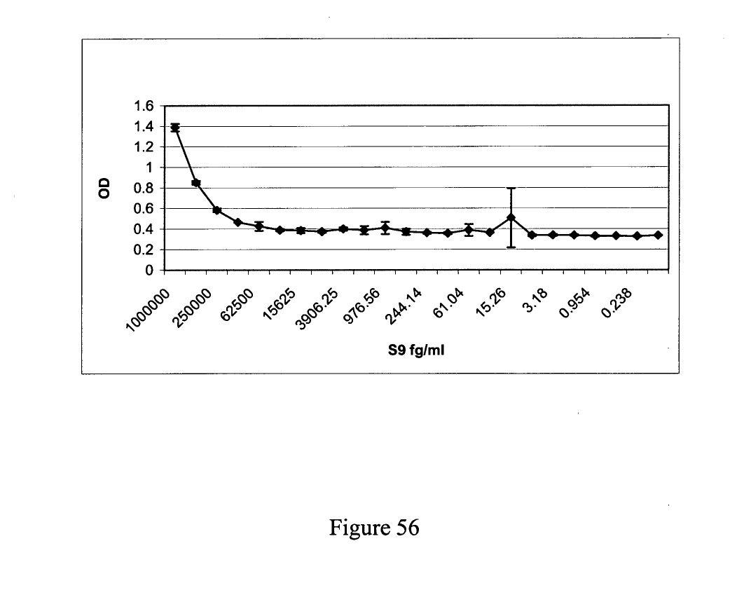

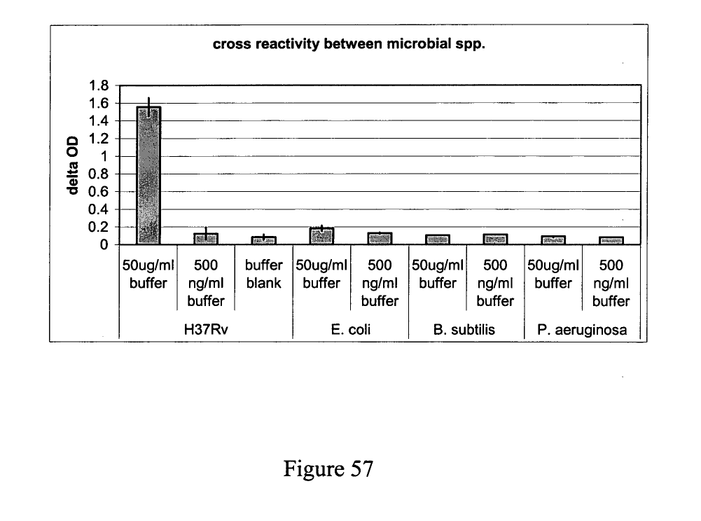

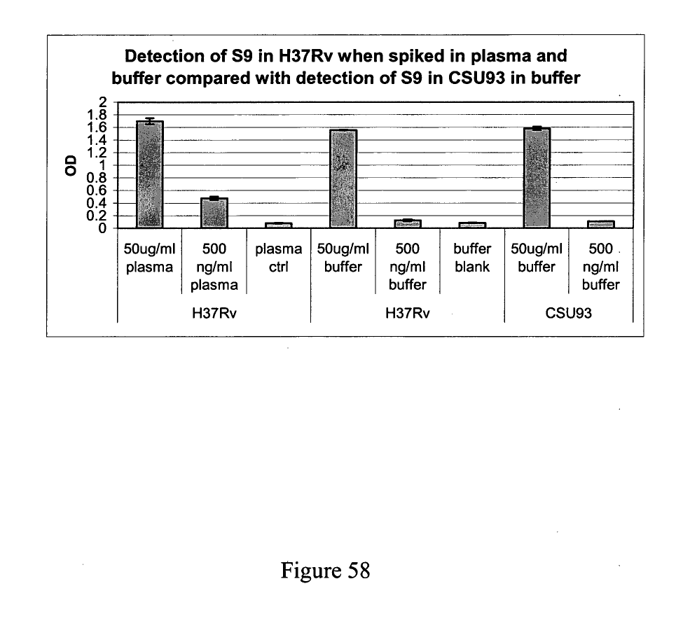

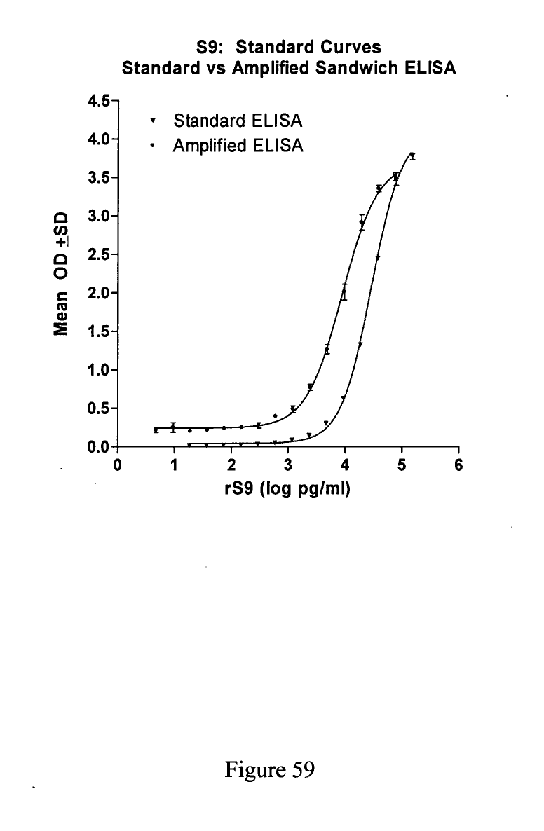

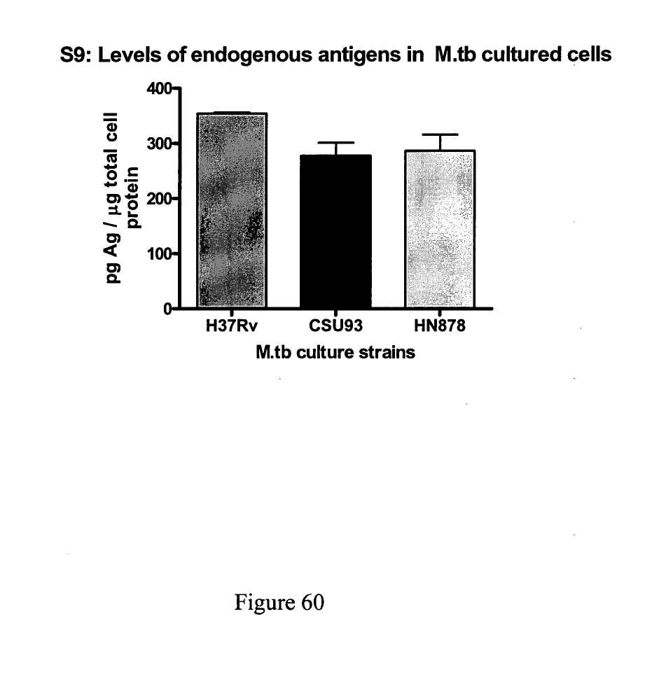

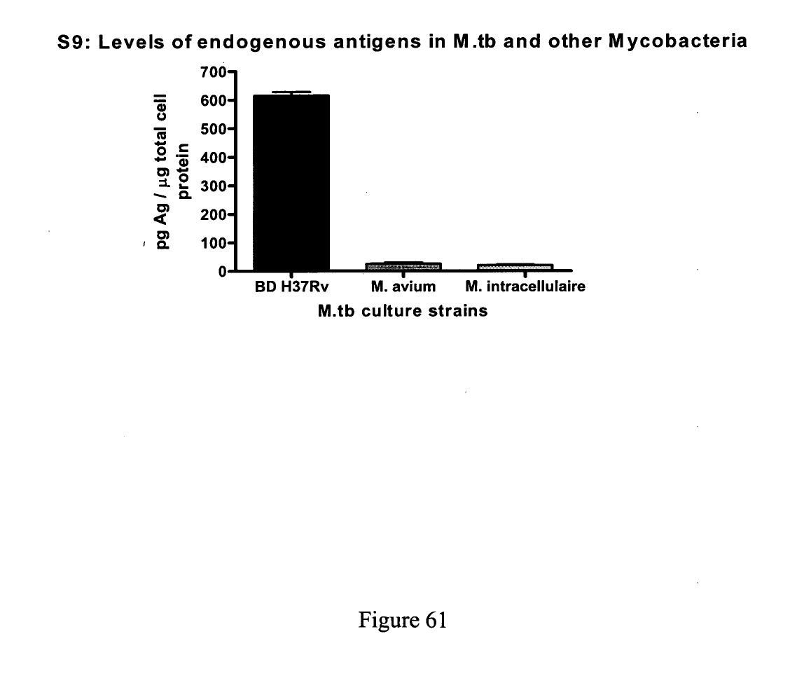

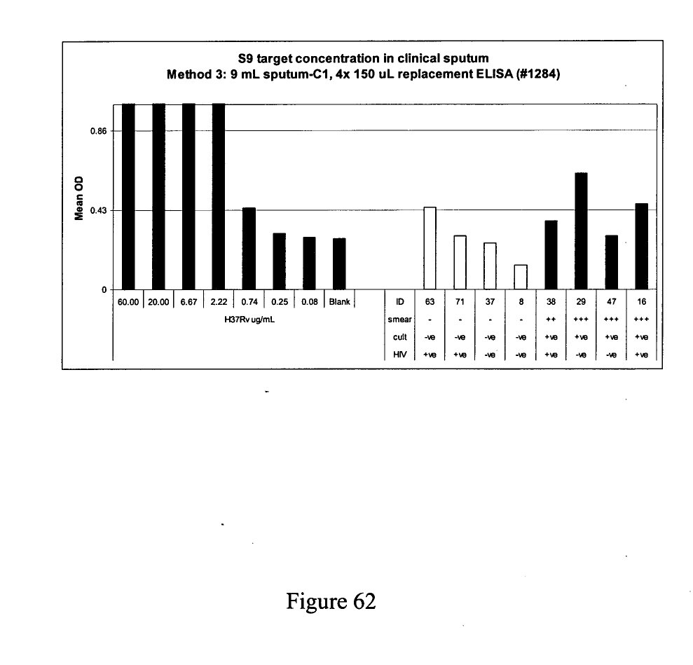

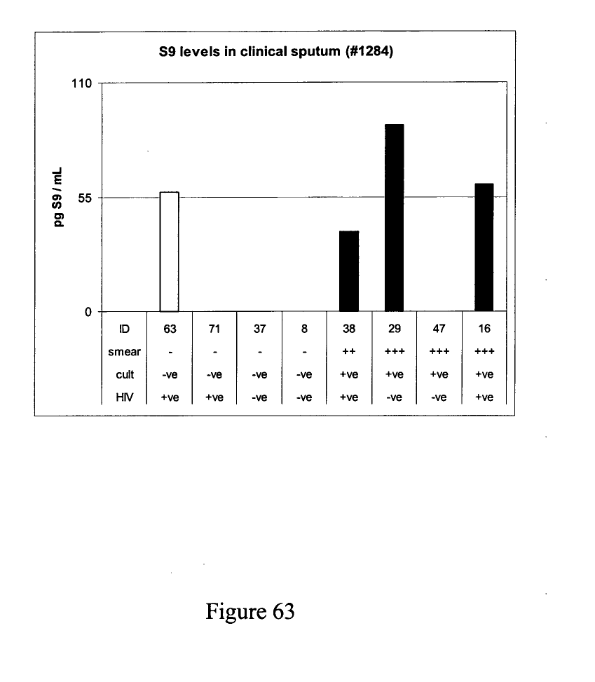

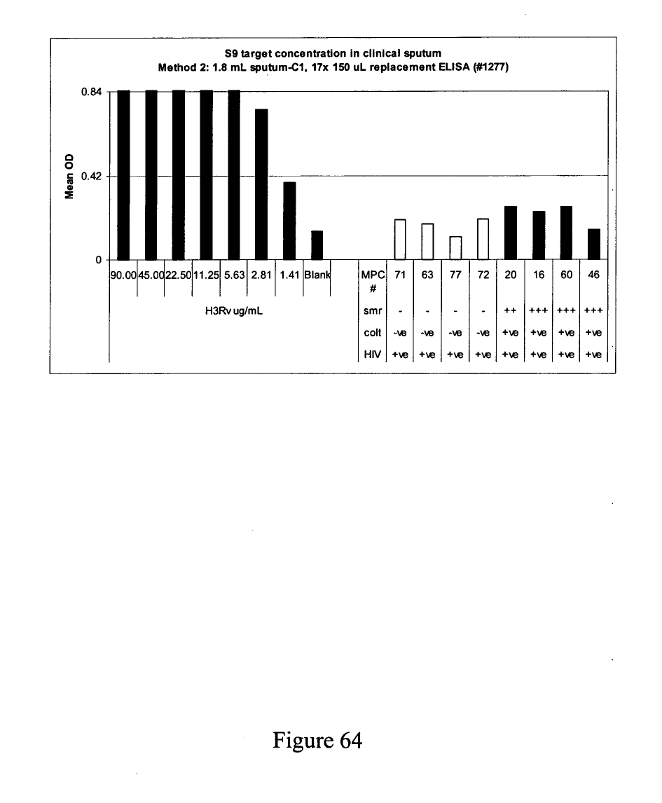

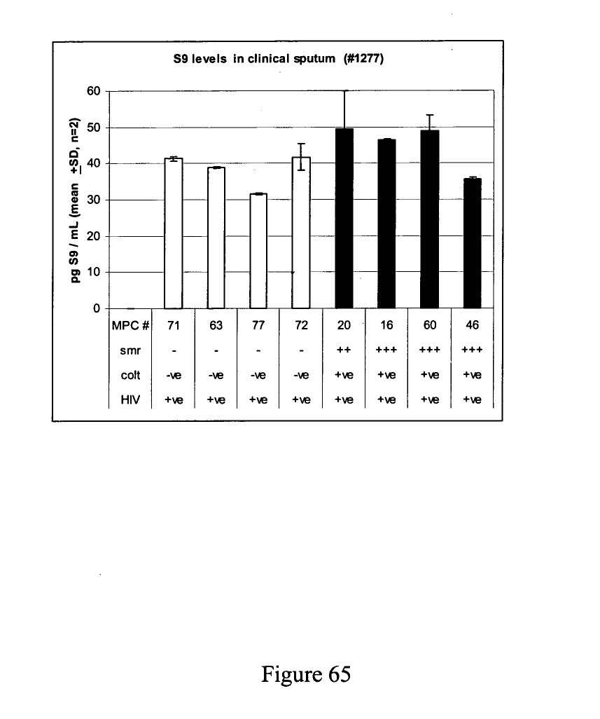

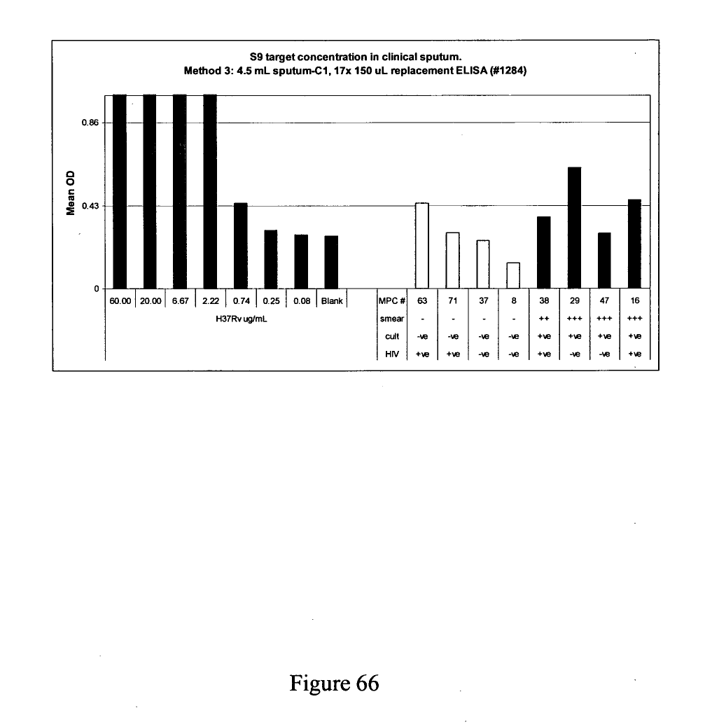

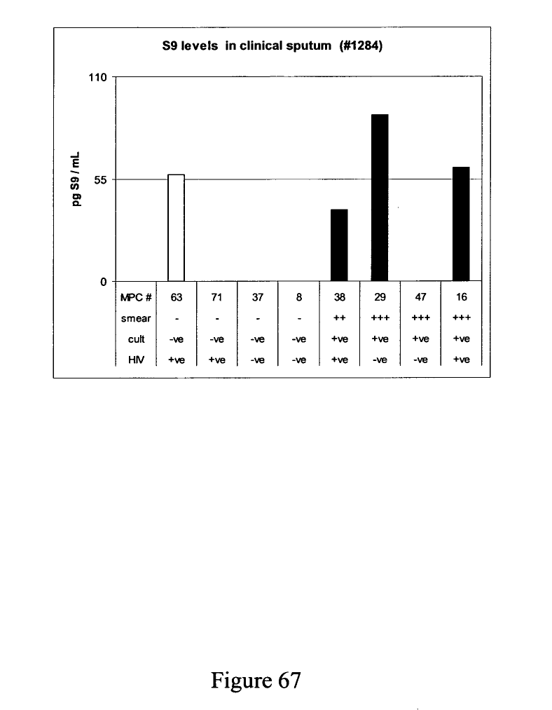

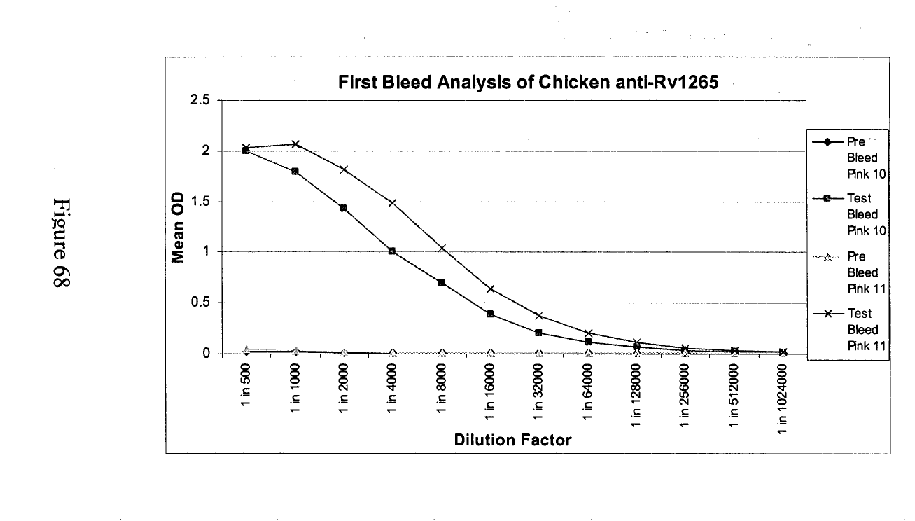

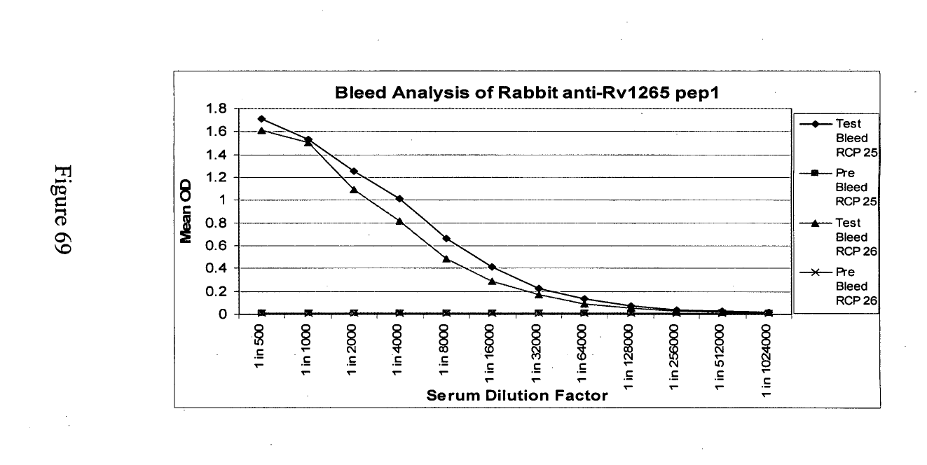

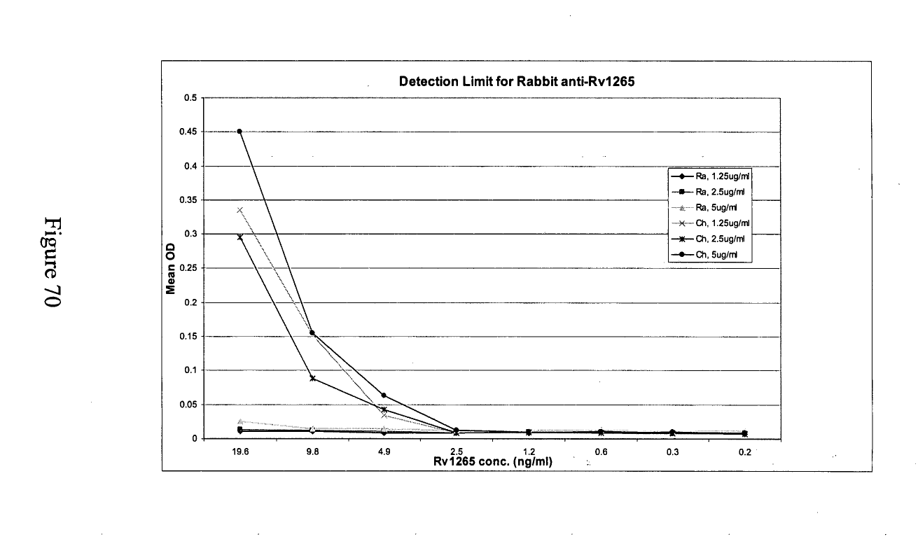

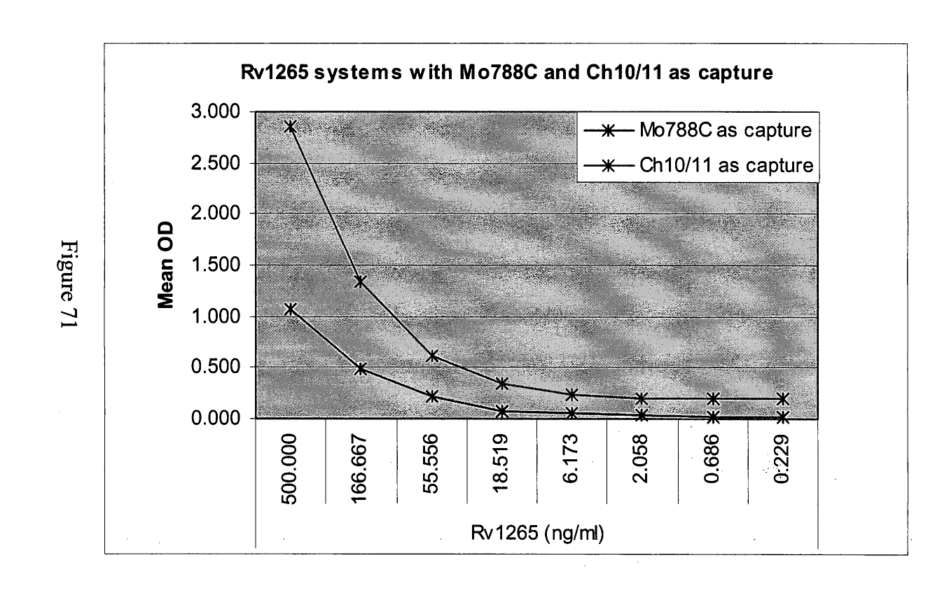

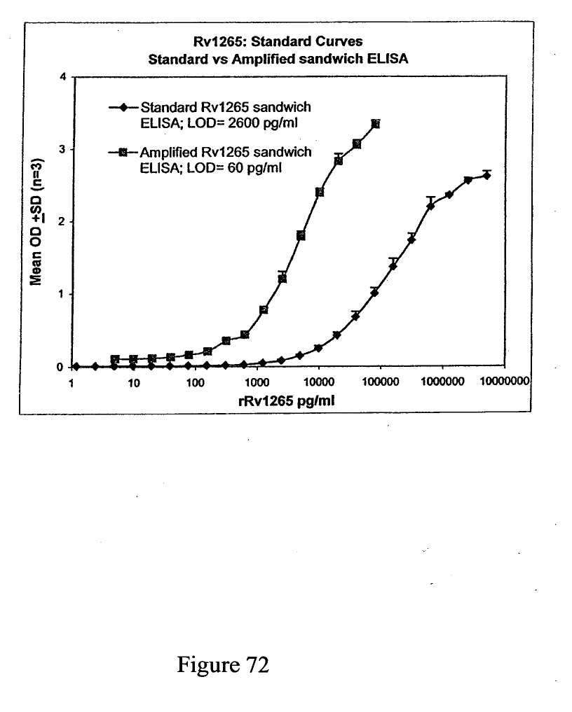

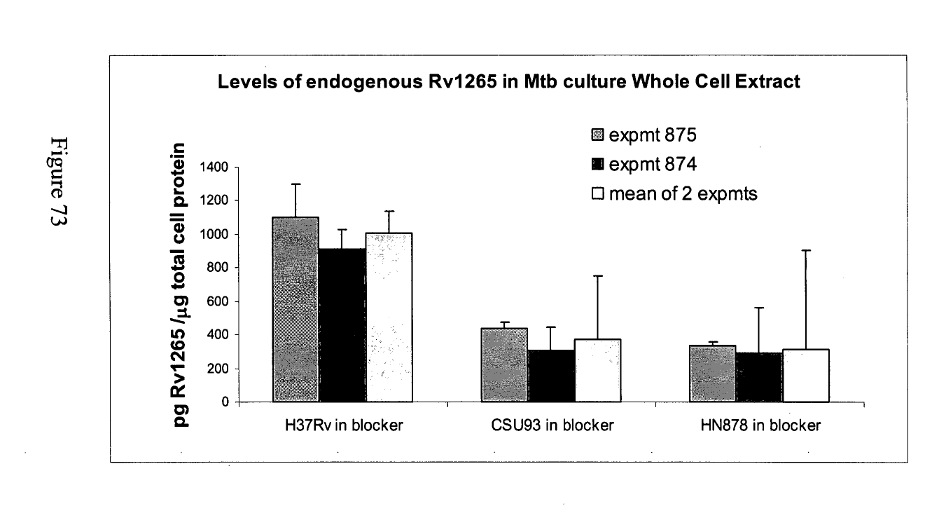

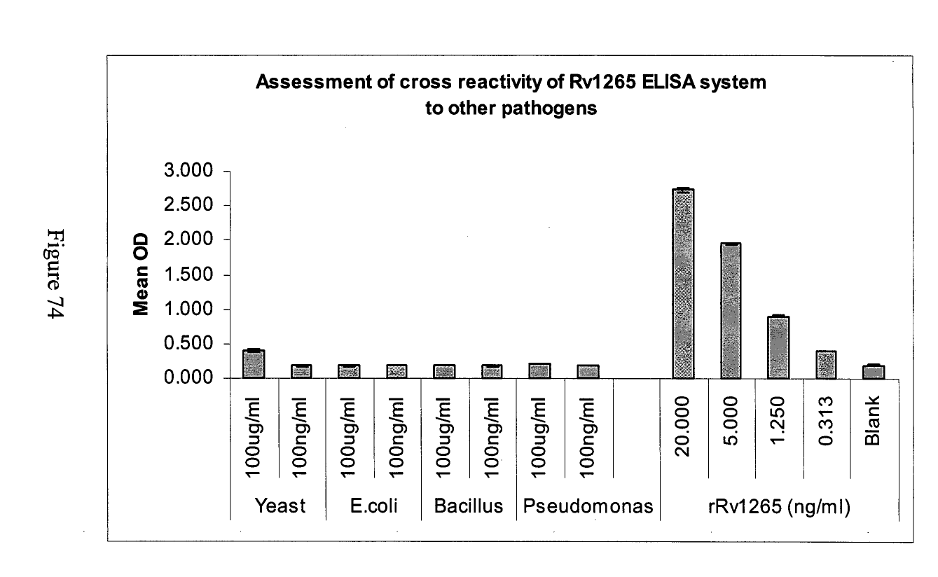

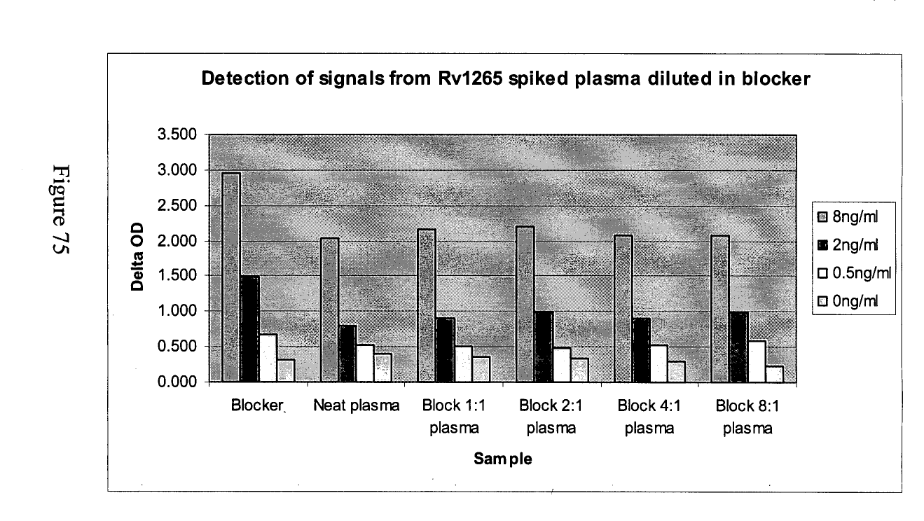

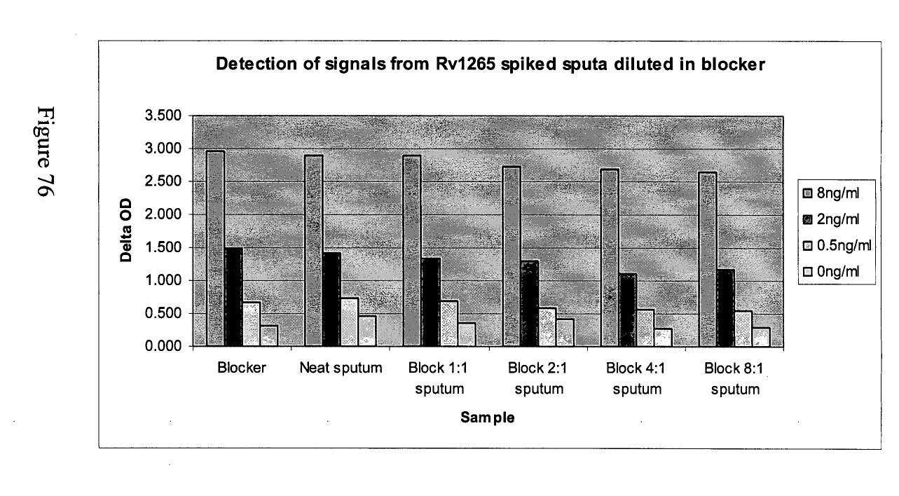

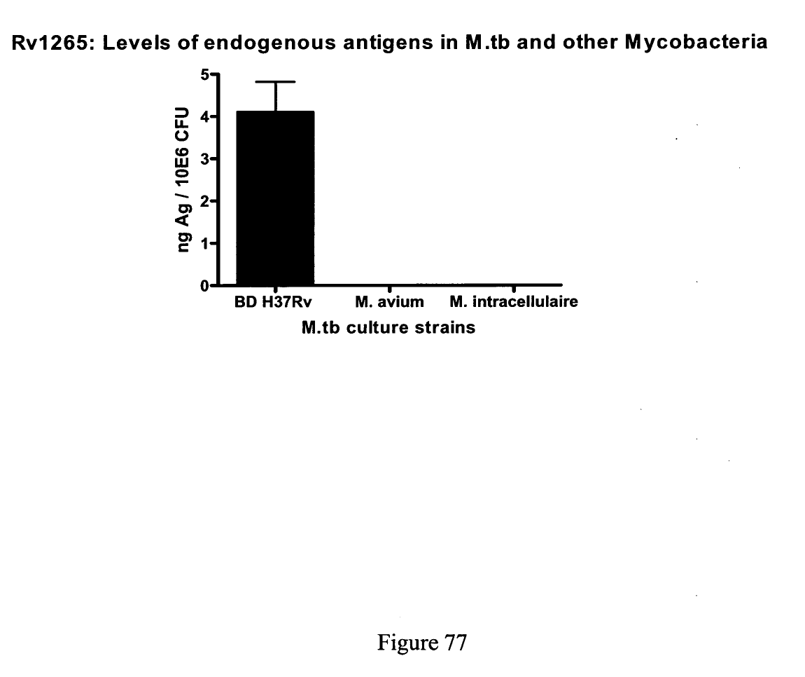

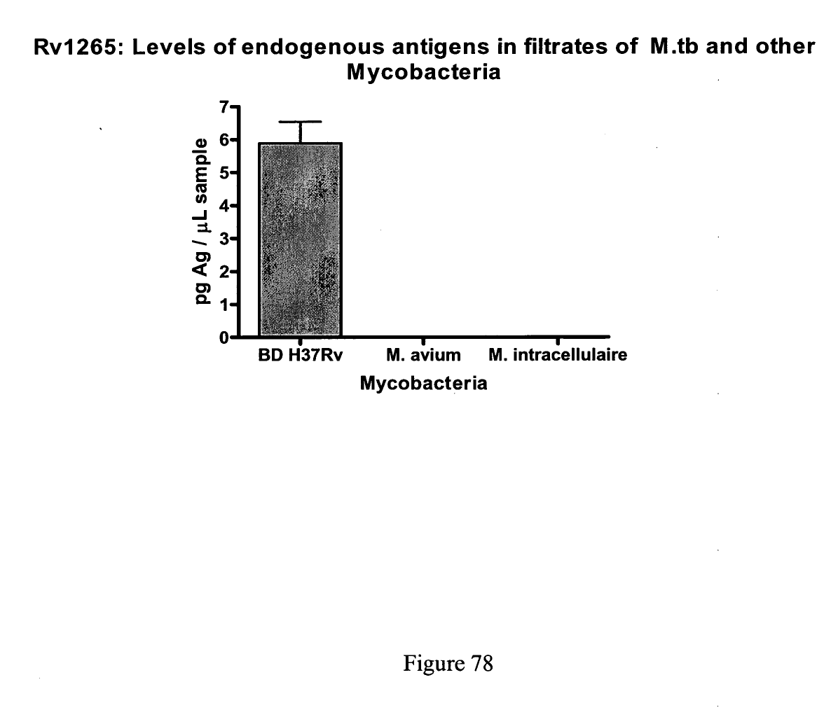

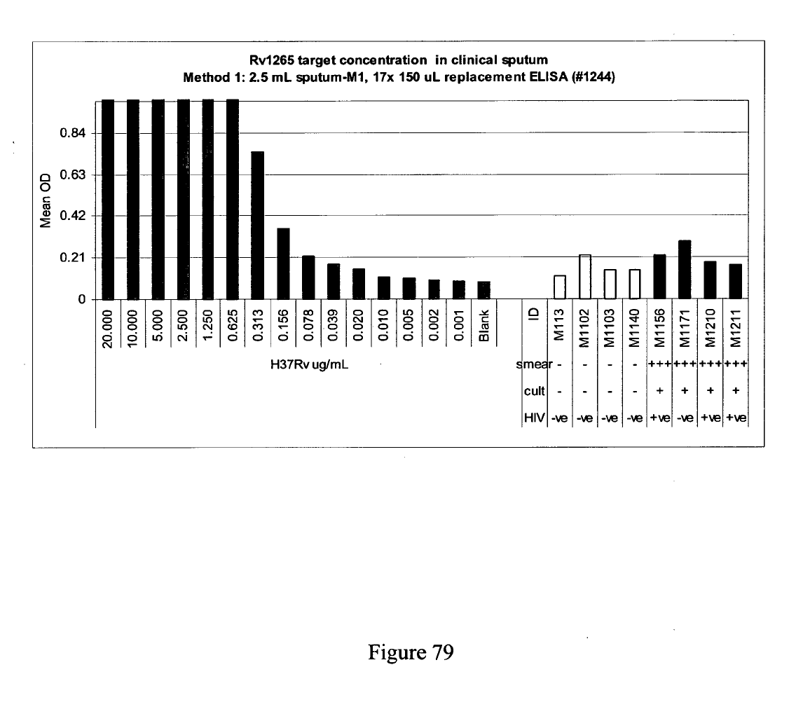

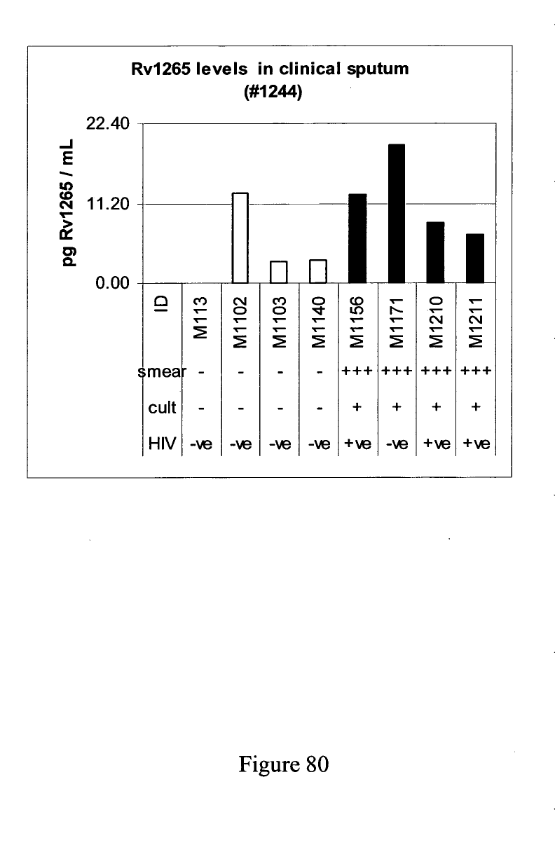

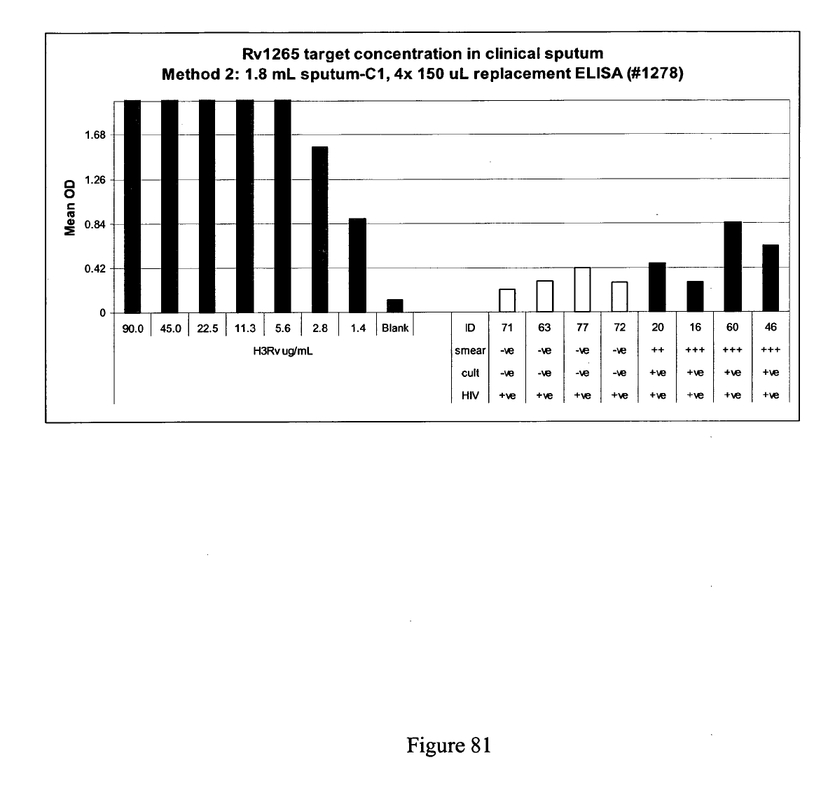

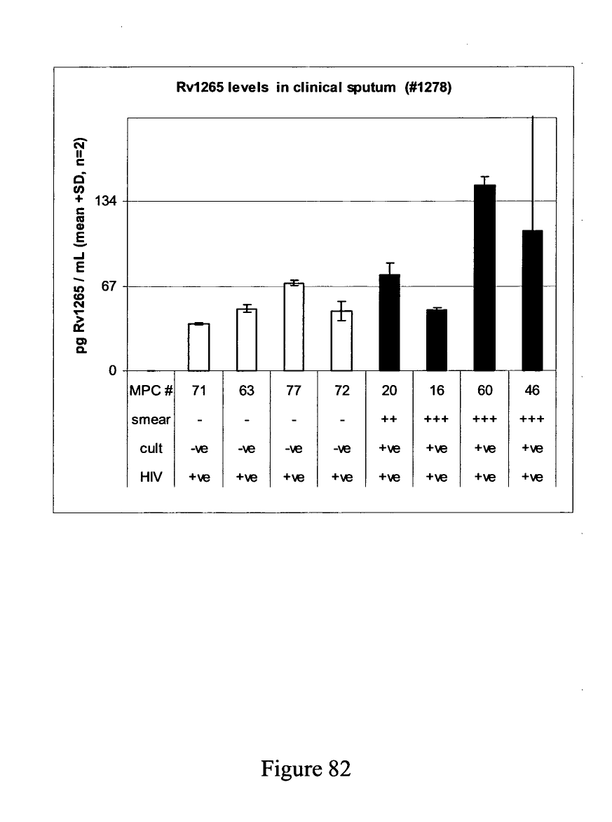

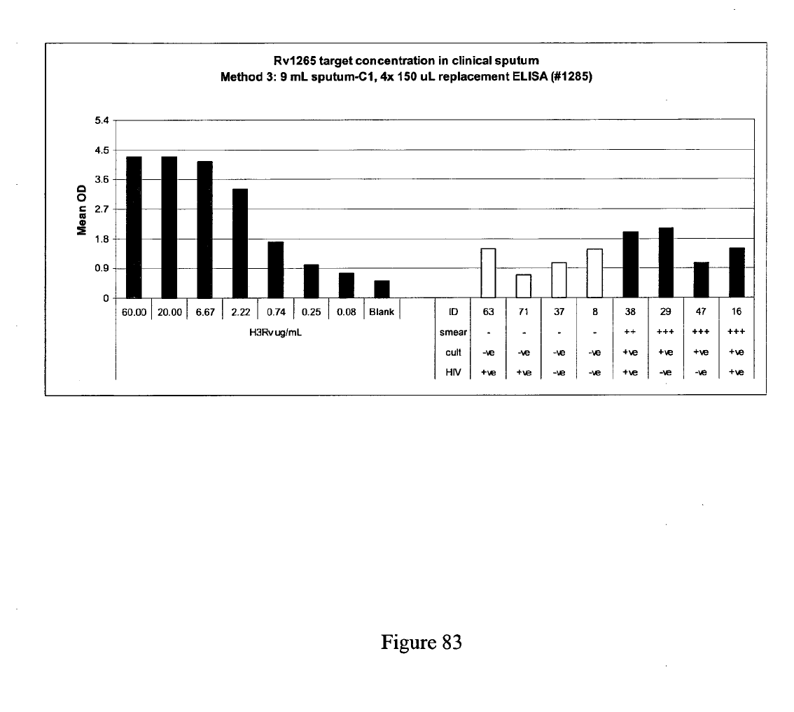

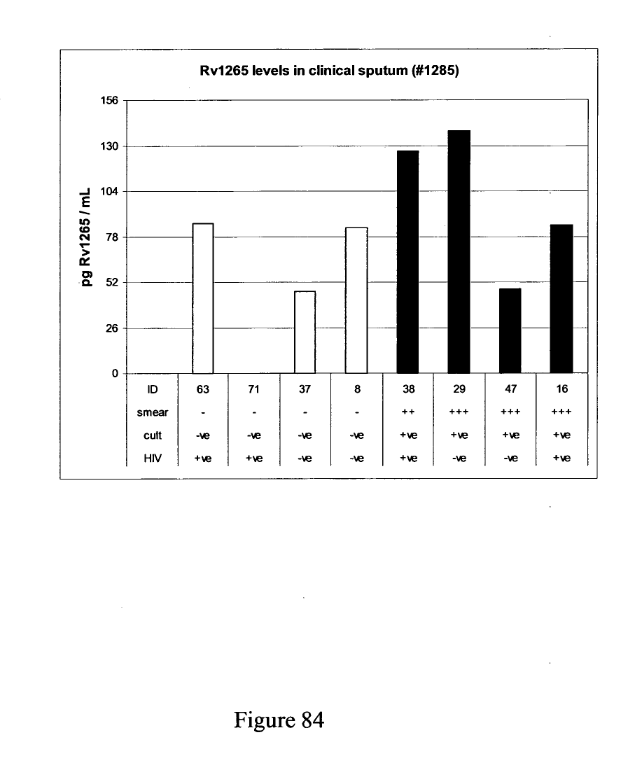

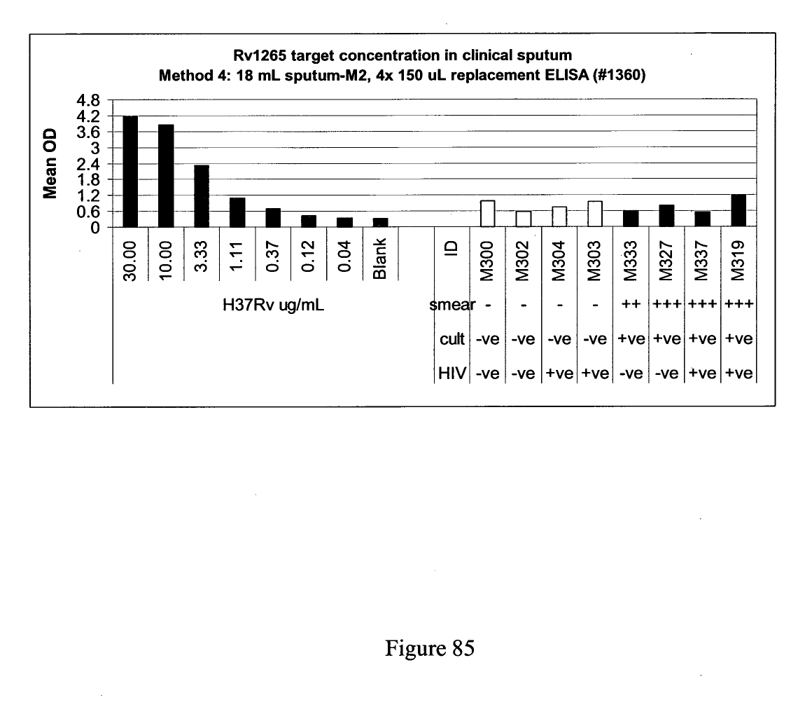

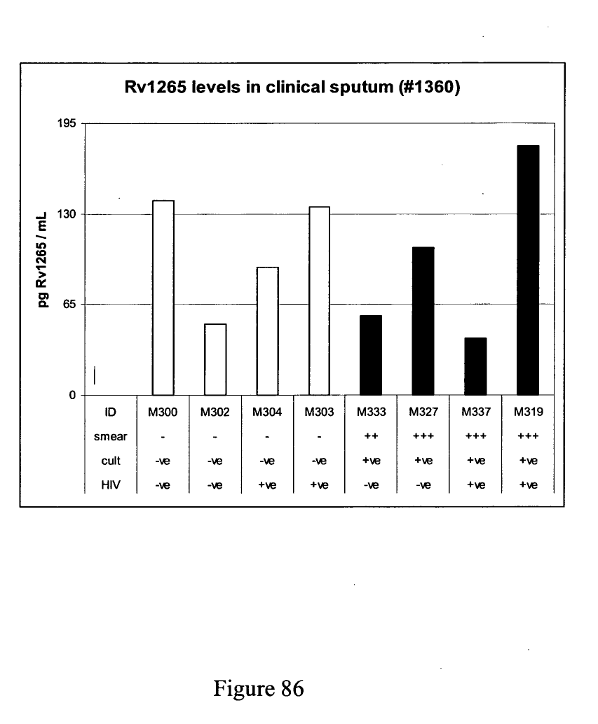

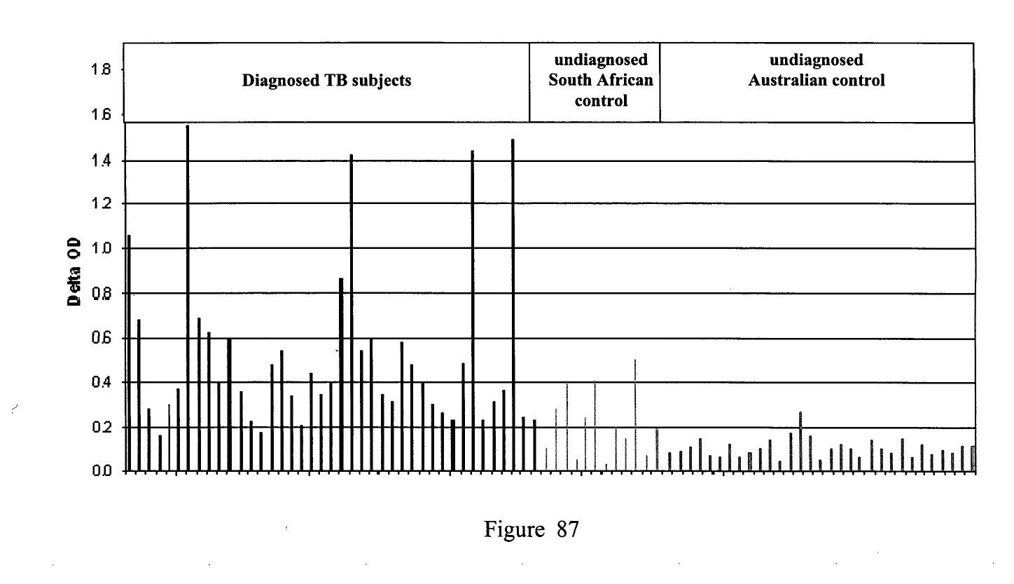

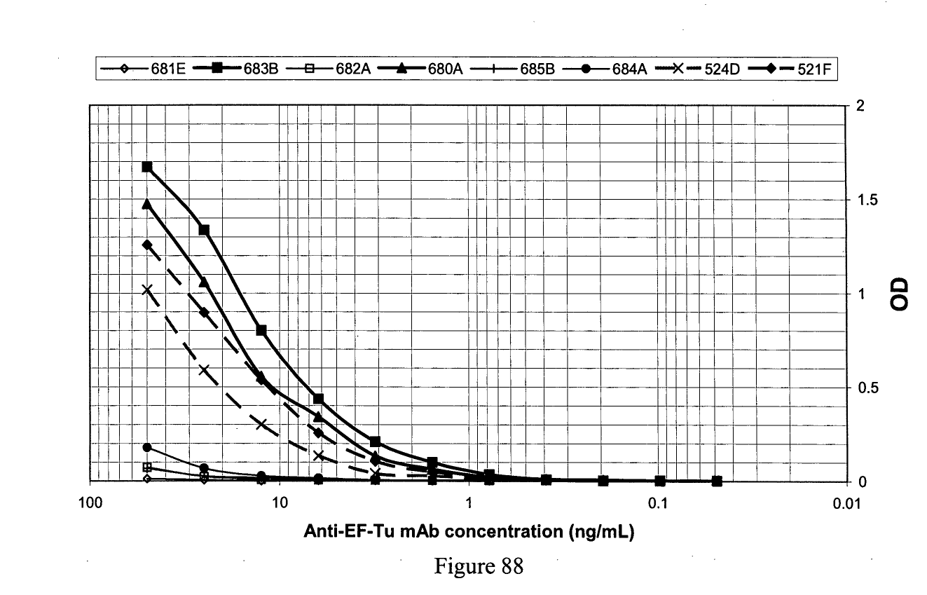

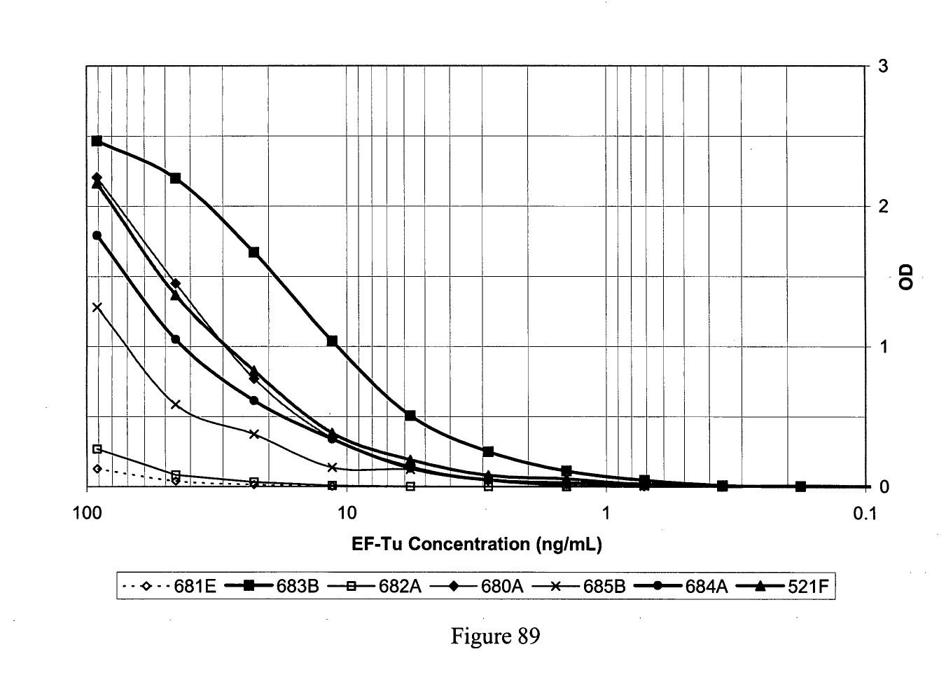

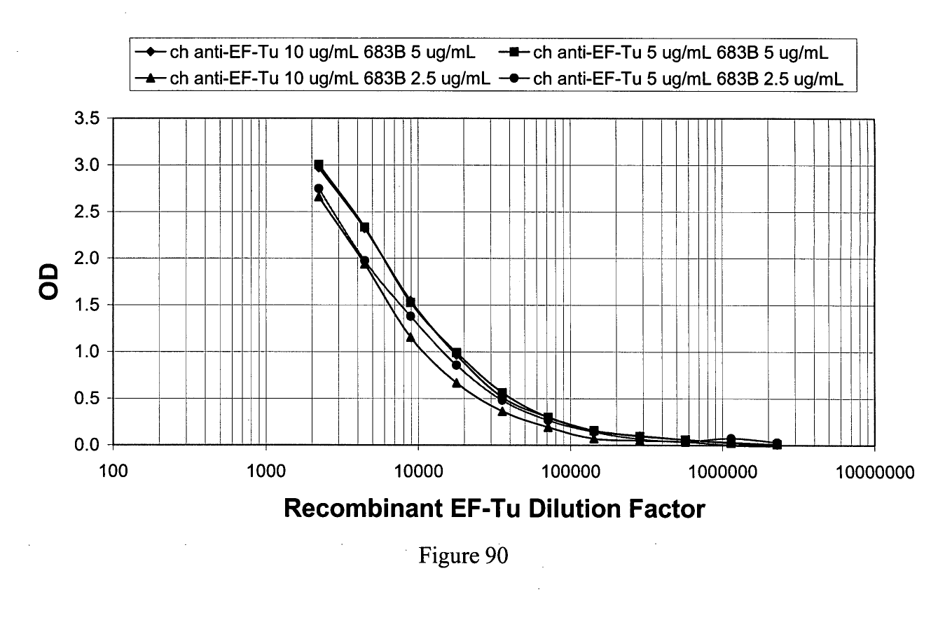

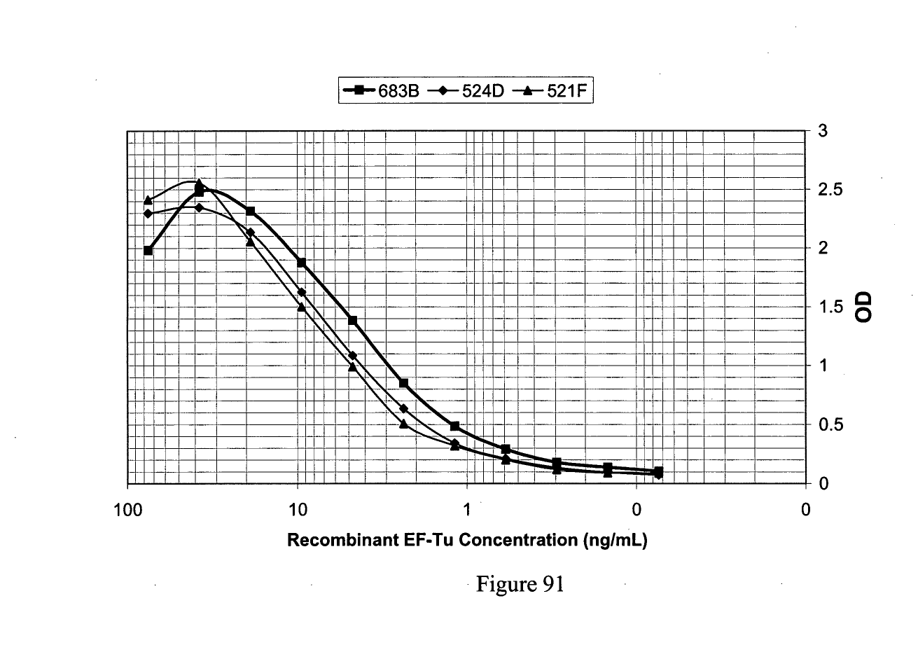

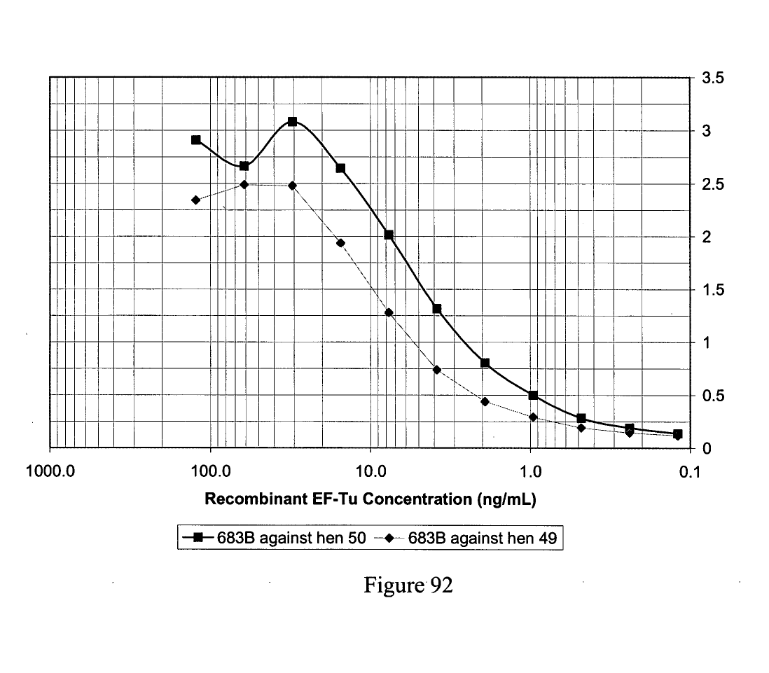

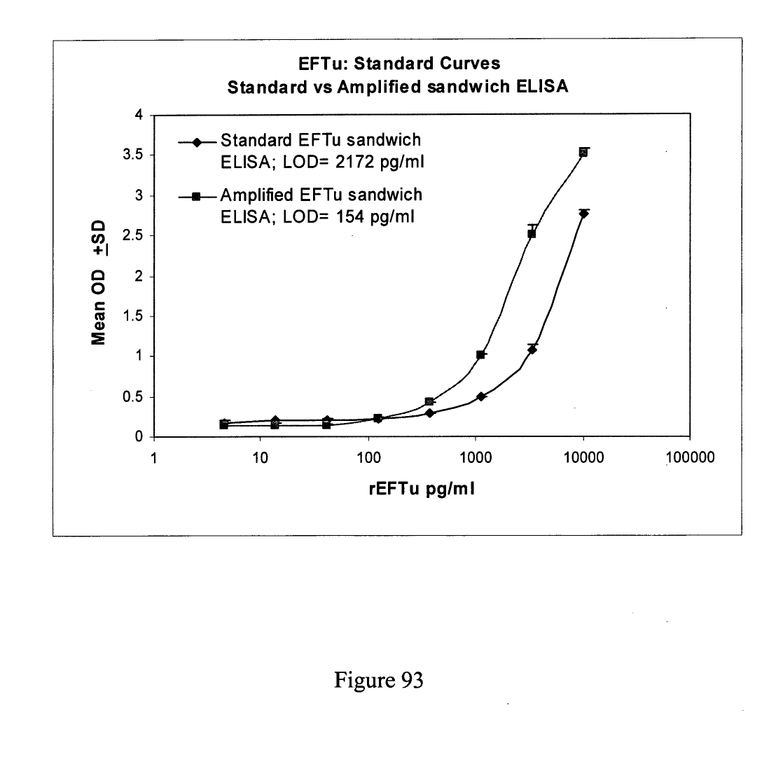

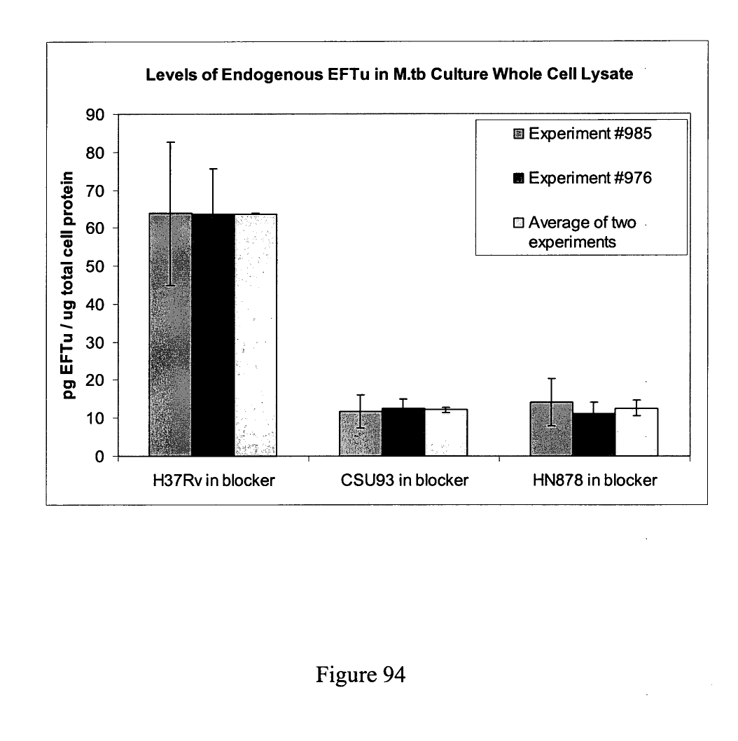

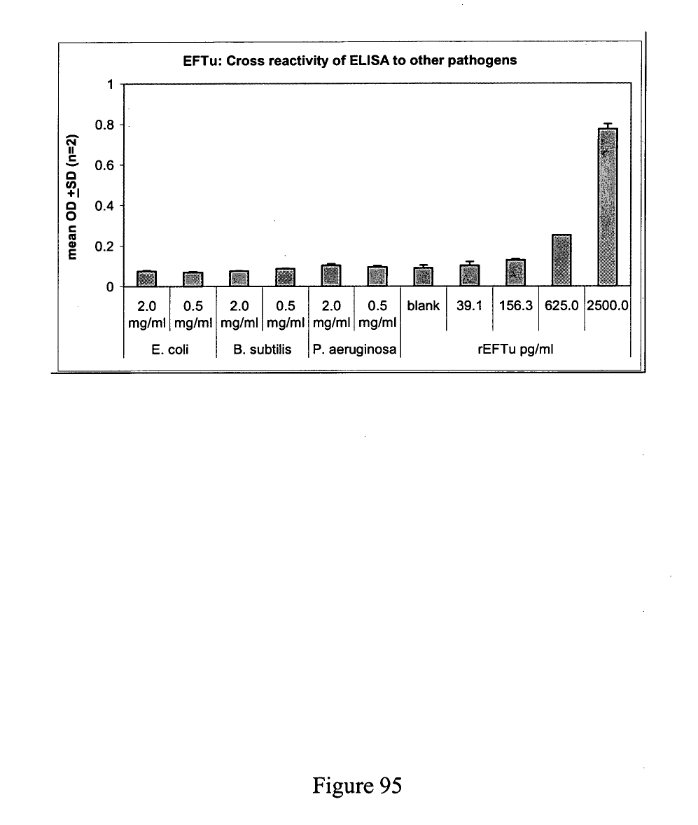

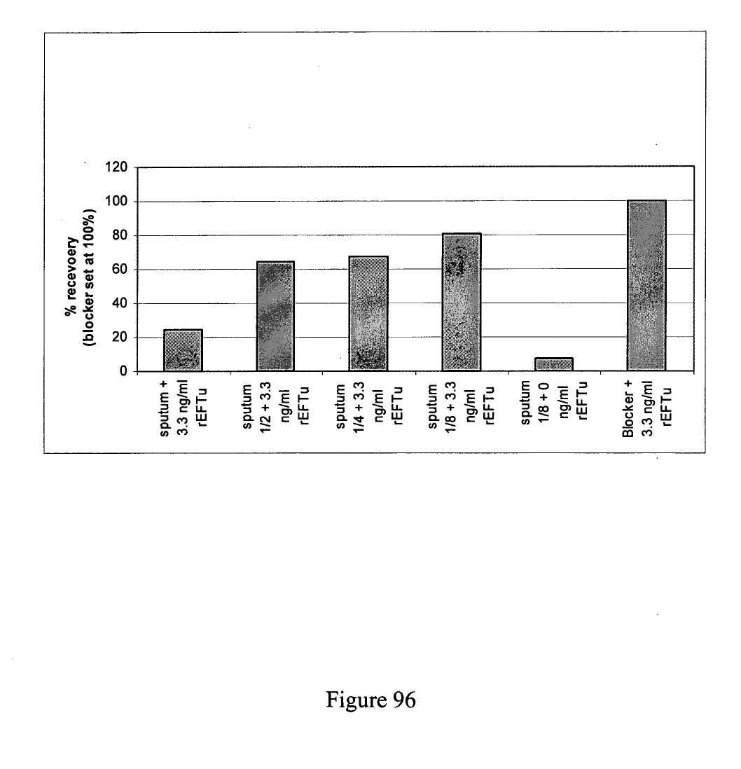

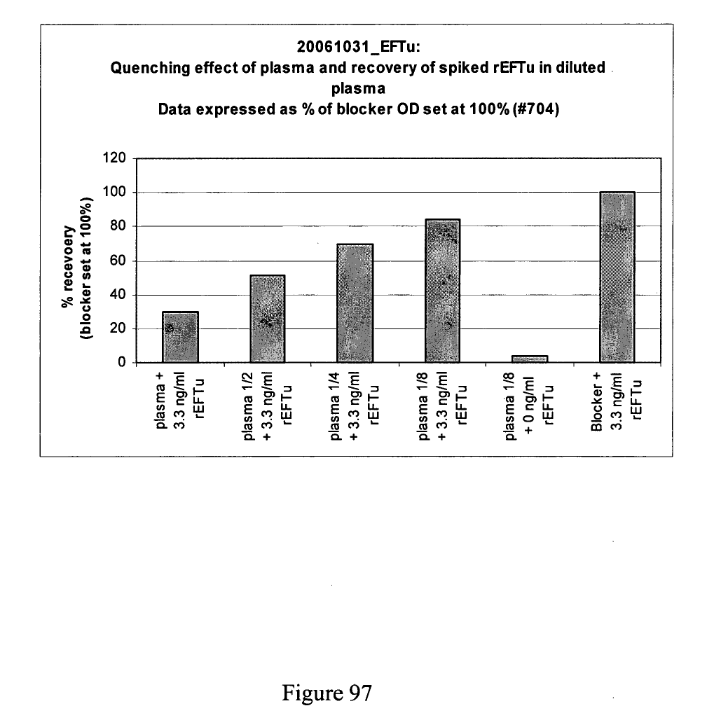

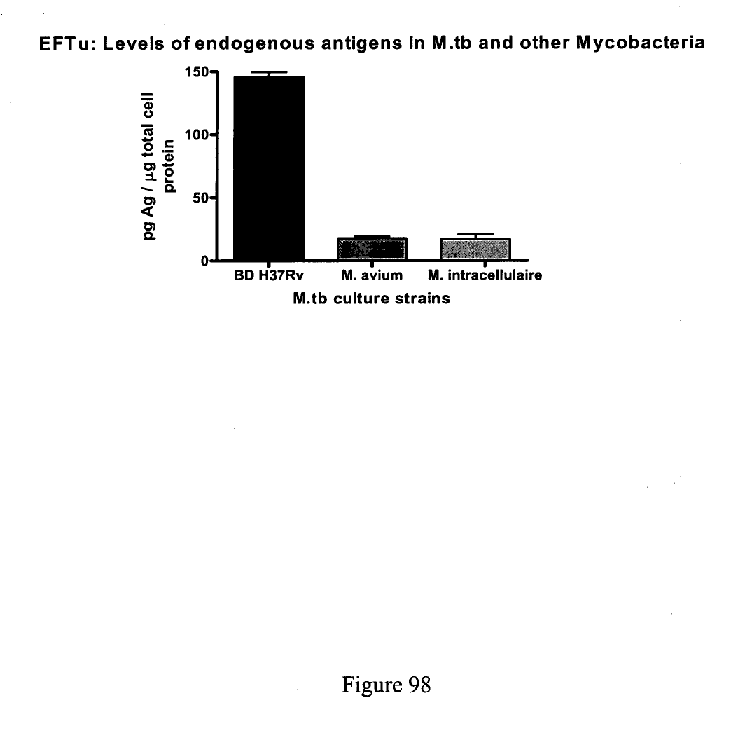

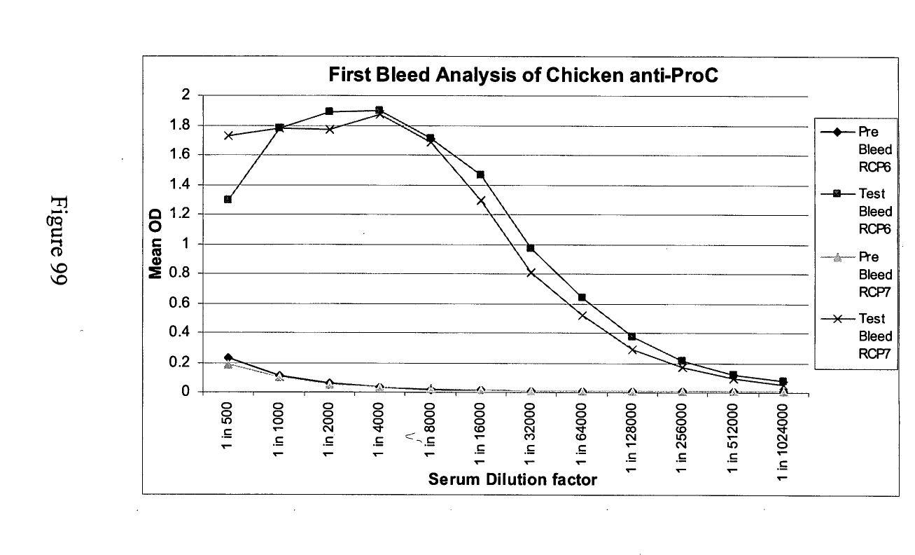

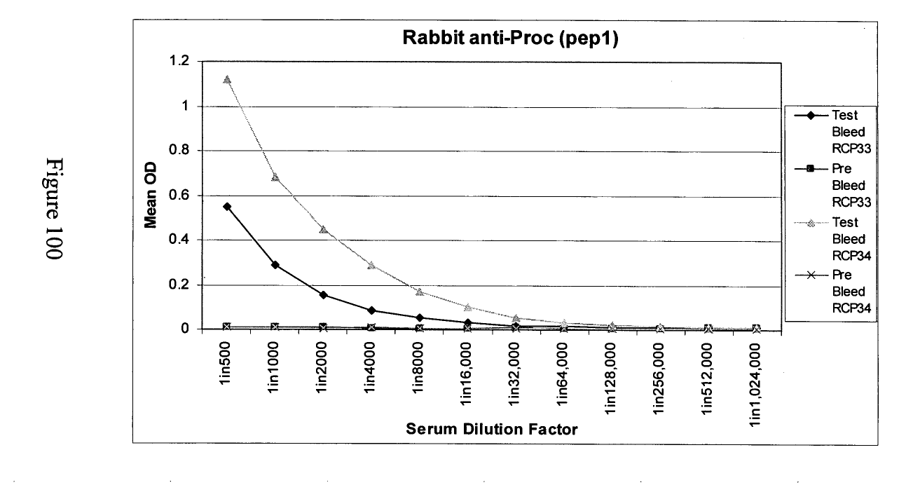

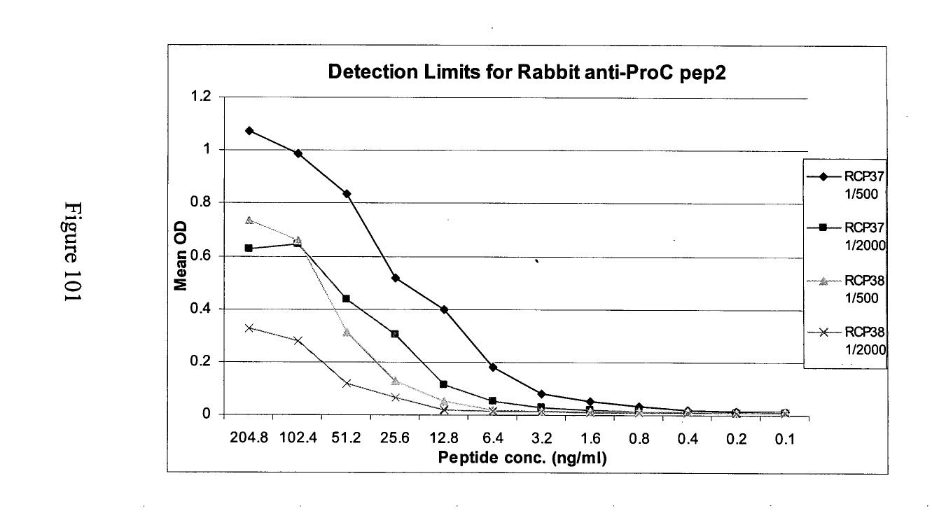

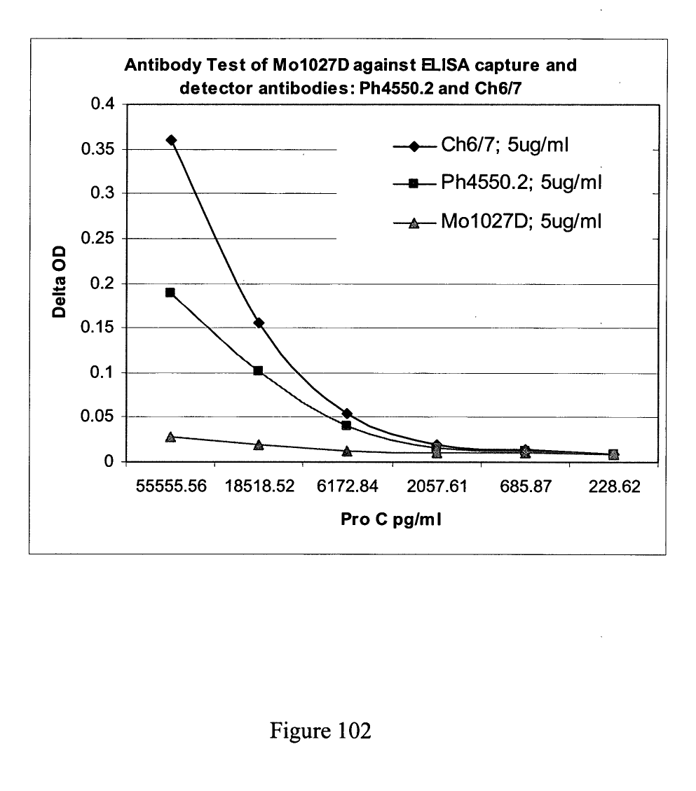

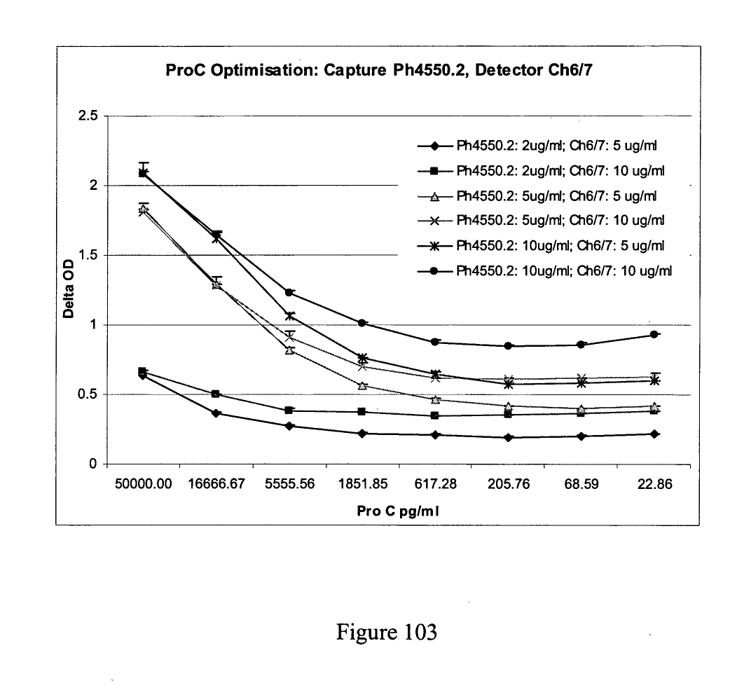

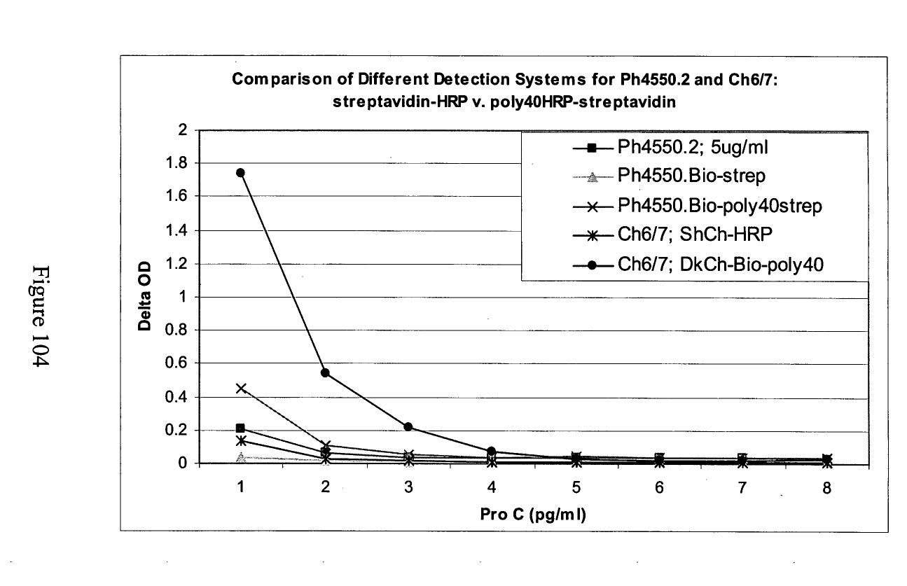

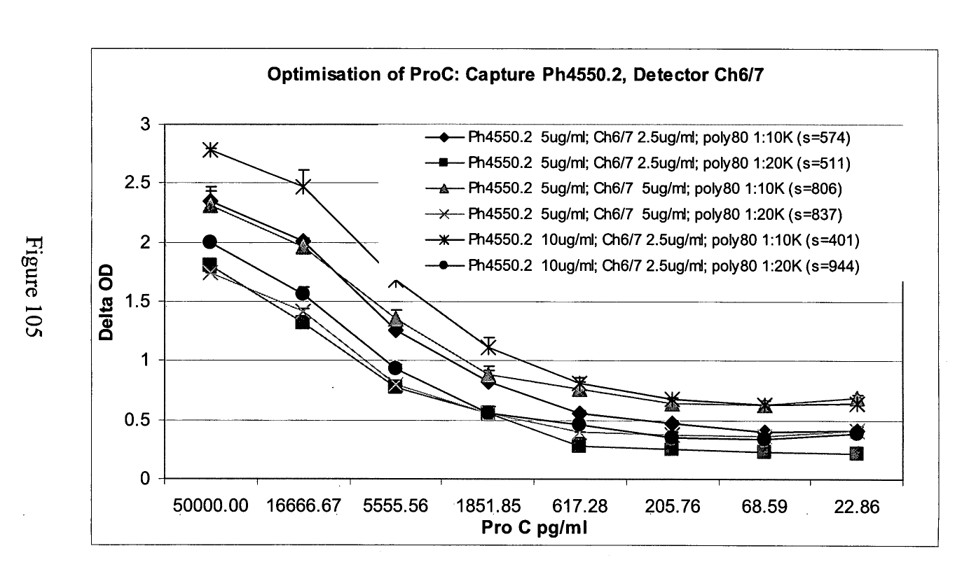

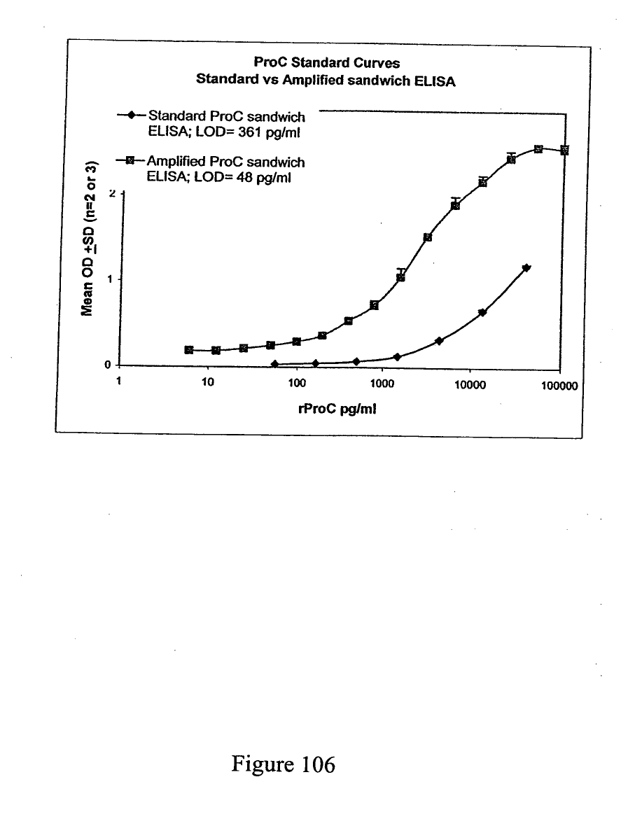

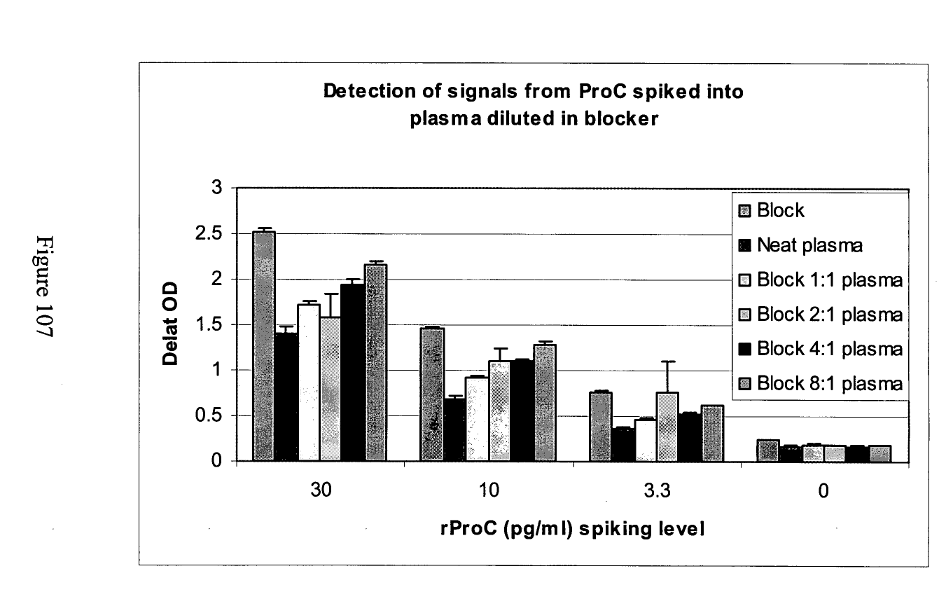

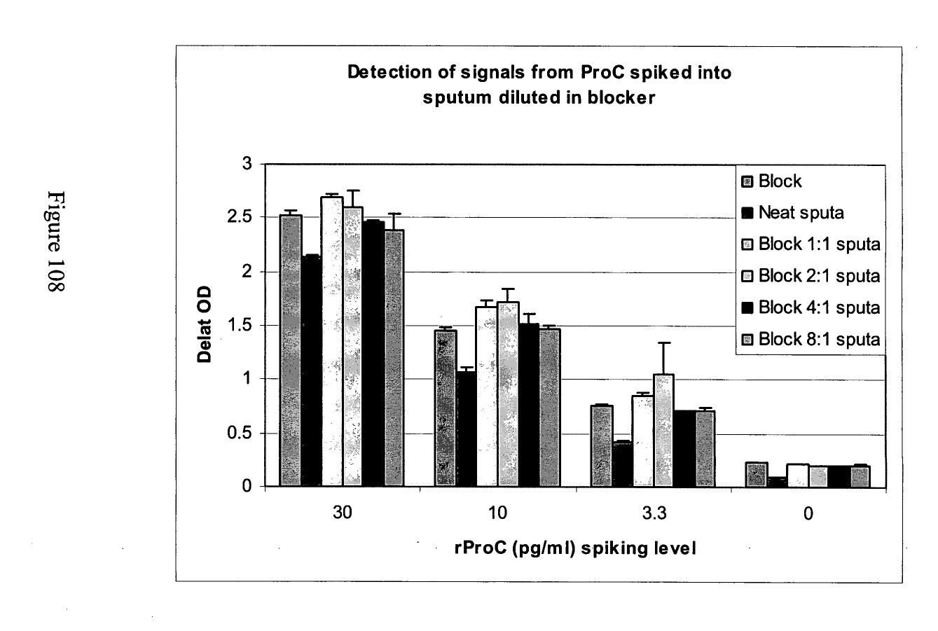

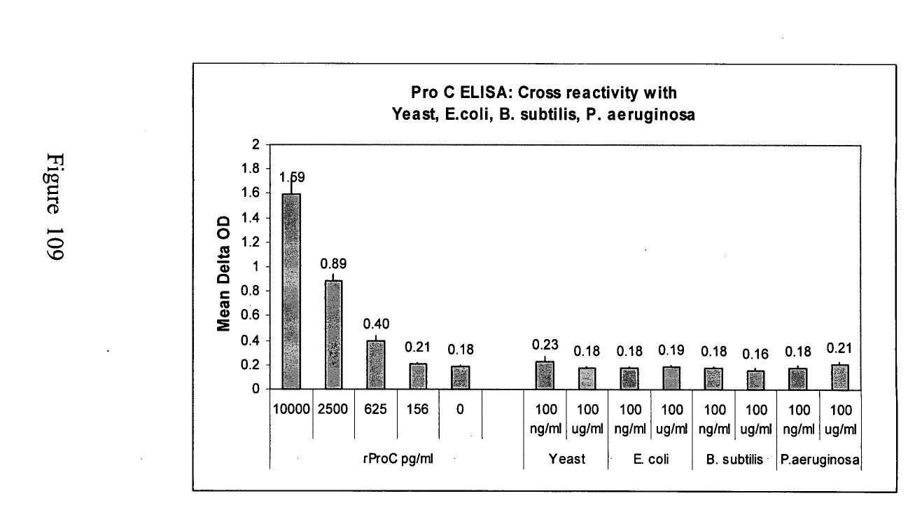

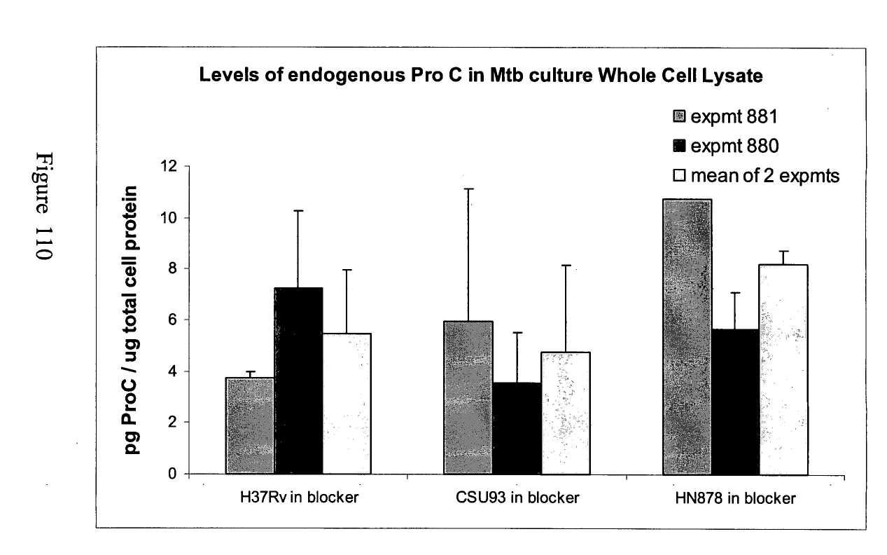

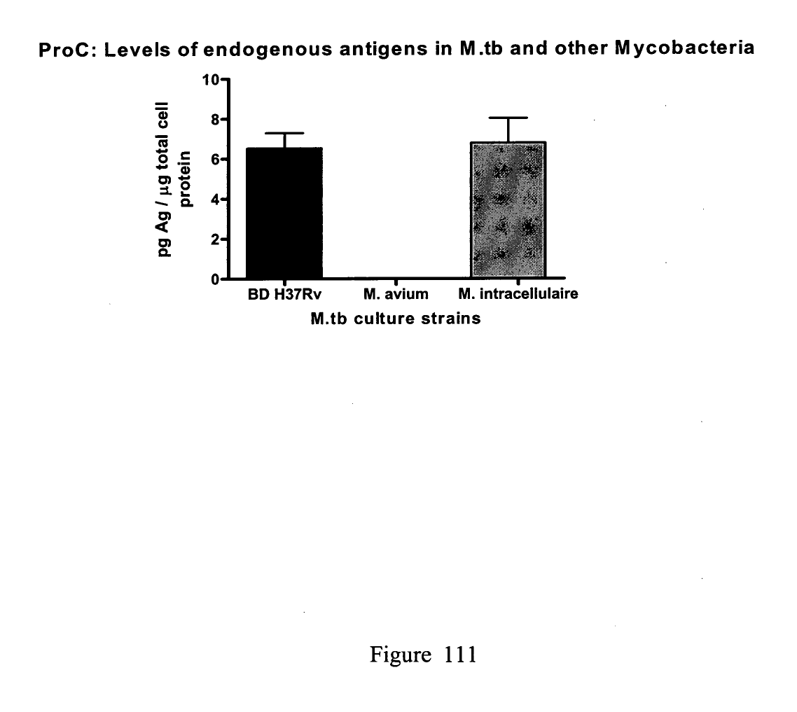

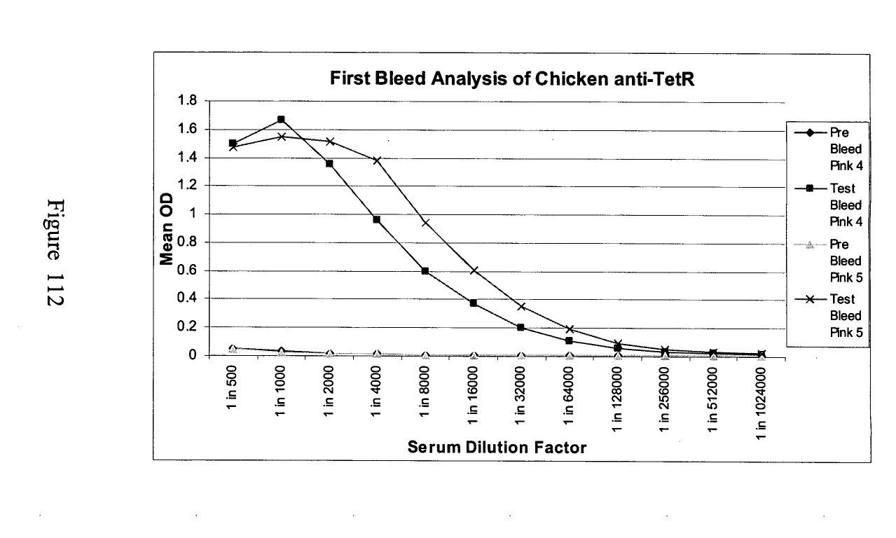

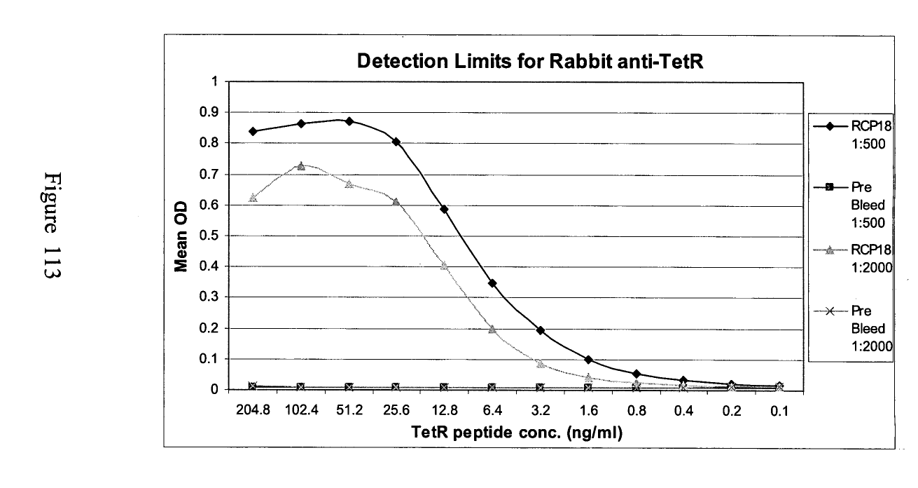

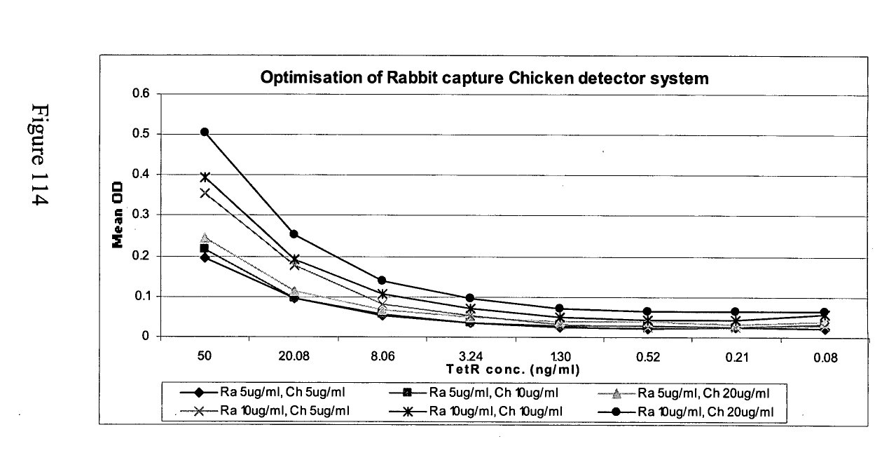

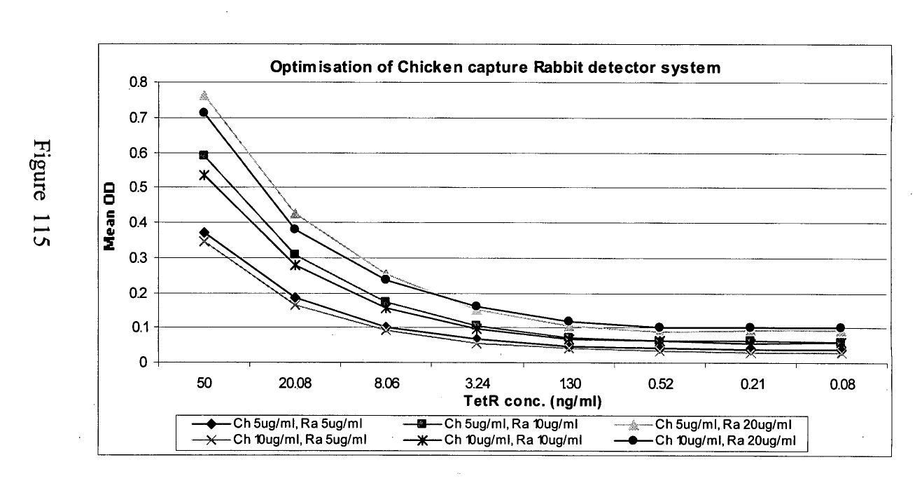

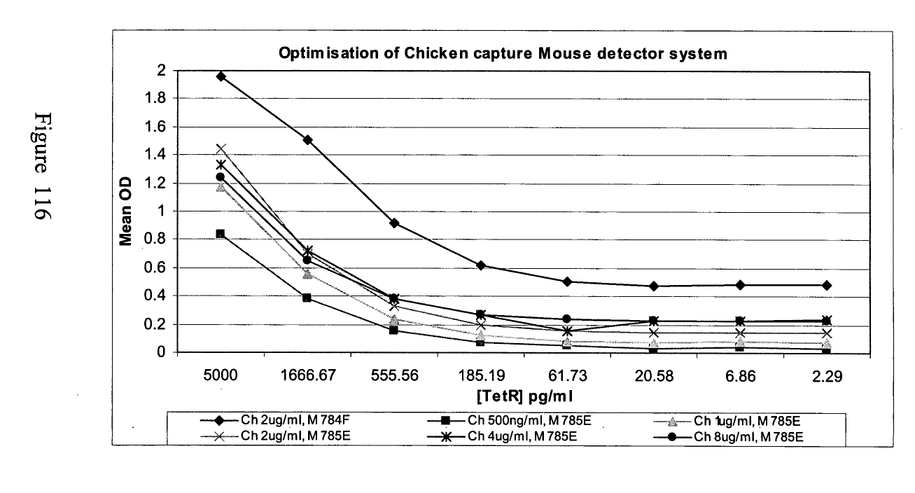

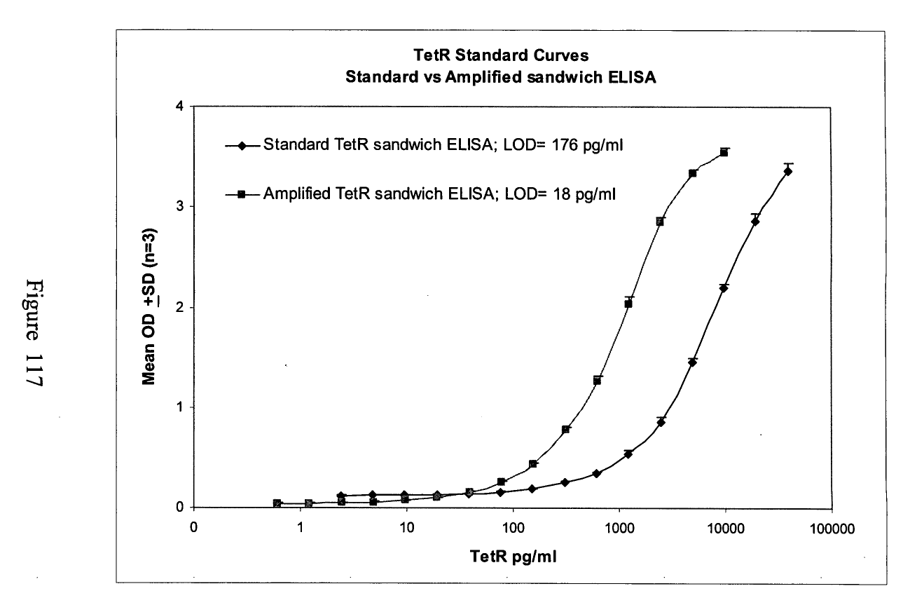

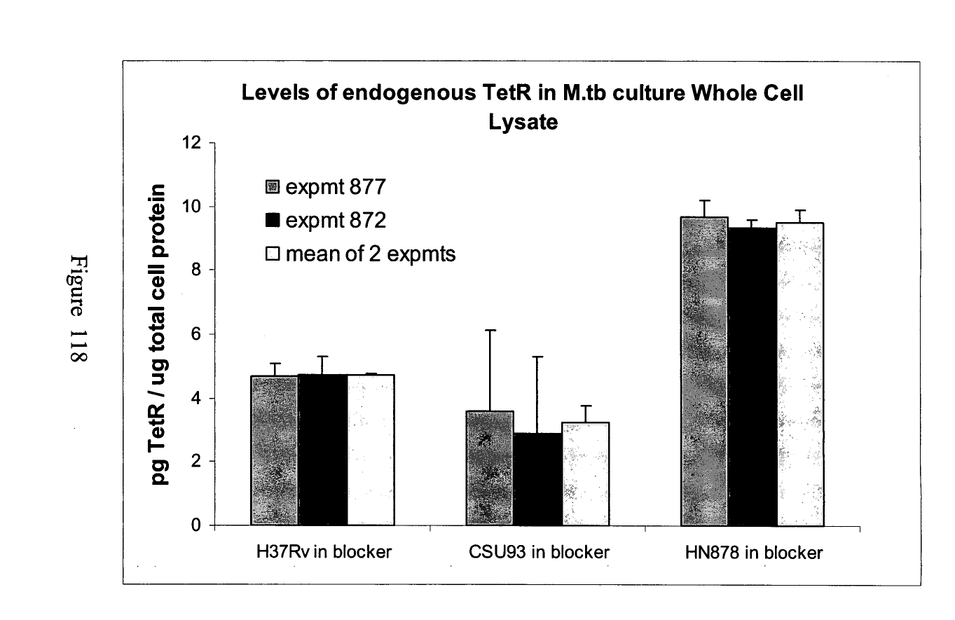

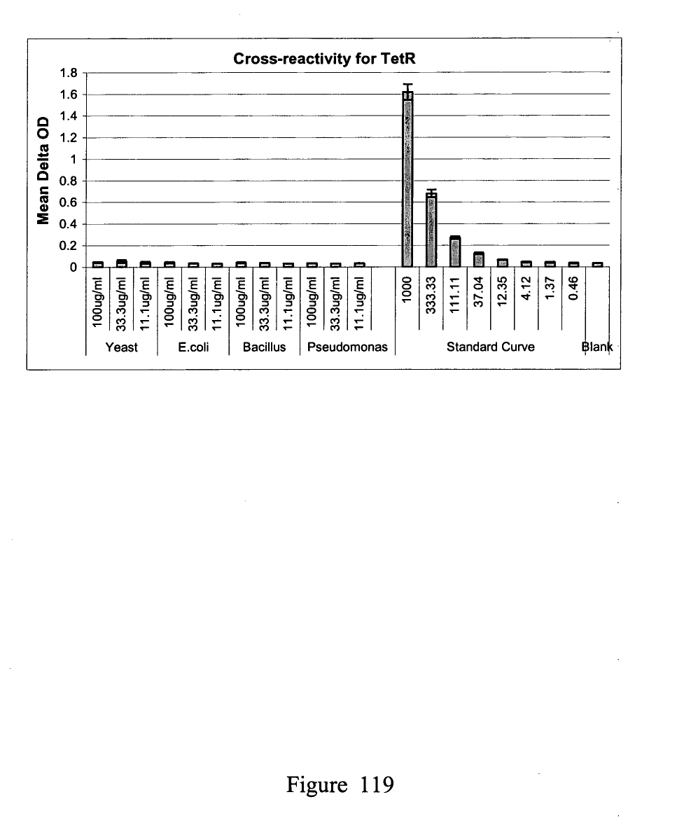

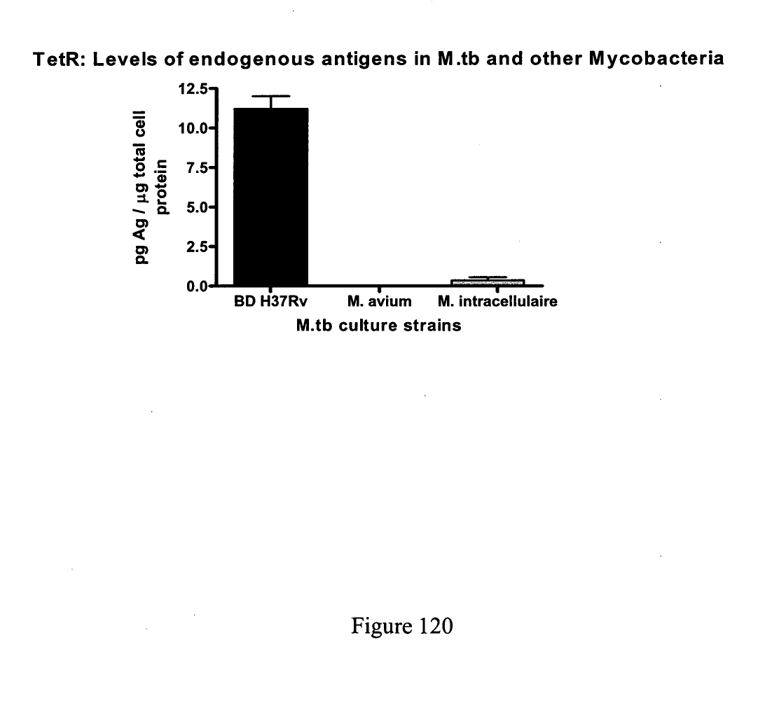

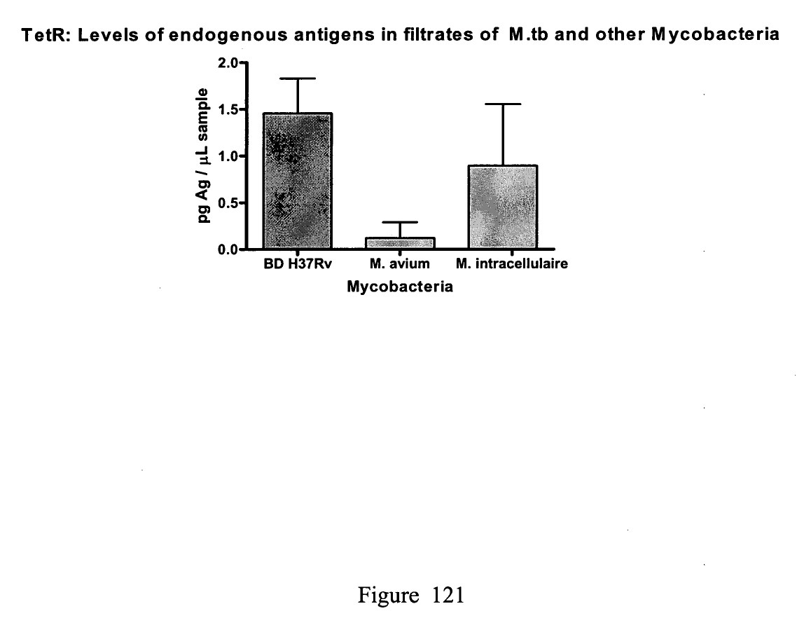

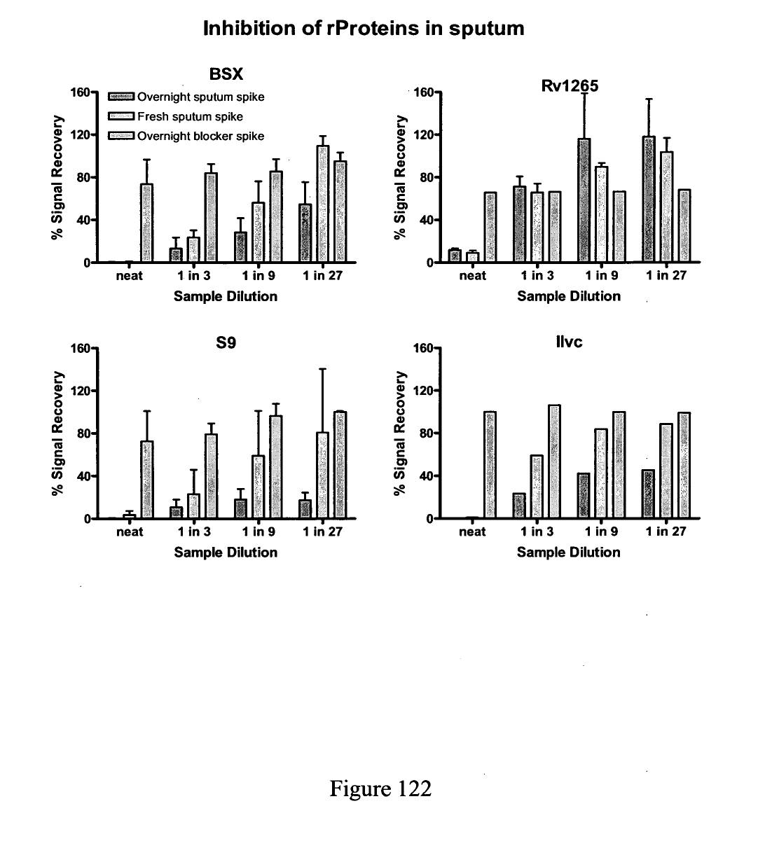

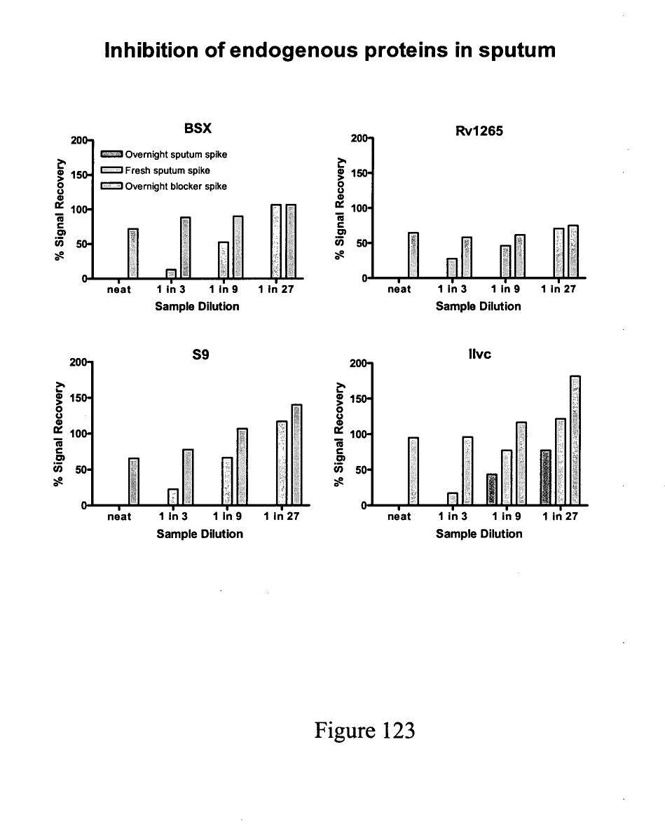

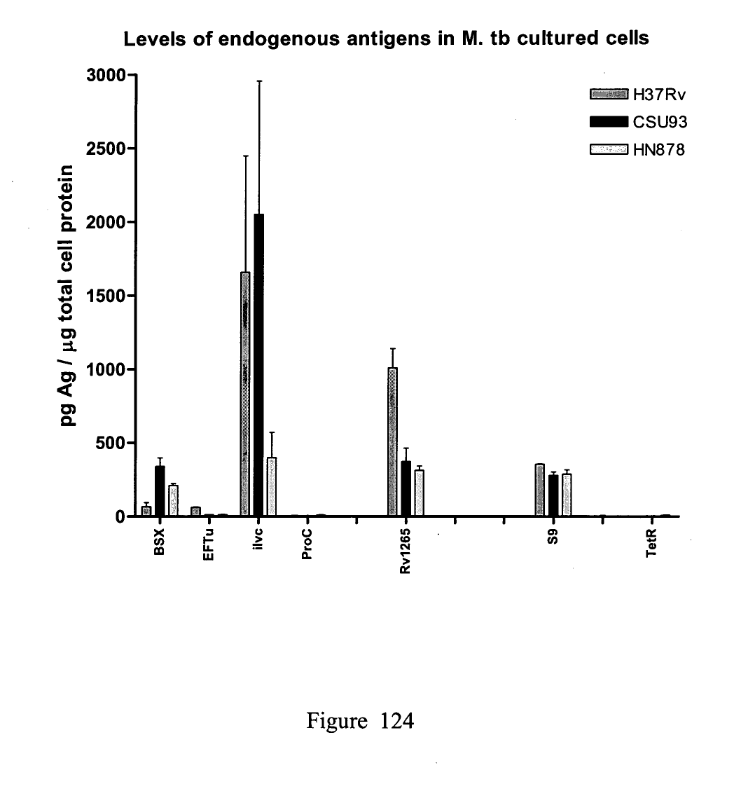

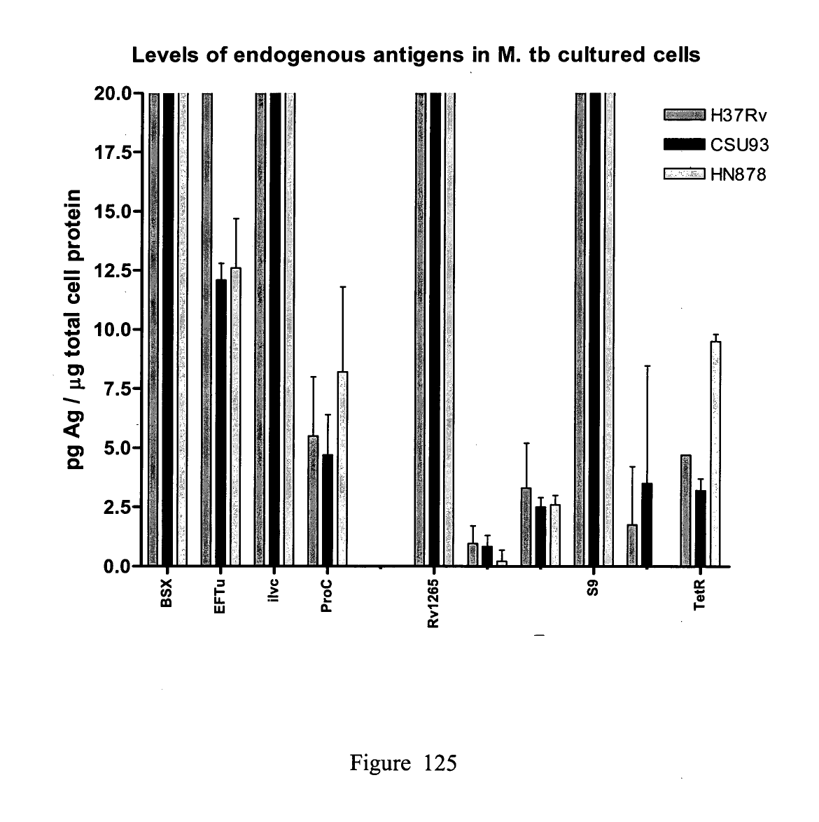

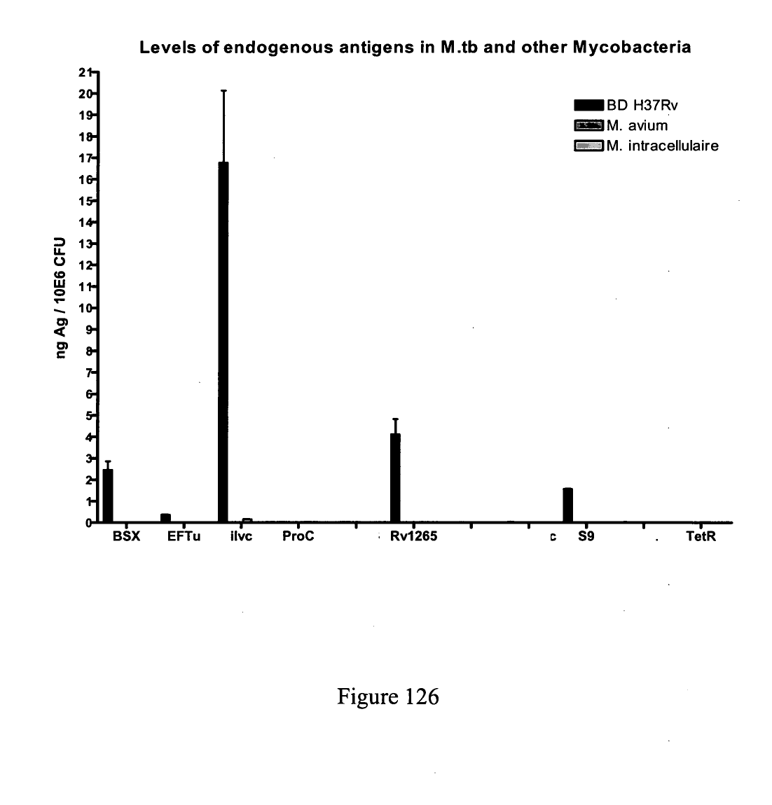

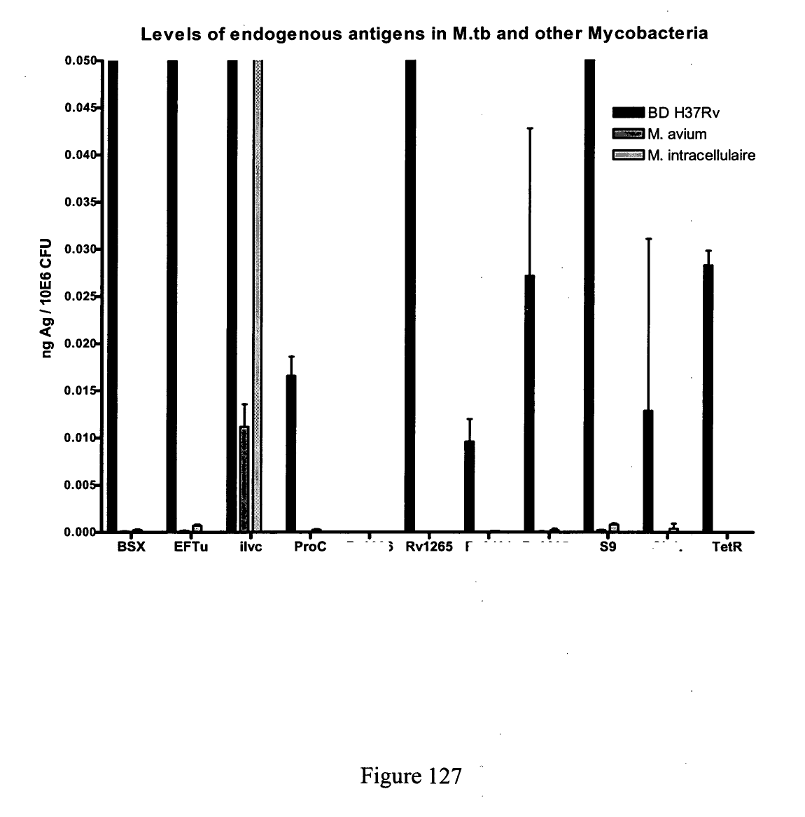

FIG. 45 is a graphical representation showing the expression in clinical sputa obtained from patients categorized according to their TB smear test results, TB culture test results and HIV status expressed as pg BSX protein/ml sample volume. Data shown in FIG. 22 were converted to pg antigen based on BSX protein calibration values therein which permitted interpolation of ug/mL BSX protein for whole cell extracts of M. tuberculosis H37Rv into pg/mL rBSX protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of BSX protein cross-reactivity in smear positive/culture positive samples independent of HIV status of the subject. LOD for assay=˜67 pg/mL.

FIG. 46 is a copy of a photographic representation showing a polyacrylamide gel within which proteins isolated from an immune-globulin fraction isolated from a TB subject have been separated using two-dimensional gel electrophoresis. The position of M. tuberculosis ribosomal protein S9 is indicated.

FIG. 47 is a graphical representation showing the titration of polyclonal antibody R9 its corresponding biotinylated peptide coated onto a 5 μg/ml streptavidin plate at 3 μg/ml.

FIG. 48 is a graphical representation showing the titration of the peptide comprising the amino acid sequence MTETT PAPQT PAAPA GPAQS FGSGL-Biotin from 20,480 pg/ml to 10 pg/ml against the rabbit sera raised against this peptide linked to KHL. Solid diamonds represent 40 μg/ml of antibody. Solid squares represent 20 μg/ml of antibody. Grey triangles represent 10 μg/ml of antibody. Grey squares represent 0 μg/ml of antibody.

FIG. 49 is a copy of a photographic representation showing a Western blot to detect M. tuberculosis ribosomal protein S9 in samples from subjects suffering from TB. The position of a band corresponding to S9 is indicated by the arrow at the right-hand side of the figure. The sample number is indicated at the top of the figure and the HIV status of each patient is indicated at the base of the figure. The molecular weight is indicated at the left-hand side of the figure.

FIG. 50 is a copy of a photographic representation showing a Western blot to detect M. tuberculosis ribosomal protein S9 in samples from control subjects, i.e., subjects that do not suffer from TB. The position of a band corresponding to S9 is indicated by the arrow at the right-hand side of the figure. The sample number is indicated at the top of the figure and the molecular weight is indicated at the left-hand side of the figure.

FIG. 51 is a graphical representation showing the binding affinities of different antibodies prepared against recombinant M. tuberculosis ribosomal protein S9 for the immunizing antigen, as determined by ELISA. Recombinant S9 protein was diluted serially 1:2 (v/v) from 500 ng/ml starting concentration to 7.8 ng/ml, and 50 μl aliquots of each dilution were used to coat the wells of an ELISA plate (x-axis). Following washing to remove unbound antigen, distinct antibodies prepared by immunization of chickens (i.e., a polyclonal antibody designated Ch27) or mice (i.e., an antibody designated Mo1025F) with recombinant full-length S9 protein were contacted separately with the adsorbed antigen at a concentration of 5 μg/ml. Following incubation at room temperature for 1 hour, plates were washed, incubated with 50 μl of a 1:5000 (v/v) dilution of secondary antibody (i.e., sheep anti-chicken IgG for detection of bound Ch27 antibody; and donkey anti-mouse IgG for detection of bound Mo1025F antibody) conjugated to horseradish peroxidase (HRP), washed, incubated with TMB for 30 mins, and absorbance at 450-620 nm was determined (y-axis). Data show that both antibodies detect recombinant S9 protein by ELISA.

FIG. 52 is a graphical representation showing sandwich ELISA results using antibody Ch27 as capture antibody and antibody Mo1025F as detection antibody for assaying recombinant M. tuberculosis ribosomal protein S9. An ELISA plate was coated overnight with capture antibody Ch27 at 5 μg/ml and 2.5 μg/ml concentrations. Following washing to remove unbound antibody, recombinant S9 protein was diluted serially 1:2 (v/v) from 500 ng/ml starting concentration to 7.8 ng/ml, and 50 μl aliquots of each dilution were added the wells of the antibody-coated ELISA plates (x-axis). Following incubation for 1 hour and washing to remove unbound antigen, detection antibody Mo1025F was contacted with the bound antigen-body complexes at concentrations in the range of 1.25 μg/ml to 5 μg/ml. Following incubation at room temperature for 1 hour, plates were washed, incubated with 50 μl of a 1:5000 (v/v) dilution of secondary antibody (i.e., donkey anti-mouse IgG) conjugated to horseradish peroxidase (HRP), washed, incubated with TMB for 30 mins, and absorbance at 450-620 nm was determined (y-axis). Data show no background signal with this antibody combination. Optimum signal was detected using capture antibody at a concentration of 5 μg/ml with detection antibody in the concentration range of 1.25 μg/ml to 5 μg/ml, which conditions provided a half-maximum detection of about 24 ng/ml M. tuberculosis ribosomal protein S9.

FIG. 53 is a graphical representation showing sandwich ELISA results using antibody Mo1025F as capture antibody and antibody Ch27 as detection antibody for assaying recombinant M. tuberculosis ribosomal protein S9. An ELISA plate was coated overnight with capture antibody Mo1025F at 5 μg/ml and 2.5 μg/ml concentrations. Following washing to remove unbound antibody, recombinant S9 protein was diluted serially 1:2 (v/v) from 500 ng/ml starting concentration to 7.8 ng/ml, and 50 μl aliquots of each dilution were added the wells of the antibody-coated ELISA plates (x-axis). Following incubation for 1 hour and washing to remove unbound antigen, detection antibody Ch27 was contacted with the bound antigen-body complexes at concentrations in the range of 1.25 μg/ml to 5 μg/ml. Following incubation at room temperature for 1 hour, plates were washed, incubated with 50 μl of a 1:5000 (v/v) dilution of secondary antibody (i.e., sheep anti-chicken IgG) conjugated to horseradish peroxidase (HRP), washed, incubated with TMB for 30 mins, and absorbance at 450-620 nm was determined (y-axis). Data show significant background cross-reactivity in the absence of added antigen using this antibody combination. Optimum signal was detected using capture antibody at a concentration of 2.5 μg/ml or 5 μg/ml with detection antibody at a concentration of 5 μg/ml under the conditions tested.

FIG. 54 is a graphical representation showing sandwich ELISA results using antibody Ch27 as capture antibody, antibody Mo1025F as detection antibody and an HRP-conjugated secondary antibody, for assaying low concentrations of recombinant M. tuberculosis ribosomal protein S9. An ELISA plate was coated overnight with capture antibody Ch27 at 5 μg/ml concentration. Following washing to remove unbound antibody, recombinant S9 protein was diluted serially 1:2 (v/v) from 150 ng/ml starting concentration to 18.31 pg/ml, and 50 μl aliquots of each dilution were added the wells of the antibody-coated ELISA plates (x-axis). Following incubation for 1 hour and washing to remove unbound antigen, detection antibody Mo1025F was contacted with the bound antigen-body complexes at 2.5 μg/ml concentration. Following incubation at room temperature for 1 hour, plates were washed, incubated with 50 μl of a 1:5000 (v/v) dilution of secondary antibody (i.e., donkey anti-mouse IgG) conjugated to horseradish peroxidase (HRP), washed, incubated with TMB for 30 mins, and absorbance at 450-620 nm was determined (y-axis). Data show no background signal with this antibody combination, a detection limit of 996 pg/ml M. tuberculosis ribosomal protein S9, and half-maximum detection of about 28 ng/ml M. tuberculosis ribosomal protein S9 under the conditions tested. Error bars show one standard deviation from the mean (n=3).

FIG. 55 is a graphical representation showing sandwich ELISA results using antibody Ch27 as capture antibody, antibody Mo1025F as detection antibody and a biotinylated secondary antibody for assaying low concentrations of recombinant M. tuberculosis ribosomal protein S9. ELISA was performed essentially as described in the legend to FIG. 32 except that recombinant S9 protein was diluted serially 1:2 (v/v) from 20 ng/ml starting concentration to 4.77 pg/ml concentration (x-axis); the incubation with secondary antibody was or 1 hour with a biotinylated donkey anti-mouse Ig followed by incubation with a modified streptavidin-HRP conjugate (poly-40) at 1:5000 (v/v) dilution; and bound antibody-antigen-antibody complexes were detected by washing plates, incubating with TMB for 10 mins, and measuring absorbance at 450-620 nm (y-axis). Data show low background signal, a detection limit of about 150 pg/ml M. tuberculosis ribosomal protein S9, and half-maximum detection of about 6 ng/ml M. tuberculosis ribosomal protein S9 using the biotinylated secondary antibody. Error bars show one standard deviation from the mean (n=3).

FIG. 56 is a graphical representation showing sandwich ELISA results using antibody Ch27 as capture antibody, antibody Mo1025F as detection antibody, a biotinylated secondary antibody and iterative antigen binding (also termed herein “replacement amplification”) for assaying low concentrations of recombinant M. tuberculosis ribosomal protein S9. ELISA was performed essentially as described in the legend to FIG. 33 except that recombinant S9 protein was diluted serially 1:2 (v/v) from 1.0 μg/ml starting concentration to 0.238 fg/ml concentration (x-axis); and antigen binding was repeated 5 times i.e., an aliquot of antigen in blocking buffer was incubated with immobilized capture antibody for 1 hour, removed, another aliquot added, and the procedure repeated until five aliquots had been added. Absorbance at 450-620 nm is indicated on the y-axis. Data show low background signal, and a detection limit of about 84 pg/ml M. tuberculosis ribosomal protein S9 using the biotinylated secondary antibody in combination with iterative antigen binding. Error bars show one standard deviation from the mean (n=3).

FIG. 57 is a graphical representation of sandwich ELISA results showing lack of significant cross-reactivity of antibodies against M. tuberculosis ribosomal protein S9 with Escherichia coli, Bacillus subtilis or Pseudomonas aeruginosa. Assay conditions were essentially as described in the legend to FIG. 33 except that purified recombinant S9 protein was replaced with 500 ng/ml or 50 μg/ml of a cellular extract as indicated on the x-axis. As a positive control, cellular extract from the M. tuberculosis laboratory strain H37Rv was used. As a negative control for each assay, buffer without cellular extract was used. Data show the change in absorbance at 450-620 nm i.e., following subtraction of background absorbance for each sample. Error bars show one standard deviation from the mean (n=3).

FIG. 58 is a graphical representation of sandwich ELISA results showing detection of M. tuberculosis ribosomal protein S9 in the clinical M. tuberculosis isolate CSU93, and lack of signal suppression in plasma. Assay conditions were essentially as described in the legend to FIG. 58 except that cellular extracts were from M. tuberculosis laboratory strain H37Rv and CSU93, as indicated on the x-axis. For determination of signal suppression by plasma, cellular extract at the concentration indicated was diluted into plasma, as indicated on the x-axis. As a negative control for each assay, buffer or plasma without cellular extract was used. The change in absorbance at 450-620 nm i.e., following subtraction of background absorbance for each sample is shown on the y-axis. Error bars show one standard deviation from the mean (n=3). Data show that plasma does not suppress signal in this assay, and that the assay is capable of detecting both clinical and laboratory isolates of M. tuberculosis.

FIG. 59 is a graphical representation showing standard and amplified sandwich ELISA standard curves for detection of M. tuberculosis S9 protein. Standard curves were generated using optimised ELISA conditions for detection of S9 in buffer as described in the examples. The concentration of recombinant S9 protein (log pg/ml) is indicated on the X-axis i.e., in logarithmic scale, and the mean OD is shown on the Y-axis. The capture antibody (5 μg/mL Ch27) and detector antibodies (2 μg/mL Mo1025F for standard ELISA and 2 μg/mL biotinylated Mo1025F (i.e., “Mo1025F-bio”) for amplified ELISA) were used. The data were plotted as an X-Y graph for mean OD+SD (n=2 or 3) against log pg/mL of rS9, and a curve fit used to determine the LOD for the assays (#733 standard and amplified ELISA, LOD=552 and 89 pg/mL respectively).

FIG. 60 is a graphical representation showing S9 protein expression (relative to total cellular protein) in one laboratory strain (H37Rv) and two clinical strains (CSU93 and HN878) of M. tuberculosis, as determined by sandwich ELISA. Whole cell lysates (WCL) from M. tuberculosis strain H37Rv (left), M. tuberculosis strains CSU93 (middle) and HN878 (right) were analysed by sandwich ELISA. The concentration of endogenous protein was calculated by interpolation from the standard curve and was corrected for the spiking level. Data were obtained from replicate experiments for which each sample was analyzed in duplicate. The level of endogenous protein (expressed as pg/μg total cell protein) was plotted as mean±SD for each of the three culture strains. M. tuberculosis strains were obtained courtesy of Colorado State University.

FIG. 61 is a graphical representation showing S9 protein expression (relative to total cell protein) in M. tuberculosis, M. intracellulaire and M. avium, as determined by sandwich ELISA. Whole cell lysates from M. tuberculosis strain H37Rv (left), and from M. avium (middle) and M. intracellulaire (right) were assayed in duplicate in two independent experiments. The concentration of endogenous protein was calculated by interpolation from the standard curve and was corrected for dilution factor. The level of endogenous protein expressed as pg/μg total cellular protein was plotted as mean±SD for each of the three Mycobacteria tested.

FIG. 62 is a graphical representation showing the expression of S9 protein in clinical sputa obtained from patients categorized according to their TB smear test results, TB culture test results and HIV status. Sputa were from each of 4 TB-positive and TB-negative subjects collected and processed as described in the examples (Method 3: 9.0 mL sputum-C1, 4×150 μL replacement amplification ELISA). Briefly, samples were size-fractionated to remove contaminants less than 100 kDa molecular weight, equilibrated to 50 mM Tris, pH 7.8, 5 mM MgCl₂, concentrated and analyzed as 4×150 uL aliquots by replacement amplification ELISA. The series of histograms to the left of the figure show mean OD values for ELISA assays of S9 protein present in calibration standards comprising serial dilutions of M. tuberculosis strain H37Rv whole cell lysates: 60 μg/ml column 1; 20 μg/ml column 2; 6.67 μg/ml column 3; 2.22 μg/ml column 4; 0.74 μg/ml column 5; 0.25 μg/ml column 6; 0.08 μg/ml column 7; 0 μg/ml column 8. The series of histograms to the left of the figure show mean OD values for ELISA assays of S9 protein present in patient samples prepared as described in the examples. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of S9 protein cross-reactivity in smear positive/culture positive samples.

FIG. 63 is a graphical representation showing the expression in clinical sputa obtained from patients categorized according to their TB smear test results, TB culture test results and HIV status expressed as pg S9 protein/ml sample volume. Data shown in FIG. 40 were converted to pg antigen based on S9 protein calibration values therein which permitted interpolation of ug/mL S9 protein for whole cell extracts of M. tuberculosis H37Rv into pg/mL rS9 protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of S9 protein cross-reactivity in smear positive/culture positive samples independent of HIV status of the subject. LOD for assay=˜55 pg/mL.

FIG. 64 is a graphical representation showing the expression of S9 protein in clinical sputa obtained from patients categorized according to their TB smear test results, TB culture test results and HIV status. Sputa were from each of 4 TB-positive and TB-negative subjects collected and processed as described in the examples (Method 2: 1.8 mL sputum-C1, 17×150 μL replacement amplification ELISA). Briefly, samples were precipitated using acetone, resolubilized and analyzed as 17×150 uL aliquots by replacement amplification ELISA. The series of histograms to the left of the figure show mean OD values for ELISA assays of S9 protein present in calibration standards comprising serial dilutions of M. tuberculosis strain H37Rv whole cell lysates: 90 μg/ml column 1; 45 μg/ml column 2; 22.50 μg/ml column 3; 11.25 μg/ml column 4; 5.63 μg/ml column 5; 2.81 μg/ml column 6; 1.41 μg/ml column 7; 0 μg/ml column 8. The series of histograms to the left of the figure show mean OD values for ELISA assays of S9 protein present in patient samples prepared as described in the examples. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples.

FIG. 65 is a graphical representation showing the expression in clinical sputa obtained from patients categorized according to their TB smear test results, TB culture test results and HIV status expressed as pg S9 protein/ml sample volume. Data shown in FIG. 42 were converted to pg antigen based on S9 protein calibration values therein which permitted interpolation of ug/mL S9 protein for whole cell extracts of M. tuberculosis H37Rv into pg/mL rS9 protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. LOD for assay=˜100 pg/mL.

FIG. 66 is a graphical representation showing the expression of S9 protein in clinical sputa obtained from patients categorized according to their TB smear test results, TB culture test results and HIV status. Sputa were from each of 4 TB-positive and TB-negative subjects collected and processed as described in the examples (Method 3: 9.0 mL sputum-C1, 4×150 μL replacement amplification ELISA). Briefly, samples were size-fractionated to remove contaminants less than 100 kDa molecular weight, equilibrated to 50 mM Tris, pH 7.8, 5 mM MgCl₂, concentrated and analyzed as 4×150 uL aliquots by replacement amplification ELISA. The series of histograms to the left of the figure show mean OD values for ELISA assays of S9 protein present in calibration standards comprising serial dilutions of M. tuberculosis strain H37Rv whole cell lysates: 60 μg/ml column 1; 20 μg/ml column 2; 6.67 μg/ml column 3; 2.22 μg/ml column 4; 0.74 μg/ml column 5; 0.25 mg/ml column 6; 0.08 μg/ml column 7; 0 μg/ml column 8. The series of histograms to the left of the figure show mean OD values for ELISA assays of S9 protein present in patient samples prepared as described in the examples. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of S9 protein cross-reactivity in smear positive/culture positive samples.

FIG. 67 is a graphical representation showing the expression in clinical sputa obtained from patients categorized according to their TB smear test results, TB culture test results and HIV status expressed as pg S9 protein/ml sample volume. Data shown in FIG. 44 were converted to pg antigen based on S9 protein calibration values therein which permitted interpolation of ug/mL S9 protein for whole cell extracts of M. tuberculosis H37Rv into pg/mL rS9 protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of S9 protein cross-reactivity in smear positive/culture positive samples independent of HIV status of the subject. LOD for assay=˜55 pg/mL.

FIG. 68 is a graphical representation showing the titration of polyclonal antibodies prepared in chickens against SEQ ID NO: 6. Recombinant protein Rv1265/MT1303 (SEQ ID NO: 21) was immobilized onto ELISA plate at a concentration of 5 μg/ml. Dilutions of antisera designated “Pink 10” (▪) and “Pink 11” (X) as indicated on the x-axis, and dilutions of pre-immune sera from the same animals (♦ for Pink 10; ▴ for Pink 11) as indicated on the x-axis, were contacted with the immobilized recombinant protein Rv1265/MT1303 for a time and under conditions sufficient for an antigen: antibody complex to form. The ELISA plate was washed and complexes detected by binding sheep anti-chicken IgG horseradish peroxidase (HRP) conjugate diluted 1:5000 (v/v) using TMB to detect bound HRP activity. Optical density (OD) was determined for each sample (y-axis). Data indicate antibody titers of at least about 1:64,000 (v/v) for Pink 10 and at least about 1:128,000 (v/v) for Pink 11.

FIG. 69 is graphical representation showing the titration of polyclonal antibodies prepared in rabbits against SEQ ID NO: 26. Streptavidin was immobilized onto an ELISA plate at a concentrate of 5 μg/ml. Biotin conjugated to a peptide consisting of the sequence set forth in SEQ ID NO: 26 (3 μg/ml) was contacted with the plate for a time and under conditions sufficient to immobilize the peptide via a biotin streptavidin interaction. Dilutions of rabbit antisera or pre-immune sera were added for a time and under conditions sufficient to form an antigen: antibody complexes, and bound antibodies were then detected as described in the legend of FIG. 46, except that the secondary antibody was a sheep anti-rabbit IgG HRP conjugate. Rabbit sera were designated RCP25 (▪ for preimmune sera; ♦ for immune sera) and RCP26 (X for preimmune sera; ▴ for immune sera). Sera dilutions are indicated on the x-axis. Optical density (OD) is indicated on the y-axis. Data suggest antibody titers of at least about 1:64,000 (v/v) for both preparations.

FIG. 70 is a graphical representation showing the detection limits of a purified rabbit anti-protein Rv1265/MT1303 antibody preparation. Recombinant Rv1265/MT1303 protein comprising the sequence set forth in SEQ ID NO: 21 was bound to an ELISA plate essentially as described in the legend to FIG. 47 except that the concentration of protein was varied from 19.6 ng/ml to 200 pg/ml (x-axis). Purified rabbit antibody at a concentration of 1.25 μg/ml, 2.5 μg/ml or 5 μg/ml was then bound to the recombinant protein and detected using a sheep anti-rabbit IgG HRP conjugate as described in the legend to FIG. 47. Data indicate that the limits of detection of the rabbit antibody is about' 2.5 ng/ml at all concentrations tested.

FIG. 71 is a graphical representation of a standard sandwich ELISA using the Ch10/11 pool of polyclonal antibodies designated Ch10 (=antibody “Pink 10” referred to herein) and Ch11 (=antibody “Pink 11” referred to herein) and monoclonal antibody designated Mo788C prepared against the full-length recombinant M. tuberculosis RV1265 protein (SEQ ID NO: 21). The figure shows the effect of using these two antibodies in different orientations in the sandwich ELISA i.e., as capture and detection antibodies. Wells of an ELISA plate were coated overnight with 50 μl of a 5 μg/ml concentration of Ch10/11 or Mo788C antibody. Following blocking and washing to remove unbound antibody, recombinant Rv1265 protein was diluted serially 1:3 (v/v) from 500 ng/ml starting concentration to 22.86 pg/ml, and 50 μl aliquots of each dilution were added the wells of the antibody-coated ELISA plates (x-axis). Following incubation for 1 hour and washing to remove unbound antigen, the alternate detection antibody i.e., Ch10/11 for detecting Rv1265-Mo788C complexes and Mo788C for detecting Rv1265-Ch10/11 complexes, was contacted with the bound antigen-body complexes at a concentration of 2 μg/ml. Following incubation at room temperature for 1 hour, plates were washed, incubated with 50 μl of a 1:5000 (v/v) dilution of secondary antibody (i.e., sheep anti-chicken IgG for detecting Ch10/11 or sheep anti-mouse IgG for detecting Mo788C) conjugated to horseradish peroxidase (HRP), washed, incubated with TMB for 30 mins, and absorbance at 450-620 nm was determined after subtraction of background (y-axis). Without limiting the invention, data suggest that lower background effects are observed when the monoclonal antibody M0788C is used as a capture reagent and the polyclonal antibody pool Ch10/11 is used as a detection reagent in sandwich ELISA.

FIG. 72 is a graphical representation comparing an amplified sandwich ELISA to standard sandwich ELISA for detecting recombinant M. tuberculosis RV1265 protein. An ELISA plate was coated overnight with capture antibody Mo788C at 5 μg/ml concentration. Following washing to remove unbound antibody, recombinant Rv1265 protein was diluted serially 1:10 (v/v) from about 10 μg/ml starting concentration to about 1.0 pg/ml, and 50 μl aliquots of each dilution were added the wells of the antibody-coated ELISA plates (x-axis). Following incubation for 1 hour and washing to remove unbound antigen, antibody Ch10/11 was contacted with the bound antigen-body complexes at 2.0 μg/ml concentration. Following incubation at room temperature for 1 hour, plates were washed, and incubated with 50 μl of a 1:5,000 (v/v) dilution of a secondary antibody consisting of HRP-conjugated sheep anti-chicken IgG (standard sandwich ELISA) or 50 μl of a 1:50,000 (v/v) dilution of biotinylated donkey anti-chicken IgG (amplified sandwich ELISA). Following incubation at room temperature for a further one hour, the plates washed as before. For samples undergoing amplified ELISA, HRP80-streptavidin was then added to the plates which were incubated for a further one hour at room temperature, and washed as before. Finally, all samples were incubated with TMB for 5 mins. Absorbance was determined at 450-620 nm (y-axis). Data indicate significant enhancement of detection using the amplified sandwich ELISA under these conditions: The limit of detection of this amplified sandwich ELISA is about 60 pg/ml Rv1265 protein, with half-maximum detection of about 5 ng/ml Rv1265 protein. This compares favourably to the observed limit of detection of the standard sandwich ELISA of about 2.6 ng/ml Rv1265 protein, with half-maximum detection of about 100 ng/ml Rv1265 protein.

FIG. 73 is a graphical representation of sandwich ELISA results showing detection of M. tuberculosis Rv 1265 protein in whole cell extracts of the clinical M. tuberculosis isolates CSU93 and HN878, and in the laboratory strain H37Rv. Amplified sandwich ELISA conditions were essentially as described in the legend to FIG. 50, except for the following: (i) cellular extracts were assays as indicated on the x-axis; (ii) the whole cell extracts were spiked with recombinant Rv1265 protein to a final concentration of 50, 16.7, 5.6 and 1.8 μg total cell protein/ml; and (iii) the concentration of endogenous Rv1265 protein was determined by interpolation from a standard curve of Rv1265 concentration against signal strength, and corrected for the level of recombinant Rv1265 protein spike in the samples (e.g., corrected for the dilution factor). Data are presented as picograms endogenous Rv1265 protein per microgram of total protein in the cellular extract (y-axis) for two separate experiments. Average protein levels are also indicated.

FIG. 74 is a graphical representation of sandwich ELISA results showing lack of significant cross-reactivity of antibodies against M. tuberculosis Rv1265 protein with whole cell lysates from yeast, Escherichia coli, Bacillus subtilis or Pseudomonas aeruginosa. Assay conditions were essentially as described in the legend to FIG. 51 except that 0-20 ng/ml purified recombinant Rv1265 protein or 100 ng/ml or 100 μg/ml of a cellular extract was assayed, as indicated on the x-axis. Buffer without protein or cellular extract served as a negative control. Data show the change in absorbance at 450-620 nm i.e., following subtraction of background absorbance for each sample.

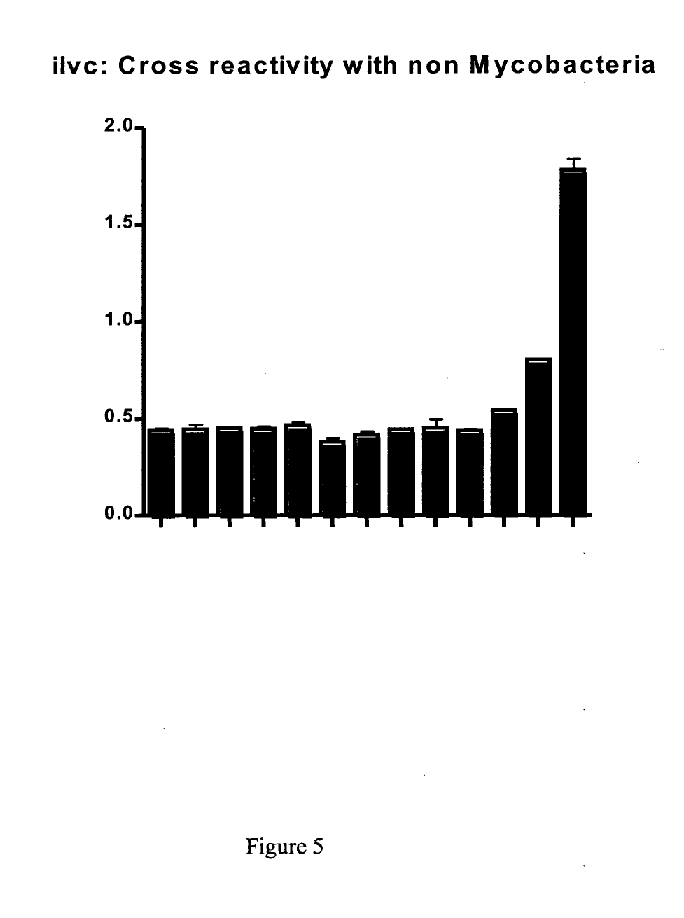

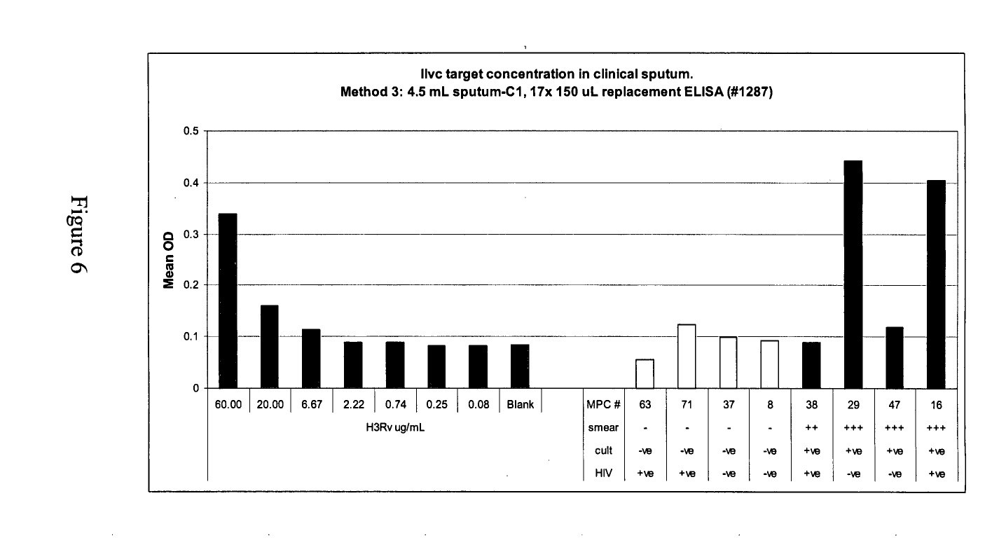

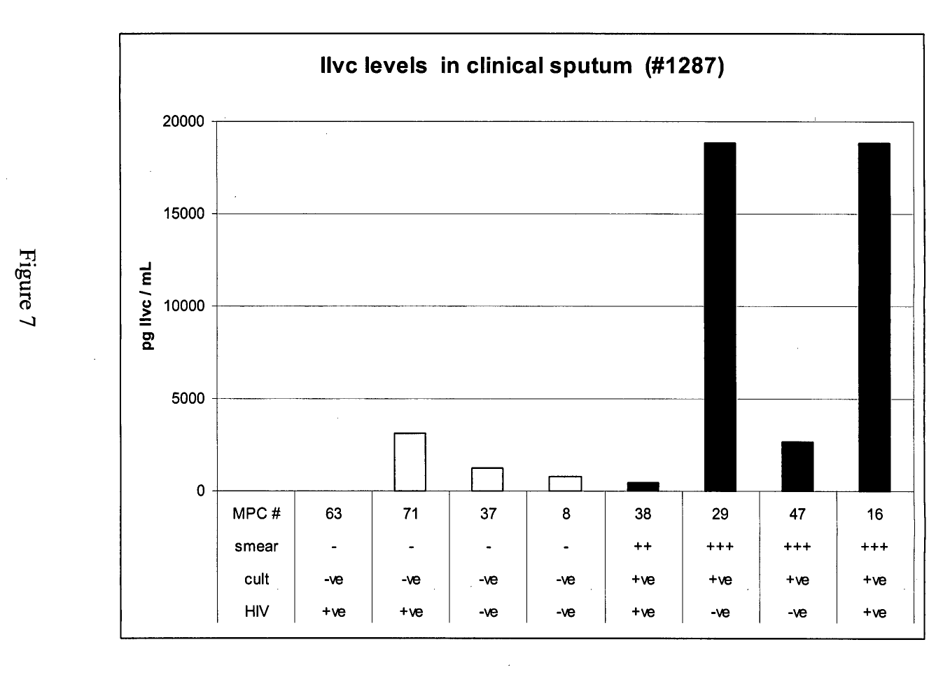

FIG. 75 is a graphical representation showing the effect of undiluted plasma in quenching detection of recombinant Rv1265 protein in the amplified sandwich ELISA assay described in preceding FIG. 5, and recovery of lost signal by dilution of the sputa. An ELISA plate was coated overnight with capture antibody Mo788C at 5 μg/ml concentration. Following washing to remove unbound antibody, recombinant Rv1265 protein was spiked at the concentrations indicated in the legend into undiluted blocking solution (“block” or “blocker”), undiluted plasma (“neat plasma”), or a dilution of plasma in blocking solution ranging from 1:1 (v/v) block:plasma to 8:1 (v/v) block:plasma, and 50 μl aliquots of each sample added the wells of the antibody-coated ELISA plates (x-axis). Following incubation for 1 hour and washing to remove unbound antigen, antibody Ch10/11 was contacted with the bound antigen-body complexes at 2.0 μg/ml concentration. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show about 30% attenuation/suppression of signal by plasma.

FIG. 76 is a graphical representation showing the effect of undiluted sputa in quenching detection of recombinant Rv1265 protein in the amplified sandwich ELISA assay described in preceding FIG. 5, and recovery of lost signal by dilution of the sputa. An ELISA plate was coated overnight with capture antibody Mo788C at 5 μg/ml concentration. Following washing to remove unbound antibody, recombinant Rv1265 protein was spiked at the concentrations indicated in the legend into undiluted blocking solution (“block” or “blocker”), undiluted sputa (“neat sputum”), or a dilution of sputa in blocking solution ranging from 1:1 (v/v) block:sputum to 8:1 (v/v) block:sputum, and 50 μl aliquots of each sample added the wells of the antibody-coated ELISA plates (x-axis). Following incubation for 1 hour and washing to remove unbound antigen, antibody Ch10/11 was contacted with the bound antigen-body complexes at 2.0 μg/ml concentration. Following incubation at room temperature for 1 hour, plates were washed, and incubated with 50 μl of a 1:50,000 (v/v) dilution of a secondary antibody consisting of biotinylated donkey anti-chicken IgG. Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show no significant attenuation/suppression of signal by sputa.

FIG. 77 is a graphical representation showing Rv1265 protein expression (relative to total cell protein) in M. tuberculosis, M. intracellulaire and M. avium, as determined by sandwich ELISA. Whole cell lysates from M. tuberculosis strain H37Rv (left), and from M. avium (middle) and M. intracellulaire (right) were assayed in duplicate in two independent experiments. The concentration of endogenous protein was calculated by interpolation from the standard curve and was corrected for dilution factor. The level of endogenous protein expressed as pg/μg total cellular protein was plotted as mean±SD for each of the three Mycobacteria tested.

FIG. 78 is a graphical representation showing Rv1265 protein expression in filtrates obtained from whole cell lysates of M. tuberculosis, M. intracellulaire and M. avium, as determined by sandwich ELISA. Filtrates obtained from cell lysates of M. tuberculosis strain H37Rv (left), M. avium (middle) and M. intracellulaire (right) were assayed in duplicate. The concentration of endogenous protein was calculated by interpolation from the standard curve and was corrected for dilution factor (if any). The level of endogenous protein expressed as pg/μL filtrate was plotted as mean±SD for each of the three Mycobacteria.

FIG. 79 is a graphical representation showing the expression of Rv1265 protein in clinical sputa obtained from patients categorized according to their TB smear test results, TB culture test results and HIV status. Sputa were from each of 4 TB-positive and TB-negative subjects collected and processed as described in the examples (Method 1: 2.5 mL sputum-M1, 17×150 μL replacement amplification ELISA). The series of histograms to the left of the figure show mean OD values for ELISA assays of RV1265 protein present in calibration standards comprising serial dilutions of M. tuberculosis strain H37Rv whole cell lysates: 20 μg/ml column 1; 10 μg/ml column 2; 5 μg/ml column 3; 2.5 μg/ml column 4; 1.25 μg/ml column 5; 0.625 μg/ml column 6; 0.313 μg/ml column 7; 0.156 μg/ml column 8; 0.078 μg/ml column 9; 0.039 μg/ml column 10; 0.02 μg/ml column 11; 0.01 μg/ml column 12; 0.005 μg/ml column 13; 0.002 μg/ml column 14; 0.001 μg/ml column 15; 0 μg/ml column 16. The series of histograms to the right of the figure show mean OD values for ELISA assays of RV1265 protein present in patient samples prepared as described in the examples. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of RV1265 protein cross-reactivity in smear positive/culture positive samples.

FIG. 80 is a graphical representation showing the expression in clinical sputa obtained from patients categorized according to their TB smear test results, TB culture test results and HIV status expressed as pg RV1265 protein/ml sample volume. Data shown in FIG. 57 were converted to pg antigen based on RV1265 protein calibration values therein which permitted interpolation of ug/mL RV1265 protein for whole cell extracts of M. tuberculosis H37Rv into pg/mL rRV1265 protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of RV1265 protein cross-reactivity in smear positive/culture positive samples independent of HIV status of the subject. LOD for assay=˜11.2 pg/mL.

FIG. 81 is a graphical representation showing the expression of Rv1265 protein in clinical sputa obtained from patients categorized according to their TB smear test results, TB culture test results and HIV status. Sputa were from each of 4 TB-positive and TB-negative subjects collected and processed as described in the examples (Method 2: 1.8 mL sputum-C1, 4×150 μL replacement amplification ELISA). Briefly, samples were precipitated using acetone, resolubilized and analyzed as 4×150 uL aliquots by replacement amplification ELISA. The series of histograms to the left of the figure show mean OD values for ELISA assays of Rv1265 protein present in calibration standards comprising serial dilutions of M. tuberculosis strain H37Rv whole cell lysates: 90 μg/ml column 1; 45 μg/ml column 2; 22.50 μg/ml column 3; 11.25 μg/ml column 4; 5.63 μg/ml column 5; 2.81 μg/ml column 6; 1.41 μg/ml column 7; 0 μg/ml column 8. The series of histograms to the right of the figure show mean OD values for ELISA assays of RV1265 protein present in patient samples prepared as described in the examples. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples.

FIG. 82 is a graphical representation showing the expression in clinical sputa obtained from patients categorized according to their TB smear test results, TB culture test results and HIV status expressed as pg RV1265 protein/ml sample volume. Data shown in FIG. 59 were converted to pg antigen based on RV1265 protein calibration values therein which permitted interpolation of ug/mL RV1265 protein for whole cell extracts of M. tuberculosis H37Rv into pg/mL rRV1265 protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of Rv1265 protein cross-reactivity in smear positive/culture positive samples independent of HIV status of the subject. LOD for assay=˜67 pg/mL.

FIG. 83 is a graphical representation showing the expression of RV1265 protein in clinical sputa obtained from patients categorized according to their TB smear test results, TB culture test results and HIV status. Sputa were from each of 4 TB-positive and TB-negative subjects collected and processed as described in the examples (Method 3: 9.0 mL sputum-C1, 4×150 μL replacement amplification ELISA). Briefly, samples were size-fractionated to remove contaminants less than 100 kDa molecular weight, equilibrated to 50 mM Tris, pH 7.8, 5 mM MgCl₂, concentrated and analyzed as 4×150 uL aliquots by replacement amplification ELISA. The series of histograms to the left of the figure show mean OD values for ELISA assays of RV1265 protein present in calibration standards comprising serial dilutions of M. tuberculosis strain H37Rv whole cell lysates: 60 μg/ml column 1; 20 μg/ml column 2; 6.67 μg/ml column 3; 2.22 μg/ml column 4; 0.74 μg/ml column 5; 0.25 μg/ml column 6; 0.08 μg/ml column 7; 0 μg/ml column 8. The series of histograms, to the right of the figure show mean OD values for ELISA assays of RV1265 protein present in patient samples prepared as described in the examples. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples.

FIG. 84 is a graphical representation showing the expression in clinical sputa obtained from patients categorized according to their TB smear test results, TB culture test results and HIV status expressed as pg RV1265 protein/ml sample volume. Data shown in FIG. 61 were converted to pg antigen based on RV1265 protein calibration values therein which permitted interpolation of ug/mL RV1265 protein for whole cell extracts of M. tuberculosis H37Rv into pg/mL rRV 1265 protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. Data show significantly higher levels of Rv1265 protein cross-reactivity in smear positive/culture positive samples independent of HIV status of the subject. LOD for assay=˜26 pg/mL.

FIG. 85 is a graphical representation showing the expression of RV1265 protein in clinical sputa obtained from patients categorized according to their TB smear test results, TB culture test results and HIV status. Sputa were from each of 4 TB-positive and TB-negative subjects collected and processed as described in the examples (Method 4: 18.0 mL sputum-M2, 4×150 μL replacement amplification ELISA). Briefly, samples were size-fractionated to remove contaminants less than 100 kDa molecular weight, equilibrated to 50 mM Tris, pH 7.8, 5 mM MgCl₂, concentrated and analyzed as 4×150 uL aliquots by replacement amplification ELISA. The series of histograms to the left of the figure show mean OD values for ELISA assays of RV1265 protein present in calibration standards comprising serial dilutions of M. tuberculosis strain H37Rv whole cell lysates: 30 μg/ml column 1; 10 μg/ml column 2; 3.33 μg/ml column 3; 1.11 μg/ml column 4; 0.37 μg/ml column 5; 0.12 μg/ml column 6; 0.04 μg/ml column 7; 0 μg/ml column 8. The series of histograms to the right of the figure show mean OD values for ELISA assays of RV1265 protein present in patient samples prepared as described in the examples. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples.

FIG. 86 is a graphical representation showing the expression in clinical sputa obtained from patients categorized according to their TB smear test results, TB culture test results and HIV status expressed as pg RV1265 protein/ml sample volume. Data shown in FIG. 63 were converted to pg antigen based on RV1265 protein calibration values therein which permitted interpolation of ug/mL RV1265 protein for whole cell extracts of M. tuberculosis H37Rv into pg/mL rRV1265 protein. “MPC” indicates the sample identification code; “smear” indicates smear test result: “cult” indicates the culture test result; and “HIV” indicates HIV status. Open bars indicate smear negative/culture negative samples. Filled bars indicate smear positive/culture positive samples. LOD for assay=˜65 pg/mL.

FIG. 87 is a graphical representation showing the detection of anti-EF-Tu antibodies in serum or plasma from subjects suffering from tuberculosis or control subjects. Recombinant EF-Tu was immobilised onto an ELISA plate at 50 μl per well at a concentration 2 μg/ml. Plasma or serum samples diluted 1/100 in blocking buffer were then contacted to the immobilised protein for a time and under conditions sufficient for an antibody: antigen complex to form. The ELISA plate was washed and the complexes detected by binding sheep anti-human IgG horseradish peroxidise (HRP) conjugate diluted 1:50,000 using TMB to detect bound HRP activity. Optical density (OD) was determined for each sample (y-axis). Black bars indicate sample s from subjects suffering from tuberculosis. Grey bars indicate samples from control subjects.

FIG. 88 is a graphical representation showing the titration of monoclonal antibodies produced by plasmacytomas against full-length EF-Tu fused to NUS or against SEQ ID NO: 35. Recombinant EF-Tu protein was immobilised onto an ELISA plate at a concentration of 17 μg/ml. Dilutions of antibodies designated 681E (⋄), 683B (▪), 682A (□), 685B (+), 684A (), 524D (X) and 521F (♦) as indicated on the x-axis, were contacted with the immobilized recombinant EF-Tu protein for a time and under conditions sufficient for an antigen: antibody complex to form. The ELISA plate was washed and complexes detected by binding sheep anti-mouse Ig horseradish peroxidase (HRP) conjugate diluted 1:5000 (v/v) using TMB to detect bound HRP activity. Optical density (OD) was determined for each sample (y-axis).

FIG. 89 is a graphical representation showing the titration of monoclonal antibodies produced by plasmacytomas against full-length EF-Tu fused to NUS or against SEQ ID NO: 35. Dilutions of recombinant EF-Tu protein as indicated on the x-axis, were immobilised onto an ELISA plate. Antibodies designated 681E (⋄), 683B (▪), 682A (□), 680A (◯), 685B (X), 684A () and 521F (▴) at a concentration of 2.5 μg/ml were contacted with the immobilized recombinant EF-Tu protein for a time and under conditions sufficient for an antigen: antibody complex to form. The ELISA plate was washed and complexes detected by binding sheep anti-mouse Ig horseradish peroxidase (HRP) conjugate diluted 1:5000 (v/v) using TMB to detect bound HRP activity. Optical density (OD) was determined for each sample (y-axis).

FIG. 90 is a graphical representation showing the detection of recombinant EF-Tu using a sandwich ELISA using a chicken polyclonal anti-EF-Tu antibody as a capture reagent and monoclonal antibody 683B as a detection reagent. Various concentrations of each antibody were used as indicated in the legend of the figure. Titrating amounts of recombinant EF-Tu from dilutions of 1:2000 to 1:2,275,328 from a stock solution at A280=0.235 as indicated on the X-axis were screened. The ELISA plate was washed and complexes detected by binding sheep anti-mouse Ig horseradish peroxidase (HRP) conjugate diluted 1:5000 (v/v) using TMB to detect bound HRP activity. Optical density (OD) was determined for each sample (y-axis).

FIG. 91 is a graphical representation showing the detection of recombinant EF-Tu using a sandwich ELISA using a chicken polyclonal anti-EF-Tu antibody as a capture reagent and a monoclonal antibody 683B (▪) or 524D (♦) or 521F (▴) as a detection reagent. The polyclonal antibody was immobilised on an ELSA plate at a concentration of 5 μg/ml. Titrating amounts of recombinant EF-Tu as indicated on the x-axis were contacted to the immobilised antibody for a time and under conditions sufficient for an antibody: antigen complex to form. Each monoclonal antibody at a concentration of 5 μg/ml was then contacted to the immobilised recombinant EF-Tu for a time and under conditions sufficient for an antibody: antigen complex to form. The ELISA plate was washed and complexes detected by binding sheep anti-mouse Ig horseradish peroxidase (HRP) conjugate diluted 1:5000 (v/v) using TMB to detect bound HRP activity. Optical density (OD) was determined for each sample (y-axis).

FIG. 92 is a graphical representation showing the detection of recombinant EF-Tu using a sandwich ELISA using a chicken polyclonal anti-EF-Tu antibody from hen 49 (#) or hen 50 (▪) as a capture reagent and a monoclonal antibody designated 683B. For the purposes of nomenclature, the polyclonal antibody from hen 49 is also designated herein as “Ch49”, and the polyclonal antibody form hen 50 is also designated herein as “Ch50”. The polyclonal antibodies were immobilised on an ELISA plate at a concentration of 2.5 μg/ml. Titrating amounts of recombinant EF-Tu as indicated on the x-axis were contacted to the immobilised antibody for a time and under conditions sufficient for an antibody: antigen complex to form. The monoclonal antibody at a concentration of 2.5 μg/ml was then contacted to the immobilised recombinant EF-Tu for a time and under conditions sufficient for an antibody: antigen complex to form. The ELISA plate was washed and complexes detected by binding sheep anti-mouse Ig horseradish peroxidase (HRP) conjugate diluted 1:5000 (v/v) using TMB to detect bound HRP activity. Optical density (OD) was determined for each sample (y-axis).

FIG. 93 is a graphical representation comparing an amplified sandwich ELISA to standard sandwich ELISA for detecting recombinant M. tuberculosis EF-Tu protein. An ELISA plate was coated overnight with capture antibody Ch49 at 2 μg/ml concentration. Following washing to remove unbound antibody, recombinant EF-Tu protein was diluted from 100 ng/ml starting concentration to 1.0 pg/ml, and 50 μl aliquots of each dilution were added the wells of the antibody-coated ELISA plates (x-axis). Following incubation for 1 hour, plates were washed to remove unbound antigen. For both standard and amplified ELISA, monoclonal antibody 683B was biotinylated and the biotinylated monoclonal antibody (designated “Mo683B-bi”) was then contacted with the bound antigen-body complexes at 2.0 μg/ml concentration. Following incubation at room temperature for 1 hour, plates were washed, and incubated with 50 μl of a 1:5,000 (v/v) dilution of a secondary antibody consisting of HRP-conjugated sheep anti-mouse IgG (standard sandwich ELISA) or 50 μl of a 1:2,500 (v/v) dilution of HRP80-streptavidin (also termed “poly80-HRP-streptavidin”). Plates were then incubated for a further one hour at room temperature, and washed as before. Finally, all samples were incubated with TMB for 30 mins (standard ELISA) or 10 mins (amplified ELISA). Absorbance was determined at 450-620 nm (y-axis). Data indicate significant enhancement of detection using the amplified sandwich ELISA under these conditions: The limit of detection of this amplified sandwich ELISA is about 154 pg/ml EF-Tu protein. This compares favourably to the observed limit of detection of the standard sandwich ELISA of about 2.172 ng/ml EF-Tu protein.

FIG. 94 is a graphical representation of sandwich ELISA results showing detection of M. tuberculosis EF-Tu protein in whole cell extracts of the clinical M. tuberculosis isolates CSU93 and HN878, and in the laboratory strain H37Rv. Amplified sandwich ELISA conditions were essentially as described in the legend to FIG. 73, except for the following: (i) cellular extracts were assayed as indicated on the x-axis; (ii) the whole cell extracts were spiked with recombinant EF-Tu protein to a final concentration of 50, 16.7, 5.6 and 1.8 μg/ml; and (iii) the concentration of endogenous EF-Tu protein was determined by interpolation from a standard curve of EF-Tu concentration against signal strength, and corrected for the level of recombinant EF-Tu protein spike in the samples. Data are presented as picograms endogenous EF-Tu protein per microgram of total protein in the cellular extract (y-axis) for two separate experiments. Average protein levels are also indicated.

FIG. 95 is a graphical representation of sandwich ELISA results showing lack of significant cross-reactivity of antibodies against M. tuberculosis EF-Tu protein with whole cell lysates from Escherichia coli, Bacillus subtilis or Pseudomonas aeruginosa. Assay conditions were essentially as described in the legend to FIG. 74. As a standard, a serial dilution ranging from 39.1 pg/ml to 2.5 μg/ml purified recombinant EF-Tu protein was also created for comparison to signal strength from cellular extracts, as indicated on the x-axis. A negative control consisting of blocking buffer was also used., as indicated by “blank” on the x-axis. Data show low or no cross-reactivity between M. tuberculosis and whole cell lysates from Escherichia coli, Bacillus subtilis or Pseudomonas aeruginosa.

FIG. 96 is a graphical representation showing the effect of undiluted sputa in quenching detection of recombinant EF-Tu protein in the amplified sandwich ELISA assay described in preceding FIG. 7, and recovery of lost signal by dilution of the sputa. An ELISA plate was coated overnight with capture antibody Ch49 at 2.0 μg/ml concentration. Following washing to remove unbound antibody, recombinant EF-Tu protein was spiked to a concentration of 3.3 ng/ml into undiluted blocking solution (“blocker”), undiluted sputa (“sputum”), or a dilution of sputa in blocking solution ranging from 1:1 (v/v) block: sputum (“½”) to 8:1 (v/v) block: sputum (“⅛”) as indicated on the x-axis. Then, 50 μl aliquots of each sample was added the wells of the antibody-coated ELISA plates (x-axis). Following incubation for 1 hour and washing to remove unbound antigen, biotinylated monoclonal antibody Mo683B-bi was contacted with the bound antigen-body complexes at 2.0 μg/ml concentration. Following incubation at room temperature for 1 hour, plates were washed, and incubated with 50 μl of a 1:2,500 (v/v) dilution of HRP80-streptavidin (also termed “poly80-HRP-streptavidin”), incubated and then washed as before, and finally incubated with TMB for 10 mins. Absorbance was determined at 450-620 nm (y-axis). Data show quenching of signal by undiluted sputa, however there is significant signal recovery i.e., greater than 70% recovery in signal achieved by diluting the sputum.

FIG. 97 is a graphical representation showing the effect of undiluted plasma in quenching detection of recombinant EF-Tu protein in the amplified sandwich ELISA assay described in preceding FIG. 7, and recovery of lost signal by dilution of the plasma. An ELISA plate was coated overnight with capture antibody Ch49 at 2.0 μg/ml concentration. Following washing to remove unbound antibody, recombinant EF-Tu protein was spiked to a concentration of 3.3 ng/ml into undiluted blocking solution (“blocker”), undiluted plasma (“plasma”), or a dilution of plasma in blocking solution ranging from 1:1 (v/v) block: plasma (“½”) to 8:1 (v/v) block: plasma (“⅛”) as indicated on the x-axis. Then, 50 μl aliquots of each sample was added the wells of the antibody-coated ELISA plates (x-axis). Following incubation for 1 hour and washing to remove unbound antigen, biotinylated monoclonal antibody Mo683B-bi was contacted with the bound antigen-body complexes at 2.0 μg/ml concentration. Following incubation at room temperature for 1 hour, plates were washed, and incubated with 50 μl of a 1:2,500 (v/v) dilution of HRP80-streptavidin (also termed “poly80-HRP-streptavidin”), incubated and then washed as before, and finally incubated with TMB for 10 mins. Absorbance was determined at 450-620 nm (y-axis). Data show quenching of signal by undiluted plasma, however there is significant signal recovery i.e., greater than 70% recovery in signal achieved by diluting the plasma.

FIG. 98 is a graphical representation showing EF-Tu protein expression (relative to total cell protein) in M. tuberculosis, M. intracellulaire and M. avium, as determined by sandwich ELISA. Whole cell lysates from M. tuberculosis strain H37Rv (left), and from M. avium (middle) and M. intracellulaire (right) were assayed in duplicate in two independent experiments. The concentration of endogenous protein was calculated by interpolation from the standard curve and was corrected for dilution factor. The level of endogenous protein expressed as pg/μg total cellular protein was plotted as mean±SD for each of the three Mycobacteria tested.

FIG. 99 is a graphical representation showing the titration of polyclonal antibodies prepared in chickens against SEQ ID NO: 36. Recombinant P5CR (rP5CR) protein (SEQ ID NO: 36) was immobilized onto ELISA plate at a concentration of 5 μg/ml. Dilutions of antisera designated “Pink 6” (▪) and “Pink 7” (X) as indicated on the x-axis, and dilutions of pre-immune sera from the same animals (♦ for Pink 6; ▴ for Pink 7) as indicated on the x-axis, were contacted with the immobilized rP5CR protein for a time and under conditions sufficient for an antigen: antibody complex to form. The ELISA plate was washed and complexes detected by binding sheep anti-chicken IgG horseradish peroxidase (HRP) conjugate diluted 1:5000 (v/v) using TMB to detect bound HRP activity. Optical density (OD) was determined for each sample (y-axis). Data indicate antibody titers of at least about 1:32,000 (v/v) for both antibody preparations. The antibodies designated “Pink 6” are also referred to herein as “Ch6”, and the antibodies designated “Pink 7” are also referred to herein as “Ch7”.

FIG. 100 is graphical representation showing the titration of polyclonal antibodies prepared in rabbits against SEQ ID NO: 43. Streptavidin was immobilized onto an ELISA plate at a concentrate of 5 μg/ml. Biotin conjugated to a peptide consisting of the sequence set forth in SEQ ID NO: 43 (3 μg/ml) was contacted with the plate for a time and under conditions sufficient to immobilize the peptide via a biotin streptavidin interaction. Dilutions of rabbit antisera or pre-immune sera were added for a time and under conditions sufficient to form an antigen: antibody complexes, and bound antibodies were then detected as described in the legend of FIG. 79, except that the secondary antibody was a sheep anti-rabbit IgG HRP conjugate. Rabbit sera were designated Rb33 (=RCP33) (▪ for immune sera; ♦ for preimmune sera) and Rb34 (═RCP34) (X for immune sera; ▴ for preimmune sera). Sera dilutions are indicated on the x-axis. Optical density (OD) is indicated on the y-axis.

FIG. 101 is a graphical representation showing the detection limits of rabbit anti-P5CR antibodies Rb37 (=RCP37) and Rb38 (=RCP38). A biotinylated peptide comprising the sequence set forth in SEQ ID NO: 42 was bound to an ELISA plate as described in the legend to FIG. 80 except that the concentration of peptide was varied from 204.8 ng/ml to 100 pg/ml (x-axis). Antibodies Rb37 (♦,▪) and Rb38 (♦, X) and dilutions of 1:500 (v/v)(♦, ▴) and 1:2000 (v/v)(▪, X) were bound to the peptide and detected using a sheep anti-rabbit IgG HRP conjugate as described in the legend to FIG. 80. Data indicate that the limits of detection of Rb37 is about 0.8 ng/ml at 1:500 (v/v) dilution and about 1-3 ng/ml at 1:2000 (v/v) dilution; and that the limit of detection of Rb38 is about 1-5 ng/ml at dilutions at least up to about 1:2000 (v/v).

FIG. 102 is a graphical representation showing the binding of different antibodies to recombinant M. tuberculosis P5CR protein (SEQ ID NO: 36), as determined by ELISA. Recombinant P5CR protein was diluted serially 1:3 (v/v) from 55.555 ng/ml starting concentration to 228.62 pg/ml, and 50 μl aliquots of each dilution were used to coat the wells of an ELISA plate (x-axis). Following washing to remove unbound antigen, distinct antibodies, prepared by immunization of chickens (i.e., a polyclonal antibody pool designated Ch6/7, produced by combining polyclonal antibodies “Pink 6” and “Pink 7” referred to herein) or mice (i.e., a monoclonal antibody designated Mo1027D) or by phage display (Ph4550.2) with antigen, were contacted separately with the adsorbed recombinant P5CR protein at a concentration of 5 μg/ml. Following incubation at room temperature for 1 hour, plates were washed, incubated with 50 μl of a 1:5000 (v/v) dilution of secondary antibody (e.g., sheep anti-chicken IgG for detection of bound Ch6/7 antibody; and donkey anti-mouse IgG for detection of bound Mo1027D antibody) conjugated to horseradish peroxidase (HRP), washed, incubated with TMB for 30 mins, and absorbance at 450-620 nm was determined (y-axis). Data show significant binding of Ch6/7 polyclonal antibody and recombinant phage display antibody Ph4550.2 to recombinant P5CR.

FIG. 103 is a graphical representation showing optimization of sandwich ELISA results using antibody Ph4550.2 as capture antibody and polyclonal antibody pool Ch6/7 as detection antibody for assaying recombinant M. tuberculosis P5CR protein. An ELISA plate was coated overnight with capture antibody at 2 μg/ml, 5 μg/ml and 10 μg/ml concentrations. Following washing to remove unbound antibody, recombinant P5CR protein was diluted serially 1:3 (v/v) from 50 ng/ml starting concentration to 22.86 pg/ml, and 50 μl aliquots of each dilution were added the wells of the antibody-coated ELISA plates (x-axis). Following incubation for 1 hour and washing to remove unbound antigen, detection antibody Ch6/7 was contacted with the bound antigen-body complexes at concentrations in the range of 5 μg/ml or 10 μg/ml. Following incubation at room temperature for 1 hour, plates were washed, incubated with 50 μl of a 1:5000 (v/v) dilution of secondary antibody (i.e., sheep anti-chicken IgG) conjugated to horseradish peroxidase (HRP), washed, incubated with TMB for 30 mins, and absorbance at 450-620 nm was determined (y-axis). Without limiting the invention, data show optimum signal was detected using capture antibody at a concentration of 5 μg/ml or 10 μg/ml with detection antibody at a concentration of 5 μg/ml.

FIG. 104 is a graphical representation comparing the detection of horseradish peroxidise (HRP)-conjugated secondary antibodies to the detection of biotinylated secondary antibodies using streptavidin-HRP or streptavidin poly-40 HRP. An ELISA plate was coated overnight with 1-8 pg/ml recombinant P5CR protein, blocked, and then incubated with either biotinylated Ph4550.2 or unlabelled Ch6/7 antibody at 5 μg/ml concentration. Following washing to remove unbound antibody, a secondary sheep anti-chicken IgG antibody conjugated to HRP or donkey anti-chicken antibody conjugated to streptavidin poly-40 HRP was added to wells containing the Ch6/7 antibodies. In parallel reactions, secondary antibodies capable of binding to the biotinylated Ph4550.2 and labelled with streptavidin or streptavidin poly-40 HRP were added to wells containing the Ph4550.2 antibody. Following incubation, plates were washed, incubated with TMB for 30 mins, and absorbance at 450-620 nm was determined (y-axis). Data show significant signal enhancement using streptavidin poly-40 HRP as a detection reagent for Ch6/7 antibody compared to HRP.

FIG. 105 is a graphical representation showing optimization of amplified sandwich ELISA for amounts of capture and detection antibodies and dilution of a secondary streptavidin poly80 HRP conjugate, for assaying low concentrations of recombinant M. tuberculosis P5CR protein. An ELISA plate was coated overnight with capture antibody Ph4550.2 at 5 μg/ml or 10 μg/ml concentration. Following washing to remove unbound antibody, recombinant P5CR protein was diluted serially 1:3 (v/v) from 500 ng/ml starting concentration to 22.86 pg/ml, and 50 μl aliquots of each dilution were added the wells of the antibody-coated ELISA plates (x-axis). Following incubation for 1 hour and washing to remove unbound antigen, detection antibody Ch6/7 was contacted with the bound antigen-body complexes at 2.5 μg/ml or 5 μg/ml concentration. Following incubation at room temperature for 1 hour, plates were washed, incubated with 50 μl of a 1:200,000 (v/v) dilution of secondary antibody (i.e., biotinylated donkey anti-chicken IgG), washed as before, and then incubated with 50 μl of a 1:10,000 (v/v) dilution or 1:20,000 (v/v) dilution of HRP80-streptavidin conjugate. Plates were again washed and incubated with TMB for 30 mins, and absorbance at 450-620 nm was determined (y-axis). Without limiting the invention, data indicate that at eh concentration of secondary antibody employed herein, optimum detection limits for P5CR protein in sandwich ELISA using 10 μg/ml Ph4550.2 capture antibody and 2.5 μg/ml Ch6/7 detection antibody with a 1:10,000 dilution (v/v) amplified streptavidin poly-80 HRP.

FIG. 106 is a graphical representation comparing an amplified sandwich ELISA to standard sandwich ELISA for detecting recombinant M. tuberculosis P5CR protein. An ELISA plate was coated overnight with capture antibody Ph4550.2 at 5 μg/ml concentration. Following washing to remove unbound antibody, recombinant P5CR protein was diluted serially 1:10 (v/v) from 100 ng/ml starting concentration to 1.0 pg/ml, and 50 μl aliquots of each dilution were added the wells of the antibody-coated ELISA plates (x-axis). Following incubation for 1 hour and washing to remove unbound antigen, antibody Ch6/7 was contacted with the bound antigen-body complexes at 2.0 μg/ml concentration. Following incubation at room temperature for 1 hour, plates were washed, and incubated with 50 μl of a 1:20,000 (v/v) dilution of a secondary antibody consisting of unlabelled sheep anti-chicken IgG (standard sandwich ELISA) or biotinylated donkey anti-chicken IgG (amplified sandwich ELISA). Following incubation at room temperature for a further one hour, the plates washed as before. HRP (standard ELISA) or HRP80-streptavidin (amplified ELISA) was then added to the plates which were incubated for a further one hour at room temperature, washed as before and finally incubated with TMB for 30 mins. Absorbance was determined at 450-620 nm (y-axis). Data indicate significant enhancement of detection using the amplified sandwich ELISA under these conditions: The limit of detection of this amplified sandwich ELISA is about 48 pg/ml P5CR protein, with half-maximum detection of about 1 ng/ml P5CR protein.

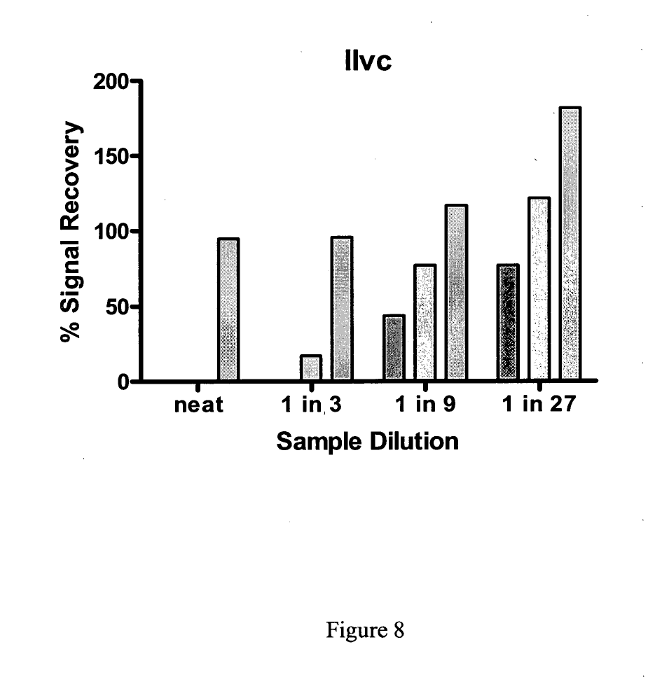

FIG. 107 is a graphical representation showing the effect of undiluted plasma in quenching detection of recombinant P5CR protein in the amplified sandwich ELISA assay described in preceding FIG. 8, and recovery of lost signal by dilution of the plasma. An ELISA plate was coated overnight with capture antibody Ph4550.2 at 5 μg/ml concentration. Following washing to remove unbound antibody, recombinant P5CR protein was spiked at the concentrations indicated on the x-axis into undiluted blocking solution (“block”), undiluted plasma (“neat plasma”), or a dilution of plasma in blocking solution ranging from 1:1 (v/v) block:plasma to 8:1 (v/v) block:plasma, and 50 μl aliquots of each sample added the wells of the antibody-coated ELISA plates (x-axis). Following incubation for 1 hour and washing to remove unbound antigen, antibody Ch6/7 was contacted with the bound antigen-body complexes at 2.0 μg/ml concentration. Following incubation at room temperature for 1 hour, plates were washed, and incubated with 50 μl of a 1:20,000 (v/v) dilution of a secondary antibody consisting of biotinylated donkey anti-chicken IgG (amplified sandwich ELISA). Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show that, notwithstanding there is some attenuation/suppression of signal by plasma, dilution of the clinical sample matrix does provide restoration of about more than 70% of the signal strength and up to about 88% of the signal strength.

FIG. 108 is a graphical representation showing the effect of undiluted sputum in quenching detection of recombinant P5CR protein in the amplified sandwich ELISA assay described in preceding FIG. 8, and complete recovery of lost signal by dilution of the clinical sample matrix. An ELISA plate was coated overnight with capture antibody Ph4550.2 at 5 μg/ml concentration. Following washing to remove unbound antibody, recombinant P5CR protein was spiked at the concentrations indicated on the x-axis into undiluted blocking solution (“block”), undiluted sputum (“neat sputa”), or a dilution of sputa in blocking solution ranging from 1:1 (v/v) block:sputa to 8:1 (v/v) block:sputa, and 50 ml aliquots of each sample added the wells of the antibody-coated ELISA plates (x-axis). Following incubation for 1 hour and washing to remove unbound antigen, antibody Ch6/7 was contacted with the bound antigen-body complexes at 2.0 μg/ml concentration. Following incubation at room temperature for 1 hour, plates were washed, and incubated with 50 μl of a 1:20,000 (v/v) dilution of a secondary antibody consisting of biotinylated donkey anti-chicken IgG (amplified sandwich ELISA). Following incubation at room temperature for a further one hour, the plates washed as before. HRP80-streptavidin (amplified ELISA) was then added to the plates which were incubated for a further one hour at room temperature, washed as before and finally incubated with TMB for 30 mins. Absorbance was determined at 450-620 nm (y-axis). Data show that, notwithstanding there is some minor attenuation/suppression of signal by sputa, dilution of the clinical sample matrix provides complete restoration and even some enhancement of the signal strength.

FIG. 109 is a graphical representation of sandwich ELISA results showing lack of significant cross-reactivity of antibodies against M. tuberculosis P5CR protein with whole cell lysates from yeast, Escherichia coli, Bacillus subtilis or Pseudomonas aeruginosa. Assay conditions were essentially as described in the legend to FIG. 86 except that 0-10 ng/ml purified recombinant P5CR protein or 100 ng/ml or 100 μg/ml of a cellular extract was assayed, as indicated on the x-axis. Buffer without protein or cellular extract served as a negative control. Data show the change in absorbance at 450-620 nm i.e., following subtraction of background absorbance for each sample.

FIG. 110 is a graphical representation of sandwich ELISA results showing detection of M. tuberculosis P5CR protein in whole cell extracts of the clinical M. tuberculosis isolates CSU93 and HN878, and in the laboratory strain H37Rv. Assay conditions were essentially as described in the legend to FIG. 89, except for the following: (i) the source of cellular extracts was as indicated on the x-axis; (ii) the whole cell extracts were spiked with recombinant P5CR protein to a final concentration of 50, 16.7, 5.6 and 1.8 μg/ml; and (iii) the concentration of endogenous P5CR protein was determined by interpolation from a standard curve of P5CR concentration against signal strength, and corrected for the level of recombinant P5CR protein spike in the samples. Data are presented as level of endogenous P5CR protein per microgram of total protein in the cellular extract (y-axis) for two separate experiments. Average protein levels are also indicated.

FIG. 111 is a graphical representation showing P5CR protein expression (relative to total cell protein) in M. tuberculosis, M. intracellulaire and M. avium, as determined by sandwich ELISA. Whole cell lysates from M. tuberculosis strain H37Rv (left), and from M. avium (middle) and M. intracellulaire (right) were assayed in duplicate in two independent experiments. The concentration of endogenous protein was calculated by interpolation from the standard curve and was corrected for dilution factor. The level of endogenous protein expressed as pg/μg total cellular protein was plotted as mean±SD for each of the three Mycobacteria tested.

FIG. 112 is a graphical representation showing the titration of polyclonal antibodies prepared in chickens against recombinant protein comprising SEQ ID NO: 44. Recombinant TetR (SEQ ID NO: 44) was immobilized onto ELISA plate at a concentration of 5 μg/ml. Dilutions of antisera designated “Pink 4” (▪) and “Pink 5” (X) as indicated on the x-axis, and dilutions of pre-immune sera from the same animals (♦ for Pink 4; ▴ for Pink 5) as indicated on the x-axis, were contacted with the immobilized TetR for a time and under conditions sufficient for an antigen: antibody complex to form. The ELISA plate was washed and complexes detected by binding sheep anti-chicken IgG horseradish peroxidase (HRP) conjugate diluted 1:5000 (v/v) using TMB to detect bound HRP activity. Optical density (OD) was determined for each sample (y-axis). Data indicate antibody titers of at least about 1:64,000 (v/v) for Pink 4 and at least about 1:128,000 (v/v) for Pink 5 for both antibody preparations. The antibody “Pink 4” is also referred to herein as “Ch4”; and the antibody “Pink 5” is also referred to herein as “Ch5”.

FIG. 113 is graphical representation showing the detection limits of polyclonal antibodies prepared in rabbits against SEQ ID NO: 55. Streptavidin was immobilized onto an ELISA plate at a concentrate of 5 μg/ml. Biotin conjugated to a peptide consisting of the sequence set forth in SEQ ID NO: 55 at concentrations in the range 204.8 μg/ml to 100 pg/ml as shown on the x-axis was contacted with the plate for a time and under conditions sufficient to immobilize the peptide via a biotin-streptavidin interaction. Dilutions (1:500 (v/v) or 1:2,000 (v/v)) of rabbit antisera or pre-immune sera were added for a time and under conditions sufficient to form an antigen: antibody complexes, and bound antibodies were then detected as described in the legend of FIG. 92, except that the secondary antibody was a sheep anti-rabbit IgG HRP conjugate. Rabbit sera was designated RCP18 (▪ for preimmune sera at 1:500 (v/v) dilutions; ♦ for immune sera at 1:500 (v/v) dilutions; X for preimmune sera at 1:2,000 (v/v) dilutions; and ▴ for immune sera at 1:2,000 (v/v) dilutions). Optical density (OD) is indicated on the y-axis. Data indicate that the limits of detection of RPC18 is about 0.1-0.5 ng/ml.

FIG. 114 is a graphical representation of a standard sandwich ELISA using the polyclonal antisera RCP 18 (=Rb18 in the figure) as a capture antibody and a pool of polyclonal antibodies designated “Ch4/5” which comprises the polyclonal antibodies Ch4 (=antibody “Pink 4” referred to herein) and Ch5 (=antibody “Pink 5” referred to herein) as detector antibody. This figure shows the effect of using these two antibody preparations in the sandwich ELISA. Wells of an ELISA plate were coated overnight with 50 μl of RCP18 (Rb18) antibody at 5 μg/ml or 10 μg/ml concentration. Following blocking and washing to remove unbound antibody, recombinant TetR-like protein was diluted from 50 ng/ml starting concentration to 80 pg/ml, and 50 μl aliquots of each dilution were added the wells of the antibody-coated ELISA plates (x-axis). Following incubation for 1 hour and washing to remove unbound antigen, the detection antibody i.e., Ch4/5 for detecting TetR-RCP 18 complexes was contacted with the bound antigen-body complexes at a concentration of 5 μg/ml or 10 μg/ml or 20 μg/ml. Following incubation at room temperature for 1 hour, plates were washed, incubated with 50 μl of a 1:5000 (v/v) dilution of secondary antibody (i.e., sheep anti-chicken IgG for detecting Ch4/5) conjugated to horseradish peroxidase (HRP), washed, incubated with TMB for 30 mins, and absorbance at 450-620 nm was determined after subtraction of background (y-axis). Without limiting the invention, data suggest that the combination of 5 μg/ml RCP 18 as capture antibody and 5 μg/ml Ch4/5 as detector antibody is preferred in this sandwich ELISA format and detects TetR to at least 5 ng/ml of protein.

FIG. 115 is a graphical representation of a standard sandwich ELISA using a pool of polyclonal antibodies designated “Ch4/5” which comprises the polyclonal antibodies Ch4 (=antibody “Pink 4” referred to herein) and Ch5 (=antibody “Pink 5” referred to herein) as capture antibody, and the polyclonal antisera RCP 18 (=Rb18 in the figure) as a detector antibody. The figure shows the effect of using these two antibody preparations in the sandwich ELISA. Wells of an ELISA plate were coated overnight with 50 μl of Ch4/5 antibody at 5 μg/ml or 10 μg/ml concentration. Following blocking and washing to remove unbound antibody, recombinant TetR-like protein was diluted from 50 ng/ml starting concentration to 80 pg/ml, and 50 μl aliquots of each dilution were added the wells of the antibody-coated ELISA plates (x-axis). Following incubation for 1 hour and washing to remove unbound antigen, the detection antibody i.e., RCP18 for detecting TetR-Ch4/5 complexes was contacted with the bound antigen-body complexes at a concentration of 5 μg/ml or 10 μg/ml or 20 μg/ml. Following incubation at room temperature for 1 hour, plates were washed, incubated with 50 μl of a 1:5000 (v/v) dilution of secondary antibody (i.e., sheep anti-rabbit IgG for detecting Ch4/5) conjugated to horseradish peroxidase (HRP), washed, incubated with TMB for 30 mins, and absorbance at 450-620 nm was determined after subtraction of background (y-axis). Without limiting the invention, data suggest that the combination of 5 μg/ml Ch4/5 as capture antibody and 5 μg/ml RCP18 as detector antibody is preferred in this sandwich ELISA format, and marginally improved compared to the reverse orientation of antibodies shown in FIG. 3.

FIG. 116 is a graphical representation of a standard sandwich ELISA using a pool of polyclonal antibodies designated “Ch4/5” which comprises the polyclonal antibodies Ch4 (=antibody “Pink 4” referred to herein) and Ch5 (=antibody “Pink 5” referred to herein) as capture antibody, and one of two monoclonal antibody preparations designated 784F and Mo785E as detector antibody. The figure shows the effect of using these antibody preparations in the sandwich ELISA. Wells of an ELISA plate were coated overnight with 50 μl of Ch4/5 antibody at 500 ng/ml or 1 μg/ml or 2 μg/ml or 4 μg/ml or 8 μg/ml concentration. Following blocking and washing to remove unbound antibody, recombinant TetR-like protein was diluted from 5 ng/ml starting concentration to 2.29 pg/ml, and 50 μl aliquots of each dilution were added the wells of the antibody-coated ELISA plates (x-axis). Following incubation for 1 hour and washing to remove unbound antigen, the detection antibody i.e., M 784F or M Mo785E for detecting TetR-Ch4/5 complexes was contacted with the bound antigen-body complexes at a concentration of 2 μg/ml. Following incubation at room temperature for 1 hour, plates were washed, incubated with 50 μl of a 1:5000 (v/v) dilution of secondary antibody (i.e., sheep anti-mouse IgG for detecting the mouse monoclonal antibodies) conjugated to horseradish peroxidase (HRP), washed, incubated with TMB for 30 mins, and absorbance at 450-620 nm was determined after subtraction of background (y-axis). Without limiting the invention, data suggest that the Mo785E monoclonal antibody provides the lowest background signal and, when combined with the Ch4/5 capture antibody, provides higher signals than the rabbit polyclonal RCP 18. The combination of 500 ng/ml Ch4/5 as capture antibody and 2 μg/ml Mo785E as detector antibody provided the lowest background signal, however the combination of 2 μg/ml Ch4/5 as capture antibody and 2 μg/ml Mo785E as detector antibody provided the highest signal: noise ratio in this sandwich ELISA format.

FIG. 117 is a graphical representation comparing an amplified sandwich ELISA to standard sandwich ELISA for detecting recombinant M. tuberculosis TetR-like protein. An ELISA plate was coated overnight with capture antibody Ch4/5 at 2 μg/ml concentration. Following washing to remove unbound antibody, recombinant TetR-like protein was diluted from 100 ng/ml starting concentration to 490 fg/ml, and 50 μl aliquots of each dilution were added the wells of the antibody-coated ELISA plates (x-axis). Following incubation for 1 hour, plates were washed to remove unbound antigen. Unlabelled monoclonal antibody Mo785E was contacted with the bound antigen-body complexes at 2.5 μg/ml concentration for standard sandwich ELISA. For amplified sandwich ELISA, monoclonal antibody Mo785E was biotinylated and the biotinylated antibody contacted with the bound antigen-body complexes at 2.5 μg/ml concentration. Following incubation at room temperature for 1 hour, plates were washed, and incubated with 50 μl of a 1:5,000 (v/v) dilution of a secondary antibody consisting of HRP-conjugated sheep anti-mouse IgG (standard sandwich ELISA) or 50 μl of a 1:2,500 (v/v) dilution of HRP80-streptavidin. Plates were then incubated for a further one hour at room temperature, and washed as before. Finally, all samples were incubated with TMB for 30 mins (standard ELISA) or 10 mins (amplified ELISA). Absorbance was determined at 450-620 nm (y-axis). Data indicate significant enhancement of detection using the amplified sandwich ELISA under these conditions: The limit of detection of this amplified sandwich ELISA is about 18 pg/ml TetR-like protein, with half-maximum detection of about 1 ng/ml TetR-like protein. This compares favourably to the observed limit of detection of the standard sandwich ELISA of about 176 pg/ml TetR-like protein.

FIG. 118 is a graphical representation of sandwich ELISA results showing detection of M. tuberculosis TetR-like protein in whole cell extracts of the clinical M. tuberculosis isolates CSU93 and HN878, and in the laboratory strain H37Rv. Amplified sandwich ELISA conditions were essentially as described in the legend to FIG. 97, except for the following: (i) cellular extracts were assayed as indicated on the x-axis; (ii) the whole cell extracts were spiked with recombinant TetR-like protein to a final concentration of 50, 16.7, 5.6 and 1.8 μg/ml; and (iii) the concentration of endogenous TetR-like protein was determined by interpolation from a standard curve of TetR concentration against signal strength, and corrected for the level of recombinant TetR-like protein spike in the samples. Data are presented as picograms endogenous TetR-like protein per microgram of total protein in the cellular extract (y-axis) for two separate experiments. Average protein levels are also indicated.

FIG. 119 is a graphical representation of sandwich ELISA results showing lack of significant cross-reactivity of antibodies against M. tuberculosis TetR-like protein with whole cell lysates from yeast, Escherichia coli, Bacillus subtilis or Pseudomonas aeruginosa. Assay conditions were essentially as described in the legend to FIG. 98 except that HRP40-streptavidin as opposed to HRP80-streptavidin was used at 1:2500 (v/v) dilution, TMB was developed for 15 min for signal detection, and 450 fg/ml to 1 ng/ml purified recombinant TetR-like protein or a serial dilution [1:3 (v/v)] of cellular extract i.e., 11.1 μg/ml or 33.3 μg/ml or 100 μg/ml was assayed as indicated on the x-axis. Buffer without protein or cellular extract served as a negative control. Data show no cross-reactivity between M. tuberculosis and whole cell lysates from yeast, Escherichia coli, Bacillus subtilis or Pseudomonas aeruginosa.

FIG. 120 is a graphical representation showing TetR-like protein expression (relative to total cell protein) in M. tuberculosis, M. intracellulaire and M. avium, as determined by sandwich ELISA. Whole cell lysates from M. tuberculosis strain H37Rv (left), and from M. avium (middle) and M. intracellulaire (right) were assayed in duplicate in two independent experiments. The concentration of endogenous protein was calculated by interpolation from the standard curve and was corrected for dilution factor. The level of endogenous protein expressed as pg/μg total cellular protein was plotted as mean±SD for each of the three Mycobacteria tested.

FIG. 121 is a graphical representation showing TetR-like protein expression in filtrates obtained from whole cell lysates of M. tuberculosis, M. intracellulaire and M. avium, as determined by sandwich ELISA. Filtrates obtained from whole cell lysates of M. tuberculosis strain H37Rv (left), M. avium (middle) and M. intracellulaire (right) were assayed in duplicate. The concentration of endogenous protein was calculated by interpolation from the standard curve and was corrected for dilution factor (if any). The level of endogenous protein expressed as pg/μL filtrate was plotted as mean±SD for each of the three Mycobacteria.

FIG. 122 provides graphical representations showing inhibition of antibody binding to recombinant BSX (top left), Rv1265 (top right), S9 (lower left) and KARI (lower right) proteins by sputum. An amplified ELISA system was used to analyze the degree of inhibition of antibody binding to recombinant protein in TB-negative sputum. The sputum was spiked with 10 ng/mL of each recombinant protein (columns 1-2 in each panel), a 1:3 (v/v) dilution of the mixture in blocking buffer (columns 4-5 in each panel), a 1:9 (v/v) dilution of the mixture in blocking buffer (columns 7-8 in each panel), and a 1:27 (v/v) dilution of the mixture in blocking buffer (columns 10-11 in each panel). Samples were incubated overnight at 4° C. before assay (columns 1, 4, 7, 10 in each panel) or assayed immediately (columns 2, 5, 8, 11 in each panel). Positive control samples lacked sputum and were incubated overnight before assay (columns 3, 6, 9, 12 in each panel). The samples were assayed in duplicate wells in each of 2 separate experiments. The concentration of recombinant protein detected in sputum at each dilution was calculated by interpolation from the standard curve, and expressed as a % of the spiked concentration of recombinant protein (% signal recovery). The level of recovery was plotted as mean % signal recovery±SD for each of the 4 dilution factors for the three treatments. Data are presented as percentage signal recovery (Y-axis) for each dilution shown on the x-axis.

FIG. 123 provides graphical representations showing the inhibition of antibody binding to endogenous M. tuberculosis BSX (top left), Rv1265 (top right), S9 (lower left) and KARI (lower right) proteins by sputum, as determined by amplified sandwich ELISA. An amplified ELISA system was used to analyse the levels of quenching and masking of antibody binding to endogenous BSX, S9, Rv1265 and KARI proteins in whole cell lysates of M. tuberculosis H37Rv spiked into TB-negative sputum. The sputum was spiked with whole cell lysates to achieve target concentrations in sputum of BSX=9 ng/mL, Rv1265=2.8 ng/mL, S9=1.2 ng/mL and KARI=31 ng/mL (columns 1-2 in each panel), a 1:3 (v/v) dilution of the mixture in blocking buffer (columns 4-5 in each panel), a 1:9 (v/v) dilution of the mixture in blocking buffer (columns 7-8 in each panel), and a 1:27 (v/v) dilution of the mixture in blocking buffer (columns 10-11 in each panel). Samples were incubated overnight at 4° C. before assay (columns 1, 4, 7, 10 in each panel) or assayed immediately (columns 2, 5, 8, 11 in each panel). Positive control samples lacked sputum and were incubated overnight before assay (columns 3, 6, 9, 12 in each panel). The samples were assayed in duplicate wells in each of 2 separate experiments. The concentration of endogenous protein detected in sputum at each dilution was calculated by interpolation from the standard curve (H37Rv-WCL serially diluted in blocking buffer), and expressed as a % of the spiked concentration (% signal recovery). The level of recovery was plotted as mean % signal recovery for each of the 4 dilution factors for the three treatments.

FIG. 124 is a graphical representation showing relative expression of BSX (columns 1-3), EF-Tu (columns 4-6), KARI (columns 7-9), P5CR (columns 10-12), antigen A (columns 13-15), Rv1265 (columns 16-18), antigen B (columns 19-21), antigen C (columns 22-24), S9 (columns 25-27), antigen D (columns 28-30) and antigen E (columns 31-33) expressed on the basis of total cellular protein in M. tuberculosis strain H37Rv (first column in each group of 3 columns), CSU93 (second column in each group of 3 columns) and HN878 (third column in each group of 3 columns), as determined by sandwich ELISA. The concentration of endogenous protein was calculated by interpolation from the standard curve and was corrected for the dilution factor. Data were obtained from replicate experiments in which each sample was analysed in duplicate. The levels of endogenous protein (expressed as pg/μg total cell protein) was plotted as mean±SD for each of 11 TB antigens analysed.

FIG. 125 is an expanded view of the graphical representation set forth in FIG. 124 showing the expression levels for some low expressing antigens.

FIG. 126 is a graphical representation showing relative expression of BSX (columns 1-3), EF-Tu (columns 4-6), KARI (columns 7-9), P5CR (columns 10-12), antigen A (columns 13-15), Rv1265 (columns 16-18), antigen B (columns 19-21), antigen C (columns 22-24), S9 (columns 25-27), antigen D (columns 28-30) and antigen E (columns 31-33) expressed as ng protein per 1×10⁶ CFU M. tuberculosis strain H37Rv (first column in each group of 3 columns), M. avium (second column in each group of 3 columns) and in M. intracellulaire (third column in each group of 3 columns), as determined by sandwich ELISA. The concentration of endogenous protein was calculated by interpolation from the standard curve and was corrected for the dilution factor. Data were obtained from replicate experiments in which each sample was analysed in duplicate. The levels of endogenous protein were plotted as mean±SD for each of 11 TB antigens analyzed. Data indicate specific expression of BSX, EF-Tu, KARI, Rv1265 and S9 in M. tuberculosis.

FIG. 127 is an expanded view of the graphical representation set forth in FIG. 126 showing the expression levels for some low expressing antigens. Data indicate specific expression of BSX, EF-Tu, P5CR, Rv1265 and S9 in M. tuberculosis with detectable expression of KARI in M. intracellulaire and M. avium at these low detection limits.

FIG. 128 is a graphical representation showing relative expression of BSX (columns 1-3), EF-Tu (columns 4-6), KARI (columns 7-9), P5CR (columns 10-12), antigen A (columns 13-15), Rv1265 (columns 16-18), antigen B (columns 19-21), antigen C (columns 22-24), S9 (columns 25-27), antigen D (columns 28-30) and antigen E (columns 31-33) expressed as pg antigen per μg total cell protein of M. tuberculosis strain H37Rv (first column in each group of 3 columns), M. avium (second column in each group of 3 columns) and in M. intracellulaire (third column in each group of 3 columns), as determined by sandwich ELISA. The concentration of endogenous protein was calculated by interpolation from the standard curve and was corrected for the dilution factor. Data were obtained from replicate experiments in which each sample was analysed in duplicate. The levels of endogenous protein were plotted as mean±SD for each of 11 TB antigens analyzed. Data indicate specific expression of BSX, EF-Tu, Rv1265 and S9 in M. tuberculosis. Detectable expression of KARI was apparent in M. intracellulaire and M. avium under these conditions.

FIG. 129 is an expanded view of the graphical representation set forth in FIG. 128 showing the expression levels for some low expressing antigens. Data indicate specific expression of Rv1265 in M. tuberculosis with detectable expression of most other antigens tested in M. intracellulaire and M. avium at these low detection limits.

FIG. 130 is a graphical representation showing relative expression of BSX (columns 1-3), EF-Tu (columns 4-6), KARI (columns 7-9), P5CR (columns 10-12), antigen A (columns 13-15), Rv1265 (columns 16-18), antigen B (columns 19-21), antigen C (columns 22-24), S9 (columns 25-27), antigen D (columns 28-30) and antigen E (columns 31-33) expressed as pg antigen per μL filtrate of a whole cell lysate of M. tuberculosis strain H37Rv (first column in each group of 3 columns), M. avium (second column in each group of 3 columns) and in M. intracellulaire (third column in each group of 3 columns), as determined by sandwich ELISA. The concentration of endogenous protein was calculated by interpolation from the standard curve and was corrected for the dilution factor. Data were obtained from replicate experiments in which each sample was analysed in duplicate. The levels of endogenous protein were plotted as mean±SD for each of 11 TB antigens analyzed.

FIG. 131 is an expanded view of the graphical representation set forth in FIG. 130 showing the expression levels for some low expressing antigens.

FIG. 132 provides graphical representations showing assay working ranges and limits of detection for recombinant M. tuberculosis KARI, (IlvC), BSX, Rv1265 and S9 proteins (left panel) and endogenous M. tuberculosis KARI, (IlvC), BSX, Rv1265 and S9 proteins in whole cell lysates (WCL) of M. tuberculosis strain H37Rv (right panel). Recombinant protein and whole cell lysate protein concentrations are shown on the x-axis (μg/ml) and absorbance in amplified sandwich ELISA performed under standard conditions using antibody pairs as described herein are shown on the y-axis. Data indicate that all four antigens are capable of being detected significantly at nanogram concentrations, corresponding to microgram concentrations of whole cell lysates.

### DETAILED DESCRIPTION OF THE PREFERRED EXAMPLES

### Isolated or Recombinant KARI Protein and Immunogenic Fragments and Epitopes Thereof

One aspect of the present invention provides an isolated or recombinant KARI protein or an immunogenic fragment or epitope thereof.

This aspect of the invention encompasses any synthetic or recombinant peptides derived from a KARI protein referred to herein, including the full-length KARI protein, and/or a derivative or analogue of a KARI protein or an immunogenic fragment or epitope thereof.

A preferred KARI protein is a peptide, polypeptide, or protein having at least about 80% amino acid sequence identity to the amino acid sequence set forth in SEQ ID NO: 1. Preferably, the percentage identity of a KARI protein to SEQ ID NO: 1 is at least about 85%, more preferably at least about 90%, even more preferably at least about 95% and still more preferably at least about 99%. The present invention is not to be restricted to the use of the exemplified M. tuberculosis KARI protein because, as will be known to those skilled in the art, it is possible to define a fragment of a protein having sequence identity and immune-logical equivalence to a region of the exemplified M. tuberculosis KARI protein without undue experimentation.

In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Madison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximise the number of identical/similar residues and to minimise the number and/or length of sequence gaps in the alignment. Amino acid sequence alignments can also be performed using a variety of other commercially available sequence analysis programs, such as, for example, the BLAST program available at NCBI.

Particularly preferred fragments include those that include an epitope, in particular a B cell epitope or T cell epitope.

A B-cell epitope is conveniently derived from the amino acid sequence of an immunogenic KARI protein. Idiotypic and anti-idiotypic B cell epitopes against which an immune response is desired are specifically encompassed by the invention, as are lipid-modified B cell epitopes or a Group B protein. A preferred B-cell epitope will be capable of eliciting the production of antibodies when administered to a mammal, preferably neutralizing antibody against M. tuberculosis, and more preferably, a high titer neutralizing antibody. Shorter B cell epitopes are preferred, to facilitate peptide synthesis. Preferably, the length of the B cell epitope will not exceed about 30 amino acids in length. More preferably, the B cell epitope sequence consists of about 25 amino acid residues or less, and more preferably less than 20 amino acid residues, and even more preferably about 5-20 amino acid residues in length derived from the sequence of the full-length protein.

A CTL epitope is also conveniently derived from the full length amino acid sequence of a KARI protein and will generally consist of at least about 9 contiguous amino acids of said KARI protein and have an amino acid sequence that interacts at a significant level with a MHC Class I allele as determined using a predictive algorithm for determining MHC Class I-binding epitopes, such as, for example, the SYFPEITHI algorithm of the University of Tuebingen, Germany, or the algorithm of the HLA Peptide Binding Predictions program of the BioInformatics and Molecular Analysis Section (BIMAS) of the National Institutes of Health of the Government of the United States of America. More preferably, the CTL epitope will have an amino acid sequence that binds to and/or stabilizes a MHC Class 1 molecule on the surface of an antigen presenting cell (APC). Even more preferably, the CTL epitope will have a sequence that induces a memory CTL response or elicits IFN-γ expression by a T cell, such as, for example, CD8⁺ T cell, cytotoxic T cell (CTL). Still even more preferably, the CTL will have a sequence that stimulates CTL activity in a standard cytotoxicity assay. Particularly preferred CTL epitopes of a KARI protein are capable of eliciting a cellular immune response against M. tuberculosis in human cells or tissues, such as, for example, by recognizing and lysing human cells infected with M. tuberculosis, thereby providing or enhancing cellular immunity against M. tuberculosis.

Suitable fragments will be at least about 5, e.g., 10, 12, 15 or 20 amino acids in length. They may also be less than 200, 100 or 50 amino acids in length.

The amino acid sequence of a KARI protein or immunogenic fragment or epitope thereof may be modified for particular purposes according to methods known to those of skill in the art without adversely affecting its immune function. For example, particular peptide residues may be derivatized or chemically modified in order to enhance the immune response or to permit coupling of the peptide to other agents, particularly lipids. It also is possible to change particular amino acids within the peptides without disturbing the overall structure or antigenicity of the peptide. Such changes are therefore termed “conservative” changes and tend to rely on the hydrophilicity or polarity of the residue. The size and/or charge of the side chains also are relevant factors in determining which substitutions are conservative.

The present invention clearly encompasses a covalent fusion between one or more immunogenic KARI peptides, including a homo-dimer, homo-trimer, homo-tetramer or higher order homo-multimer of a peptide, or a hetero-dimer, hetero-trimer, hetero-tetramer or higher order hetero-multimer comprising two or more different immunogenic peptides.

The present invention also encompasses a non-covalent aggregate between one or more immunogenic KARI peptides, e.g., held together by ionic, hydrostatic or other interaction known in the art or described herein.

It is well understood by the skilled artisan that, inherent in the definition of a biologically functional equivalent protein is the concept that there is a limit to the number of changes that may be made within a defined portion of the molecule and still result in a molecule with an acceptable level of equivalent biological activity. Biologically functional equivalent proteins are thus defined herein as those proteins in which specific amino acids are substituted. Particular examples encompass variants that have one, two, three, four, five or more variations in the amino acid sequence of the peptide. Of course, a plurality of distinct proteins/peptides with different substitutions may easily be made and used in accordance with the invention.

Those skilled in the art are well aware that the following substitutions are permissible conservative substitutions (i) substitutions involving arginine, lysine and histidine; (ii) substitutions involving alanine, glycine and serine; and (iii) substitutions involving phenylalanine, tryptophan and tyrosine. Derivatives incorporating such conservative substitutions are defined herein as biologically or immune-logically functional equivalents.

The importance of the hydropathic amino acid index in conferring interactive biological function on a protein is generally understood in the art (Kyte & Doolittle, J. Mol. Biol. 157, 105-132, 1982). It is known that certain amino acids may be substituted for other amino acids having a similar hydropathic index or score and still retain a similar biological activity. The hydropathic index of amino acids also may be considered in determining a conservative substitution that produces a functionally equivalent molecule. Each amino acid has been assigned a hydropathic index on the basis of their hydrophobicity and charge characteristics, as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making changes based upon the hydropathic index, the substitution of amino acids whose hydropathic indices are within +/−0.2 is preferred. More preferably, the substitution will involve amino acids having hydropathic indices within +/−0.1, and more preferably within about +/−0.05.

It is also understood in the art that the substitution of like amino acids is made effectively on the basis of hydrophilicity, particularly where the biological functional equivalent protein or peptide thereby created is intended for use in immune-logical examples, as in the present case (e.g. U.S. Pat. No. 4,554,101). In fact, the greatest local average hydrophilicity of a protein, as governed by the hydrophilicity of its adjacent amino acids, correlates with its immunogenicity and antigenicity. As detailed in U.S. Pat. No. 4,554,101, the following hydrophilicity values have been assigned to amino acid residues: arginine (+3.0); lysine (+3.0); aspartate (+3.0+/−0.1); glutamate (+3.0+/−0.1); serine (+0.3); asparagine (+0.2); glutamine (+0.2); glycine (0); threonine (−0.4); proline (−0.5+/−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, it is preferred to substitute amino acids having hydrophilicity values within about +/−0.2 of each other, more preferably within about +/−0.1, and even more preferably within about +/−0.05

The KARI polypeptide or peptide fragment thereof comprising an epitope is readily synthesized using standard techniques, such as the Merrifield method of synthesis (Merrifield, J Am Chem Soc, 85, 2149-2154, 1963) and the myriad of available improvements on that technology (see e.g., Synthetic Peptides: A User's Guide, Grant, ed. (1992) W.H. Freeman & Co., New York, pp. 382; Jones (1994) The Chemical Synthesis of Peptides, Clarendon Press, Oxford, pp. 230.); Barany, G. and Merrifield, R. B. (1979) in The Peptides (Gross, E. and Meienhofer, J. eds.), vol. 2, pp. 1-284, Academic Press, New York; Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474.d

As is known in the art, synthetic peptides can be produced with additional hydrophilic N-terminal and/or C-terminal amino acids added to the sequence of a fragment or B-cell epitope derived from the full-length KARI protein, such as, for example, to facilitate synthesis or improve peptide solubility. Glycine and/or serine residues are particularly preferred for this purpose. Such peptides may be modified to include additional spacer sequences flanking the KARI fragments, said spacers comprising hetero-polymers (trimers or tetramers) comprising glycine and serine.

The peptides of the invention are readily modified for diagnostic purposes, for example, by addition of a natural or synthetic hapten, an antibiotic, hormone, steroid, nucleoside, nucleotide, nucleic acid, an enzyme, enzyme substrate, an enzyme inhibitor, biotin, avidin, streptavidin, polyhistidine tag, glutathione, GST, polyethylene glycol, a peptidic polypeptide moiety (e.g. tuftsin, poly-lysine), a fluorescence marker (e.g. FITC, RITC, dansyl, luminol or coumarin), a bioluminescence marker, a spin label, an alkaloid, biogenic amine, vitamin, toxin (e.g. digoxin, phalloidin, amanitin, tetrodotoxin), or a complex-forming agent. Biotinylated peptides are especially preferred.

In another example, a KARI protein or immunogenic fragment or epitope thereof is produced as a recombinant protein.

For expressing protein by recombinant means, a protein-encoding nucleotide sequence is placed in operable connection with a promoter or other regulatory sequence capable of regulating expression in a cell-free system or cellular system. In one example of the invention, nucleic acid comprising a sequence that encodes a KARI protein (e.g. as set forth in SEQ ID NO: 1) or an epitope thereof in operable connection with a suitable promoter sequence, is expressed in a suitable cell for a time and under conditions sufficient for expression to occur. Nucleic acid encoding the KARI protein, including the ilvC gene of M. tuberculosis and any variants thereof encoding a KARI protein as described herein, is readily derived from the publicly available amino acid sequence.

In another example, a KARI protein is produced as a recombinant fusion protein, such as for example, to aid in extraction and purification. To produce a fusion polypeptide, the open reading frames are covalently linked in the same reading frame, such as, for example, using standard cloning procedures as described by Ausubel et al. (Current Protocols in Molecular Biology, Wiley Interscience, ISBN 047150338, 1992), and expressed under control of a promoter. Examples of fusion protein partners include glutathione-S-transferase (GST), FLAG (Asp-Tyr-Lys-Asp-Asp-Asp-Asp-Lys), hexa-histidine, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the immune function of the KARI protein.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or external stimuli, or in a tissue-specific manner. In the present context, the term “promoter” is also used to describe a recombinant, synthetic or fusion molecule, or derivative which confers, activates or enhances the expression of a nucleic acid to which it is operably connected, and which encodes the polypeptide or peptide fragment. Preferred promoters can contain additional copies of one or more specific regulatory elements to further enhance expression and/or to alter the spatial expression and/or temporal expression of the said nucleic acid molecule.

Placing a nucleic acid under the regulatory control of, i.e., “in operable connection with”, a promoter means positioning said nucleic acid such that expression is controlled by the promoter sequence. Promoters are generally positioned 5′ (upstream) to the coding sequence that they control.

The prerequisite for producing intact polypeptides and peptides in bacteria such as E. coli is the use of a strong promoter with an effective ribosome binding site. Typical promoters suitable for expression in bacterial cells such as E. coli include, but are not limited to, the lacZ promoter, temperature-sensitive λ_{L} or λ_{R} promoters, T7 promoter or the IPTG-inducible tac promoter. A number of other vector systems for expressing the nucleic acid molecule of the invention in E. coli are known in the art and are described, for example, in Ausubel et al (In: Current Protocols in Molecular Biology. Wiley Interscience, ISBN 047150338, 1987) or Sambrook et al (In: Molecular cloning, A laboratory manual, second edition, Cold Spring Harbor Laboratory, Cold Spring Harbor, N.Y., 1989). Numerous plasmids with suitable promoters for expression in bacteria and efficient ribosome binding sites have been described, such as for example, pKC30 (λ_{L}: Shimatake and Rosenberg, Nature 292, 128, 1981); pKK173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, CA), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (eg. 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing a secreted form of a KARI protein or a derivative thereof in 293T cells, wherein the expressed peptide or protein can be purified free of conspecific proteins, using standard affinity techniques that employ a Nickel column to bind the protein via the His tag.

A wide range of additional host/vector systems suitable for expressing the diagnostic protein of the present invention or an immune-logical derivative (e.g., an epitope or other fragment) thereof are available publicly, and described, for example, in Sambrook et al (In: Molecular cloning, A laboratory manual, second edition, Cold Spring Harbor Laboratory, Cold Spring Harbor, N.Y., 1989).

Means for introducing the isolated nucleic acid molecule or a gene construct comprising same into a cell for expression are known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

Proteins of the invention can be produced in an isolated form, preferably substantially free of conspecific protein. Antibodies and other affinity ligands are particularly preferred for producing isolated protein. Preferably, the protein will be in a preparation wherein more than about 90% (e.g. 95%, 98% or 99%) of the protein in the preparation is a KARI protein or an epitope thereof.

### Isolated or Recombinant Secondary Analyte Protein, Peptides and Epitopes Thereof

It is to be understood that methods described herein above for the production of isolated and recombinant KARI protein or immunogenic fragments thereof apply mutatis mutandis to the production of secondary analyte proteins, peptides and fragments that are to be used in an immune-assay format e.g., for the purposes of diagnosis or prognosis of tuberculosis or infection by M. tuberculosis, antibody production, analyte purification, vaccine formulation, etc. As will be understood by the skilled artisan, such extrapolation is dependent on substituting the KARI protein immunogen for the secondary analyte in question e.g., M. tuberculosis BSX protein or S9 protein or GS protein or immunogenic fragment thereof according to any example hereof or a combination or mixture of said peptides or epitopes or fragments. Such substitution is readily performed without undue experimentation from the disclosure herein.

For convenience, preferred secondary analytes e.g., for use in multi-analyte antigen-based tests, will comprise an amino acid sequence selected from the group set forth in SEQ ID NOs: 3-60, and combinations/mixtures thereof.

For example, the M. tuberculosis BSX protein can be expressed and fragments obtained there from by standard means, or alternatively, synthetic peptides can be synthesized based on the sequence of the full-length protein (e.g., comprising the sequence set forth in SwissProt Database Accession No. 053759). Exemplary immunogenic peptides from the full-length BSX protein will comprise a sequence selected from the group consisting of MRQLAERSGVSNPYL (SEQ ID NO: 3), ERGLRKPSADVLSQI (SEQ ID NO: 4), LRKPSADVLSQIAKA (SEQ ID NO: 5), PSADVLSQIAKALRV (SEQ ID NO: 6), SQIAKALRVSAEVLY (SEQ ID NO: 7), AKALRVSAEVLYVRA (SEQ ID NO: 8), VRAGILEPSETSQVR (SEQ ID NO: 9), TAITERQKQILLDIY (SEQ ID NO: 10), SQIAKALRVSAEVLYVRAC (SEQ ID NO: 11), MSSEEKLCDPTPTDD (SEQ ID NO: 12) and VRAGILEPSETSQVRC (SEQ ID NO: 13). Methods for producing such fragments are described in detail in the instant applicant's International Patent Application No. PCT/AU2005/001254 filed Aug. 19, 2005 (WO 2006/01792) the disclosure of which is incorporated herein in its entirety.

Alternatively, or in addition, M. tuberculosis glutamine synthetase (GS) protein can be expressed and fragments obtained there from by standard means, or alternatively, synthetic peptides can be synthesized based on the sequence of the full-length protein (e.g., comprising the sequence set forth in SwissProt Database Accession No. O33342). Exemplary immunogen fragments of the GS protein are derived from a surface-exposed region of a GS protein, or comprise the sequence RGTDGSAVFADSNGPHGMSSMFRSF (SEQ ID NO: 57) or WASGYRGLTPASDYNIDYAI (SEQ ID NO: 58). Methods for producing such fragments are described in detail in the instant in the instant applicant's International Patent Application No. PCT/AU2005/000930 filed Jun. 24, 2005 (WO 2006/000045) the disclosure of which is incorporated herein in its entirety.

Antibodies that Bind to a KARI Protein or an Epitope Thereof

A second aspect of the present invention provides an antibody that binds specifically to a KARI protein or an immunogenic fragment or epitope thereof, such as, for example, a monoclonal or polyclonal antibody preparation suitable for use in the assays described herein.

Reference herein to antibody or antibodies includes whole polyclonal and monoclonal antibodies, and parts thereof, either alone or conjugated with other moieties. Antibody parts include Fab and F(ab)₂ fragments and single chain antibodies. The antibodies may be made in vivo in suitable laboratory animals, or, in the case of engineered antibodies (Single Chain Antibodies or SCABS, etc) using recombinant DNA techniques in vitro.

In accordance with this aspect of the invention, the antibodies may be produced for the purposes of immunizing the subject, in which case high titer or neutralizing antibodies that bind to a B cell epitope will be especially preferred. Suitable subjects for immunization will, of course, depend upon the immunizing antigen or antigenic B cell epitope. It is contemplated that the present invention will be broadly applicable to the immunization of a wide range of animals, such as, for example, farm animals (e.g. horses, cattle, sheep, pigs, goats, chickens, ducks, turkeys, and the like), laboratory animals (e.g. rats, mice, guinea pigs, rabbits), domestic animals (cats, dogs, birds and the like), feral or wild exotic animals (e.g. possums, cats, pigs, buffalo, wild dogs and the like) and humans.

Alternatively, the antibodies may be for commercial or diagnostic purposes, in which case the subject to whom the KARI protein or immunogenic fragment or epitope thereof is administered will most likely be a laboratory or farm animal. A wide range of animal species are used for the production of antisera. Typically the animal used for production of antisera is a rabbit, mouse, rabbit, rat, hamster, guinea pig, goat, sheep, pig, dog, horse, or chicken. Because of the relatively large blood volumes of rabbits and sheep, these are preferred choice for production of polyclonal antibodies. However, as will be known to those skilled in the art, larger amounts of immunogen are required to obtain antibodies from large animals as opposed to smaller animals such as mice. In such cases, it will be desirable to isolate the antibody from the immunized animal.

Preferably, the antibody is a high titer antibody. By “high titer” means a sufficiently high titer to be suitable for use in diagnostic or therapeutic applications. As will be known in the art, there is some variation in what might be considered “high titer”. For most applications a titer of at least about 10³-10⁴ is preferred. More preferably, the antibody titer will be in the range from about 10⁴ to about 10⁵, even more preferably in the range from about 10⁵ to about 10⁶.

More preferably, in the case of B cell epitopes from pathogens, viruses or bacteria, the antibody is a neutralizing antibody (i.e. it is capable of neutralizing the infectivity of the organism from which the B cell epitope is derived).

To generate antibodies, the KARI protein or immunogenic fragment or epitope thereof, optionally formulated with any suitable or desired carrier, adjuvant, BRM, or pharmaceutically acceptable excipient, is conveniently administered in the form of an injectable composition. Injection may be intranasal, intramuscular, sub-cutaneous, intravenous, intradermal, intraperitoneal, or by other known route. For intravenous injection, it is desirable to include one or more fluid and nutrient replenishers. Means for preparing and characterizing antibodies are well known in the art. (See, e.g., ANTIBODIES: A LABORATORY MANUAL, Cold Spring Harbor Laboratory, 1988, incorporated herein by reference).

Preferred immunogenic peptides for generating polyclonal or monoclonal antibodies are covalently coupled to an immunogenic carrier protein, such as Diphtheria toxoid (DT), Keyhole Limpet Hemocyanin (KLH), tetanus toxoid (TT) or the nuclear protein of influenza virus (NP), using one of several conjugation chemistries known in the art. This enhances the immunogenicity of peptides that are otherwise not highly immunogenic in animals e.g., mice, rats, chickens etc.

Methods of preparing carrier proteins for such coupling are well known in the art. For instance, DT is preferably produced by purification of the toxin from a culture of Corynebacterium diphtheriae followed by chemical detoxification, but is alternatively made by purification of a recombinant, or genetically detoxified analogue of the toxin (for example, CRM197, or other mutants as described in U.S. Pat. Nos. 4,709,017, 5,843,711, 5,601,827, and 5,917,017). Preferably, the toxoid is derivatized using as a spacer a bridge of up to 6 carbons, such as provided by use of the adipic acid hydrazide derivative of diphtheria toxoid (D-AH).

For coupling, peptides derived from the full-length KARI protein can be synthesized chemically or produced by recombinant expression means, treated with hydroxylamine to form free sulfhydryl groups, and cross-linked via the free sulfhydryl groups to a maleimide-modified diphtheria toxoid, tetanus toxoid or influenza NP protein or other carrier molecule. One of the most specific and reliable conjugation chemistries uses a cysteine residue in the peptide and a maleimide group added to the carrier protein, to form a stable thioether bond (Lee, A. C., et al., Mol. Immune-l. 17, 749-756 1980). For example, if no sulfhydryl groups are present in the peptide, the KARI protein-derived peptides can be prior modified by the addition of a C-terminal cysteine residue to facilitate this procedure. The immunogenic KARI peptides are preferably produced under non-denaturing conditions treated with hydroxylamine, thiol reducing agents or by acid or base hydrolysis to generate free sulfhydryl groups and the free sulfhydryl-containing peptide is conjugated to a carrier by chemical bonding via the free sulfhydryl groups. Such conjugation may be by use of a suitable bis-maleimide compound. Alternatively, the conjugation of the HA protein may be to a maleimide-modified carrier protein, such as diphtheria toxoid, tetanus toxoid or influenza (NP) protein or to a carbohydrate, such as alginic acid, dextran or polyethylene glycol. Such maleimide-modified carrier molecules may be formed by reaction of the carrier molecule with a hetero-bifunctional cross-linker of the maleimide-N-hydroxysuccinimide ester type. Examples of such bifunctional esters include maleimido-caproic-N-hydroxysuccinimide ester (MCS), maleimido-benzoyl-N-hydroxysuccinimide ester (MBS), maleimido-benzoylsul-fosuccinimide ester (sulfo-MBS), succinimidyl-4-(N-maleimidomethyl)cyclohexane-1-carboxylate (SMCC), succinimidyl-4-(p-maleimido-phenyl)butyrate (SMPP), sulfosuccinimidyl-4-(N-maleimidomethyl)cyclohexane-1-carboxylate (sulfo-SMCC) and sulfosuccinimidyl-4-(p-maleimidophenyl) butyrate (sulfo-SMPP). The N-hydroxy-succinimide ester moiety reacts with the amine groups of the carrier protein leaving the maleimide moiety free to react with the sulfhydryl groups on the antigen to form the cross-linked material.

The conjugate molecules so produced may be purified and employed in immunogenic compositions to elicit, upon administration to a host, an immune response to the KARI peptide which is potentiated in comparison to KARI peptide alone.

Diphtheria toxoid is obtained commercially or prepared from Corynebacterium diphtheriae grown in submerged culture by standard methods. The production of Diphtheria Toxoid is divided into five stages, namely maintenance of the working seed, growth of Corynebacterium diphtheriae, harvest of Diphtheria Toxin, detoxification of Diphtheria Toxin and concentration of Diphtheria Toxoid. The purified diphtheria toxoid (DT) used as carrier in the preparation is preferably a commercial toxoid modified (derivatized) by the attachment of a spacer molecule, such as adipic acid dihydrazide (ADH), using the water-soluble carbodiimide condensation method. The modified toxoid, typically the adipic hydrazide derivative D-AH, is then freed from unreacted ADH.

The efficacy of the KARI protein or immunogenic fragment or epitope thereof in producing an antibody is established by injecting an animal, for example, a mouse, chicken, rat, rabbit, guinea pig, dog, horse, cow, goat or pig, with a formulation comprising the KARI protein or immunogenic fragment or epitope thereof, and then monitoring the immune response to the B cell epitope, as described in the Examples. Both primary and secondary immune responses are monitored. The antibody titer is determined using any conventional immune-assay, such as, for example, ELISA, or radio immune-assay.

The production of polyclonal antibodies may be monitored by sampling blood of the immunized animal at various points following immunization. A second, booster injection, may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (Mabs).

Monoclonal antibodies are particularly preferred. For the production of monoclonal antibodies (Mabs) any one of a number of well-known techniques may be used, such as, for example, the procedure exemplified in U.S. Pat. No. 4,196,265, incorporated herein by reference.

For example, a suitable animal will be immunized with an effective amount of the KARI protein or immunogenic fragment or epitope thereof under conditions sufficient to stimulate antibody producing cells. Rodents such as rabbits, mice and rats are preferred animals, however, the use of sheep or frog cells is also possible. The use of rats may provide certain advantages, but mice or rabbits are preferred, with the BALB/c mouse being most preferred as the most routinely used animal and one that generally gives a higher percentage of stable fusions. Rabbits are known to provide high affinity monoclonal antibodies.

Following immunization, somatic cells with the potential for producing antibodies, specifically B lymphocytes (B cells), are selected for use in the MAb generating protocol.

These cells may be obtained from biopsies of spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer removed. Spleen lymphocytes are obtained by homogenizing the spleen with a syringe. Typically, a spleen from an immunized mouse contains approximately 5×10⁷ to 2×10⁸ lymphocytes.

The B cells from the immunized animal are then fused with cells of an immortal myeloma cell, generally derived from the same species as the animal that was immunized with the KARI protein or immunogenic fragment or epitope thereof. Myeloma cell lines suited for use in hybridoma-producing fusion procedures preferably are non-antibody-producing, have high fusion efficiency and enzyme deficiencies that render them incapable of growing in certain selective media which support the growth of only the desired fused cells, or hybridomas. Any one of a number of myeloma cells may be used and these are known to those of skill in the art (e.g. murine P3-X63/Ag8, X63-Ag8.653, NS1/1.Ag41, Sp210-Ag14, FO, NSO/U, MPC-11, MPC11-X45-GTG 1.7 and S194/5XX0; or rat R210.RCY3, Y3-Ag 1.2.3, IR983F and 4B210; and U-266, GM1500-GRG2, LICR-LON-HMy2 and UC729-6). A preferred murine myeloma cell is the NS-1 myeloma cell line (also termed P3-NS-1-Ag4-1), which is readily available from the NIGMS Human Genetic Mutant Cell Repository under Accession No. GM3573. Alternatively, a murine myeloma SP2/0 non-producer cell line that is 8-azaguanine-resistant is used.

To generate hybrids of antibody-producing spleen or lymph node cells and myeloma cells, somatic cells are mixed with myeloma cells in a proportion between about 20:1 to about 1:1 (v/v), respectively, in the presence of an agent or agents (chemical or electrical) that promote the fusion of cell membranes. Fusion methods using Sendai virus have been described by Kohler and Milstein, Nature 256, 495-497, 1975; and Kohler and Milstein, Eur. J. Immune-l. 6, 511-519, 1976. Methods using polyethylene glycol (PEG), such as 37% (v/v) PEG, are described in detail by Gefter et al., Somatic Cell Genet. 3, 231-236, 1977. The use of electrically induced fusion methods is also appropriate.

Hybrids are amplified by culture in a selective medium comprising an agent that blocks the de novo synthesis of nucleotides in the tissue culture media. Exemplary and preferred agents are aminopterin, methotrexate and azaserine. Aminopterin and methotrexate block de novo synthesis of both purines and pyrimidines, whereas azaserine blocks only purine synthesis.

Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT, because only those hybridomas capable of operating nucleotide salvage pathways are able to survive in HAT medium, whereas myeloma cells are defective in key enzymes of the salvage pathway, (e.g., hypoxanthine phosphoribosyl transferase or HPRT), and they cannot survive. B cells can operate this salvage pathway, but they have a limited life span in culture and generally die within about two weeks. Accordingly, the only cells that can survive in the selective media are those hybrids formed from myeloma and B cells.

The amplified hybridomas are subjected to a functional selection for antibody specificity and/or titer, such as, for example, by immune-assay (e.g. radioimmune-assay, enzyme immune-assay, cytotoxicity assay, plaque assay, dot immune-assay, and the like).

The selected hybridomas are serially diluted and cloned into individual antibody-producing cell lines, which clones can then be propagated indefinitely to provide MAbs. The cell lines may be exploited for MAb production in two basic ways. A sample of the hybridoma is injected, usually in the peritoneal cavity, into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they are readily obtained in high concentrations. MAbs produced by either means may be further purified, if desired, using filtration, centrifugation and various chromatographic methods such as HPLC or affinity chromatography.

Alternatively, ABL-MYC technology (NeoClone, Madison Wis. 53713, USA) is used to produce cell lines secreting monoclonal antibodies (mAbs) against immunogenic KARI peptide antigens. In this process, BALB/cByJ female mice are immunized with an amount of the peptide antigen over a period of about 2 to about 3 months. During this time; test bleeds are taken from the immunized mice at regular intervals to assess antibody responses in a standard ELISA. The spleens of mice having antibody titers of at least about 1,000 are used for subsequent ABL-MYC infection employing replication-incompetent retrovirus comprising the oncogenes v-abl and c-myc. Splenocytes are transplanted into naive mice which then develop ascites fluid containing cell lines producing monoclonal antibodies (mAbs) against the KARI peptide antigen. The mAbs are purified from ascites using protein G or protein A, e.g., bound to a solid matrix, depending on the isotype of the mAb. Because there is no hybridoma fusion, an advantage of the ABL-MYC process is that it is faster, more cost effective, and higher yielding than conventional mAb production methods. In addition, the diploid plasmacytomas produced by this method are intrinsically more stable than polyploid hybridomas, because the ABL-MYC retrovirus infects only cells in the spleen that have been stimulated by the immunizing antigen. ABL-MYC then transforms those activated B-cells into immortal, mAb-producing plasma cells called plasmacytomas. A “plasmacytoma” is an immortalized plasma cell that is capable of uncontrolled cell division. Since a plasmacytoma begins with just one cell, all of the plasmacytomas produced from it are therefore identical, and moreover, produce the same desired “monoclonal” antibody. As a result, no sorting of undesirable cell lines is required. The ABL-MYC technology is described generically in detail in the following disclosures which are incorporated by reference herein:
1. Largaespada et al., Curr. Top. Microbiol. Immune-l., 166, 91-96. 1990;
2. Weissinger et al., Proc. Natl. Acad. Sci. USA, 88, 8735-8739, 1991;
3. Largaespada et al., Oncogene, 7, 811-819, 1992;
4. Weissinger et al., J. Immune-l. Methods 168, 123-130, 1994;
5. Largaespada et al., J. Immune-l. Methods. 197(1-2), 85-95, 1996; and
6. Kumar et al., Immune-l. Letters 65, 153-159, 1999.

Monoclonal antibodies of the present invention also include anti-idiotypic antibodies produced by methods well-known in the art. Monoclonal antibodies according to the present invention also may be monoclonal heteroconjugates, (i.e., hybrids of two or more antibody molecules). In another example, monoclonal antibodies according to the invention are chimeric monoclonal antibodies. In one approach, the chimeric monoclonal antibody is engineered by cloning recombinant DNA containing the promoter, leader, and variable-region sequences from a mouse anti-KARI producing cell and the constant-region exons from a human antibody gene. The antibody encoded by such a recombinant gene is a mouse-human chimera. Its antibody specificity is determined by the variable region derived from mouse sequences. Its isotype, which is determined by the constant region, is derived from human DNA.

In another example, the monoclonal antibody according to the present invention is a “humanized” monoclonal antibody, produced by any one of a number of techniques well-known in the art. That is, mouse complementary determining regions (“CDRs”) are transferred from heavy and light V-chains of the mouse Ig into a human V-domain, followed by the replacement of some human residues in the framework regions of their murine counterparts. “Humanized” monoclonal antibodies in accordance with this invention are especially suitable for use in vivo in diagnostic and therapeutic methods.

As stated above, the monoclonal antibodies and fragments thereof according to this invention are multiplied according to in vitro and in vivo methods well-known in the art. Multiplication in vitro is carried out in suitable culture media such as Dulbecco's modified Eagle medium or RPMI 1640 medium, optionally replenished by a mammalian serum such as fetal calf serum or trace elements and growth-sustaining supplements, e.g., feeder cells, such as normal mouse peritoneal exudate cells, spleen cells, bone marrow macrophages or the like. In vitro production provides relatively pure antibody preparations and allows scale-up to give large amounts of the desired antibodies. Techniques for large scale hybridoma cultivation under tissue culture conditions are known in the art and include homogenous suspension culture, (e.g., in an airlift reactor or in a continuous stirrer reactor or immobilized or entrapped cell culture).

Large amounts of the monoclonal antibody of the present invention also may be obtained by multiplying hybridoma cells in vivo. Cell clones are injected into mammals which are histocompatible with the parent cells, (e.g., syngeneic mice, to cause growth of antibody-producing tumors. Optionally, the animals are primed with a hydrocarbon, especially oils such as Pristane (tetramethylpentadecane) prior to injection.

In accordance with the present invention, fragments of the monoclonal antibody of the invention are obtained from monoclonal antibodies produced as described above, by methods which include digestion with enzymes such as pepsin or papain and/or cleavage of disulfide bonds by chemical reduction. Alternatively, monoclonal antibody fragments encompassed by the present invention are synthesized using an automated peptide synthesizer, or they may be produced manually using techniques known in the art.

The monoclonal conjugates of the present invention are prepared by methods known in the art, e.g., by reacting a monoclonal antibody prepared as described above with, for instance, an enzyme in the presence of a coupling agent such as glutaraldehyde or periodate. Conjugates with fluorescein markers are prepared in the presence of these coupling agents, or by reaction with an isothiocyanate. Conjugates with metal chelates are similarly produced. Other moieties to which antibodies may be conjugated include radionuclides such as, for example, ³H, ¹²⁵I, ³²P, ³⁵S, ¹⁴C, ⁵¹Cr, ³⁶Cl, ⁵⁷Co, ⁵⁸Co, ⁵⁹Fe, ⁷⁵Se, and ¹⁵²Eu.

The present invention clearly includes antibodies when coupled to any detectable ligand or reagent, including, for example, an enzyme such as horseradish peroxidase or alkaline phosphatase, or a fluorophore, radionuclide, coloured dye, gold particle, colloidal gold, etc.

Radioactively labelled monoclonal antibodies of the present invention are produced according to known methods in the art. For instance, monoclonal antibodies are iodinated by contact with sodium or potassium iodide and a chemical oxidizing agent such as sodium hypochlorite, or an enzymatic oxidizing agent, such as lactoperoxidase. Monoclonal antibodies according to the invention may be labelled with technetium⁹⁹ by ligand exchange process, for example, by reducing pertechnate with stannous solution, chelating the reduced technetium onto a Sephadex column and applying the antibody to this column or by direct labelling techniques, (e.g., by incubating pertechnate, a reducing agent such as SNCl₂, a buffer solution such as sodium-potassium phthalate solution, and the antibody).

Any immune-assay may be used to monitor antibody production by the KARI protein or immunogenic fragment or epitope thereof. Immune-assays, in their most simple and direct sense, are binding assays. Certain preferred immune-assays are the various types of enzyme linked immune-sorbent assays (ELISAs) and radioimmune-assays (RIA) known in the art. Immune-histochemical detection using tissue sections is also particularly useful. However, it will be readily appreciated that detection is not limited to such techniques, and Western blotting, dot blotting, FACS analyses, and the like may also be used.

Most preferably, the assay will be capable of generating quantitative results.

For example, antibodies are tested in simple competition assays. A known antibody preparation that binds to the B cell epitope and the test antibody are incubated with an antigen composition comprising the B cell epitope, preferably in the context of the native antigen. “Antigen composition” as used herein means any composition that contains some version of the B cell epitope in an accessible form. Antigen-coated wells of an ELISA plate are particularly preferred. In one example, one would pre-mix the known antibodies with varying amounts of the test antibodies (e.g., 1:1 (v/v), 1:10 (v/v) and 1:100 (v/v)) for a period of time prior to applying to the antigen composition. If one of the known antibodies is labelled, direct detection of the label bound to the antigen is possible; comparison to an unmixed sample assay will determine competition by the test antibody and, hence, cross-reactivity. Alternatively, using secondary antibodies specific for either the known or test antibody, one will be able to determine competition.

An antibody that binds to the antigen composition will be able to effectively compete for binding of the known antibody and thus will significantly reduce binding of the latter. The reactivity of the known antibodies in the absence of any test antibody is the control. A significant reduction in reactivity in the presence of a test antibody is indicative of a test antibody that binds to the B cell epitope (i.e., it cross-reacts with the known antibody).

In one exemplary ELISA, the antibodies that bind to the KARI protein or immunogenic fragment or B cell epitope are immobilized onto a selected surface exhibiting protein affinity, such as a well in a polystyrene microtiter plate. Then, a composition containing a peptide comprising the B cell epitope is added to the wells. After binding and washing to remove non-specifically bound immune complexes, the bound epitope may be detected. Detection is generally achieved by the addition of a second antibody that is known to bind to the B cell epitope and is linked to a detectable label. This type of ELISA is a simple “sandwich ELISA”. Detection may also be achieved by the addition of said second antibody, followed by the addition of a third antibody that has binding affinity for the second antibody, with the third antibody being linked to a detectable label.

In an alternative example (i.e., amplified ELISA), antibodies that bind to the KARI protein or immunogenic fragment or B cell epitope are immobilized onto a selected surface exhibiting protein affinity, such as a well in a polystyrene microtiter plate. Then, a composition containing a peptide comprising the B cell epitope is added to the wells. After binding and washing to remove non-specifically bound immune complexes, antibodies that bind to the B cell epitope are contacted with the bound peptide for a time and under conditions sufficient for a complex to form. The signal is then amplified using secondary and preferably tertiary, antibodies that bind to the antibodies recognising the B cell epitope. Detection is then achieved by the addition of a further antibody that is known to bind to the secondary or tertiary antibodies, linked to a detectable label.

In another exemplary immune-assay format applicable to both flow through and solid phase ELISA, antibodies that bind to the immunogenic KARI protein or immunogenic KARI peptide or immunogenic KARI fragment or B cell epitope are immobilized onto a selected surface exhibiting protein affinity, such as a well in a polystyrene microtiter plate or a column. A sample comprising the immunogenic KARI protein or immunogenic peptide or immunogenic fragment comprising the B cell epitope to which the antibody binds is added for a time and under conditions sufficient for an antigen-antibody complex to form. In this case, the added KARI protein, peptide or fragment is complexed with human Ig. In the case of patient sera, for example, the peptide is preferably complexed with human Ig by virtue of an immune response of the patient to the M. tuberculosis KARI protein. After binding and washing to remove non-specifically bound immune complexes, the bound epitope is detected by the addition of a second antibody that is known to bind to human Ig in the immune complex and is linked to a detectable label. This is a modified “sandwich ELISA”. Detection may also be achieved by the addition of said second antibody, followed by the addition of a third antibody that has binding affinity for the second antibody, with the third antibody being linked to a detectable label.

Antibodies of the invention may be bound to a solid support and/or packaged into kits in a suitable container along with suitable reagents, controls, instructions and the like.

Antibodies that Bind to a Secondary Analyte

It is to be understood that methods described herein above for the production of antibodies against the KARI protein or an immunogenic fragment thereof apply mutatis mutandis to the production of antibodies against a secondary analyte that is to be used in an immune-assay format e.g., for the purposes of diagnosis or prognosis of tuberculosis or infection by M. tuberculosis. As will be understood by the skilled artisan, such extrapolation is dependent on substituting the KARI protein immunogen for the secondary analyte in question e.g., M. tuberculosis BSX protein or GS protein or S9 protein or immunogenic fragment thereof according to any example hereof or a combination or mixture of said peptides or epitopes or fragments. Such substitution is readily performed without undue experimentation from the disclosure herein.

For convenience, preferred immunizing peptides for the production of antibodies against secondary analytes e.g., for use in multi-analyte antigen-based tests, will comprise an amino acid sequence selected from the group set forth in SEQ ID NOs: 3-60 and combinations or mixtures thereof.

For example, antibodies that bind to M. tuberculosis BSX protein can be prepared from the full-length protein (e.g., comprising the sequence set forth in SwissProt Database Accession No. O53759) or from a peptide fragment thereof e.g., comprising a sequence selected from the group consisting of MRQLAERSGVSNPYL (SEQ ID NO: 3), ERGLRKPSADVLSQI (SEQ ID NO: 4), LRKPSADVLSQIAKA (SEQ ID NO: 5), PSADVLSQIAKALRV (SEQ ID NO: 6), SQIAKALRVSAEVLY (SEQ ID NO: 7), AKALRVSAEVLYVRA (SEQ ID NO: 8), VRAGILEPSETSQVR (SEQ ID NO: 9), TAITERQKQILLDIY (SEQ ID NO: 10), SQIAKALRVSAEVLYVRAC (SEQ ID NO: 11), MSSEEKLCDPTPTDD (SEQ ID NO: 12) and VRAGILEPSETSQVRC (SEQ ID NO: 13) and combinations thereof. Antibodies that bind to an immunogenic M. tuberculosis BSX protein or peptide for detecting tuberculosis or infection by M. tuberculosis are also described in detail in the instant applicant's International Patent Application No. PCT/AU2005/001254 filed Aug. 19, 2005 (WO 2006/01792) the disclosure of which is incorporated herein in its entirety.

Alternatively, or in addition, antibodies that bind to M. tuberculosis glutamine synthetase (GS) protein (e.g., comprising the sequence set forth in SwissProt Database Accession No. O33342) or from an immunogenic peptide derived thereof, e.g., comprising a surface-exposed region of a GS protein, or comprising the sequence RGTDGSAVFADSNGPHGMSSMFRSF (SEQ ID NO: 57) or WASGYRGLTPASDYNIDYAI (SEQ ID NO: 58) or a combinations thereof. Antibodies that bind to an immunogenic M. tuberculosis GS or peptide for detecting tuberculosis or infection by M. tuberculosis are also described in detail in the instant applicant's International Patent Application No. PCT/AU2005/000930 filed Jun. 24, 2005 (WO 2006/000045) the disclosure of which is incorporated herein in its entirety.

The present invention clearly contemplates antibodies against secondary analytes other than BSX or GS or S9 or immunogenic fragments thereof, the description of which is provided for the purposes of exemplification.

### Diagnostic/Prognostic Methods for Detecting Tuberculosis or M. Tuberculosis Infection

### 1. Antigen-Based Assays

This invention provides a method of diagnosing tuberculosis or an infection by M. tuberculosis in a subject comprising detecting in a biological sample from said subject a KARI protein or an immunogenic fragment or epitope thereof, wherein the presence of said protein or immunogenic fragment or epitope in the sample is indicative of infection.

One advantage of detecting M. tuberculosis antigen, as opposed to an antibody-based assay is that severely immune-compromised patients may not produce antibody at detectable levels, and the level of the antibody in any patient does not reflect bacilli burden. On the other hand antigen levels should reflect bacilli burden and, being a product of the bacilli, are a direct method of detecting its presence.

In one example of the diagnostic assays of the invention, there is provided a method for detecting M. tuberculosis infection in a subject, the method comprising contacting a biological sample derived from the subject with an antibody capable of binding to a KARI protein or an immunogenic fragment or epitope thereof, and detecting the formation of an antigen-antibody complex.

Preferably, the subject is suspected of suffering from tuberculosis or an infection by M. tuberculosis and/or is at risk of developing tuberculosis, or at risk of being infected by M. tuberculosis.

In another example, the diagnostic assays of the invention are useful for determining the progression of tuberculosis or an infection by M. tuberculosis in a subject. In accordance with these prognostic applications of the invention, the level of KARI protein or an immunogenic fragment or epitope thereof in a biological sample is positively correlated with the infectious state. For example, a level of the KARI protein or an immunogenic fragment thereof that is less than the level of the KARI protein or fragment detectable in a subject suffering from the symptoms of tuberculosis or an infection indicates that the subject is recovering from the infection. Similarly, a higher level of the protein or fragment in a sample from the subject compared to a healthy individual indicates that the subject has not been rendered free of the disease or infection.

Accordingly, a further example of the present invention provides a method for determining the response of a subject having tuberculosis or an infection by M. tuberculosis to treatment with a therapeutic compound for said tuberculosis or infection, said method comprising detecting a KARI protein or an immunogenic fragment or epitope thereof in a biological sample from said subject, wherein a level of the protein or fragment or epitope that is enhanced compared to the level of that protein or fragment or epitope detectable in a normal or healthy subject indicates that the subject is not responding to said treatment or has not been rendered free of disease or infection.

In an alternative example, the present invention provides a method for determining the response of a subject having tuberculosis or an infection by M. tuberculosis to treatment with a therapeutic compound for said tuberculosis or infection, said method comprising detecting a KARI protein or an immunogenic fragment or epitope thereof in a biological sample from said subject, wherein a level of the protein or fragment or epitope that is lower than the level of the protein or fragment or epitope detectable in a subject suffering from tuberculosis or infection by M. tuberculosis indicates that the subject is responding to said treatment or has been rendered free of disease or infection. Clearly, if the level of the KARI protein or fragment or epitope thereof is not detectable in the subject, the subject has responded to treatment.

In a further example, the amount of a KARI protein in a biological sample derived from a patient is compared to the amount of the same protein detected in a biological sample previously derived from the same patient. As will be apparent to a person skilled in the art, this method may be used to continually monitor a patient with a latent infection or a patient that has developed tuberculosis. In this way a patient may be monitored for the onset or progression of an infection or disease, with the goal of commencing treatment before an infection is established, particularly in an HIV+ individual.

Alternatively, or in addition, the amount of a protein detected in a biological sample derived from a subject with tuberculosis may be compared to a reference sample, wherein the reference sample is derived from one or more tuberculosis patients that do not suffer from an infection or disease or alternatively, one or more tuberculosis patients that have recently received successful treatment for infection and/or one or more subjects that do not have tuberculosis and that do not suffer from an infection or disease.

In one example, a KARI protein or immunogenic fragment thereof is not detected in a reference sample, however, said KARI protein or immunogenic fragment thereof is detected in the patient sample, indicating that the patient from whom the sample was derived is suffering from tuberculosis or infection by M. tuberculosis or will develop an acute infection.

Alternatively, the amount of KARI protein or immunogenic fragment thereof may be enhanced in the patient sample compared to the level detected in a reference sample. Again, this indicates that the patient from whom the biological sample was isolated is suffering from tuberculosis or infection by M. tuberculosis or will develop an acute infection.

In one example of the diagnostic/prognostic methods described herein, the biological sample is obtained previously from the subject. In accordance with such an example, the prognostic or diagnostic method is performed ex vivo.

In yet another example, the subject diagnostic/prognostic methods further comprise processing the sample from the subject to produce a derivative or extract that comprises the analyte (e.g., pleural fluid or sputum or serum).

Suitable samples include extracts from tissues such as brain, breast, ovary, lung, colon, pancreas, testes, liver, muscle and bone tissues, or body fluids such as sputum, serum, plasma, whole blood, sera or pleural fluid.

Preferably, the biological sample is a bodily fluid or tissue sample selected from the group consisting of saliva, plasma, blood, serum, sputum, urine, and lung. Other samples are not excluded.

It will be apparent from the description herein that preferred samples may comprise circulating immune complexes comprising the KARI protein or fragments thereof complexed with human immune-globulin. The detection of such immune complexes is clearly within the scope of the present invention. In accordance with this example, a capture reagent e.g., a capture antibody is used to capture the KARI antigen (KARI protein, polypeptide or an immune-active fragment or epitope thereof) complexed with the subject's immune-globulin, in addition to isolated antigen in the subject's circulation. Anti-Ig antibodies, optionally conjugated to a detectable label, are used to specifically bind the captured CIC thereby detecting CIC patient samples. Within the scope of this invention, the anti-Ig antibody binds preferentially to IgM, IgA or IgG in the sample. In a particularly preferred example, the anti-Ig antibody binds to human Ig, e.g., human IgA, human IgG or human IgM. The anti-Ig antibody may be conjugated to any standard detectable label known in the art. This is particularly useful for detecting infection by a pathogenic agent, e.g., a bacterium or virus, or for the diagnosis of any disease or disorder associated with CICs. Accordingly, the diagnostic methods described according to any example herein are amenable to a modification wherein the sample derived from the subject comprises one or more circulating immune complexes comprising immune-globulin (Ig) bound to KARI protein of Mycobacterium tuberculosis or one or more immunogenic KARI peptides, fragments or epitopes thereof and wherein detecting the formation of an antigen-antibody complex comprises contacting an anti-Ig antibody with an immune-globulin moiety of the circulating immune complex(es) for a time and under conditions sufficient for a complex to form than then detecting the bound anti-Ig antibody.

The present invention clearly encompasses multianalyte tests for diagnosing infection by M. tuberculosis. For example, assays for detecting antibodies that bind to M. tuberculosis KARI protein can be combined with assays for detecting M. tuberculosis BSX or glutamine synthetase (GS) protein. In this respect, the present inventors have also produced plasmacytomas producing monoclonal antibodies that bind to an immunogenic fragment or peptide or epitope of BSX or GS.

### 2. Antibody-Based Assays

The present invention provides a method of diagnosing tuberculosis or an infection by M. tuberculosis in a subject comprising detecting in a biological sample from said subject antibodies that bind to a KARI protein or an immunogenic fragment or epitope thereof, wherein the presence of said antibodies in the sample is indicative of infection. The infection may be a past or present infection, or a latent infection.

Preferably, the subject is suspected of suffering from tuberculosis or an infection by M. tuberculosis and/or is at risk of developing tuberculosis and/or at risk of being infected by M. tuberculosis.

Antibody-based assays are primarily used for detecting active infections by M. tuberculosis. Preferably, the clinical history of the subject is considered due to residual antibody levels that may persist in recent past infections or chronic infections by M. tuberculosis.

The format is inexpensive and highly sensitive, however not as useful as an antigen-based assay format for detecting infection in immune-compromised individuals. However, antibody-based assays are clearly useful for detecting M. tuberculosis infections in HIV or HIV+ individuals who are not immune-compromised.

In one alternative example, the present invention provides a method for detecting M. tuberculosis infection in a subject, the method comprising contacting a biological sample derived from the subject with a KARI protein or an immunogenic fragment or epitope thereof and detecting the formation of an antigen-antibody complex.

In the antibody based assays described herein, it is preferred that the KARI protein or immunogenic fragment or epitope thereof used to detect the antibodies is not highly cross-reactive with anti-sera from non-infected subjects. Accordingly, isolated or recombinant KARI is preferred for use in the antibody-based platforms described herein.

In another example, the diagnostic assays of the invention are useful for determining the progression of tuberculosis or an infection by M. tuberculosis in a subject. In accordance with these prognostic applications of the invention, the amount of antibodies that bind to a KARI protein or fragment or epitope in blood or serum, plasma, or an immune-globulin fraction from the subject is positively correlated with the infectious state. For example, a level of the anti-KARI protein antibodies thereto that is less than the level of the anti-KARI protein antibodies detectable in a subject suffering from the symptoms of tuberculosis or an infection indicates that the subject is recovering from the infection. Similarly, a higher level of the antibodies in a sample from the subject compared to a healthy individual indicates that the subject has not been rendered free of the disease or infection.

In a further example of the present invention provides a method for determining the response of a subject having tuberculosis or an infection by M. tuberculosis to treatment with a therapeutic compound for said tuberculosis or infection, said method comprising detecting antibodies that bind to a KARI protein or an immunogenic fragment or epitope thereof in a biological sample from said subject, wherein a level of the antibodies that is enhanced compared to the level of the antibodies detectable in a normal or healthy subject indicates that the subject is not responding to said treatment or has not been rendered free of disease or infection. Again, isolated or recombinant KARI protein is preferred.

In an alternative example, the present invention provides a method for determining the response of a subject having tuberculosis or an infection by M. tuberculosis to treatment with a therapeutic compound for said tuberculosis or infection, said method comprising detecting antibodies that bind to a KARI protein or an immunogenic fragment or epitope thereof in a biological sample from said subject, wherein a level of the antibodies that is lower than the level of the antibodies detectable in a subject suffering from tuberculosis or infection by M. tuberculosis indicates that the subject is responding to said treatment or has been rendered free of disease or infection.

The amount of an antibody against the KARI protein or fragment that is detected in a biological sample from a subject with tuberculosis may be compared to a reference sample, wherein the reference sample is derived from one or more healthy subjects who have not been previously infected with M. tuberculosis or not recently-infected with M. tuberculosis. Such negative control subjects will have a low circulating antibody titer making them suitable standards in antibody-based assay formats. For example, antibodies that bind to a KARI protein or immunogenic fragment thereof are not detected in the reference sample and only in a patient sample, indicating that the patient from whom the sample was derived is suffering from tuberculosis or infection by M. tuberculosis or will develop an acute infection. Isolated or recombinant KARI protein is preferred for use in such examples. In one example of the diagnostic/prognostic methods described herein, the biological sample is obtained previously from the subject. In accordance with such an example, the prognostic or diagnostic method is performed ex vivo.

In yet another example, the subject diagnostic/prognostic methods further comprise processing the sample from the subject to produce a derivative or extract that comprises the analyte (e.g., blood, serum, plasma, or any immune-globulin-containing sample).

Suitable samples include, for example, extracts from tissues comprising an immune-globulin such as blood, bone, or body fluids such as serum, plasma, whole blood, an immune-globulin-containing fraction of serum, an immune-globulin-containing fraction of plasma, an immune-globulin-containing fraction of blood.

### 3. Detection Systems

Preferred detection systems contemplated herein include any known assay for detecting proteins or antibodies in a biological sample isolated from a human subject, such as, for example, SDS/PAGE, isoelectric focusing, 2-dimensional gel electrophoresis comprising SDS/PAGE and isoelectric focusing, an immune-assay, a detection based system using an antibody or non-antibody ligand of the protein, such as, for example, a small molecule (e.g. a chemical compound, agonist, antagonist, allosteric modulator, competitive inhibitor, or non-competitive inhibitor, of the protein). In accordance with these examples, the antibody or small molecule may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, particularly a high throughput spectroscopy resonance method (e.g. MALDI-TOF, electrospray MS or nano-electrospray MS), are particularly contemplated.

Immune-assay formats are particularly preferred, e.g., selected from the group consisting of, an immune-blot, a Western blot, a dot blot, an enzyme linked immune-sorbent assay (ELISA), radioimmune-assay (RIA), enzyme immune-assay. Modified immune-assays utilizing fluorescence resonance energy transfer (FRET), isotope-coded affinity tags (ICAT), mass spectrometry, e.g., matrix-assisted laser desorption/ionization time of flight (MALDI-TOF), electrospray ionization (ESI), biosensor technology, evanescent fiber-optics technology or protein chip technology are also useful.

Preferably, the assay is a semi-quantitative assay or quantitative assay.

Standard solid phase ELISA formats are particularly useful in determining the concentration of a protein or antibody from a variety of patient samples.

In one form such as an assay involves immobilising a biological sample comprising anti-KARI protein antibodies, or alternatively KARI protein or an immunogenic fragment thereof, onto a solid matrix, such as, for example a polystyrene or polycarbonate microwell or dipstick, a membrane, or a glass support (e.g. a glass slide).

In the case of an antigen-based assay, an immobilised antibody that specifically binds a KARI protein is brought into direct contact with the biological sample, and forms a direct bond with any of its target protein present in said sample. For an antibody-based assay, an immobilised isolated or recombinant KARI protein or an immunogenic fragment or epitope thereof will be contacted with the biological sample. The added antibody or protein in solution is generally labelled with a detectable reporter molecule, such as for example, colloidal gold, a fluorescent label (e.g. FITC or Texas Red) or an enzyme (e.g. horseradish peroxidase (HRP)), alkaline phosphatase (AP) or β-galactosidase). Alternatively, or in addition, a second labelled antibody can be used that binds to the first antibody or to the isolated/recombinant KARI antigen. Following washing to remove any unbound antibody or KARI antigen, the label may be detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal).

Such ELISA based systems are particularly suitable for quantification of the amount of a protein or antibody in a sample, such as, for example, by calibrating the detection system against known amounts of a standard.

In another form, an ELISA consists of immobilizing an antibody that specifically binds a KARI protein on a solid matrix, such as, for example, a membrane, a polystyrene or polycarbonate microwell, a polystyrene or polycarbonate dipstick or a glass support. A patient sample is then brought into physical relation with said antibody, and the antigen in the sample is bound or ‘captured’. The bound protein can then be detected using a labelled antibody. For example if the protein is captured from a human sample, an anti-human Ig antibody is used to detect the captured protein.

One example of this example of the invention comprises:

(i) immobilizing an antibody that specifically binds an immunogenic KARI peptide or KARI protein to a solid matrix or support;
(ii) contacting the bound antibody with a sample obtained from a subject, preferably an antibody-containing sample such as blood, serum or Ig-containing fraction thereof for a time and under conditions sufficient for the immobilized antibody to bind to a KARI protein or fragment thereof in the sample thereby forming an antigen-antibody complex; and
(iii) detecting the antigen-antibody complex formed in a process comprising contacting said complex with an antibody that recognizes human Ig, wherein the presence of said human Ig indicates the presence of M. tuberculosis in the patient sample.

In accordance with this example, specificity of the immobilized antibody ensures that isolated or immune-complexed KARI protein or fragments comprising the epitope that the antibody recognizes actually bind, whilst specificity of anti-human Ig ensures that only immune-complexed KARI protein or fragment is detected. In this context, the term “immune-complexed” shall be taken to mean that the KARI protein or fragments thereof in the patient sample are complexed with human Ig such as human IgA or human IgM or human IgG, etc. Accordingly, this example is particularly useful for detecting the presence of M. tuberculosis or an infection by M. tuberculosis that has produced an immune response in a subject. By appropriately selecting the detection antibody, e.g., anti-human IgA or anti-human IgG or anti-human IgM, it is further possible to isotype the immune response of the subject. Such detection antibodies that bind to human IgA, IgM and IgG are publicly available to the art.

Alternatively or in addition to the preceding examples, a third labelled antibody can be used that binds the second (detecting) antibody.

It will be apparent to the skilled person that the assay formats described herein are amenable to high throughput formats, such as, for example automation of screening processes, or a microarray format as described in Mendoza et al, Biotechniques 27(4): 778-788, 1999. Furthermore, variations of the above described assay will be apparent to those skilled in the art, such as, for example, a competitive ELISA.

Alternatively, the presence of anti-KARI protein antibodies, or alternatively a KARI protein or an immunogenic fragment thereof, is detected using a radioimmune-assay (RIA). The basic principle of the assay is the use of a radiolabelled antibody or antigen to detect antibody antigen interactions. For example, an antibody that specifically binds to a KARI protein can be bound to a solid support and a biological sample brought into direct contact with said antibody. To detect the bound antigen, an isolated and/or recombinant form of the antigen is radiolabelled is brought into contact with the same antibody. Following washing the amount of bound radioactivity is detected. As any antigen in the biological sample inhibits binding of the radiolabelled antigen the amount of radioactivity detected is inversely proportional to the amount of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

Western blotting is also useful for detecting a KARI protein or an immunogenic fragment thereof. In such an assay, protein from a biological sample is separated using sodium dodecyl sulphate (SDS) polyacrylamide gel electrophoresis (SDS-PAGE) using techniques well known in the art and described in, for example, Scopes (In: Protein Purification: Principles and Practice, Third Edition, Springer Verlag, 1994). Separated proteins are then transferred to a solid support, such as, for example, a membrane or more specifically, nitrocellulose membrane, nylon membrane or PVDF membrane, using methods well known in the art, for example, electrotransfer. This membrane may then be blocked and probed with a labelled antibody or ligand that specifically binds a KARI protein. Alternatively, a labelled secondary, or even tertiary, antibody or ligand can be used to detect the binding of a specific primary antibody.

High-throughput methods for detecting the presence or absence of anti-KARI protein antibodies, or alternatively KARI protein or an immunogenic fragment thereof are particularly preferred.

In one example, mass spectrometry, e.g., MALDI-TOF is used for the rapid identification of a protein that has been separated by either one- or two-dimensional gel electrophoresis. Accordingly, there is no need to detect the proteins of interest using an antibody or ligand that specifically binds to the protein of interest. Rather, proteins from a biological sample are separated using gel electrophoresis using methods known in the art and those proteins at approximately the correct molecular weight and/or isoelectric point are analysed using MALDI-TOF to determine the presence or absence of a protein of interest.

Alternatively, mass spectrometry, e.g., MALDI or ESI, or a combination of approaches is used to determine the concentration of a particular protein in a biological sample, such as, for example sputum. Such proteins are preferably well characterised previously with regard to parameters such as molecular weight and isoelectric point.

Biosensor devices generally employ an electrode surface in combination with current or impedance measuring elements to be integrated into a device in combination with the assay substrate (such as that described in U.S. Pat. No. 5,567,301). An antibody or ligand that specifically binds to a protein of interest is preferably incorporated onto the surface of a biosensor device and a biological sample isolated from a patient (for example sputum that has been solubilized using the methods described herein) contacted to said device. A change in the detected current or impedance by the biosensor device indicates protein binding to said antibody or ligand. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several epitopes in a small amount of body fluids.

Evanescent biosensors are also preferred as they do not require the pre-treatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the diagnostic protein to the antibody or ligand.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application Nos. 20020136821, 20020192654, 20020102617 and U.S. Pat. No. 6,391,625. In order to bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al. Anal. Biochem. 278:123-131, 2000.

A protein chip is preferably generated such that several proteins, ligands or antibodies are arrayed on said chip. This format permits the simultaneous screening for the presence of several proteins in a sample.

Alternatively, a protein chip may comprise only one protein, ligand or antibody, and be used to screen one or more patient samples for the presence of one polypeptide of interest. Such a chip may also be used to simultaneously screen an array of patient samples for a polypeptide of interest.

Preferably, a sample to be analysed using a protein chip is attached to a reporter molecule, such as, for example, a fluorescent molecule, a radioactive molecule, an enzyme, or an antibody that is detectable using methods well known in the art. Accordingly, by contacting a protein chip with a labelled sample and subsequent washing to remove any unbound proteins the presence of a bound protein is detected using methods well known in the art, such as, for example using a DNA microarray reader.

Alternatively, biomolecular interaction analysis-mass spectrometry (BIA-MS) is used to rapidly detect and characterise a protein present in complex biological samples at the low- to sub-femptamole (fmol) level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology to characterise a protein bound to the protein chip. Alternatively, the protein chip is analysed using ESI as described in U.S. Patent Application 20020139751.

As will be apparent to the skilled artisan, protein chips are particularly amenable to multiplexing of detection reagents. Accordingly, several antibodies or ligands each able to specifically bind a different peptide or protein may be bound to different regions of said protein chip. Analysis of a biological sample using said chip then permits the detecting of multiple proteins of interest, or multiple B cell epitopes of the KARI protein. Multiplexing of diagnostic and prognostic markers is particularly contemplated in the present invention.

In a further example, the samples are analysed using ICAT or ITRAC, essentially as described in US Patent Application No. 20020076739. This system relies upon the labelling of a protein sample from one source (i.e. a healthy individual) with a reagent and the labelling of a protein sample from another source (i.e. a tuberculosis patient) with a second reagent that is chemically identical to the first reagent, but differs in mass due to isotope composition. It is preferable that the first and second reagents also comprise a biotin molecule. Equal concentrations of the two samples are then mixed, and peptides recovered by avidin affinity chromatography. Samples are then analysed using mass spectrometry. Any difference in peak heights between the heavy and light peptide ions directly correlates with a difference in protein abundance in a biological sample. The identity of such proteins may then be determined using a method well known in the art, such as, for example MALDI-TOF, or ESI.

In a particularly preferred example, a biological sample comprising anti-KARI protein antibodies, or alternatively KARI protein or an immunogenic fragment thereof, is subjected to 2-dimensional gel electrophoresis. In accordance with this example, it is preferable to remove certain particulate matter from the sample prior to electrophoresis, such as, for example, by centrifugation, filtering, or a combination of centrifugation and filtering. Proteins in the biological sample are then separated. For example, the proteins may be separated according to their charge using isoelectric focussing and/or according to their molecular weight. Two-dimensional separations allow various isoforms of proteins to be identified, as proteins with similar molecular weight are also separated by their charge. Using mass spectrometry, it is possible to determine whether or not a protein of interest is present in a patient sample.

As will be apparent to those skilled in the art a diagnostic or prognostic assay described herein may be a multiplexed assay. As used herein the term “multiplex”, shall be understood not only to mean the detection of two or more diagnostic or prognostic markers in a single sample simultaneously, but also to encompass consecutive detection of two or more diagnostic or prognostic markers in a single sample, simultaneous detection of two or more diagnostic or prognostic markers in distinct but matched samples, and consecutive detection of two or more diagnostic or prognostic markers in distinct but matched samples. As used herein the term “matched samples” shall be understood to mean two or more samples derived from the same initial biological sample, or two or more biological samples isolated at the same point in time.

Accordingly, a multiplexed assay may comprise an assay that detects several anti-KARI protein antibodies and/or KARI epitopes in the same reaction and simultaneously, or alternatively, it may detect other one or more antigens/antibodies in addition to one or more anti-KARI protein antibodies and/or KARI epitopes.

The present invention clearly contemplates multiplexed assays for detecting KARI protein antibodies and KARI epitopes in addition to detecting CD4+ T-helper cells via one or more receptors on the cell surface and/or one or more HIV-1 and/or HIV-2 antigens. Such assays are particularly useful for simultaneously obtaining information on co-infection with M. tuberculosis and HIV-1 and/or HIV-2, and/or for determining whether or not a subject with M. tuberculosis is immune-compromised. Clearly, such multiplexed assay formats are useful for monitoring the health of an HIV+/TB+ individual.

As will be apparent to the skilled artisan, if such an assay is antibody or ligand based, both of these antibodies must function under the same conditions.

### 4. Biological Samples and Reference Samples

Preferably, the biological sample in which a KARI protein or anti-KARI protein antibody is detected is a sample selected from the group consisting of lung, lymphoid tissue associated with the lung, paranasal sinuses, bronchi, a bronchiole, alveolus, ciliated mucosal epithelia of the respiratory tract, mucosal epithelia of the respiratory tract, broncheoalveolar lavage fluid (BAL), alveolar lining fluid, sputum, mucus, saliva, blood, serum, plasma, urine, peritoneal fluid, pericardial fluid, pleural fluid, squamous epithelial cells of the respiratory tract, a mast cell, a goblet cell, a pneumocyte (type 1 or type 2), an intra epithelial dendritic cell, a PBMC, a neutrophil, a monocyte, or any immune-globulin-containing fraction of any one or more of said tissues, fluids or cells.

In one example a biological sample is obtained previously from a subject.

Preferably, the subject from which the sample is obtained is suspected of suffering from tuberculosis or being infected by M. tuberculosis and/or is at risk of developing tuberculosis and/or at risk of being infected by M. tuberculosis.

In one example a biological sample is obtained from a subject by a method selected from the group consisting of surgery or other excision method, aspiration of a body fluid such as hypertonic saline or propylene glycol, broncheoalveolar lavage, bronchoscopy, saliva collection with a glass tube, salivette (Sarstedt AG, Sevelen, Switzerland), Ora-sure (Epitope Technologies Pty Ltd, Melbourne, Victoria, Australia), omni-sal (Saliva Diagnostic Systems, Brooklyn, N.Y., USA) and blood collection using any method well known in the art, such as, for example using a syringe.

It is particularly preferred that a biological sample is sputum, isolated from lung of a patient using, for example the method described in Gershman, N. H. et al, J Allergy Clin Immune-l, 10(4): 322-328, 1999. Preferably, the sputum is expectorated i.e., coughed naturally.

In another preferred example a biological sample is plasma that has been isolated from blood collected from a patient using a method well known in the art.

In one example, a biological sample is treated to lyse a cell in said sample. Such methods include the use of detergents, enzymes, repeatedly freezing and thawing said cells, sonication or vortexing said cells in the presence of glass beads, amongst others.

In another example, a biological sample is treated to denature a protein present in said sample. Methods of denaturing a protein include heating a sample, treating a sample with 2-mercaptoethanol, dithiotreitol (DTT), N-acetylcysteine, detergent or other compound such as, for example, guanidinium or urea. For example, the use of DTT is preferred for liquefying sputum.

In yet another example, a biological sample is treated to concentrate a protein is said sample. Methods of concentrating proteins include precipitation, freeze drying, use of funnel tube gels (TerBush and Novick, Journal of Biomolecular Techniques, 10(3); 1999), ultrafiltration or dialysis.

As will be apparent, the diagnostic and prognostic methods provided by the present invention require a degree of quantification to determine either, the amount of a protein that is diagnostic or prognostic of an infection or disease. Such quantification can be determined by the inclusion of appropriate reference samples in the assays described herein, wherein said reference samples are derived from healthy or normal individuals.

In one example, the reference sample comprises for example cells, fluids or tissues from a healthy subject who has not been previously or recently infected and is not suffering from an infection or disease. Conveniently, such reference samples are from fluids or tissues that do not require surgical resection or intervention to obtain them. Accordingly, bodily fluids and derivatives thereof are preferred. Highly preferred reference samples comprise sputum, mucus, saliva, blood, serum, plasma, urine, BAL fluid, peritoneal fluid, pericardial fluid, pleural fluid, a PBMC, a neutrophil, a monocyte, or any immune-globulin-containing fraction of any one or more of said tissues, fluids or cells.

A reference sample and a test (or patient) sample are processed, analysed or assayed and data obtained for a reference sample and a test sample are compared. In one example, a reference sample and a test sample are processed, analysed or assayed at the same time. In another example, a reference sample and a test sample are processed, analysed or assayed at a different time.

In an alternate example, a reference sample is not included in an assay. Instead, a reference sample may be derived from an established data set that has been previously generated. Accordingly, in one example, a reference sample comprises data from a sample population study of healthy individuals, such as, for example, statistically significant data for the healthy range of the integer being tested. Data derived from processing, analysing or assaying a test sample is then compared to data obtained for the sample population.

Data obtained from a sufficiently large number of reference samples so as to be representative of a population allows the generation of a data set for determining the average level of a particular parameter. Accordingly, the amount of a protein that is diagnostic or prognostic of an infection or disease can be determined for any population of individuals, and for any sample derived from said individual, for subsequent comparison to levels of the expression product determined for a sample being assayed. Where such normalized data sets are relied upon, internal controls are preferably included in each assay conducted to control for variation.

### Diagnostic Assay Kits

The present invention provides a kit for detecting M. tuberculosis infection in a biological sample. In one example, the kit comprises:

(i) one or more isolated antibodies that bind to a KARI protein or an immunogenic fragment or epitope thereof; and
(ii) means for detecting the formation of an antigen-antibody complex.

In an alternative example, the kit comprises:

(i) an isolated or recombinant KARI protein or an immunogenic fragment or epitope thereof; and
(ii) means for detecting the formation of an antigen-antibody complex.

The antibodies, immunogenic KARI peptide, and detection means of the subject kit are preferably selected from the antibodies and immunogenic KARI peptides described herein above and those examples shall be taken to be incorporated by reference herein from the description. The selection of compatible kit components for any assay format will be readily apparent to the skilled artisan from the description.

In a particularly preferred example, the subject kit comprises:

(i) an antibody that binds to an isolated or recombinant KARI protein or an immunogenic fragment or epitope thereof; and
(ii) anti-human Ig.

Preferably, the kit further comprises an amount of one or more immunogenic peptide fragments of a full-length KARI protein, or a fusion between any two or more of said peptides.

Optionally, the kit further comprises means for the detection of the binding of an antibody, fragment thereof or a ligand to a KARI protein. Such means include a reporter molecule such as, for example, an enzyme (such as horseradish peroxidase or alkaline phosphatase), a substrate, a cofactor, an inhibitor, a dye, a radionucleotide, a luminescent group, a fluorescent group, biotin or a colloidal particle, such as colloidal gold or selenium. Preferably such a reporter molecule is directly linked to the antibody or ligand.

In yet another example, a kit may additionally comprise a reference sample. Such a reference sample may for example, be a protein sample derived from a biological sample isolated from one or more tuberculosis subjects. Alternatively, a reference sample may comprise a biological sample isolated from one or more normal healthy individuals. Such a reference sample is optionally included in a kit for a diagnostic or prognostic assay.

In another example, a reference sample comprises a peptide that is detected by an antibody or a ligand. Preferably, the peptide is of known concentration. Such a peptide is of particular use as a standard. Accordingly various known concentrations of such a peptide may be detected using a prognostic or diagnostic assay described herein.

In yet another example, a kit optionally comprises means for sample preparations, such as, for example, a means for cell lysis. Preferably such means are means of solubilizing sputum, such as, for example, a detergent (e.g., tributyl phosphine, C7BZO, dextran sulfate, DTT, N-acetylcysteine, or polyoxyethylenesorbitan monolaurate).

In yet another example, a kit comprises means for protein isolation (Scopes (in: Protein Purification: Principles and Practice, Third Edition, Springer Verlag, 1994).

### Prophylactic and Therapeutic Method

The KARI protein or immunogenic fragment or epitope thereof can induce the specific production of a high titer antibody when administered to an animal subject.

Accordingly, the invention provides a method of eliciting the production of an antibody against M. tuberculosis comprising administering an isolated KARI protein or an immunogenic fragment or epitope thereof to said subject for a time and under conditions sufficient to elicit the production of antibodies, such as, for example, neutralizing antibodies that bind to M. tuberculosis.

It is within the scope of the present invention to further administer one or more second antigens e.g., M. tuberculosis BSX or S9 or GS or immunogenic fragment thereof for a time and under conditions sufficient to elicit the production of antibodies, such as, for example, neutralizing antibodies that bind to M. tuberculosis. Such administration may be at the same time as administering KARI protein or fragment (i.e., co-administration) or alternatively, before or after the KARI protein or fragment is administered to a subject.

Preferably, the neutralizing antibodies according to any of the preceding examples are high titer neutralizing antibodies.

The effective amount of KARI protein or other protein or epitope thereof to produce antibodies varies upon the nature of the immunogenic B cell epitope, the route of administration, the animal used for immunization, and the nature of the antibody sought. All such variables are empirically determined by art-recognized means.

In a preferred example, the invention provides a method of inducing immunity against M. tuberculosis in a subject comprising administering to said subject an isolated or recombinant KARI protein or immunogenic fragment or epitope thereof for a time and under conditions sufficient to elicit a humoral immune response against said an isolated or recombinant KARI protein or immunogenic fragment or epitope.

It is also within the scope of the present invention to further administer one or more second antigens e.g., M. tuberculosis BSX or S9 or GS or immunogenic fragment thereof for a time and under conditions sufficient to elicit a humoral immune response against that antigen. Such administration may be at the same time as administering KARI protein or fragment (i.e., co-administration) or alternatively, before or after the KARI protein or fragment is administered to a subject.

The immunizing antigen may be administered in the form of any convenient formulation as described herein.

By “humoral immune response” means that a secondary immune response is generated against the immunizing antigen sufficient to prevent infection by M. tuberculosis.

Preferably, the humoral immunity generated includes eliciting in the subject a sustained level of antibodies that bind to a B cell epitope in the immunizing antigen. By a “sustained level of antibodies” is meant a sufficient level of circulating antibodies that bind to the B cell epitope to prevent infection by M. tuberculosis.

Preferably, antibodies levels are sustained for at least about six months or 9 months or 12 months or 2 years.

In an alternative example, the present invention provides a method of enhancing the immune system of a subject comprising administering an immune-logically active KARI protein or an epitope thereof or a vaccine composition comprising said KARI protein or epitope for a time and under conditions sufficient to confer or enhance resistance against M. tuberculosis in said subject.

It is also within the scope of the present invention to further administer one or more second antigens e.g., M. tuberculosis BSX or GS or immunogenic fragment thereof for a time and under conditions sufficient to confer or enhance resistance against M. tuberculosis in said subject. Such administration may be at the same time as administering KARI protein or fragment (i.e., co-administration) or alternatively, before or after the KARI protein or fragment is administered to a subject.

By “confer or enhance resistance” is meant that a M. tuberculosis-specific immune response occurs in said subject, said response being selected from the group consisting of:

(i) an antibody against a KARI protein of M. tuberculosis or an epitope of said protein is produced in said subject;
(ii) neutralizing antibodies that bind to M. tuberculosis are produced in said subject;
(iii) a cytotoxic T lymphocyte (CTL) and/or a CTL precursor that is specific for a KARI protein of M. tuberculosis is activated in the subject; and
(iv) the subject has enhanced immunity to a subsequent M. tuberculosis infection or reactivation of a latent M. tuberculosis infection.

The invention will be understood to encompass a method of providing or enhancing immunity against M. tuberculosis in an uninfected human subject comprising administering to said subject an immune-logically active KARI protein or an epitope thereof or a vaccine composition comprising said KARI protein or epitope for a time and under conditions sufficient to provide immune-logical memory against a future infection by M. tuberculosis.

It is also within the scope of the present invention to further administer one or more second antigens e.g., M. tuberculosis BSX or GS or immunogenic fragment thereof for a time and under conditions sufficient to provide immune-logical memory against a future infection by M. tuberculosis. Such administration may be at the same time as administering KARI protein or fragment (i.e., co-administration) or alternatively, before or after the KARI protein or fragment is administered to a subject.

The present invention provides a method of treatment of tuberculosis in a subject comprising performing a diagnostic method or prognostic method as described herein.

In one example, the present invention provides a method of prophylaxis comprising:

(i) detecting the presence of M. tuberculosis infection in a biological sample from a subject; and
(ii) administering a therapeutically effective amount of a pharmaceutical composition described herein to reduce the number of pathogenic bacilli in the lung, blood or lymph system of the subject.

As will be apparent from the disclosure herein, suitable compositions according to this example comprise KARI protein or immunogenic fragment thereof optionally with on or more other immunogen M. tuberculosis proteins or peptide fragments, in combination with a pharmaceutically acceptable carrier or excipient. It is clearly within the scope of the present invention for such compositions to include KARI protein or fragment thereof according to any example hereof or a combination or mixture of peptides or epitopes or fragments, and one or more second antigens e.g., M. tuberculosis BSX and/or S9 and/or Rv1265 and/or EF-Tu and/or P5CR and/or TetR and/or GS or immunogenic fragments thereof e.g., as set forth in any one of SEQ ID Nos: 3-60 or a combination/mixture thereof.

Preferably, the composition is administered to a subject harboring a latent or active M. tuberculosis infection.

Without being bound by any theory or mode of action, the therapeutic method enhances the ability of a T cell to recognize and lyse a cell harboring M. tuberculosis, or that the ability of a T cell to recognize a T cell epitope of an antigen of M. tuberculosis is enhanced, either transiently or in a sustained manner. Similarly, reactivation of a T cell population may occur following activation of a latent M. tuberculosis infection, or following re-infection with M. tuberculosis, or following immunization of a previously-infected subject with a KARI protein or epitope or vaccine composition of the invention. Standard methods can be used to determine whether or not CTL activation has occurred in the subject, such as, for example, using cytotoxicity assays, ELISPOT, or determining IFN-γ production in PBMC of the subject.

Preferably, the peptide or derivative or variant or vaccine composition is administered for a time and under conditions sufficient to elicit or enhance the expansion of CD8⁺ T cells. Still more preferably, the peptide or derivative or variant or vaccine composition is administered for a time and under conditions sufficient for M. tuberculosis-specific cell mediated immunity (CMI) to be enhanced in the subject.

By “M. tuberculosis-specific CMI” is meant that the activated and clonally expanded CTLs are MHC-restricted and specific for a CTL epitope of the invention. CTLs are classified based on antigen specificity and MHC restriction, (i.e., non-specific CTLs and antigen-specific, MHC-restricted CTLs). Non-specific CTLs are composed of various cell types, including NK cells and antibody-dependent cytotoxicity, and can function very early in the immune response to decrease pathogen load, while antigen-specific responses are still being established. In contrast, MHC-restricted CTLs achieve optimal activity later than non-specific CTL, generally before antibody production. Antigen-specific CTLs inhibit or reduce the spread of M. tuberculosis and preferably terminate infection.

CTL activation, clonal expansion, or CMI can be induced systemically or compartmentally localized. In the case of compartmentally localized effects, it is preferred to utilize a vaccine composition suitably formulated for administration to that compartment. On the other hand, there are no such stringent requirements for inducing CTL activation, expansion or CMI systemically in the subject.

The effective amount of KARI protein or epitope thereof, optionally in combination with one or more other proteins or epitopes e.g., derived from BSX or GS or S9 proteins of M. tuberculosis, to be administered solus or in a vaccine composition to elicit CTL activation, clonal expansion or CMI, varies upon the nature of the immunogenic epitope, the route of administration, the weight, age, sex, or general health of the subject immunized, and the nature of the CTL response sought. All such variables are empirically determined by art-recognized means.

The KARI protein or epitope thereof, optionally in combination with one or more other proteins or epitopes e.g., derived from BSX or S9 or GS proteins of M. tuberculosis, and optionally formulated with any suitable or desired carrier, adjuvant, BRM, or pharmaceutically acceptable excipient, is conveniently administered in the form of an injectable composition. Injection may be intranasal, intramuscular, sub-cutaneous, intravenous, intradermal, intraperitoneal, or by other known route. For intravenous injection, it is desirable to include one or more fluid and nutrient replenishers.

The optimum dose to be administered and the preferred route for administration are established using animal models, such as, for example, by injecting a mouse, rat, rabbit, guinea pig, dog, horse, cow, goat or pig, with a formulation comprising the peptide, and then monitoring the CTL immune response to the epitope using any conventional assay.

Adoptive transfer techniques may also be used to confer or enhance resistance against M. tuberculosis infection or to prevent or reduce the severity of a reactivated latent infection. Accordingly, in a related example, there is provided a method of enhancing or conferring immunity against M. tuberculosis in an uninfected human subject comprising contacting ex vivo a T cell obtained from a human subject with an immune-logically active KARI protein or an epitope thereof or a vaccine composition comprising said protein or epitope for a time and under conditions sufficient to confer M. tuberculosis activity on said T cells.

In a preferred example, the invention provides a method of enhancing the M. tuberculosis-specific cell mediated immunity of a human subject, said method comprising:

(i) contacting ex vivo a T cell obtained from a human subject with an immune-logically active KARI protein or a CTL epitope thereof or a vaccine composition comprising said protein or epitope for a time and under conditions sufficient to confer M. tuberculosis activity on said T cells; and
(ii) introducing the activated T cells autologously to the subject or allogeneically to another human subject.

As with other examples described herein, the present invention encompasses the administration of additional immunogenic proteins or epitopes e.g., derived from BSX or S9 or GS proteins of M. tuberculosis.

The T cell may be a CTL or CTL precursor cell.

The human subject from whom the T cell is obtained may be the same subject or a different subject to the subject being treated. The subject being treated can be any human subject carrying a latent or active M. tuberculosis infection or at risk of M. tuberculosis infection or reactivation of M. tuberculosis infection or a person who is otherwise in need of obtaining vaccination against M. tuberculosis or desirous of obtaining vaccination against M. tuberculosis.

Such adoptive transfer is preferably carried out and M. tuberculosis reactivity assayed essentially as described by Einsele et al., Blood 99, 3916-3922, 2002, which procedures are incorporated herein by reference.

By “M. tuberculosis activity” is meant that the T cell is rendered capable of being activated as defined herein above (i.e. the T cell will recognize and lyse a cell harboring M. tuberculosis or able to recognize a T cell epitope of an antigen of M. tuberculosis, either transiently or in a sustained manner). Accordingly, it is particularly preferred for the T cell to be a CTL precursor which by the process of the invention is rendered able to recognize and lyse a cell harboring M. tuberculosis or able to recognize a T cell epitope of an antigen of M. tuberculosis, either transiently or in a sustained manner.

For such an ex vivo application, the T cell is preferably contained in a biological sample obtained from a human subject, such as, for example, a biopsy specimen comprising a primary or central lymphoid organ (eg. bone marrow or thymus) or a secondary or peripheral lymphoid organ (eg. blood, PBMC or a buffy coat fraction derived there from).

Preferably, the T cell or specimen comprising the T cell was obtained previously from a human subject, such as, for example, by a consulting physician who has referred the specimen to a pathology laboratory for analysis.

Preferably, the subject method further comprises obtaining a sample comprising the T cell of the subject, and more preferably, obtaining said sample from said subject.

### Formulations

The present invention clearly contemplates the use of the KARI protein or an immunogenic fragment or epitope thereof in the preparation of a therapeutic or prophylactic subunit vaccine against M. tuberculosis infection in a human or other animal subject.

Accordingly, the invention provides a pharmaceutical composition or vaccine comprising a KARI protein or an immunogenic fragment or epitope thereof in combination with a pharmaceutically acceptable diluent.

In a preferred example, the composition according to this example comprises KARI protein or immunogenic fragment thereof optionally with on or more other immunogenic M. tuberculosis proteins or peptide fragments, in combination with a pharmaceutically acceptable carrier or excipient. It is clearly within the scope of the present invention for such compositions to include KARI protein or fragment thereof according to any example hereof or a combination or mixture of peptides or epitopes or fragments, and one or more second antigens e.g., M. tuberculosis BSX and/or S9 and/or Rv1265 and/or EF-Tu and/or P5CR and/or TetR and/or GS or immunogenic fragments thereof e.g., as set forth in any one of SEQ ID Nos: 3-60 or a combination/mixture thereof.

The KARI protein, and optional other protein, or immunogenic fragment or epitope thereof is conveniently formulated in a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the pharmaceutical composition are adjusted according to routine skills in the art.

In certain situations, it may also be desirable to formulate the KARI protein and optional other protein or an immunogenic fragment or epitope thereof, with an adjuvant to enhance the immune response to the B cell epitope. Again, this is strictly not essential. Such adjuvants include all acceptable immune-stimulatory compounds such as, for example, a cytokine, toxin, or synthetic composition. Exemplary adjuvants include IL-1, IL-2, BCG, aluminium hydroxide, N-acetyl-muramyl-L-threonyl-D-isoglutamine (thur-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP) 1983A, referred to as MTP-PE), lipid A, MPL and RIBI, which contains three components extracted from bacteria, monophosphoryl lipid A, trehalose dimycolate and cell wall skeleton (MPL+TDM+CWS) in a 2% squalene/Tween 80 emulsion.

Particularly preferred adjuvants for use in a vaccine against M. tuberculosis are described for example by Elhay and Andersen Immune-l. Cell Biol. 75, 595-603, 1997; or Lindblad et al., Infect. Immun. 65, 1997.

It may be desirable to co-administer biologic response modifiers (BRM) with the KARI protein or immunogenic fragment or epitope thereof, to down regulate suppressor T cell activity. Exemplary BRM's include, but are not limited to, Cimetidine (CIM; 1200 mg/d) (Smith/Kline, PA, USA); Indomethacin (IND; 150 mg/d) (Lederle, N.J., USA); or low-dose Cyclophosphamide (CYP; 75, 150 or 300 mg/m.sup.2) (Johnson/Mead, NJ, USA).

Preferred vehicles for administration of the KARI protein and optional other protein, or immunogenic fragment or epitope thereof, include liposomes. Liposomes are microscopic vesicles that consist of one or more lipid bilayers surrounding aqueous compartments. (Bakker-Woudenberg et al., Eur. J. Clin. Microbiol. Infect. Dis. 12(Suppl. 1), S61 (1993); and Kim, Drugs 46, 618 (1993)). Liposomes are similar in composition to cellular membranes and as a result, liposomes generally are administered safely and are biodegradable.

Techniques for preparation of liposomes and the formulation (e.g., encapsulation) of various molecules, including peptides and oligonucleotides, with liposomes are well known to the skilled artisan.

Depending on the method of preparation, liposomes may be unilamellar or multilamellar, and can vary in size with diameters ranging from 0.02 μm to greater than 10 μm. A variety of agents are encapsulated in liposomes. Hydrophobic agents partition in the bilayers and hydrophilic agents partition within the inner aqueous space(s) (Machy et al., LIPOSOMES IN CELL BIOLOGY AND PHARMACOLOGY (John Libbey 1987), and Ostro et al., American J. Hosp. Pharm. 46, 1576 (1989)).

Liposomes can also adsorb to virtually any type of cell and then release the encapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446, 368 (1985)). In the present context, the KARI protein or immunogenic fragment or epitope thereof may be localized on the surface of the liposome, to facilitate antigen presentation without disruption of the liposome or endocytosis. Irrespective of the mechanism or delivery, however, the result is the intracellular disposition of the associated KARI protein or immunogenic fragment or epitope thereof.

Liposomal vectors may be anionic or cationic. Anionic liposomal vectors include pH sensitive liposomes which disrupt or fuse with the endosomal membrane following endocytosis and endosome acidification. Cationic liposomes are preferred for mediating mammalian cell transfection in vitro, or general delivery of nucleic acids, but are used for delivery of other therapeutics, such as peptides or lipopeptides.

Cationic liposome preparations are made by conventional methodologies (Feigner et al, Proc. Nat'l Acad. Sci. USA 84, 7413 (1987); Schreier, Liposome Res. 2, 145 (1992)). Commercial preparations, such as Lipofectin (Life Technologies, Inc., Gaithersburg, Md. USA), are readily available. The amount of liposomes to be administered are optimized based on a dose response curve Feigner et al., supra.

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these liposomes are well known in the art. (See COLLOIDAL DRUG DELIVERY SYSTEMS, vol. 66, J. Kreuter, ed., Marcel Dekker, Inc. 1994).

Other forms of delivery particle, for example, microspheres and the like, also are contemplated for delivery of the KARI protein and optional other protein, or immunogenic fragment or epitope thereof.

Guidance in preparing suitable formulations and pharmaceutically effective vehicles, are found, for example, in REMINGTON'S PHARMACEUTICAL SCIENCES, chapters 83-92, pages 1519-1714 (Mack Publishing Company 1990) (Remington's), which are hereby incorporated by reference.

Alternatively, the peptide or derivative or variant is formulated as a cellular vaccine via the administration of an autologous or allogeneic antigen presenting cell (APC) or a dendritic cell that has been treated in vitro so as to present the peptide on its surface.

Nucleic acid-based vaccines that comprise nucleic acid, such as, for example, DNA or RNA, encoding the immune-logically active KARI protein and optional other protein, or epitope(s) thereof, and cloned into a suitable vector (eg. vaccinia, canary pox, adenovirus, or other eukaryotic virus vector) are also contemplated. Preferably, DNA encoding a KARI protein and optional other protein, is formulated into a DNA vaccine, such as, for example, in combination with the existing Calmette-Guerin (BCG) or an immune adjuvant such as vaccinia virus, Freund's adjuvant or another immune stimulant.

The present invention is further described with reference to the following non-limiting examples.

### Example 1

### Sample Collection and Processing, Antibody Production and Immune-Assays

Subject to the disclosure in the subsequent examples i.e., Example 2 et seq., the following general methods were employed for sample collection and processing, antibody production and evaluation. A method referred to in this example has been utilized unless an alternate method is specifically recited in a subsequent example i.e., Example 2 et seq. Methods referred to in the subsequent examples are to be construed with reference to that specific example.

### 1. Collection of Patient Sputum Samples

TB-negative and TB-positive sputa were used to evaluate antibody pairs for a TB diagnostic as described in the subsequent examples. Eighty (80) patient sputa samples were recruited from Cameroon in 2007. Samples are treated with protease inhibitors and frozen at −30° C.

Similarly unprocessed sputa were also obtained from Becton, Dickinson & CO., Research Triangle Park, Durham, N.C., USA and are referred to herein as “Sputum-BD”. Additional samples are collected from alternate sites in Johannesburg, South Africa, and from Health Concepts International Limited, Thailand.

### 2. Pre-Treatment of Sputa

### a) “Sputum-M1”

In one example, sputa fractions referred to herein as “sputum-M1” are prepared by diluting collected sputum 1:1 (v/v) to a final concentration of 10 mM freshly-made Dithiothreitol (DTT) in 50 mM phosphate buffer pH 7.4. EDTA-free protease inhibitor cocktail tablets (Roche Molecular Biochemicals, Cat#1873580) are added according to the manufacturer's or supplier's instructions as appropriate to provide a final 1:4 (v/v) dilution of sputum. Samples are agitated by vortexing for about 30 seconds, and then mixed for about 30 min at 4° C. using an orbital shaker or gentle vortexing, taking care to avoid substantial cell lysis. The liquefied sputum is then centrifuged at 2,000×g for about 10 min at 4° C. to pellet cells and remove insoluble material. The supernatant is centrifuged at 14,000×g for 10 min at 4° C., to pellet fine particulate matter. The supernatant is removed and filtered using a 0.2 μm pore size GD/X PVDF sterile filter and the filtrate retained and stored frozen at −20° C.

### b) “Sputum-C1”

In a further example, sputa fractions referred to herein as “sputum-C1” are prepared by diluting collected sputum 1:1 (v/v) to a final concentration of 10 mM freshly-made Dithiothreitol (DTT) in 50 mM phosphate buffer pH 7.4. EDTA-free protease inhibitor cocktail tablets (Roche Molecular Biochemicals, Cat#1873580) are added according to the manufacturer's or supplier's instructions as appropriate to provide a final 1:2 (v/v) dilution of sputum. Samples are agitated by vortexing for about 30 seconds, and then mixed for about 30 min at 4° C. using an orbital shaker or gentle vortexing, taking care to avoid substantial cell lysis. The liquefied sputum is then centrifuged at 2,000×g for about 10 min at 4° C. to pellet cells and remove insoluble material. The supernatant is centrifuged at 14,000×g for 10 min at 4° C., to pellet fine particulate matter. The supernatant is removed and stored frozen at −20° C. without filtration.

### 3. Processing of Frozen Sputa for Assay

Four alternative processes were employed for further processing of frozen pre-treated sputa prepared as described herein above.

### a) Method 1

Sputum-M1 (2.5 mL) prepared as described herein above is equivalent to about 0.6 mL of undiluted (“neat”) sputum. In this exemplary method, sputum-M1 is unprocessed further for assay in a replacement ELISA using 17×150 uL replacements.

### b) Method 2

Sputum-C1 (1.8 mL) prepared as described above is equivalent to 0.9 mL of undiluted (“neat”) sputum. In this exemplary method, sputum-C1 is reduced to 0.6 mL volume by acetone precipitation for assay in a replacement ELISA using 4×150 uL replacements. In particular, Sputum-C1 is centrifuged to remove insoluble material, the supernatant is transferred into a fresh tube and four (4) volumes of cold acetone are added, and samples incubated at −80° C. for 30 min, after which time they are centrifuged at 4,000×g at 4° C. for 30 min to collect the precipitated protein fraction. The protein pellet is retained and air-dried for about 30 min, re-dissolved gently in 0.6 mL of 50 mM Tris pH 7.8, 5 mM MgCl₂.

### c) Method 3

Sputum-C1 (9 mL) prepared as described above is equivalent to 4.5 mL of undiluted (“neat”) sputum. In this exemplary method, sputum-C1 is size fractionated, desalted and made to about 0.6 mL volume, for assay in a replacement ELISA using 4×150 uL replacements. Briefly, frozen Sputum-C1 is thawed, adjusted to a final concentration of 0.3 mM EDTA, and 4 mL of 50 mM Tris pH 7.8, 5 mM MgCl₂ added. The samples are centrifuged at 4,000×g at ambient temperature for 20 min to pellet insoluble material. The supernatant is retained, transferred to a fresh tube, diluted in an equal volume of 50 mM Tris pH 7.8, 5 mM MgCl₂, applied to a 100 kDa MW cut-off size exclusion spin column, and centrifuged at 4,000×g at ambient temperature for 25 min. The eluate is retained and transferred to a 5 kDa MW cut-off size-exclusion spin column, and centrifuged at 4,000×g (ambient temperature) for at least about 60 min or until ˜0.6 mL eluate is collected. Sample volumes are adjusted to about 0.62 mL using with 50 mM Tris pH 7.8, 5 mM MgCl₂ for assay in replacement ELISA as described above.

### d) Method 4

Sputum-M1 (18 mL) prepared as described above is equivalent to 4.5 mL of undiluted (“neat”) sputum. In this exemplary method, sputum-is size fractionated, desalted and made to about 0.6 mL volume, for assay in a replacement ELISA using 4×150 uL replacements. Briefly, frozen Sputum-M1 is thawed, adjusted to a final concentration of 0.3 mM EDTA, and 4 mL of 50 mM Tris pH 7.8, 5 mM MgCl₂ added. The samples are centrifuged at 4,000×g at ambient temperature for 20 min to pellet insoluble material. The supernatant is retained, transferred to a fresh tube, diluted in an equal volume of 50 mM Tris pH 7.8, 5 mM MgCl₂, applied to a 100 kDa MW cut-off size exclusion spin column, and centrifuged at 4,000×g at ambient temperature for 25 min. The eluate is retained and transferred to a 5 kDa MW cut-off size-exclusion spin column, and centrifuged at 4,000×g (ambient temperature) for at least about 60 min or until ˜0.6 mL eluate is collected. Sample volumes are adjusted to about 0.62 mL using with 50 mM Tris pH 7.8, 5 mM MgCl₂ for assay in replacement ELISA as described above.

4. Preparation of IgG Fractions from Sputum, Serum or Plasma

Patient sputum, serum or plasma is diluted in 8.2 ml Immune-pure IgG binding buffer (Pierce), then filtered through a 0.22 gm filter before application to a protein A column attached to an ÄKTA Explorer (Amersham Biosciences). Bound antibody is eluted with Immune-pure gentle Ag/Ab elution buffer (Pierce). The eluted fractions (IgG bound to antigens) are pooled and left on ice for 3 hours to allow dissociation of immune complexes. The IgG fraction is then separated from the antigen fraction by filtration through a 100,000 molecular weight cut off column (Millipore). Both fractions and the flow through from the protein A column are dialysed with benzoylated dialysis membrane (Sigma) against 4 litres of phosphate buffered saline pH 7.2 overnight at 4° C., then another 4 litres for 3 hours. All fractions, (flow through and retentate from the 100,000 cut off column and flow through from the protein A column) are acetone precipitated at a ratio of 10 parts acetone to 1 part sample for one hour at −20° C., then spun at 4000 g for 20 minutes. The precipitated samples are solubilized in sample buffer containing 5M urea, 2M thiourea, 2% CHAPS, 2% SB3-10 and 40 mM Tris to a final concentration of approximately 2 mg/ml, and then reduced with 5 mM tributyl phosphine and alkylated with 10 mM acrylamide for 1.5 h. The alkylation reaction is quenched with the addition of DTT to a final concentration of 10 mM. The samples are divided into 200 μl aliquots and stored at −20° C.

5. Selection of M. tuberculosis Antigens for Diagnostic Assays

The primary criteria for selection of M. tuberculosis antigens for diagnostic antigen-based assays is their presence in TB-positive sputa and immunogenicity to provide for a simply diagnostic test. The candidate antigens were identified in TB-positive sputa as described in the following paragraphs.

### a) Determination of Protein Content

The protein content of the samples is estimated using a Bradford assay. Prior to rehydration of IPG strips, samples are centrifuged at 21000×g for 10 minutes. The supernatant is collected and 10 μl of 1% Orange G (Sigma) per ml added as an indicator dye.

### b) Two-Dimensional Gel Electrophoresis

### First Dimension

Dry 11 cm IPG strips (Amersham-Biosciences) are rehydrated for 16-24 hours with 180 μl of protein sample. Rehydrated strips are focussed on a Protean IEF Cell (Bio-Rad, Hercules, Calif.) or Proteome System's IsoElectrIQ electrophoresis equipment for approx 140 kVhr at a maximum of 10 kV. Focussed strips are then equilibrated in urea/SDS/Tris-HCl/bromophenol blue buffer.

### c) Second Dimension

Equilibrated strips are inserted into loading wells of 6-15% (w/v) Tris-acetate SDS-PAGE pre-cast 10 cm×15 cm GelChips (Tyrian Diagnostics, Sydney Australia). Electrophoresis is performed at 50 mA per gel for 1.5 hours, or until the tracking dye reached the bottom of the gel. Proteins from the retentate fraction or flow-through fraction are stained using SyproRuby (Molecular Probes). Proteins from the eluate fraction are stained with silver according to the protocol of Shevchenko et al. (Anal Chem. 68(5): 850-8, 1996). Gel images are scanned after destaining using an AlphaImager System (Alpha Innotech Corp.). Gels are then stained with Coomassie G-250 to assist visualisation of protein spots in subsequent analyses.

### d) Mass Spectrometry:

Prior to mass spectrometry protein samples are prepared by in-gel tryptic digestion. Protein gel pieces are excised, destained, digested and desalted using an Xcise™, an excision/liquid handling robot (Tyrian Diagnostics, Sydney, Australia and Shimadzu-Biotech, Kyoto, Japan) in association with the Montage In-Gel Digestion Kit (developed by Tyrian Diagnostics and distributed by Millipore, Billerica, Ma, 01821, USA). Prior to spot cutting, the 2-D gel is incubated in water to maintain a constant size and prevent drying. Subsequently, the 2-D gel is placed on the Xcise, a digital image was captured and the spots to be cut are selected. After automated spot excision, gel pieces are subjected to automated liquid handling and in-gel digestion. Briefly, each spot is destained with 100 μl of 50% (v/v) acetonitrile in 100 mM ammonium bicarbonate. The gel pieces are dried by adding 100% acetonitrile, the acetonitrile is removed after 5 seconds and the gels dried completely by evaporating the residual acetonitrile at 37° C. Proteolytic digestion is performed by rehydrating the dried gel pieces with 30 μl of 50 mM ammonium bicarbonate (pH 7.8) containing 5 μg/mL modified porcine trypsin and incubated at 37° C. overnight.

Ten microliters (10 μl) of the tryptic peptide mixture is removed to a clean microtitre plate in the event that additional analysis by Liquid Chromatography (LC)-Electrospray Ionisation (ESI) MS was required.

Automated desalting and concentration of tryptic peptides prior to MALDI MS is performed using R2-based chromatography. Adsorbed peptides are eluted from the tips onto a 384-position MALDI-TOF sample target plate (Kratos, Manchester, UK or Bruker Daltronics, Germany) using 2 μl of 2 mg/ml α-cyano-4-hydroxycinnamic acid in 90% (v/v) acetonitrile and 0.085% (v/v) TFA.

Digests are analyzed using an Axima-CFR MALDI MS mass spectrometer (Kratos, Manchester, UK) in positive ion reflectron mode. A nitrogen laser with a wavelength of 337 nm is used to irradiate the sample. The spectra are acquired in automatic mode in the mass range 600 Da to 4000 Da applying a 64-point raster to each sample spot. Only spectra passing certain criteria are saved. All spectra undergo an internal two point calibration using an autodigested trypsin peak mass, m/z 842.51 Da and spiked adrenocorticotropic hormone (ACTH) peptide, m/z 2465.117 Da. Software designed by Tyrian Diagnostics, as contained in the web-based proteomic data management system BioinformatIQ™ (Tyrian Diagnostics), is used to extract isotopic peaks from MS spectra.

Protein identification is performed by matching the monoisotopic masses of the tryptic peptides (i.e. the peptide mass fingerprint) with the theoretical masses from protein databases using IonIQ or MASCOT database search software (Proteome System Limited, North Ryde, Sydney, Australia). Querying is done against the non-redundant SwissProt (Release 40) and TrEMBL (Release 20) databases (June 2002 version), and protein identities are ranked through a modification of the MOWSE scoring system. Propionamide-cysteine (cys-PAM) or carboxyamidomethyl-cysteine (cys-CAM) and oxidized methionine modifications are taken into account and a mass tolerance of 100 ppm is allowed.

Miscleavage sites are only considered after an initial search without miscleavages had been performed. The following criteria are used to evaluate the search results: the MOWSE score, the number and intensity of peptides matching the candidate protein, the coverage of the candidate protein's sequence by the matching peptides and the gel location.

In addition, or alternatively, proteins are analysed using LC-ESI-MS. Tryptic digest solutions of proteins (10 μl) are analysed by nanoflow LC/MS using an LCQ Deca Ion Trap mass spectrometer (ThermoFinnigan, San Jose, Calif.) equipped with a Surveyor LC system composed of an autosampler and pump. Peptides are separated using a PepFinder kit (Thermo-Finnigan) coupled to a C18 PicoFrit column (New Objective). Gradient elution from water containing 0.1% (v/v) formic acid (mobile phase A) to 90% (v/v) acetonitrile containing 0.1% (v/v) formic acid (mobile phase B) is performed over a 30-60-minute period. The mass spectrometer is set up to acquire three scan events—one full scan (range from 400 to 2000 amu) followed by two data dependant MS/MS scans.

### e) Bioinformatic Analysis:

Following automated collection of mass spectra peaks, data are processed as follows. All spectra are firstly checked for correct calibration of peptide masses. Spectra are then processed to remove background noise including masses corresponding to trypsin peaks and matrix. The data are then searched against publicly-available SwissProt and TrEMBL databases using Tyrian Diagnostics search engine IonIQ v69 and/or MASCOT. PSD data is searched against the same databases using the in-house search engine FragmentastIQ. LC MS-MS data is also searched against the databases using the SEQUEST search engine software.

### 6. Validation of M. Tuberculosis Antigens and Antibodies for Diagnostic Assays

Validation of candidate diagnostic markers and antibodies was performed by determining the presence of the corresponding endogenous antigen in whole cell lysates (WCL) derived from cultures of the laboratory strain of M. tuberculosis designated H37Rv, and in whole cell lysates (WCL) derived from cultures of two M. tuberculosis clinical strains designated CSU93, HN878 and CDC1551, using an amplified ELISA system as described according to any example hereof. Filtrates of whole cell lysates, cell membranes, cell walls and cytosolic fractions of cells were also employed. Antigens and antibodies that were detectable in all strains were selected for further validation.

Validation of candidate diagnostic markers and antibodies was also performed by determining the specific expression of the corresponding endogenous antigen in whole cell lysates (WCL) derived from cultures of non-Mycobacterial organisms e.g., Escherichia coli, Bacillus subtilis, Pseudomonas aeruginosa, and Saccharomyces cerevisiae using an amplified ELISA system as described herein below. Filtrates of whole cell lysates were also employed. Antigens and antibodies that were detected specifically in M. tuberculosis were preferred.

Validation of candidate diagnostic markers and antibodies was also performed by determining the specific expression of the corresponding endogenous antigen in whole cell lysates (WCL) derived from cultures of the laboratory strain of M. tuberculosis designated H37Rv, as opposed to expression in other Mycobacteria species e.g., M. avium and M. intracellulaire, using an amplified ELISA system as described herein below. Filtrates of whole cell lysates were also employed. Antigens that were detected specifically by particular antibodies in M. tuberculosis were preferred, however those that were also expressed in M. avium and/or M. intracellulaire were not discarded. This is because a diagnostic test that tests any Mycobacteria in a sample has utility as a primary generic assay and can be employed in conjunction with species-specific tests for M. tuberculosis e.g., employing specific diagnostic markers as disclosed herein and/or a culture test to determine the presence or absence of M. tuberculosis.

### 7. Preparation of Whole Cell Lysates

Proteins were extracted from lyophilised M. tuberculosis cells. The cells were resuspended in an extraction buffer and processed in a bead mill to rupture the cells and release the proteins. The cell debris was pelleted by centrifugation and the supernatant used as the whole cell lysate (WCL). A Bradford colorimetric assay was done to estimate the protein concentrations. In some instances, a cytosolic extract obtained from Colorado State University was used.

### 8. Antibody Production Methods

Antibodies are prepared by immunization with a synthetic immunogenic peptide derived from a specific immunogenic protein of M. tuberculosis identified as described in the subsequent examples, or alternatively, by immunization with the full-length immunogenic protein of M. tuberculosis or an immunogenic fragment of the M. tuberculosis immunogenic protein produced by recombinant means using standard procedures. For recombinant protein or fragment production, a DNA sequence of M. tuberculosis strain H37Rv that encodes the immunogen was isolated and cloned into a suitable vector for expression in Escherichia coli, and the expressed protein or fragment was purified by standard chromatography techniques.

### b) Monoclonal Antibodies

Full-length recombinant protein, or a peptide fragment derived from the full-length sequence, was used as an antigen for antibody production, according to standard procedures. Approximately 5 mg of peptide/protein was provided to NeoClone, Madison, Wis., USA for generation of monoclonal antibodies according to their standard protocol. About 1 mg of the peptide/protein was provided as a biotinylated product for quality control. BALB/cByJ female mice were immunized with protein according to Neoclone's standard immunization process. Test bleeds of the immunized mice were performed at regular intervals for use in the quality control sera ELISAs using biotinylated peptide. Polyclonal sera having the highest titer were determined using ELISA. Mice having polyclonal antibody titers of at least 1,000 were used for the ABL-MYC infection process. The spleens of about 3-5 mice having the highest titer of polyclonal antibodies cross-reactive with peptide antigen were used for the ABL-MYC infection, according to NeoClone's standard infection procedure. The splenocytes of the ABL-MYC-infected mice were transplanted into approximately 20 naive mice. Ascites fluid developed in the transplanted mice were isolated and screened for cells producing monoclonal antibodies (mAbs) that bind to the target peptide antigen.

Cell lines (i.e., plasmacytomas) producing mAbs were isolated. Binding affinities and isotype specificities were confirmed using ELISA. The mAbs were provided in 1 ml aliquots (approximately) in ascites, together with the associated cell lines. Those monoclonal antibody preparations having high titers when assayed using standard procedures were subject to further diagnostic testing as described herein, using the monoclonal sera as a capture and/or a detection reagent. The mAbs are purified from ascites using protein G or protein A columns.

### c) Polyclonal Antibodies

Polyclonal antibody preparations were prepared against the full length recombinant M. tuberculosis proteins, by immunization of chickens or rabbits using standard procedures. For the purposes of nomenclature, polyclonal antisera designated “ChX/Y” refer to pooled preparations comprising separate batches “ChX” and “ChY” prepared in chickens. Polyclonal antibody preparations against recombinant M. tuberculosis proteins were selected for their high titer when assayed using standard procedures e.g., ELISA, and were subjected to further diagnostic testing as described herein, using the polyclonal sera as a capture and/or a detection reagent.

### 8. Antibody Selection Criteria

Antibodies are selected based on their sensitivity and specificity towards the immunogen in ELISA with a preferred limit of detection (LOD) of e.g., less than about 100 ng/mL recombinant antigen in a single-site ELISA and/or less than about 500 pg/mL antigen in an amplified two-site ELISA. Antibodies are also selected that detected the M. tuberculosis antigen in M. tuberculosis culture with little or no cross-reactivity to other Mycobacteria species or non-Mycobacteria pathogens.

Antibodies are screened initially by reactivity against the immunogen in each case using one-site ELISA. The skilled artisan will be aware that one-site ELISA requires unlabelled recombinant immunogen bound to the surface of a solid substrate and a labelled detector antibody e.g., an antibody conjugated to a detectable marker such as colloidal gold or biotin, wherein the detector antibody specifically binds to an epitope on a target antigen contained in the immobilized immunogen. The detector antibody binds to the immobilized immunogen such that it is immobilized on the solid substrate and labelled indirectly by binding of the label on the detector antibody.

Alternatively, or in addition a two-site ELISA is performed, subject to availability of antibodies with which to pair the test antibody in a two-site test. Two-site ELISA requires an unlabelled capture antibody bound to the surface of a solid substrate and a labelled detector antibody e.g., an antibody conjugated to a detectable marker such as colloidal gold or biotin, wherein both the capture antibody and detector antibody specifically bind to a target antigen albeit to different or non-interfering epitopes on the target antigen. When the antigen is present in a test sample, the detector antibody and capture antibody “sandwich” the antigen such that it is immobilized on the solid substrate and labelled indirectly by binding of the label on the detector antibody.

Generally, for antibodies having a LOD in two-site ELISA of less than about 500 pg/mL, Western blot (WB) immune-electrophoresis was performed to confirm the specificity of antibodies for the recombinant protein and an endogenous M. tuberculosis protein of the expected molecular weight that is present in whole cell lysates. Briefly, recombinant proteins and whole cell lysates were separated by electrophoresis on 1-dimensional SDS/polyacrylamide gels (10% polyacrylamide Nu-PAGE gels) using a MOPS or MES buffer system according to the manufacturer's instructions. The resolved proteins were transferred from the gels onto PVDF membranes using a semi-dry electro blotting system. The membranes were then probed with primary antibodies to the corresponding antigens followed by HRP-conjugated secondary antibodies capable of binding the antibody probe used according to standard procedures. Specific signals were then visualized by a chemiluminescence detection system and an image acquired using either X-ray film followed by scanning or using the Fuji-LAS-3000 imager to acquire an image directly from the treated blot. For each antigen tested, Western blot conditions were optimized in terms of dilution of the primary and secondary antibodies required to provide optimum signal to noise ratio (data not shown). Replica blots were probed with primary and secondary antibodies (positive control) or secondary antibody alone (negative control).

These Western data were confirmed by competition experiments wherein a primary antibody is pre-incubated with a molar excess of recombinant immunogen in solution prior to conducting the Western blot to thereby compete for epitopes on the resolved proteins. Thus, loss of signal in Western blotting confirms an initial conclusion of antibody specificity. Briefly, Western Blot analysis was performed as described in the preceding paragraph, except that primary antibody was pre-incubated with a 100- to 200-molar excess of the corresponding recombinant protein. Blots were probed in the usual manner.

### 9. Selection of Antibody Pairs

For selection of antibody pairs, two-site ELISA is performed using the most sensitive antibodies available that meet the antibody selection criteria described in the preceding section. In performing two-site ELISA in the context of selecting antibody pairs, the inventors employed antibody candidates in both configurations as detector and capture antibody, to thereby determine optimum configurations of capture and detector antibodies. In general, detector antibody was employed at different dilutions against a titration of recombinant immunogen for each capture antibody concentration tested. Preferred detector antibodies for this purpose are biotinylated antibodies that are detectable using poly-HRP-conjugated streptavidin. When biotinylated detector antibody is not available, a preferred detector antibody comprises an unlabelled detector antibody that is detectable by sequential binding of (i) biotinylated secondary antibody (e.g., anti-rabbit Ig or anti-chicken Ig or anti-mouse Ig) to the detector antibody and (ii) poly-HRP-conjugated streptavidin to the bound biotinylated secondary antibody.

### 10. ELISA Formats

### a) One-Site ELISA

A NUNC plate was coated with a serial dilution of the recombinant protein and incubated overnight at 4° C. After blocking, the plate was incubated with various dilutions of the test antibody followed by HRP-conjugated secondary antibody and TMB. The volume of each reaction was 50 μl. The plate was washed between each addition. The immune reaction was stopped by the addition of 0.5 M H₂SO₄ after an appropriate time based on visual examination of color development (usually about 30 min), and the OD read in a microplate reader at wavelengths of 450 nm and 620 nm.

The resulting data were exported to Microsoft Excel where the Delta OD (OD 450-620), referred to as OD, was recorded for data analysis. The sensitivity of the assay was determined as described below and expressed as LODAbT. An antibody with an LODAbT of less than about 100 ng/mL was further tested for suitability as either a capture or detector antibody in a sandwich ELISA.

### b) Standard Two-Site or “Sandwich” ELISA

A standard sandwich ELISA was performed using selected antibody pairs e.g., to determine their suitability as either capture or detector antibodies. NUNC immune-plates were coated with various dilutions of capture antibody, then incubated sequentially with the relevant recombinant protein or whole cell lysate of filtrate comprising a test immunogen, and various dilutions of detector antibody, HRP-conjugated secondary antibody and SIGMA TMB. The volume of each reaction was 50 μl. The plate was washed between each addition. The immune reaction was stopped by the addition of 0.5 M H₂SO₄ after an appropriate time based on visual examination of color development (usually about 30 min), and the OD read in a microplate reader at wavelengths of 450 nm and 620 nm.

The resulting data were exported to Microsoft Excel for analysis. The sensitivity of the assay was determined as described below and referred to as the LOD; antibody pairs producing the lowest LOD scores (e.g., less than about 3 ng/mL) were selected for further optimization in an amplified sandwich ELISA.

### c) Amplified Sandwich ELISA

Amplified sandwich ELISA is performed as for standard sandwich ELISA as described herein above and analysed by the same procedures, except plates are incubated with either a biotinylated detector antibody or a detector antibody followed by a biotinylated secondary antibody. Amplification is achieved by the addition of 50 μl of various dilutions of poly80-HRP-streptavidin then 50 μl of Pierce TMB. Antibody pairs producing the lowest LOD scores (e.g., less than about 500 pg/mL) were selected.

### d) ELISA Data Analysis

ELISA data were exported to Microsoft Excel for analysis. A standard curve was plotted using an X-Y graph with the mean OD+SD (OD=OD_{450 nm}−OD_{620 nm}) on the Y axis and the recombinant protein/peptide concentration (e.g., pg/mL) on the X axis (logarithmic scale). The coefficient of variation, calculated as the standard deviation divided by the mean and expressed as a percent (CV %), was used as a measure of intra-assay and inter-assay variability.

GraphPad Prism software was employed to fit a 4-parameter logistic curve to the standard curve data points. Antigen concentrations for unknown samples were determined by interpolation of the OD values off the generated curve.

### e) Determination of Limit of Detection (LOD Values

The Limit of Detection (LOD) for an antibody pair in a two-site “sandwich” ELISA where a suitable calibration curve was available was defined as the concentration of immunogen that produced an OD value equivalent to the average baseline value plus 3×SD, using functions available in GraphPad Prism software. For one-site ELISA, or for sandwich ELISA where calibration curves were not available, the LOD of the antibody or antibody pair was estimated using Microsoft Excel. For each immunogenic protein being analyzed, the optical density data from ELISA dose response curves were used to calculate LOD values.

For one-site ELISA: For one-site ELISA data or two-site ELISA data where there were insufficient data points for software to apply a non-linear regression curve fit function, an estimate of the LOD was obtained in Excel. The start of the baseline of the dose response curve and the baseline mean OD+3×SD were determined as follows: Increments in optical density, calculated as the difference between optical density for a given immunogen concentration and the optical density for the next higher immunogen concentration, were determined. The data point where the increment in optical density was less than about 0.05 OD units was deemed to be the start of the baseline. Average and SD optical density values for replicate samples at the deemed baseline start point and at the next two incremental points were used to calculate the average+3×SD for the series. The concentration of immunogen producing an optical density greater than the mean baseline OD+3×SD was deemed the LOD value.

For Sandwich ELISA: In one approach, the concentration of recombinant protein/peptide immunogen i.e., the value “log₁₀ [immunogen]” and the replicate optical density values obtained from sandwich ELISA were transferred into an Excel worksheet template that has been generated to automatically calculate the necessary values from the raw ELISA data. An R² value was generated as an estimate of the goodness of fit of the standard curve, and a value greater than about 0.99 was accepted as a good fit. The 99% Confidence Interval (CI) value ranges were also calculated. The maximum value in the range for the bottom asymptote of the fitted curve was interpolated from the fitted standard curve. The interpolated value, when un-logged, represented the recombinant protein concentration having an OD value equivalent to the average baseline value plus 3×SD. That value, referred to as the LOD value, was deemed to indicate sensitivity of the ELISA.

Alternatively, the log₁₀ [immunogen] and the corresponding replicate optical density values were exported into GraphPad Prism where a non-linear regression curve fit function was used to fit a 4-parameter logistic curve to the data points. A non-linear regression curve fit function was used to fit a sigmoid curve to the data. An R² value was generated as an estimate of the goodness of fit of the standard curve, and a value greater than about 0.99 was accepted as a good fit. The concentration of recombinant protein/peptide immunogen corresponding to the baseline mean optical density (OD) plus 3×SD was interpolated from the standard curve.

### Example 2

### Antigen-Based Diagnosis of Tuberculosis or Infection by M. Tuberculosis Using Antibodies that Bind to Ketol-Acid M. Tuberculosis Reductoisomerase (KARI)

### 1. Identification of KARI Protein in TB-Positive Subjects

A protein having a molecular weight of about 36 kDa was recognized in TB+ samples. The sequences of ten peptides from MALDI-TOF data matched a sequence encoded by the ilvC gene of M. tuberculosis set forth in SEQ ID NO: 1. The percent coverage of SEQ ID NO: 1 by these 10 peptides was about 37%, suggesting that the peptide fragments were derived from this same protein marker.

The identified protein having the amino acid sequence set forth in SEQ ID NO: 1 is a putative Ketol-Acid Reducto Isomerase and was designated as “KARI”.

### 2. Antibodies

Polyclonal antibodies were prepared against recombinant KARI protein encoded by the ilvC gene of M. tuberculosis using standard procedures. Monoclonal antibodies were prepared using ABL-MYC technology (NeoClone, Madison Wis. 53713, USA) to produce cell lines secreting monoclonal antibodies (mAbs) against the full length recombinant KARI protein encoded by the ilvC gene of M. tuberculosis, and against peptide fragments of the full-length protein, essentially as described herein. Peptide fragments used for preparation of monoclonal antibodies comprised the following amino acid regions of the full-length protein:

a) residues 40-56 of SEQ ID NO: 1 with optional C-terminal cysteine residue added;
b) residues 290-300 of SEQ ID NO: 1 with optional C-terminal cysteine residue added; and
c) residues 298-310 of SEQ ID NO: 1 with optional C-terminal cysteine residue added.

More than ten antibodies, including polyclonal and monoclonal antibody preparations, were produced and screened for their suitability as described in Example 1. In particular, the polyclonal antibody preparation designated “Ch34/35”, and several monoclonal antibody preparations e.g., designated Mo1283F, Mo1E7, Mo2C7; Mo3A2, Mo2B1, Mo4F7, Mo3C3, Mo1C10, Mo4C10, Mo1F6, Mo2D6, Mo3H3 and Mo4D11, were produced.

Antibodies designated Ch34/35, Mo1283F, Mo1F6 and Mo2B1 were prepared against recombinant KARI protein.

Antibodies designated Mo4F7 and Mo4C10 were produced against a synthetic peptide comprising residues 40-56 of SEQ ID NO: 1. The antibody designated Mo2D6 was prepared against a synthetic peptide comprising residues 290-300 of SEQ ID NO: 1. Monoclonal antibodies designated Mo1 A4, Mo1H2, Mo2E5, Mo2G2, Mo3H3, Mo4C3, Mo4D2 and Mo4D11 were prepared against a synthetic peptide comprising residues 298-310 of SEQ ID NO: 1 and, of these preparations, Mo3H3 and Mo4D11 had the highest titers in ELISA.

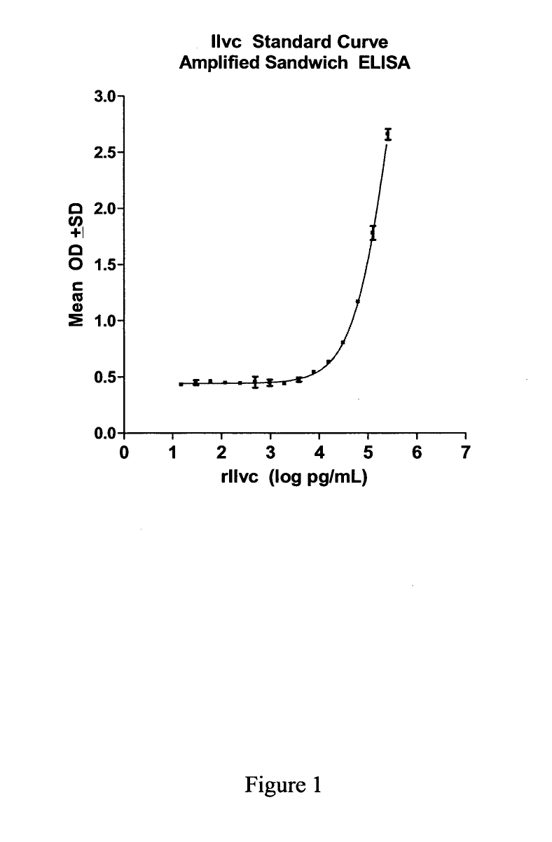

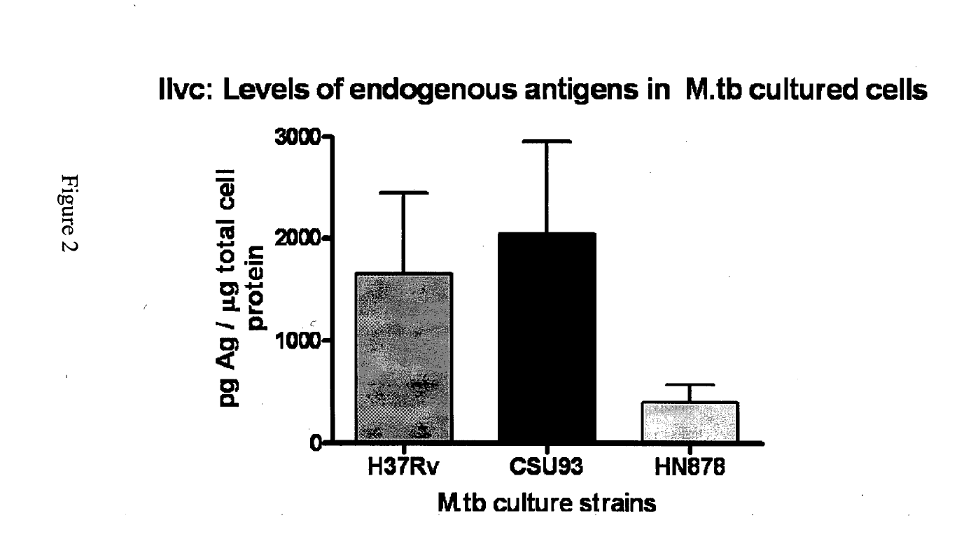

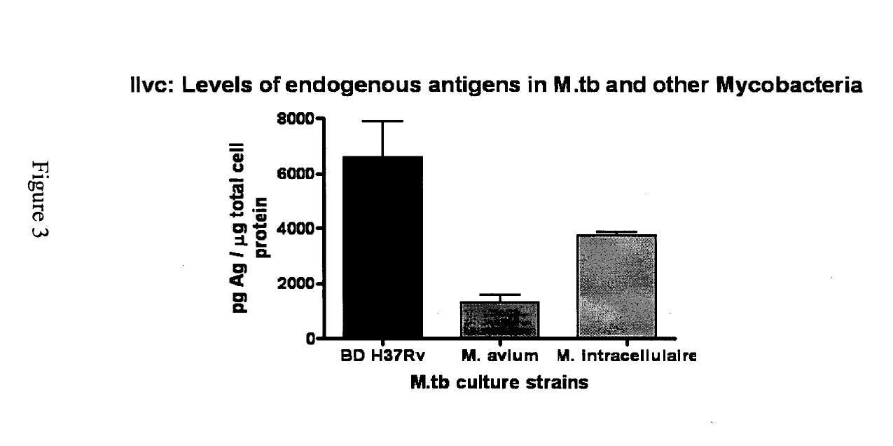

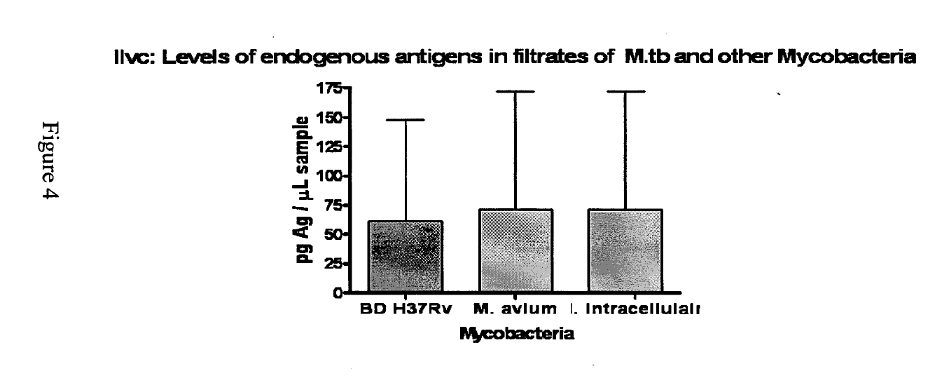

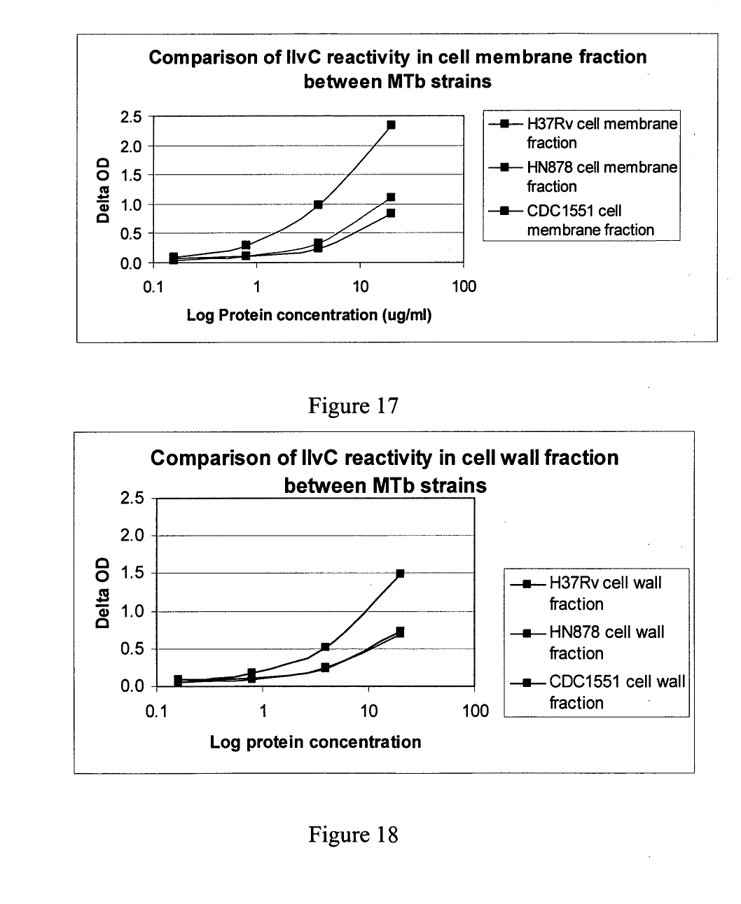

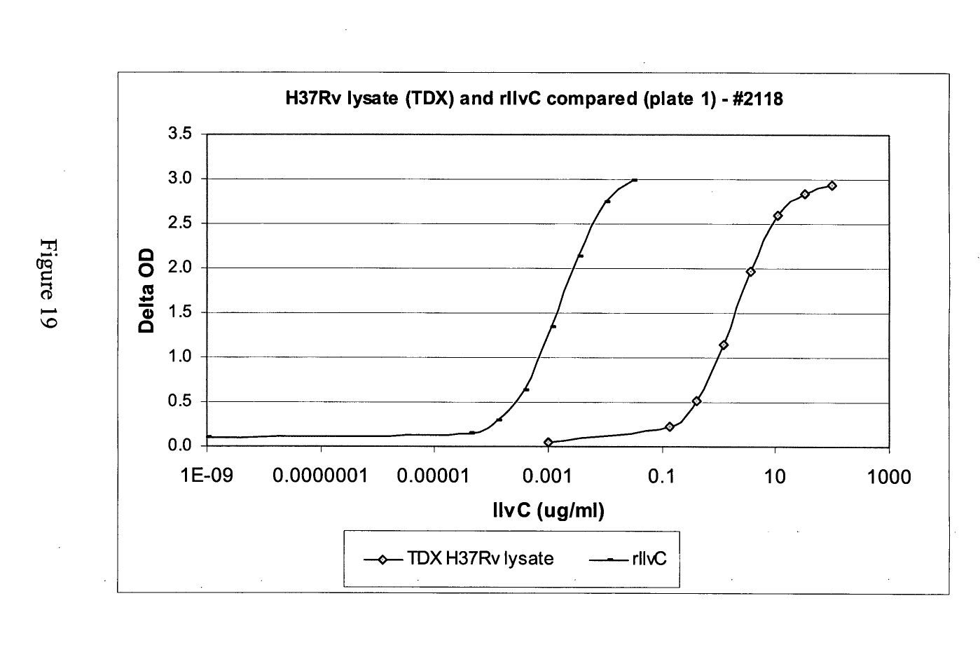

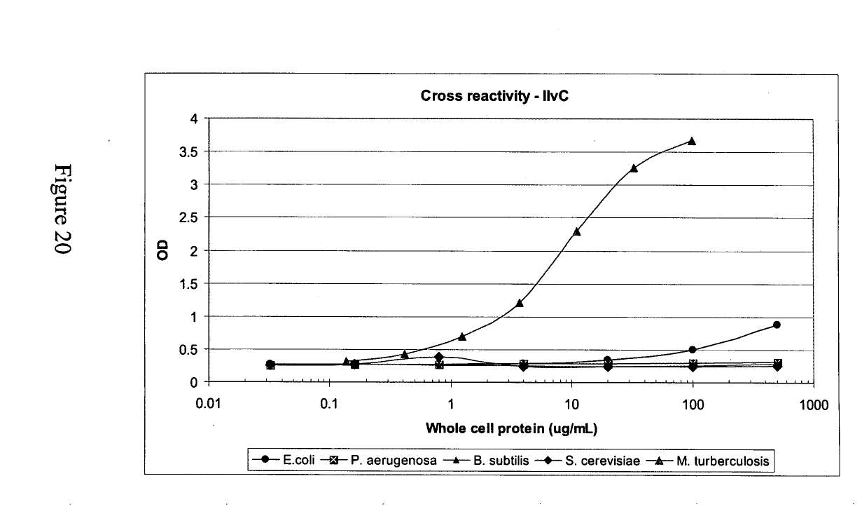

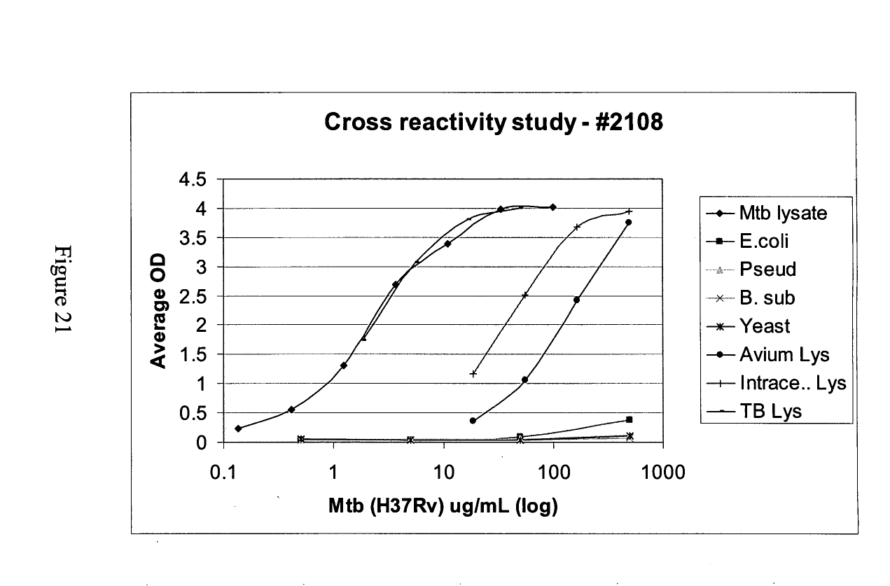

The antibodies were screened as described in Example 1 to determine optimum antibody pairs and preferred orientation in two-site ELISA. In one example, the mouse-derived monoclonal antibody Mo1283F is employed as a capture antibody and the chicken-derived polyclonal antibody Ch34/35 as a detector antibody e.g., see FIGS. 1-14. In another example, the mouse-derived monoclonal antibody Mo2B1 (or simply “2B1”) is employed as a capture antibody and paired with chicken-derived antibody Ch34/35 as a detector antibody e.g., see FIGS. 15-23e. In another example, the mouse-derived monoclonal antibody Mo1F6 (or simply “1F6”) is employed as a capture antibody and paired with the mouse-derived monoclonal antibody Mo2B1 as a detector antibody e.g., see FIGS. 24 and 28. In another example, the mouse-derived monoclonal antibody Mo2D6 (or simply “2D6”) is employed as a capture antibody and paired with the mouse-derived monoclonal antibody Mo2B1 as a detector antibody e.g., FIG. 28. In another example, chicken-derived antibody Ch34/35 is employed as a capture antibody and paired with monoclonal antibody Mo2B1 as a detector antibody e.g., see FIG. 29.

In yet other examples, the monoclonal antibody 2B1 is employed as a capture antibody and paired with the monoclonal antibody 1F6 as a detector antibody. In another example, the monoclonal antibody 2B1 is employed as a capture antibody and paired with the monoclonal antibody 2D6 as a detector antibody. In another example, the monoclonal antibody 2D6 is employed as a capture antibody and paired with the monoclonal antibody 1F6 as a detector antibody. In another example, the monoclonal antibody 1F6 employed as a capture antibody and paired with the monoclonal antibody 2D6 as a detector antibody.

Other orientations and antibody combinations are not excluded.

### 3. Linear Epitope Mapping of KARI Protein

The antibodies prepared against KARI protein and peptides were screened against a pepset of synthetic peptides derived from the sequence of the full-length KARI protein set forth in SEQ ID NO: 1, to map the linear B-cell epitopes in the full-length protein.

Data (not shown) suggest that the full-length KARI protein of M. tuberculosis comprises the following linear B-cell epitopes, which are useful as diagnostic peptidyl reagents and for preparing diagnostic antibodies:

a) residues 1-23 of SEQ ID NO: 1;
b) residues 40-71 of SEQ ID NO: 1, and preferably residues 57-71 of SEQ ID NO: 1;
c) residues 97-111 of SEQ ID NO: 1;
d) residues 169-199 of SEQ ID NO: 1;
e) residues 265-279 of SEQ ID NO: 1;
f) residues 290-300 of SEQ ID NO: 1, preferably residues 298-300 of SEQ ID NO: 1; and
g) residues 313-333 of SEQ ID NO: 1.

For example, the monoclonal antibody Mo1283F binds within residues 97-111 of SEQ ID NO: 1; the monoclonal antibodies Mo4F7 and Mo4C10 bind within residues 40-56 of SEQ ID NO: 1; and the monoclonal antibody Mo2D6 binds within residues 290-300 of SEQ ID NO: 1 and preferably with residues 298-300 of SEQ ID NO: 1. see e.g., FIGS. 24-28.

Fine mapping of these linear B-cell epitopic regions (a) through (g) is performed by testing the abilities of antibodies against KARI protein to bind to 5-mer peptides within or overlapping these regions, and/or the abilities of those antibodies to bind to mutant peptides containing amino acid substitutions relative to the base sequence i.e., SEQ ID NO: 1. Alternatively, or in addition, fine mapping of these linear B-cell epitopic regions (a) through (g) is performed by testing the abilities of 5-mer peptides within or overlapping these regions, and/or the abilities of mutant peptides containing amino acid substitutions relative to the base sequence i.e., SEQ ID NO: 1, to elicit production of antibodies that bind to KARI protein.

### 4. Validation of KARI and Antibodies Thereto as Diagnostic Reagents

The amino acid sequence of KARI protein from M. tuberculosis strain H37Rv is presented as SEQ ID NO: 1. The translation product has an expected molecular mass of about 36 kDa. One-dimensional SDS/PAGE analysis of a hexa-histidine-tagged rKARI protein performed essentially as described in Example 1 showed that the KARI protein migrated as a single band of approximately 37 kDa (data not shown), which is the expected mass of the fusion protein, based on the theoretical mass of the translation product and the hexahistidine tag moiety.

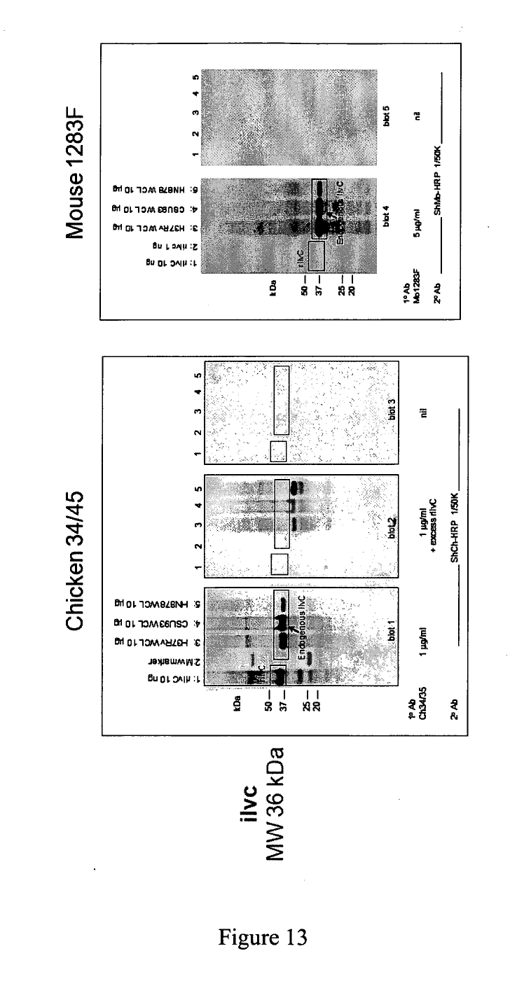

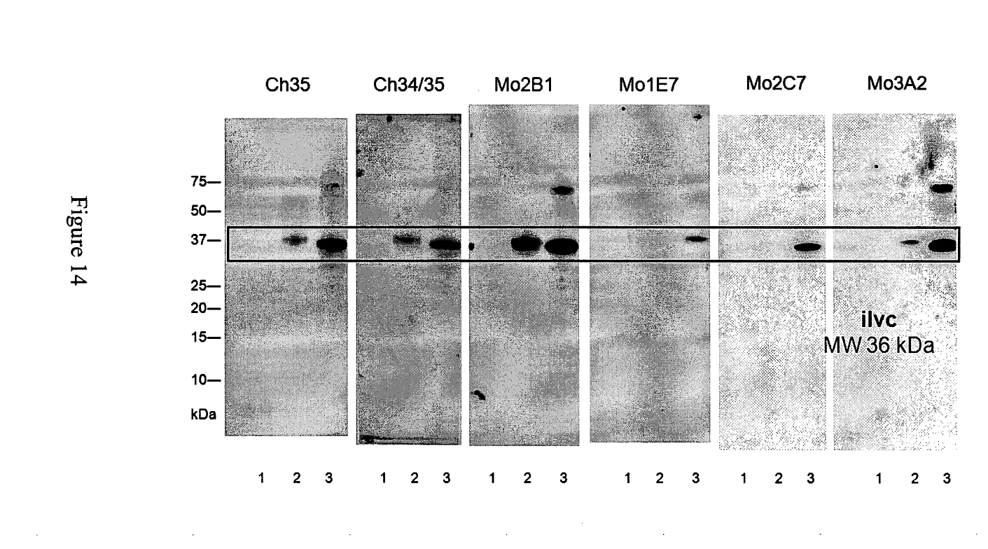

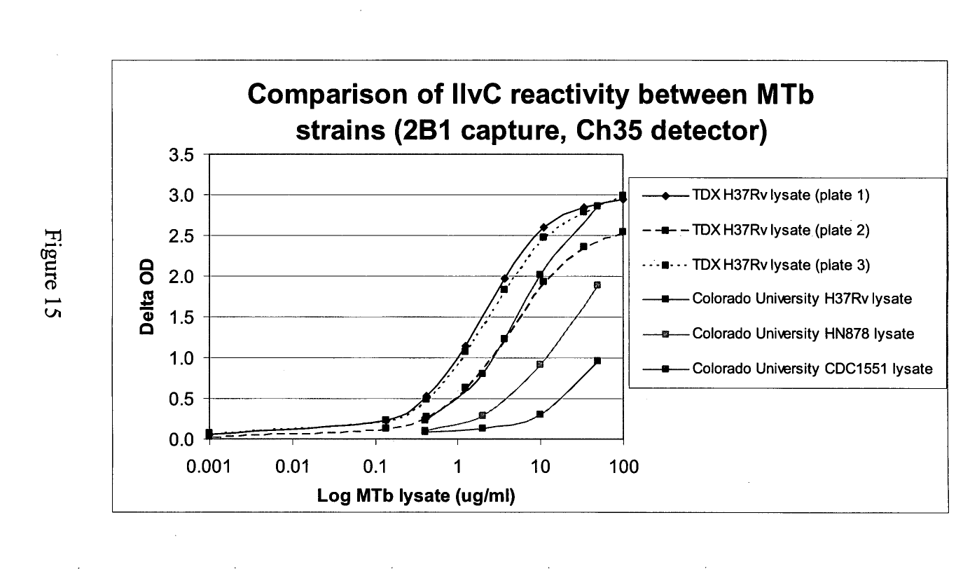

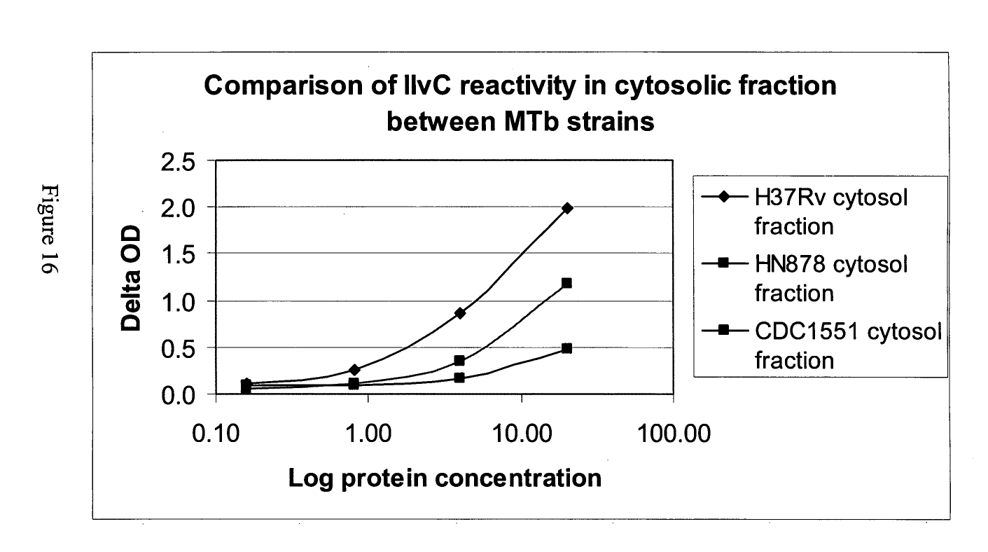

Western blot analyses were carried out using ELISA capture (Mo1283F) and detector (Ch34/35) antibodies separately, to detect recombinant KARI protein and endogenous KARI protein in whole cell lysates of M. tuberculosis H37Rv, M. tuberculosis CSU93 and M. tuberculosis HN878. Both antibodies recognized a band in whole cell lysates derived from all three M. tuberculosis strains that had the expected molecule mass of native KARI protein (i.e., about 36 kDa), as well as detecting the slightly larger recombinant KARI protein (FIG. 13). Binding was highly-specific with little background. The available data therefore confirm the specificity of the antibodies Mo1283F and Ch34/35 for detecting the M. tuberculosis KARI protein.

Competition Western blot analysis performed essentially as described in Example 1 also indicated that binding of the polyclonal antibody Ch34/35 to recombinant KARI protein and endogenous KARI protein could be ablated by pre-incubation of antibodies with excess concentration of unlabelled recombinant KARI protein (FIG. 13). In summary, the available data indicate that the antibodies Mo1283F and Ch34/35 antibodies bind to M. tuberculosis KARI protein specifically.

In similar experiments, monocolonal antibodies designated Mo2B1, Mo1 E7, Mo2C7 and Mo3A2 were shown to bind specifically to whole cell lysate (WCL) of cultured M. tuberculosis H37Rv (lane 3), and antibodies designated Mo2B1 and Mo3A2 also bound to recombinant KARI protein in Western blots FIG. 14).

### 5. Amplified Sandwich ELISA for Detection of M. Tuberculosis KARI Protein

Amplified ELISA was performed essentially as described in this example and in Example 1, using 5 μg/mL of Mo1283F antibody as a capture reagent and 2.5 μg/mL of Ch34/35 polyclonal antibody as a detector antibody, and a biotinylated secondary antibody with HRP-conjugated streptavidin to detect the bound detector antibody.

Data presented in FIG. 1 indicate that under the assay conditions tested and with this preferred, albeit not essential, orientation of antibodies, there is low background noise and a LOD of about 1690 pg/mL. Such sensitivity of detection coupled with low background in sandwich ELISA is considered by the inventors to be within useful limits.

### 6. Cross-Reactivity Between Anti-KARI Antibodies and Different M. Tuberculosis Isolates

To further assess the suitability of KARI as a diagnostic marker for the presence of M. tuberculosis in biological samples, and to assess the specificities of antibodies prepared against KARI protein, the inventors compared antibody reactivities in amplified sandwich ELISA performed as described herein above between cellular extracts of the clinical M. tuberculosis strains CSU93 and H878 and the laboratory M. tuberculosis strain H37Rv.

Briefly, an ELISA plate was coated overnight with capture antibody Mo1283F. Following washing to remove unbound antibody, a cellular extract from each isolate was added to the wells of the antibody-coated ELISA plates. As a negative control for each assay, buffer without cellular extract was used. Following incubation for 1 hour and washing to remove unbound antigen, detection antibody Ch34/35 was contacted with the bound antigen-body complexes. Following incubation at room temperature for 1 hour, plates were washed, incubated with 50 μl of diluted secondary antibody (e.g., biotinylated donkey anti-chicken IgG and poly-40 streptavidin-HRP conjugate) for 1 hour, washed again, incubated with TMB for 10 mins, and the absorbance at 450-620 nm was determined. Samples were assayed in duplicate over three dilutions of whole cell extracts. A calibration standard curve was produced based on standardized levels of KARI protein.

Data presented in FIG. 2 show that M. tuberculosis KARI protein is present in both the clinical M. tuberculosis isolate CSU93 and the laboratory strain H37Rv at comparable levels. Lower levels of KARI protein were detectable in M. tuberculosis H878, suggesting that antibodies against KARI protein may not distinguish specific M. tuberculosis clinical strains.

These data were also verified in a similar set of experiments conducted using monoclonal antibody Mo2B1 as a capture antibody and Ch34/35 as a detector antibody in amplified sandwich ELISA (FIG. 15). Data in FIG. 15 also demonstrated comparable levels between H37Rv and a further clinical isolate, CDC1551.

The data presented in FIGS. 2 and 15 do not abrogate utility of antibodies against KARI protein in a general single-analyte diagnostic test, or alternatively, as part of a multi-analyte test in conjunction with antibodies against specific strains of M. tuberculosis such as described herein or known in the art. For example, antibodies against KARI protein may be employed in conjunction with subsequent culture of M. tuberculosis from KARI-positive clinical specimens to yield information on clinically-relevant strains present in the sample, if required.

To determine whether or not KARI expression was restricted to a particular sub-cellular fraction, or (whether or not lower levels of the protein in HN878 were due to inhibitory proteins in a particular sub-cellular fraction, amplified ELISA was performed on cytosolic fractions, cell membrane fractions and cell wall fractions of H37Rv, HN878 and CDC1551, using Mo2B1 as a capture antibody and Ch34/35 as a detector antibody. Data presented in FIGS. 16-19 demonstrate that the KARI protein is detectable in each fraction, and that the relative levels of KARI protein in each fraction mirror the levels in cell lysates.

### 7. Cross-Reactivity Between Different Mycobacteria Species

To further assess the suitability of KARI as a diagnostic marker for the presence of M. tuberculosis in biological samples, and to assess the specificities of antibodies prepared against KARI protein, the inventors compared antibody reactivities in amplified sandwich ELISA performed as described herein above between cellular extracts of the Mycobacteria species M. tuberculosis, M. avium and M. intracellulaire.

Briefly, an ELISA plate was coated overnight with capture antibody Mo1283F. Following washing to remove unbound antibody, a cellular extract from each Mycobacteria species was added to the wells of the antibody-coated ELISA plates. As a negative control for each assay, buffer without cellular extract was used. Following incubation for 1 hour and washing to remove unbound antigen, detection antibody Ch34/35 was contacted with the bound antigen-body complexes. Following incubation at room temperature for 1 hour, plates were washed, incubated with 50 μl of diluted secondary antibody (e.g., biotinylated donkey anti-chicken IgG and poly-40 streptavidin-HRP conjugate) for 1 hour, washed again, incubated with TMB for 10 mins, and the absorbance at 450-620 nm was determined. Samples were assayed in duplicate over three dilutions of whole cell extracts. A calibration standard curve was produced based on standardized levels of KARI protein.

Similar experiments were conducted using monoclonal antibody Mo2B1 as a capture reagent, and Ch34/35 polyclonal antibody as a detector reagent.

Data presented in FIGS. 3, 4 and 21 show detectable albeit low cross-reactivity between the three Mycobacteria species, indicating that M. tuberculosis KARI protein may be better suited for species-specific detection of M. tuberculosis under these assay conditions using Mo2B1 as a capture reagent. This does not abrogate utility of Mo1283F or Mo2B1, or other antibodies against KARI protein in a general single-analyte diagnostic test, or alternatively, as part of a multi-analyte test in conjunction with antibodies against a species-specific marker of M. tuberculosis such as described herein or known in the art. For example, antibodies against KARI protein may be employed in conjunction with subsequent culture of M. tuberculosis from KARI-positive clinical specimens.

Alternatively, or in addition, antibodies against KARI protein may be employed simultaneously with one or more antibodies against M. tuberculosis Rv1265 and/or M. tuberculosis BSX protein and/or M. tuberculosis EF-Tu and/or M. tuberculosis S9 protein as described herein which have low cross-reactivity to the other Mycobacteria tested. In interpreting such a multi-analyte test, binding of antibodies against KARI protein indicates the presence of a Mycobacterium in the clinical sample and the additional binding of antibodies against M. tuberculosis Rv1265 and/or M. tuberculosis BSX protein and/or M. tuberculosis EF-Tu and/or M. tuberculosis S9 protein indicates a greater likelihood of M. tuberculosis infection. The combination of antibodies against M. tuberculosis KARI protein and one or more of antibodies against M. tuberculosis Rv1265 protein and antibodies against M. tuberculosis BSX protein is especially preferred for such applications, based on the low cross-reactivity of the antibodies against Rv1265 and BSX to M. avium and M. intracellulaire.

### 8. Low Cross-Reactivity Between M. Tuberculosis and Non-Mycobacteria Pathogens

To further assess the suitability of KARI as a diagnostic marker for the presence of M. tuberculosis in biological samples, the inventors compared antibody cross-reactivities in amplified sandwich ELISA performed between cellular extracts of M. tuberculosis strain H37Rv (a laboratory strain), Escherichia coli, Bacillus subtilise, Pseudomonas aeruginosa and Saccharomycese cerevisiae.

Briefly, an ELISA plate was coated overnight with capture antibody Mo1283F or Mo2B1. Following washing to remove unbound antibody, a cellular extract from each microorganism was added the wells of the antibody-coated ELISA plates. As a negative control for each assay, buffer without cellular extract was used. Following incubation for 1 hour and washing to remove unbound antigen, detection antibody Ch34/35 was contacted with the bound antigen-body complexes. Following incubation at room temperature for 1 hour, plates were washed, incubated with 50 μl of a secondary antibody (i.e., biotinylated donkey anti-chicken IgG and poly-40 streptavidin-HRP conjugate) for 1 hour, washed again, incubated with TMB for 10 mins, and the absorbance at 450-620 nm was determined.

Data presented in FIGS. 5, 20 and 21 show no significant cross-reactivity of antibodies against M. tuberculosis KARI protein with Escherichia coli, Bacillus subtilis, Pseudomonas aeruginosa or Saccharomyces cerevisiae cellular extracts under the conditions tested, indicating that the antibodies form the basis of a Mycobacterium-specific test.

### 9. Detection of KARI Protein in Clinical Samples

To further assess the suitability of KARI as a diagnostic marker for the presence of M. tuberculosis in biological samples, the inventors determined the ability of antibodies to detect endogenous KARI protein in clinical samples obtained from TB-positive subjects who had been diagnosed previously on the basis of smear test and M. tuberculosis culture assay results. Patients had been categorized on the basis of both smear and culture test results, and HIV status. All subjects tested were both smear-negative and culture-negative or alternatively, both smear-positive and culture-positive.

Briefly, sandwich ELISA was performed as described herein above on the sputum samples, which were prepared by Method 3 and assayed as 17×150 microlitre aliquots under the replacement amplification protocol (see below). ELISA plate was coated overnight with capture antibody Mo1283F or Mo2B1. Following washing to remove unbound antibody, treated sputa were added to the wells of the antibody-coated ELISA plates. As a negative control for each assay, buffer was used. Following incubation for 1 hour and washing to remove unbound antigen, detection antibody Ch34/35 was contacted with the bound antigen-body complexes. Following incubation at room temperature for 1 hour, plates were washed, incubated with 50 μl of a secondary antibody (i.e., biotinylated donkey anti-chicken IgG and poly-40 streptavidin-HRP conjugate) for 1 hour, washed again, incubated with TMB for 10 mins, and the absorbance at 450-620 nm was determined.

Data presented in FIGS. 6, 7, and 10-12 (Mo1283F:Ch34/35 antibody pair) and FIGS. 23a-e (Mo2B1:Ch34/35 antibody pair) show significantly higher levels of KARI protein detected in all TB-positive samples tested that had been shown previously to be smear-positive and/or culture-positive. Background signals were detected in the majority of TB smear-negative samples. However, a few smear-negative samples tested positive to M. tuberculosis KARI protein using both antibody combinations (FIGS. 11, 12, and FIGS. 23a-c), suggesting utility for a surrogate assay e.g., smear test and/or other antigen-based test employing antibodies to BSX and/or Rv1265 and/or S9 as described according to any example hereof to enhance specificity of the assay or to confirm results for KARI protein.

For example, as shown in Table 1 hereof, 6/7 smear-negative samples exemplified herein that tested positive for KARI protein were shown to be negative for other antigens exemplified herein, e.g., S9 protein and/or BSX protein and/or Rv1265 protein. Smear-negative samples that tested positive for KARI protein were also screened other protein markers described herein, and were recorded as positive if the average signal was greater than 3 standard deviations above the assay buffer blank.

TABLE 1
Detection of smear negative clinical samples.

| Smear | Sample ID | KARI (ilvC) | Rv1265 | BSX | S9 |
|---|---|---|---|---|---|
| 0 | MPC364 | positive | negative | negative | negative |
| 0 | MPC363 | positive | positive | positive | negative |
| 0 | MPC375 | positive | | | questionable |
| 0 | MPC388 | positive | | | negative |
| 0 | MPC339 | positive | | negative | negative |
| 0 | MPC311 | positive | positive | negative | negative |
| 0 | MPC313 | positive | negative | negative | negative |

### 10. Evaluation of Signal Inhibition by Sample

To assess whether or not inhibitory or signal-suppressing factors are present in sputa that might adversely affect assay sensitivity e.g., in ELISA or a point-of-care or field test format, sputa samples were spike with 10 ng/mL recombinant M. tuberculosis KARI protein and the resultant samples serially diluted 1:27 (v/v) over three steps. Samples were incubated overnight and assayed by amplified ELISA as described herein above, or assayed immediately.

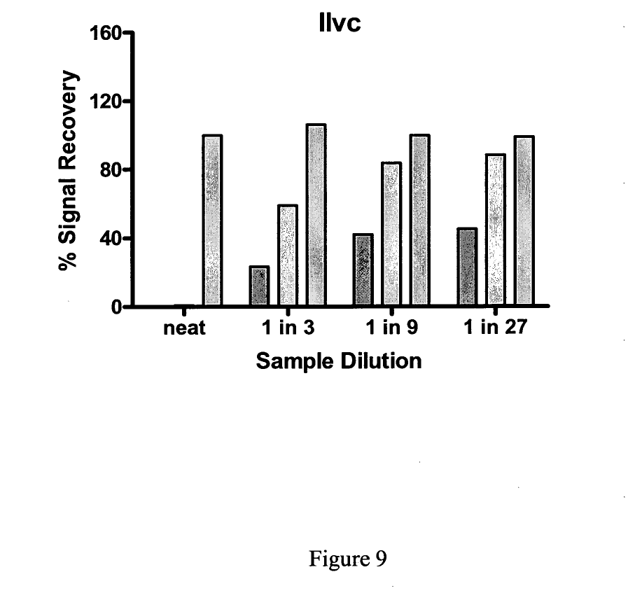

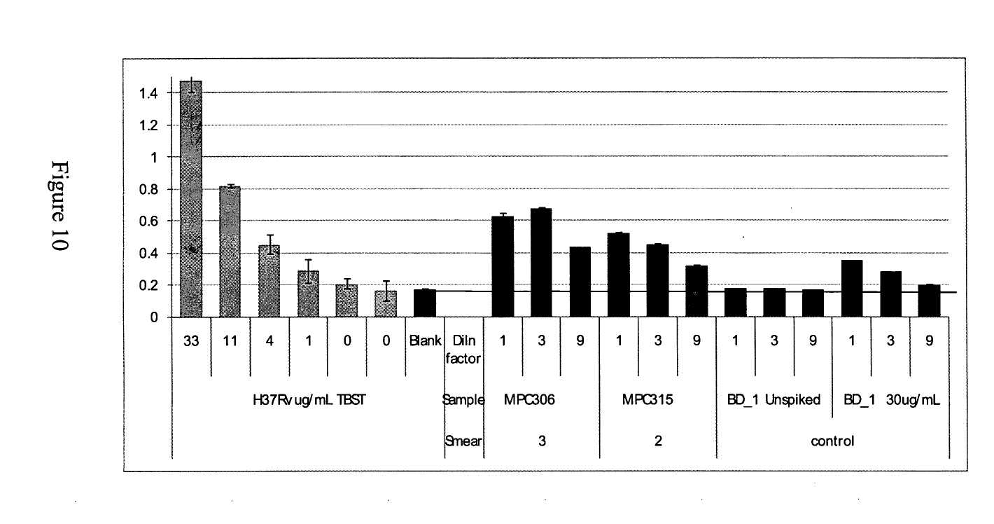

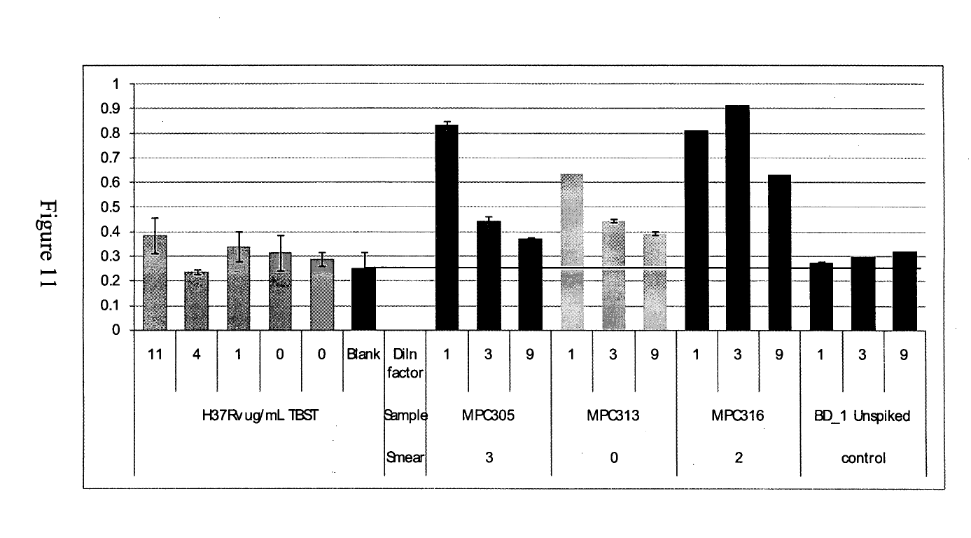

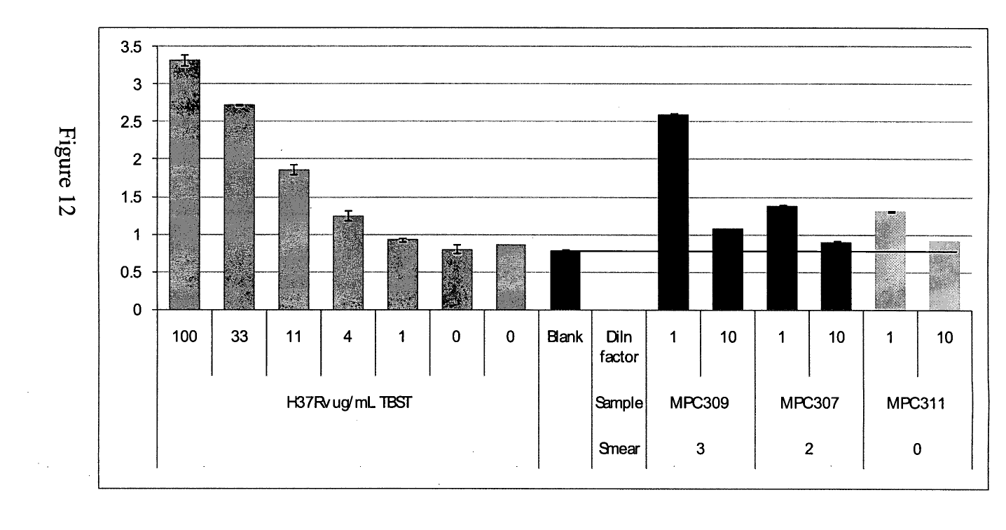

Data presented in FIGS. 8 and 9 (lower right panel marked “ilvC”) indicate that sputum contains some factors that inhibit KARI protein signal detection, since signal strength is reduced following addition of undiluted sputum, irrespective of whether or not the assay is performed immediately or following overnight incubation. However, this loss of signal strength can be progressively inhibited by diluting the sputa, and loss of signal strength is largely prevented by diluting sputum at least about 1:9 (v/v). Signal strength also declines following overnight incubation of recombinant protein in sputum, and that this loss of signal strength can also be partially prevented by dilution of sputum samples. These data indicate that a 1:9 (v/v) dilution of sputum into blocking buffer and rapid assay of samples are recommended to enhance signal strength in assaying KARI protein under these conditions.

### 11. Relative Levels of KARI Protein Detectable in Mycobacteria Cells

To further assess the suitability of KARI as a diagnostic marker for Mycobacteria infection, the levels of KARI protein were determined in whole cell lysates of the M. tuberculosis strains H37Rv, CSU93 and HN878, and in M. tuberculosis, M. avium and M. intracellulaire, relative to 10 other M. tuberculosis antigens including BSX, EF-Tu, P5CR, Rv1265, S9 and TetR-like protein described herein.

Amplified sandwich ELISA was performed essentially as described in this example and in Example 1, to identify relative levels of each antigen according to the standard protocol, with calibration standards included to permit quantitation.

Data shown in FIGS. 122-125 indicate that KARI is a relatively abundant protein in all three M. tuberculosis strains tested when expressed on the basis of total cellular protein. On this basis, M. tuberculosis Rv1265, BSX and S9 proteins are also relatively abundant among the 11 immunogenic proteins tested. Data shown in FIGS. 126-132 indicate that KARI protein is also a relatively abundant protein in Mycobacteria species generally, whereas other predominant immunogenic proteins tested i.e., BSX, Rv1265 and S9, appear to have greater specificity for M. tuberculosis compared to KARI when expressed on a per cell basis (FIGS. 104-105) or as per microgram of whole cell lysate protein (FIGS. 128-129) or as per microlitre of whole cell lysate filtrate (FIGS. 130-131). These data suggest utility of KARI as a generic single-analyte marker of mycobacteria infection, or as part of a multi-analyte test for mycobacteria infection or M. tuberculosis infection in combination with BSX and/or Rv1265 and/or S9 proteins. Other combinations are not excluded for multi-analyte testing of M. tuberculosis infection and/or combining assay of KARI protein with smear testing.

### 12. Optimizing the Limits of Detection

To further enhance sandwich ELISA sensitivity, a replacement amplification procedure is employed to employ iterative antigen binding following coating of the ELISA plate with capture antibody. Essentially, this will result in an increased amount of antigen being bound to the capture antibody notwithstanding the 50 μl volume limitations of a 96-well ELISA plate. Briefly, this iterative antigen loading involves repeating the antigen binding step in the sandwich ELISA several times, e.g., 2 or 3 or 4 or 5 times, etc. before washing and adding detection antibody. Naturally, each aliquot of antigen sample is removed following a standard incubation period before the next aliquot is added. The number of iterations can be modified to optimize the assay (e.g., parameters such as signal:noise ratio, detection limit and amount of antigen detected at half-maximum signal), depending upon the nature of the sample being tested (e.g., sample type), without undue experimentation. For example, up to about 20 iterations of sample loading (i.e., up to a 20× replacement amplification) can be employed to provide a low background signal, and a reduced detection limit of M. tuberculosis KARI protein.

### Example 3

### Antigen-Based Diagnosis of Tuberculosis or Infection by M. Tuberculosis Using Antibodies that Bind to a Putative Transcriptional Regulator of M. Tuberculosis Designated BSX

### 1. Identification of BSX Protein in TB-Positive Subjects

A protein having a molecular weight of about 15 kDa was recognized in TB+ samples. The sequences of twelve peptides from MALDI-TOF data matched a sequence encoded by the pbsX gene of M. tuberculosis set forth in SEQ ID NO: 2. The percent coverage of SEQ ID NO: 2 by these 12 peptides was about 70%, suggesting that the peptide fragments were derived from this same protein marker.

The identified protein having the amino acid sequence set forth in SEQ ID NO: 2 is a putative transcriptional regulatory protein of M. tuberculosis and was designated as “BSX”.

### 2. Antibodies

Forty-six (46) antibodies were prepared against recombinant BSX protein and several antibodies were prepared against immunogenic BSX peptides derived by linear B-cell epitope screening of a PEPSET of synthetic peptides scanning the amino acid sequence of BSX protein. Polyclonal and monoclonal antibodies were obtained and screened for their suitability as described herein or in Example 1. This process led to the identification of several antibody pairs for diagnosis of M. tuberculosis as described in this example. Multiple antibody combinations and orientations of antibodies other than those specifically described in this example are encompassed by the present invention, including any combination of the exemplified antibodies in any orientation e.g., as capture or detector antibody.

### 3. Validation of BSX as a Diagnostic Marker Using Antibodies Mo639F and Ch12/13

Among the antibodies prepared against M. tuberculosis is a mouse-derived antibody designated “Mo639F” and a chicken derived polyclonal antibody designated “Ch12/13”. These antibodies were used inter alia to validate BSX as a diagnostic marker for M. tuberculosis. In one example, the antibodies were used to demonstrate specific detection of BSX in M. tuberculosis whole cell extracts as described below.

The amino acid sequence of BSX protein from M. tuberculosis strain H37Rv is presented as SEQ ID NO: 2. The translation product has an expected molecular mass of about 16 kDa. One-dimensional SDS/PAGE analysis of a hexa-histidine-tagged rBSX protein performed essentially as described in Example 1 showed that the BSX protein migrated as a single band of approximately 17 kDa (data not shown), which is the expected mass of the fusion protein, based on the theoretical mass of the translation product and the hexahistidine tag moiety.

Western blot analyses were carried out using ELISA capture (Mo639F) and detector (Ch12/13) antibodies separately, to detect recombinant BSX protein and endogenous BSX protein in whole cell lysates of M. tuberculosis H37Rv, M. tuberculosis CSU93 and M. tuberculosis HN878. Both antibodies recognized a band in whole cell lysates derived from all three M. tuberculosis strains that had the expected molecule mass of native BSX protein (i.e., about 16 kDa), as well as detecting the slightly larger recombinant BSX protein (data not shown). There was little or not background detectable. The available data therefore confirm the specificity of the antibodies Mo639F and Ch12/13 for detecting the M. tuberculosis BSX protein.

Competition Western blot analysis performed essentially as described in Example 1 indicated that binding of polyclonal antibody Ch12/13 to recombinant BSX protein and endogenous BSX protein could be ablated by pre-incubation of antibodies with excess concentration of unlabelled recombinant BSX protein (data not shown).

In summary, the available data indicate that antibodies Mo639F and Ch12/13 can be used to detect BSX specifically in whole cell lysates of laboratory and clinical M. tuberculosis strains.

4. Titration of Antibodies R16, C44 and Mo403B Against M. tuberculosis BSX

Also among the antibodies prepared against M. tuberculosis are rabbit polyclonal anti-BSX antibody (raised against a BSX peptide) designated R16, a chicken anti-BSX polyclonal antibody designated C44 (raised against recombinant protein) and a mouse anti-BSX monoclonal antibody designated Mo403B (raised against the C-terminus of BSX).

An ELISA assay was performed using one of these anti-BSX antibodies i.e., R16 or C44 or Mo403B as a capture antibody and one other of the antibodies as a detector antibody. The ELISA plate was coated with various anti-BSX antibodies including Chicken (Ch) anti-BSX pAb C44, Rabbit (Ra) anti-BSX pAb R16, and Mouse (Mo) anti-BSX mAb Mo403B all at 20 μg/ml using 50 μl per well. Titrating amounts of recombinant BSX were added at a concentration of 50 ng/ml down to 3 pg/ml. Antigen detection was performed using either Rabbit anti-BSX at 10 μg/ml (with and without pre-incubation with the recombinant BSX protein) followed by detection using Sheep anti-Rabbit Ig HRP conjugate at a 1:5000 (v/v) dilution (for Chicken Capture system), or Chicken anti-BSX pAb C44 at 20 μg/ml followed by Sheep anti-Chicken IgG HRP conjugate at 1:5000 (v/v) dilution (for Mouse and Rabbit Capture systems). Data are presented in FIG. 30.

### 5. Limit of Detection (LOD) of Antibodies C44 and Mo403B in Sandwich ELISA

Subsequent to determining detection limits of anti-BSX mAb Mo403B and pAb C44 for detection of purified recombinant BSX our initial studies addressed optimisation of a sandwich ELISA using mAb Mo403B as a capture antibody and pAb C44 as a detector antibody. Briefly, Anti-BSX mAb Mo403B was immobilised onto an ELISA plate as a capture antibody at concentrations ranging from 10-40 μg/ml as specified above. Titrating amounts of recombinant BSX from 50 ng/ml down to 0.39 ng/ml were then screened using a purified chicken anti-BSX pAb, C44, at concentrations of either 10 or 20 μg/ml as specified above as the detector antibody followed by incubations with a Sheep anti-Chicken IgG HRP at a dilution of 1:5000 (v/v) and TMB for signal detection. Data are presented in FIG. 31.

Under these conditions, the limit of detection of recombinant BSX was ˜2-3 ng/ml.

### 6. Detection of BSX in Patient Samples Using Antibodies R16 and C44

Sputum samples (50 μl+50 μl blocking buffer) from South African TB patients and control patients with non-TB respiratory disease from South Africa (prefix ‘M’) and Australia (prefix ‘CGS’), respectively, were screened by sandwich ELISA for the presence of BSX antigen. Purified Rabbit anti-BSX (peptide 28) pAb, R16, was immobilised onto the ELISA plate as a Capture antibody at a concentration of 20 μg/ml. Purified Chicken anti-BSX pAb, C44, at a concentration of 5 μg/ml, was used as the Detector antibody. Sheep anti-Chicken IgG HRP at a dilution of 1:5000 (v/v) and TMB were used for signal detection. Sputum from control patient CGS25 was spiked with 5 ng/ml recombinant BSX as a positive control (red). Results are shown in FIG. 32.

### 7. Amplified Sandwich ELISA for Detection of M. Tuberculosis BSX Protein Using the Antibody Pairs Mo403B and C44 or R16 and C44

ELISA plates were coated with either purified anti-BSX mAb Mo403B at a concentration of 40 μg/ml or purified Chicken anti-BSX pAb C44 at a concentration of 5 μg/ml using 50 μl per well. Titrating amounts of purified recombinant BSX were added at a concentration of 50 ng/ml down to 0.39 ng/ml. Two amplification systems were performed using either Chicken anti-BSX at a concentration of 10 μg/ml followed by Donkey anti-Chicken IgG biotin conjugate at various dilutions and finally streptavidin-HRP at a 1:5000 (v/v) dilution, or anti-BSX mAb Mo403B at various concentrations followed by Goat anti-Mouse IgG at 1:30000 (v/v) dilution and Donkey anti-Goat IgG HRP conjugate at a 1:5000 (v/v) dilution. The amplified systems were used to compare to a basic antigen detecting system where Chicken anti-BSX was used at a concentration of 10 μg/ml followed by Sheep anti-Chicken IgG HRP conjugate at a 1:5000 (v/v) dilution.

As shown in FIG. 33, the amplified ELISA was approximately 10 fold more sensitive than the standard ELISA. Signal intensity is slightly higher when using the Rabbit pAb as a capture and the Chicken pAb as the first detector Ab in the amplified system (FIG. 34).

The inventors have also investigated an amplified ELISA system which, as shown in FIGS. 33 and 35, uses purified rabbit anti-BSX pAb R16 as a capture antibody and purified chicken anti-BSX pAb C44 as a detector antibody followed by amplification with a biotinylated secondary detector Ab. This system provided a further 2-fold increase in sensitivity compared the amplification systems described earlier (FIG. 35; FIG. 36).

The inventors have also performed studies using the amplified biotin-based ELISA to screen clinical sputum samples from TB and non-TB respiratory disease control patients, always keeping in mind in the non-TB respiratory disease group there may be up to 30-40% of the patients having TB co-infections due to the reduced sensitivity of smear microscopy and culture assays (FIG. 37).

To investigate if antibody sites were being saturated with endogenous BSX the inventors also compared the effect of (i) incubation time; and (ii) sequential incubations with a fresh aliquot of a sputum sample from the same respective patient. The increase from 1 hr to 2 hr incubation did not have any effect on signal intensity. In contrast, preliminary data indicates that sequential incubations with 2 different sample loads of sputum increased signal intensity (FIG. 38). Whilst the increase is not large, these preliminary observations warrant further investigation. Interestingly, the increase in signal intensity was most marked for detection of a recombinant protein as a positive control.

### 8. Amplified Sandwich ELISA Using the Antibodies Designated Mo639F and Ch12/13

A further antibody pair for diagnosis of M. tuberculosis consists of a mouse-derived antibody designated “Mo639F” as a preferred capture antibody and a chicken derived polyclonal antibody designated “Ch12/13” as a preferred detector antibody. Other orientations and antibody combinations are not excluded.

Amplified ELISA was performed essentially as described in this example and in Example 1, using 2 μg/mL of Mo639F antibody as a capture reagent and 5 μg/mL of Ch12/13 polyclonal antibody as a detector antibody, and a biotinylated donkey anti-chicken IgG secondary antibody with HRP-conjugated poly-40 streptavidin to detect the bound detector antibody.

Data presented in FIG. 39 indicate that under the assay conditions tested and with this preferred, albeit not essential, orientation of antibodies, there is low background noise and a LOD of about 89 pg/mL. Such sensitivity of detection coupled with low background in sandwich ELISA is considered by the inventors to be within useful limits.

### 9. Cross-Reactivity Between Anti-BSX Antibodies and Different M. Tuberculosis Isolates

To further assess the suitability of BSX as a diagnostic marker for the presence of M. tuberculosis in biological samples, and to assess the specificities of antibodies prepared against BSX protein, the inventors compared Mo639F and Ch12/13 antibody reactivities in amplified sandwich ELISA performed as described herein above between cellular extracts of the clinical M. tuberculosis strains CSU93 and H878 and the laboratory M. tuberculosis strain H37Rv.

Briefly, an ELISA plate was coated overnight with capture antibody Mo639F. Following washing to remove unbound antibody, a cellular extract from each isolate was added to the wells of the antibody-coated ELISA plates. As a negative control for each assay, buffer without cellular extract was used. Following incubation for 1 hour and washing to remove unbound antigen, detection antibody Ch12/13 was contacted with the bound antigen-body complexes. Following incubation at room temperature for 1 hour, plates were washed, incubated with 50 μl of diluted secondary antibody (e.g., biotinylated donkey anti-chicken IgG and poly-40 streptavidin-HRP conjugate) for 1 hour, washed again, incubated with TMB for 10 mins, and the absorbance at 450-620 nm was determined. Samples were assayed in duplicate over three dilutions of whole cell extracts. A calibration standard curve was produced based on standardized levels of BSX protein.

Data presented in FIG. 40 show that M. tuberculosis BSX protein is present at higher levels in the clinical strains compared to the laboratory strain H37Rv. Lower levels of BSX protein were detectable in M. tuberculosis H878 than CSU93, however these data indicate that antibodies against BSX protein have general utility in detecting clinical isolates of M. tuberculosis.

The data presented in FIG. 40 support the utility antibodies against BSX protein in a general single-analyte diagnostic test, or alternatively, as part of a multi-analyte test in conjunction with antibodies against specific strains of M. tuberculosis such as described herein or known in the art.

Antibodies against BSX protein may also be employed in conjunction with subsequent culture of M. tuberculosis from BSX-positive clinical specimens to yield information on clinically-relevant strains present in the sample, if required.

### 10. Cross-Reactivity Between Different Mycobacteria Species

To further assess the suitability of BSX as a diagnostic marker for the presence of M. tuberculosis in biological samples, and to assess the specificities of antibodies prepared against BSX protein, the inventors compared antibody reactivities in amplified sandwich ELISA performed as described herein above between cellular extracts of the Mycobacteria species M. tuberculosis, M. avium and M. intracellulaire.

Briefly, an ELISA plate was coated overnight with capture antibody Mo639F. Following washing to remove unbound antibody, a cellular extract from each Mycobacteria species was added to the wells of the antibody-coated ELISA plates. As a negative control for each assay, buffer without cellular extract was used. Following incubation for 1 hour and washing to remove unbound antigen, detection antibody Ch12/13 was contacted with the bound antigen-body complexes. Following incubation at room temperature for 1 hour, plates were washed, incubated with 50 μl of diluted secondary antibody (e.g., biotinylated donkey anti-chicken IgG and poly-40 streptavidin-HRP conjugate) for 1 hour, washed again, incubated with TMB for 10 mins, and the absorbance at 450-620 nm was determined. Samples were assayed in duplicate over three dilutions of whole cell extracts. A calibration standard curve was produced based on standardized levels of BSX protein.

Data presented in FIGS. 41a and 41b show almost undetectable cross-reactivity between the three Mycobacteria species, indicating that M. tuberculosis BSX protein is species-specific with respect to detection of M. tuberculosis under these assay conditions or using the selected antibody pair. This support the utility of antibodies against BSX protein in a general or species-specific single-analyte diagnostic test, or alternatively, as part of a multi-analyte test in conjunction with antibodies against other markers of M. tuberculosis such as described herein or known in the art. For example, antibodies against BSX protein may be employed simultaneously with one or more antibodies against M. tuberculosis Rv1265 and/or M. tuberculosis KARI protein and/or M. tuberculosis EF-Tu and/or M. tuberculosis S9 protein as described herein.

Antibodies against BSX protein may also be employed in conjunction with subsequent culture of M. tuberculosis from BSX-positive clinical specimens.

### 11. Low Cross-Reactivity Between M. Tuberculosis and Non-Mycobacteria Pathogens

To further assess the suitability of BSX as a diagnostic marker for the presence of M. tuberculosis in biological samples, the inventors compared antibody cross-reactivities in amplified sandwich ELISA performed between cellular extracts of M. tuberculosis strain H37Rv (a laboratory strain), Escherichia coli, Bacillus subtilis or Pseudomonas aeruginosa.

Briefly, an ELISA plate was coated overnight with capture antibody Mo639F. Following washing to remove unbound antibody, a cellular extract from each microorganism was added the wells of the antibody-coated ELISA plates. As a negative control for each assay, buffer without cellular extract was used. Following incubation for 1 hour and washing to remove unbound antigen, detection antibody Ch12/13 was contacted with the bound antigen-body complexes. Following incubation at room temperature for 1 hour, plates were washed, incubated with 50 μl of a secondary antibody (i.e., biotinylated donkey anti-chicken IgG and poly-40 streptavidin-HRP conjugate) for 1 hour, washed again, incubated with TMB for 10 mins, and the absorbance at 450-620 nm was determined.

Data presented in FIG. 42 show no significant cross-reactivity of antibodies against M. tuberculosis BSX protein with Escherichia coli, Bacillus subtilis or Pseudomonas aeruginosa cellular extracts under the conditions tested, indicating that the antibodies form the basis of a Mycobacterium-specific test.

### 12. Detection of BSX Protein in Clinical Samples

To further assess the suitability of BSX as a diagnostic marker for the presence of M. tuberculosis in biological samples, the inventors determined the ability of antibodies to detect endogenous BSX protein in clinical samples obtained from TB-positive subjects who had been diagnosed previously on the basis of smear test and M. tuberculosis culture assay results. Patients had been categorized on the basis of both smear and culture test results, and HIV status. All subjects tested were both smear-negative and culture-negative or alternatively, both smear-positive and culture-positive.

In one example, an immune-magnetic bead assay was performed using magnetic beads coated with anti-BSX Ch8 polyclonal antibody to capture BSX from treated sputa of previously-diagnosed subjects, and Mo639F monoclonal antibody as a detector antibody, subject to amplification using anti-mouse Ig conjugated to HRP, as described in the legend to FIG. 43. As shown in FIG. 43, statistically-significant higher levels of BSC protein were identified in TB smear-positive and culture-positive subjects than TB-negative subjects.

In a further example, amplified sandwich ELISA was performed as described herein above on patient sputum samples which were prepared by Method 3 and assayed as 4×150 microlitre aliquots under the replacement amplification protocol (see below). An ELISA plate is coated overnight with capture antibody Mo639F or other suitable anti-BSX antibody. Following washing to remove unbound antibody, and treated sputa are added to the wells of the antibody-coated ELISA plates. As a negative control for each assay, buffer is used. Following incubation for 1 hour and washing to remove unbound antigen, detection antibody Ch12/13 or other suitable antibody is contacted with the bound antigen-body complexes. Following incubation at room temperature for 1 hour, plates are washed, incubated with 50 μl of a secondary antibody (e.g., biotinylated donkey anti-chicken IgG and poly-40 streptavidin-HRP conjugate) for 1 hour, washed again, incubated with TMB for 10 mins, and the absorbance at 450-620 nm was determined.

Data presented in FIGS. 44 and 45 show significantly higher levels of BSX protein detected in at least 3 of the 4 TB-positive samples tested that had been shown previously to be culture-positive and smear-positive. In contrast, background signals were detected in TB-negative samples.

### 13. Evaluation of Signal Inhibition by Sample

To assess whether or not inhibitory or signal-suppressing factors are present in sputa that might adversely affect assay sensitivity e.g., in ELISA or a point-of-care or field test format, sputa samples were spiked with 10 ng/mL recombinant M. tuberculosis BSX protein and the resultant samples serially diluted 1:27 (v/v) over three steps. Samples were incubated overnight and assayed by amplified ELISA as described herein above, or assayed immediately.

Data presented in FIGS. 87 and 88 (upper left panel marked “BSX”) indicate that sputum contains some factors that inhibit BSX protein signal detection, since signal strength is reduced following addition of undiluted sputum, irrespective of whether or not the assay is performed immediately or following overnight incubation. However, this loss of signal strength can be progressively inhibited by diluting the sputa, and loss of signal strength is largely prevented by diluting sputum at least about 1:9 (v/v). Signal strength also declines following overnight incubation of recombinant protein in sputum, and that this loss of signal strength can also be partially prevented by dilution of sputum samples. These data indicate that at least 1:9 (v/v) dilution of sputum into blocking buffer and rapid assay of samples are recommended to enhance signal strength in assaying BSX protein under these conditions.

### 14. Relative Levels of BSX Protein Detectable in Mycobacterium Cells

To further assess the suitability of BSX as a diagnostic marker for Mycobacteria infection, the levels of BSX protein were determined in whole cell lysates of the M. tuberculosis strains H37Rv, CSU93 and HN878, and in M. tuberculosis, M. avium and M. intracellulaire, relative to 10 other M. tuberculosis antigens including KARI, EF-Tu, P5CR, Rv1265, S9 and TetR-like protein described herein.

Amplified sandwich ELISA was performed essentially as described in this example and in Example 1, to identify relative levels of each antigen according to the standard protocol, with calibration standards included to permit quantitation.

Data shown in FIGS. 124-125 indicate that BSX is a relatively abundant protein in all three M. tuberculosis strains tested when expressed on the basis of total cellular protein. On this basis, M. tuberculosis Rv1265, BSX and S9 proteins are also relatively abundant among the 11 immunogenic proteins tested. Data shown in FIGS. 126-127 indicate that BSX protein is also a relatively abundant protein in Mycobacteria species generally, whereas other predominant immunogenic proteins tested i.e., BSX, Rv1265 and S9, appear to have greater specificity for M. tuberculosis compared to BSX when expressed on a per cell basis (FIGS. 126-127) or as per microgram of whole cell lysate protein (FIGS. 128-129) or as per microlitre of whole cell lysate filtrate (FIGS. 130-131). These data suggest utility of BSX as a generic single-analyte marker of mycobacteria infection, or as part of a multi-analyte test for mycobacteria infection or M. tuberculosis infection in combination with BSX and/or Rv1265 and/or S9 proteins. Other combinations are not excluded for multi-analyte testing of M. tuberculosis infection.

### 11. Optimizing the Limits of Detection

To further enhance sandwich ELISA sensitivity, a replacement amplification procedure is employed to employ iterative antigen binding following coating of the ELISA plate with capture antibody. Essentially, this will result in an increased amount of antigen being bound to the capture antibody notwithstanding the 50 μl volume limitations of a 96-well ELISA plate. Briefly, this iterative antigen loading involves repeating the antigen binding step in the sandwich ELISA several times, e.g., 2 or 3 or 4 or 5 times, etc. before washing and adding detection antibody. Naturally, each aliquot of antigen sample is removed following a standard incubation period before the next aliquot is added. The number of iterations can be modified to optimize the assay (e.g., parameters such as signal:noise ratio, detection limit and amount of antigen detected at half-maximum signal), depending upon the nature of the sample being tested (e.g., sample type), without undue experimentation. For example, up to about 20 iterations of sample loading (i.e., up to a 20× replacement amplification) can be employed to provide a low background signal, and a reduced detection limit of M. tuberculosis BSX protein.

### Example 4

### Antigen-Based Diagnosis of Tuberculosis or Infection by M. Tuberculosis Using Antibodies that Bind to a Putative Ribosomal Protein of M. Tuberculosis Designated S9

### 1. Identification of S9 Protein in TB-Positive Subjects

A protein having a molecular weight of about 30.2 kDa was recognized in the immune-globulin fraction of sera from TB+ samples (FIG. 46). The sequences of four peptides from MALDI-TOF data (SEQ ID Nos: 15-18 inclusive) matched a protein having SwissProt Accession No. P66638 (SEQ ID NO: 14). The percent coverage of P66638 by these 4 peptides (SEQ ID NOs: 15-18) was about 14-15%, suggesting that the peptide fragments were derived from this same protein marker. This conclusion was supported by there being only six theoretical tryptic peptides with zero miscleavages, and fourteen theoretical tryptic peptides having one miscleavage.

The identified protein having the amino acid sequence set forth in SEQ ID NO: 14 was designated as “S9”. Interestingly, the estimated molecular weight of the S9 protein is only about 16.4 kDa, and the estimated isoelectric point of S9 is about 10.7. Since the observed molecular weight of the S9 protein was about 14 kDa higher than the estimated value, the protein is most likely post-translationally modified e.g., by glycosylation, or co-migrates with another molecular species such as nucleic acid.

### 2. Antibodies

Antibodies were prepared against recombinant S9 (rS9) protein of M. tuberculosis and against synthetic peptides derived from the full-length S9 protein sequence using procedures described herein. Approximately eight (8) antibodies were produced against rS9 protein and screened for their suitability as described in Example 1.

### a) Antibodies Against Synthetic S9 Peptides

A synthetic peptide comprising the sequence H-MTETT PAPQT PAAPA GPAQS FC—NH₂ from 30S ribosomal protein S9 was synthesized to 78% purity as determined by liquid chromatography by Mimotopes using solid phase peptide synthesis technology. This peptide was coupled to keyhole limpet Hemocyanin (KHL) via a Maleimidocaproyl-N-Hydroxysuccinimide linker.

To facilitate detection of antibodies raised against this epitope the peptide was also synthesized with a GSGL spacer and attached to biotin (PAPQT PAAPA GPAQS FGSGL-Biotin) to 93% purity by liquid chromatography.

A rabbit was injected with 600 μg per dose of the synthetic peptide comprising the amino acid sequence H-MTETT PAPQT PAAPA GPAQS FC—NH₂ linked to KHL according to the following injection protocol:

| | |
|---|---|
| Prebleed | Week 0 |
| Primary Inoculation | Week 0 |
| 1^{st} Booster | Week 3 |
| 2^{nd} Booster | Week 6 |
| Test Bleed | Week 7.5 |
| 3^{rd} Booster | Week 9 |
| Bleedout | Week 10.5 |

After 10.5 weeks the rabbit was bled out. All blood was collected in sterile containers and incubated at 37° C. to accelerate clotting. The containers were centrifuged and the serum removed and re-centrifuged to remove the remaining red cells. Serum was designated “R9”.

Streptavidin (Sigma Aldrich) was diluted to 5 μg/ml in ddH₂O and incubated in a Nunc plate overnight at 4° C. The solution was then flicked out and 250 μL of blocking buffer (1% (w/v) casein, 0.1% (v/v) Tween 20, 0.1% (w/v) sodium azide in PBS) added to each well and incubated at room temperature for 1 hour. The blocking buffer was flicked out and biotinylated peptide (corresponding to the immunogen injected into the rabbit) was added in blocking buffer at 3 μg/ml (50 μl/well) and incubated for one hour at room temperature on a shaker. The plate was washed in an Elx405 Auto Plate Washer (Bio-Tek Instruments Inc., Winooski, Vt.), with 0.5×PBS/0.05% (v/v) Tween 20 solution and excess solution tapped out on a paper towel. The rabbit sera was diluted in blocking buffer 2 fold from 1:500 to 1:1,024,000 and incubated from 1 hour at 50 μl/well at room temperature on a shaker. The plate was washed with the plate washer using 0.5×PBS/0.05% (v/v) Tween 20 solution and excess solution tapped out on a paper towel. Binding of the rabbit antibody to its corresponding epitope was detected using HRP-conjugated sheep anti-rabbit (Chemicon) diluted 1 in 10,000 in conjugate diluent buffer. Fifty millilitres were added to each well and incubated for one hour at room temperature on a shaker. The plate was washed with the plate washer using 0.5×PBS and excess solution tapped out on a paper towel. Fifty millilitres of TMB (3,3′,5′,5-Tetramethylbenzidine; Sigma) was added to each well and the plate incubated in the dark for 30 minutes. Development was stopped with 50 μL/well of 0.5M sulphuric acid. The optical density of each well was read with a microtiter plate reader (PowerWaveₓ 340 plate reader, Bio-Tek Instruments Inc., Winooski, Vt.) using a wavelength of 450 nm and an extinction at 620 nm. The titration results are shown in FIG. 47.

To titrate the peptide, a protocol essentially as described supra was used. However, the biotinylated peptide was titrated from 20,480 pg/ml to 10 pg/ml and the rabbit sera was first purified down an affinity peptide column and added to the ELISA at 10 μg/ml, 20 μg/ml and 40 μg/ml. Results of this analysis are shown in FIG. 48.

Multiple antibody combinations and orientations of antibodies other than those specifically described in this example are encompassed by the present invention, including any combination of the exemplified antibodies in any orientation e.g., as capture or detector antibody.

### b) Antibodies Against Full-Length Recombinant S9 Protein

Two antibodies (Ch27 and Mo1025F) prepared against the full length recombinant M. tuberculosis protein (SEQ ID NO: 14), by immunization of chickens and mice using standard procedures, were shown to have the highest sensitivity of detection for the S9 protein in ELISA assays, compared to other antibodies produced, including bivalent F(ab)₂ fragments produced by phage display of S9 peptides and a further polyclonal antibody (R9) raised against a purified S9 peptide (data not shown). Herein, chicken anti-S9 polyclonal sera are designated “Ch27”, and mouse anti-S9 antibodies are designated “Mo1025F”. A preferred antibody pair for diagnosis of M. tuberculosis consists of a mouse-derived antibody designated “Mo1025F” as a preferred detector antibody and a chicken derived polyclonal antibody designated “Ch27” as a preferred capture antibody.

Data presented in FIG. 51 show that the antibodies Ch27 and Mo1025F prepared against recombinant M. tuberculosis ribosomal protein S9 are capable of detecting recombinant S9 protein by standard ELISA, and suggest that the mouse antibody Mo1025F may have particular utility as a diagnostic reagent due to its higher titer (i.e., half-maximum detection of about 93 ng/ml S9 protein and detection limit of about 8 ng/ml under the conditions used) compared to antibody Ch27 (half-maximum detection of greater than 125 ng/ml S9 protein and detection limit of about 32 ng/ml under the conditions used).

Additional antibodies and antibody pairs for performing the invention are described in this example, including the rabbit polyclonal antibody designated “R9” prepared against a synthetic peptide (see below). Other antibody combinations and antibody orientations are not excluded.

### 3. Validation of S9 as a Diagnostic Marker Using Antibodies R9, Mo1025F and Ch27

The amino acid sequence of S9 protein from M. tuberculosis strain H37Rv is presented as SEQ ID NO: 15. The translation product has an expected molecular mass of about 16.4 kDa. One-dimensional SDS/PAGE analysis of a hexa-histidine-tagged rS9 protein performed essentially as described in Example 1 showed that the S9 protein migrated as a single band of approximately 17 kDa (data not shown), which is the expected mass of the fusion protein, based on the theoretical mass of the translation product and the hexahistidine tag moiety.

To confirm that the polyclonal antibody R9 was capable of detecting a protein expressed by M. tuberculosis, Western blotting was performed using protein extracted from the M. tuberculosis laboratory train H37Rv. Cytosolic and membrane proteins were extracted and analysed using Western blotting essentially as described in Example 1. Antibody R9 detected a protein of the correct molecular weight in reduced cytosolic samples, reduced membrane samples and non-reduced cytosolic/membrane samples. Accordingly, ribosomal protein S9 is expressed by M. tuberculosis, e.g., strain H37Rv, a fact that has been previously not been recognized in the art.

Western blot analyses (data not shown) were carried out using ELISA capture (Ch27) and detector (Mo1025F) antibodies separately, to detect recombinant S9 protein and endogenous S9 protein in whole cell lysates of M. tuberculosis H37Rv, M. tuberculosis CSU93 and M. tuberculosis HN878. Both antibodies recognized a band in whole cell lysates that had the Membranes were then blocked in solution containing 1% casein in 1×PBS, 0.1% Tween-20 (PBST) at room temperature (RT) for 2 hours. Membranes were then incubated with 15 μg/ml purified rabbit anti-S9 polyclonal antibody solution (i.e., R9) at RT for 2 hr, following by 3×10 min washes with PBST. Membranes were then incubated with 1:10,000 dilution of sheep anti-rabbit IgG-HRP conjugated antibody solution at RT for 1 hr, followed by 5×10 min washes with times PBST. Membranes were finally treated with ‘Femto’ chemiluminescence reagents (Pierce) for 5 min before exposure to x-ray films.

Ribosomal protein S9 was detected in 20/20 South African TB patients (Sensitivity=100%) and 5/20 Australian non-TB respiratory disease patients (Specificity=75%) using the rabbit R9 polyclonal antibody (see FIGS. 49 and 50).

### 5. Sandwich ELISA Using Antibodies Ch27 and Mo1025F

In a first set of experiments, sandwich ELISA was performed to determine optimum capture and detection antibodies, and appropriate antibody concentrations for use. Briefly, two ELISA plates were coated with either Ch27 or Mo1025F antibodies at 2.5 μg/ml and 5 μg/ml concentrations in blocking buffer. Following washing to remove unbound antibody, 50 ml aliquots of recombinant S9 protein, diluted serially in blocking buffer 1:2 (v/v) from 500 ng/ml starting concentration to 7.8 ng/ml, were added the wells of the antibody-coated ELISA plates. Following incubation for 1 hour and washing to remove unbound antigen, the alternate detection antibody (i.e., Mo1025F for detection of Ch27-S9 complexes and Ch27 for detection of Mo1025F—S9 complexes) was contacted with the plates at concentrations in the range of 1.25 μg/ml to 5 μg/ml. Following incubation at room temperature for 1 hour, plates were washed as before, incubated with 50 μl of a 1:5000 (v/v) dilution of donkey anti-mouse IgG conjugated to horseradish peroxidase (HRP), washed as before, incubated with TMB for 30 mins, and the absorbance at 450-620 nm was determined.

Data presented in FIGS. 52 and 53 indicate that the preferred, albeit not essential, orientation of antibodies to achieve higher signal per unit of recombinant S9 protein in sandwich ELISA is obtained using Ch27 as the capture antibody and Mo1025F as a detection antibody. Minimal cross-reactivity between antibodies is also observed with this expected molecule mass of native S9 protein (i.e., about 16.4 kDa), as well as detecting the slightly larger recombinant S9 protein. There was little or no background detectable.

The polyclonal antibody Ch27 detected high levels of endogenous S9 protein of the expected molecular weight in extracts of the laboratory strain H37Rv and much less albeit detectable levels in the clinical strain CSU93. In contrast, the monoclonal antibody Mo1025F reacted strongly with a protein of the expect molecular weight in both H37Rv and CSU93, and also produced a detectably-strong signal in extracts of the clinical strain HN878. As with the Ch27 antibody preparation, the signal strength obtained from binding of Mo1027 to S9 protein in extracts of H37Rv was noticeably stronger than for CSU93 cell extracts. These data suggest that differential signal strengths obtained using these two antibody preparations may be a consequence of their different antibody titres rather than an intrinsic difference in their ability to detect S9 protein in different cultures, since the trend was the same for both preparations i.e., HN878 signal<CSU93 signal<H37Rv signal.

The available data therefore confirm the specificity of the antibodies Mo1025F and Ch27 for detecting the M. tuberculosis S9 protein.

Competition Western blot analysis performed essentially as described in Example 1 indicated that binding of both antibodies to recombinant S9 protein and endogenous S9 protein could be ablated by pre-incubation of antibodies with excess concentration of unlabelled recombinant S9 protein (data not shown).

In summary, the available data indicate that ELISA capture (Ch27) and detector (Mo1025F) antibodies bind to M. tuberculosis S9 protein specifically.

4. Detection of S9 in Sputum from TB Subjects Using Polyclonal Antibody R9

The R9 antibody described herein was used to detect S9 protein in samples from 20 TB subjects and 20 subjects suffering from a non-TB subject. Briefly, sputum (12 μl) from TB or non-TB patients was loaded onto 4-12% 1D gradient SDS polyacrylamide gels and separated by electrophoresis. Proteins were then electrotransferred onto PVDF membrane. antibody orientation, as indicated by the baseline value in FIG. 52 when no S9 is present in the sample.

To determine the limits of detection of the sandwich ELISA for recombinant M. tuberculosis S9 protein, the assay was also performed using a serial dilution of S9 protein, in the concentration range from 18.31 pg/ml to 150 ng/ml. Data presented in FIG. 54 indicate that, under the assay conditions tested, there was no background signal with this antibody combination, and concentrations as low as about 996 pg/ml M. tuberculosis ribosomal protein S9 could be detected, with half-maximum detection of about 28 ng/ml M. tuberculosis ribosomal protein S9. Such sensitivity of detection coupled with low background in sandwich ELISA is considered by the inventors to be within useful limits.

6. Amplified Sandwich ELISA for Detection of M. tuberculosis S9 Protein

Amplified ELISA was performed essentially as described in this example and in Example 1, using Ch27 polyclonal antibody as a capture reagent and Mo1025F monoclonal antibody as a detector antibody, and a biotinylated donkey anti-mouse IgG secondary antibody with poly-40 streptavidin-HRP conjugate to detect the bound detector antibody. As an alternative to biotinylated secondary antibody, the monoclonal antibody Mo1025 was biotinylated directly to produce a species designated herein as “Mo1025F-Bio”, which was detectable using HRP-conjugated streptavidin directly.

As shown in FIG. 55, the use of a biotinylated secondary antibody and streptavidin poly-40 horseradish peroxidase (HRP) conjugate provided some increase in sensitivity of detection, with a statistically significant limit of detection as low as about 150 pg/ml recombinant M. tuberculosis ribosomal protein S9. Under these conditions, the sandwich ELISA was also capable of detecting about 6 ng/ml M. tuberculosis ribosomal protein S9 at half-maximal signal.

Similar results were obtained using biotinylated Mo1025F-Bio antibody as a detector antibody reagent. As shown in FIG. 59, amplified ELISA performed using the Mo1025F-Bio detector antibody directly also reduced the LOD about 4-fold to about 348 pg/mL from 1470 pg/mL, however in those experiments the LOD for the standard ELISA was higher than in the experiments described supra i.e., 1470 pg/mL cf. 996 pg/mL, suggesting that the actual results of both amplified ELISA systems may be comparable. Adjustment of data to account for variation between experiments supports this suggestion.

### 7. Replacement Amplification of ELISA

To further enhance sandwich ELISA sensitivity, the inventors further modified the basic assay by employing iterative antigen binding following coating of the ELISA plate with capture antibody. Essentially, this results in an increased amount of antigen being bound to the capture antibody notwithstanding the 50 μl volume limitations of a 96-well ELISA plate. Briefly, this iterative antigen loading involves repeating the antigen binding step in the sandwich ELISA several times, e.g., 2 or 3 or 4 or 5 times, etc. before washing and adding detection antibody. Naturally, each aliquot of antigen sample is removed following a standard incubation period before the next aliquot is added. The number of iterations can be modified to optimize the assay (e.g., parameters such as signal:noise ratio, detection limit and amount of antigen detected at half-maximum signal), depending upon the nature of the sample being tested (e.g., sample type), without undue experimentation.

As shown in FIG. 56, five iterations of sample loading (i.e., a 5× replacement amplification) provided a low background signal, and a detection limit of about 84 pg/ml M. tuberculosis ribosomal protein S9. As the assay shown in FIG. 56 was not performed under conditions reaching signal saturation, no estimation of the amount of antigen detected at half-maximum signal was possible. Notwithstanding, an approximate 2-fold increase in sensitivity of detection of recombinant M. tuberculosis ribosomal protein S9 was obtained by iterative antigen loading.

7. Low Cross-Reactivity Between M. tuberculosis and Non-Mycobacteria Pathogens

To further assess the suitability of S9 as a diagnostic marker for the presence of M. tuberculosis in biological samples, the inventors compared antibody cross-reactivities in amplified sandwich ELISA performed between cellular extracts of M. tuberculosis strain H37Rv (a laboratory strain), Escherichia coli, Bacillus subtilis or Pseudomonas aeruginosa. Briefly, an ELISA plate was coated overnight with capture antibody Ch27 at 5 μg/ml concentration. Following washing to remove unbound antibody, 500 ng/ml or 50 μg/ml of a cellular extract from each microorganism were added the wells of the antibody-coated ELISA plates. As a negative control for each assay, buffer without cellular extract was used. Following incubation for 1 hour and washing to remove unbound antigen, detection antibody Mo1025F was contacted with the bound antigen-body complexes at 2.5 μg/ml concentration. Following incubation at room temperature for 1 hour, plates were washed, incubated with 50 μl of a 1:5000 (v/v) dilution of secondary antibody (i.e., biotinylated donkey anti-mouse IgG and poly-40 streptavidin-HRP conjugate) for 1 hour, washed again, incubated with TMB for 10 mins, and the absorbance at 450-620 nm was determined. No iterative sample loading was performed in this experiment.

Data presented in FIG. 57 show low cross-reactivity of antibodies against M. tuberculosis ribosomal protein S9 with Escherichia coli, Bacillus subtilis or Pseudomonas aeruginosa cellular extracts under the conditions tested.

8. Cross-Reactivity Between Anti-S9 Antibodies and different M. tuberculosis Isolates

To further assess the suitability of S9 as a diagnostic marker for the presence of M. tuberculosis in biological samples, and to assess the specificities of antibodies prepared against S9 protein, the inventors compared antibody reactivities in amplified sandwich ELISA performed as described herein above between cellular extracts of the clinical M. tuberculosis strains CSU93 and H878 and the laboratory M. tuberculosis strain H37Rv.

Briefly, an ELISA plate is coated overnight with capture antibody Ch27. Following washing to remove unbound antibody, a cellular extract from each isolate is added to the wells of the antibody-coated ELISA plates. As a negative control for each assay, buffer without cellular extract was used. Following incubation for 1 hour and washing to remove unbound antigen, detection antibody Mo1025F or M1025F-Bio is contacted with the bound antigen-body complexes. Following incubation at room temperature for 1 hour, plates are washed, incubated with 50 μl of diluted secondary antibody if required (e.g., biotinylated donkey anti-mouse IgG when using Mo1025 as detector) for 1 hour, and then with poly-40 streptavidin-HRP conjugate for 1 hour, washed again, incubated with TMB for 10 mins, and the absorbance at 450-620 nm is determined. Samples are assayed in duplicate over three dilutions of whole cell extracts. A calibration standard curve was produced based on standardized levels of S9 protein.

Data presented in FIGS. 58 and 60 show that M. tuberculosis S9 protein is present in both the clinical M. tuberculosis isolate CSU93 and the laboratory strain H37Rv at comparable levels. Data in FIG. 61 also show comparable levels of S9 protein in H37Rv, CSU93 and HN878.

The data presented in FIG. 60 support the utility of antibodies against S9 protein in a general single-analyte diagnostic test, or alternatively, as part of a multi-analyte test in conjunction with antibodies that detect other antigens of M. tuberculosis or even specific strains of M. tuberculosis such as described herein or known in the art.

Antibodies against S9 protein may also be employed in conjunction with subsequent culture of M. tuberculosis from S9-positive clinical specimens to yield information on clinically-relevant strains present in the sample, if required.

### 9. Cross-Reactivity Between Different Mycobacteria Species

To further assess the suitability of S9 as a diagnostic marker for the presence of M. tuberculosis in biological samples, and to assess the specificities of antibodies prepared against S9 protein, the inventors compared antibody reactivities in amplified sandwich ELISA performed as described herein above between cellular extracts of the Mycobacteria species M. tuberculosis, M. avium and M. intracellulaire.

Briefly, an ELISA plate is coated overnight with capture antibody Ch27. Following washing to remove unbound antibody, a cellular extract from each Mycobacteria is added to the wells of the antibody-coated ELISA plates. As a negative control for each assay, buffer without cellular extract was used. Following incubation for 1 hour and washing to remove unbound antigen, detection antibody Mo1025F or M1025F-Bio is contacted with the bound antigen-body complexes. Following incubation at room temperature for 1 hour, plates are washed, incubated with 50 μl of diluted secondary antibody if required (e.g., biotinylated donkey anti-mouse IgG if Mo1025F is used as detection reagent) for 1 hour, and then with poly-40 streptavidin-HRP conjugate for 1 hour, washed again, incubated with TMB for 10 mins, and the absorbance at 450-620 nm is determined. Samples are assayed in duplicate over three dilutions of whole cell extracts. A calibration standard curve was produced based on standardized levels of S9 protein.

Data presented in FIG. 61 show lack of detectable cross-reactivity between the three

Mycobacteria species, indicating that M. tuberculosis S9 protein is species-specific for detection of M. tuberculosis under these assay conditions and/or using the selected antibody pair. This supports utility of antibodies against S9 protein in a single-analyte diagnostic test for M. tuberculosis, or alternatively, as part of a multi-analyte test in conjunction with antibodies against other antigenic markers of M. tuberculosis infection such as described herein or known in the art.

Antibodies against S9 protein may be employed in conjunction with subsequent culture of M. tuberculosis from S9-positive clinical specimens.

Alternatively, or in addition, antibodies against S9 protein may be employed simultaneously with one or more antibodies against M. tuberculosis Rv1265 and/or M. tuberculosis BSX protein and/or M. tuberculosis EF-Tu and/or M. tuberculosis KARI protein as described herein.

### 10. Detection of S9 Protein in Clinical Samples

To further assess the suitability of S9 as a diagnostic marker for the presence of M. tuberculosis in biological samples, the inventors determined the ability of antibodies to detect endogenous S9 protein in clinical samples obtained from TB-positive subjects who had been diagnosed previously on the basis of smear test and M. tuberculosis culture, assay results. Patients had been categorized on the basis of both smear and culture test results, and HIV status. All subjects tested were both smear-negative and culture-negative or alternatively, both smear-positive and culture-positive.

Briefly, sandwich ELISA was performed as described herein above on the sputum samples, which were prepared by Method 2 or Method 3 and assayed as 4×150 microlitre aliquots or 17×150 microlitre aliquots under the replacement amplification protocol supra. An ELISA plate is coated overnight with capture antibody Ch27. Following washing to remove unbound antibody, treated sputa are added to the wells of the antibody-coated ELISA plates. As a negative control for each assay, buffer without cellular extract was used. Following incubation for 1 hour and washing to remove unbound antigen, detection antibody Mo1025F or M1025F-Bio is contacted with the bound antigen-body complexes. Following incubation at room temperature for 1 hour, plates are washed, incubated with 50 μl of diluted secondary antibody if required (e.g., biotinylated donkey anti-mouse IgG if Mo1025F is used as detection reagent) for 1 hour, and then with poly-40 streptavidin-HRP conjugate for 1 hour, washed again, incubated with TMB for 10 mins, and the absorbance at 450-620 nm is determined. Samples are assayed in duplicate over three dilutions of whole cell extracts. A calibration standard curve was produced based on standardized levels of S9 protein.

Data presented in FIGS. 62 to 67 show significant detection of S9 protein TB-positive samples that had been shown previously to be culture-positive and smear-positive. When expressed in pg S9 protein per mL sputum, signals for TB-positive samples are consistently above background for TB-negative samples. Preferred assay results are obtained using Method 3 for preparing clinical sputa samples, with replacement amplification.

### 11. Evaluation of Signal Inhibition by Sample

To assess whether or not inhibitory or signal-suppressing factors are present in sputa that might adversely affect assay sensitivity e.g., in ELISA or a point-of-care or field test format, sputa samples were spike with 10 ng/mL recombinant M. tuberculosis S9 protein and the resultant samples serially diluted 1:27 (v/v) over three steps. Samples were incubated overnight and assayed by amplified ELISA as described herein above, or assayed immediately.

Data presented in FIGS. 87 and 88 (lower left panel marked “S9”) indicate that sputum contains some factors that inhibit S9 protein signal detection, since signal strength is reduced following addition of undiluted sputum, irrespective of whether or not the assay is performed immediately or following overnight incubation. However, this loss of signal strength can be progressively inhibited by diluting the sputa, and loss of signal strength is largely prevented by diluting sputum at least about 1:9 (v/v). Signal strength also declines following overnight incubation of recombinant protein in sputum. These data indicate that at least 1:9 (v/v) dilution of sputum into blocking buffer and rapid assay of samples may enhance signal strength in assaying S9 protein under these conditions.

In a separate set of experiments to assess whether or not factors are present in plasma, different concentrations of recombinant M. tuberculosis ribosomal protein S9 (i.e., 0.8-16 ng/ml) were mixed with serial dilutions of plasma and tested in an assay format utilizing biotinylated secondary antibody and streptavidin poly-40 horseradish peroxidase (HRP) conjugate, as described herein above.

Under the conditions tested, no significant suppression of signal was observed for plasma-containing samples, and a minimum loss in signal strength could be compensated by performing iterative sample loading for at least one round (FIG. 58).

### 12. Relative Levels of S9 Protein Detectable in Mycobacterium Cells

To further assess the suitability of S9 as a diagnostic marker for Mycobacteria infection, the levels of S9 protein were determined in whole cell lysates of the M. tuberculosis strains H37Rv, CSU93 and HN878, and in M. tuberculosis, M. avium and M. intracellulaire, relative to 10 other M. tuberculosis antigens including BSX, KARI, EF-Tu, P5CR, Rv1265 and TetR-like protein described herein.

Amplified sandwich ELISA was performed essentially as described in this example and in Example 1, to identify relative levels of each antigen according to the standard protocol, with calibration standards included to permit quantitation.

Data shown in FIGS. 130-131 indicate that S9 is a moderately-abundant protein in all three M. tuberculosis strains tested when expressed on the basis of total cellular protein. On this basis, M. tuberculosis BSX, Rv1265 and S9 proteins are expressed at similar levels, and at intermediate levels relative to KARI which is expressed at much higher levels, and EF-Tu expressed at lower levels. Data shown in FIGS. 126-131 confirm the specific expression of S9 in M. tuberculosis compared to M. avium and M. intracellulaire in which the protein is not readily detectable by sandwich ELISA under these conditions, when expression is calculated on a per cell basis (FIGS. 126-127) or per microgram of whole cell lysate protein (FIGS. 128-129) or per microlitre of whole cell lysate filtrate (FIGS. 130-131). These data suggest utility of S9 as a single-analyte marker of mycobacteria infection, or as part of a multi-analyte test for mycobacteria infection or M. tuberculosis infection in combination with KARI and/or Rv1265 and/or BSX proteins. Other combinations are not excluded for multi-analyte testing of M. tuberculosis infection.

### Example 5

### Antigen-Based Diagnosis of Tuberculosis or Infection by M. Tuberculosis Using Antibodies that Bind to the M. Tuberculosis Protein Rv1265/MT1303

### 1. Identification of Rv1265 Protein in TB-Positive Subjects

Protein fragments were recognized in the immune-globulin fraction of plasma from TB⁺ samples. The sequences of four peptides from MALDI MS data (SEQ ID Nos: 22-25 inclusive) matched a hypothetical protein having SwissProt Accession No. P64789 (SEQ ID NO: 21). The percent coverage of P64789 by these 4 peptides (SEQ ID NOs: 22-25) was about 19%, suggesting that the peptide fragments were derived from this same protein marker.

The identified hypothetical protein having the amino acid sequence set forth in SEQ ID NO: 21 was designated as “protein Rv1265/MT1303”. The estimated molecular weight of the protein Rv1265/MT1303 is about 25.2 kDa, and the estimated isoelectric point is about 7.11.

### 2. Antibodies

### a) Antibodies Prepared Against Peptide Fragments of M. Tuberculosis Rv1265/MT1303

A synthetic peptide comprising amino acid residues 13-25 of full length protein Rv1265/MT1303 (SEQ ID NO: 26) and a synthetic peptide comprising amino acid residues 90-100 of SEQ ID NO: 21 (i.e., SEQ ID NO: 27) were synthesized according to standard procedures. These peptides can be coupled separately to keyhole limpet Hemocyanin (KHL) via a maleimidocaproyl-N-hydroxysuccinimide linker.

To facilitate detection of antibodies raised against these epitopes the peptides can also be synthesized separately, each with a GSGL spacer and attached to biotin.

Chickens and rabbits were immunized with synthetic peptide comprising the amino acid sequence set forth in SEQ ID NO: 26 according to standard procedures. Animal bleeds were obtained. All blood was collected in sterile containers and serum collected after clot removal.

To titrate antisera produced in chickens, recombinant protein Rv1265/MT1303 was immobilized at a concentration of 5 μg/ml onto Nunc immune-plates. The solution was removed and wells blocked using blocking buffer (1% (w/v) casein, 0.1% (v/v) Tween 20, 0.1% (w/v) sodium azide in PBS). Blocking buffer was removed and serum diluted in PBS added and incubated for a sufficient time for antibodies to complex with bound recombinant protein Rv1265/MT1303, generally for about 1 hour at room temperature. Plates were washed and binding of the antibody to recombinant protein Rv1265/MT1303 was detected using HRP-conjugated sheep anti-chicken IgG diluted 1:5000 (v/v) in conjugate diluent buffer. Fifty millilitres (50 ml) of TMB (3,3′,5′,5-Tetramethylbenzidine; Sigma) were added to each well and the plate incubated in the dark for 30 minutes. Development was stopped by addition of 50 μL per well of 0.5M sulphuric acid. The optical density of each well was read with a microtitre plate reader (PowerWaveₓ 340 plate reader, Bio-Tek Instruments Inc., Winooski, Vt.) using a wavelength of 450 nm and an extinction at 620 nm. The titration results are shown in FIG. 68.

For testing rabbit antisera, streptavidin (Sigma Aldrich) was diluted to 5 μg/ml in double-distilled water (ddH₂O) and incubated in a Nunc plate overnight at 4° C. The solution was then flicked out of the plate and 250 μL of blocking buffer (1% (w/v) casein, 0.1% (v/v) Tween 20, 0.1% (w/v) sodium azide in PBS) added to each well and incubated at room temperature for 1 hour. The blocking buffer was flicked out and biotinylated peptide (SEQ ID NO: 26) was added in blocking buffer at 3 μg/ml to 50 μl/well and incubated for one hour at room temperature on a shaker. The plate was washed in an Elx405 Auto Plate Washer (Bio-Tek Instruments Inc., Winooski, Vt.), with 0.5×PBS/0.05% (v/v) Tween 20 solution and excess solution tapped out of the plate onto a paper towel. Rabbit sera were diluted in blocking buffer from 1:500 (v/v) to 1:1,024,000 (v/v) and incubated for 1 hour at 50 μl/well at room temperature on a shaker. Plates were washed with the plate washer using 0.5×PBS/0.05% (v/v) Tween 20 solution, and the excess solution tapped out on a paper towel. Binding of the rabbit antibodies to SEQ ID NO: 26 was detected using HRP-conjugated Sheep anti-rabbit IgG (Chemicon) diluted 1:5000 (v/v) in conjugate diluent buffer. Fifty millilitres (50 ml) were added to each well and incubated for one hour at room temperature on a shaker. Plates were washed with the plate washer using 0.5×PBS and excess solution tapped out on a paper towel. Fifty millilitres (50 ml) of TMB (3,3′,5′,5-Tetramethylbenzidine; Sigma) were added to each well and the plates incubated in the dark for 30 minutes. Development was stopped by addition of 50 μL per well of 0.5M sulphuric acid. The optical density of each well was read with a microtitre plate reader (PowerWaveₓ 340 plate reader, Bio-Tek Instruments Inc., Winooski, Vt.) using a wavelength of 450 nm and an extinction at 620 nm. The sera titration data are shown in FIG. 69.

Sera detection limits were determined by titrating recombinant Rv1265/MT1303 protein immobilized onto an ELISA plate at concentrations of 19.6 ng/ml to 200 pg/ml. Purified rabbit antibody was added at a concentration of 1.25 μg/ml, 2.5 μg/ml or 5 μg/ml. A sheep anti-rabbit Ig HRP conjugate (1:5000 (v/v) dilution) and TMB were used to detect the bound rabbit antibodies in standard ELISA. Data are presented in FIG. 70.

To titrate the peptide, a protocol essentially as described supra is used. However, a biotinylated peptide comprising SEQ ID NO: 26 is titrated from 20,480 pg/ml to 100 pg/ml and the rabbit sera are added to the ELISA at dilutions of 1:500 (v/v) and 1:2000 (v/v).

### b) Monoclonal Antibodies Prepared Against Recombinant M. Tuberculosis Rv1265 Protein

The full length recombinant Rv1265 protein (SEQ ID NO: 21) was used as an antigen for antibody production, according to standard procedures. Approximately 6 mg of protein was provided to NeoClone, Madison, Wis., USA for generation of monoclonal antibodies according to their standard protocol. About 1 mg of the protein was provided as biotinylated peptide for quality control.

Five BALB/cByJ female mice were immunized with protein according to Neoclone's standard immunization process. Test bleeds of the immunized mice were performed at regular intervals for use in the quality control sera ELISAs using biotinylated peptide. Polyclonal sera having the highest titer were determined using ELISA. Mice having polyclonal antibody titers of at least 1,000 were used for the ABL-MYC infection process. The spleens of 3 mice having the highest titer of polyclonal antibodies cross-reactive with peptide antigen were used for the ABL-MYC infection, according to NeoClone's standard infection procedure. The splenocytes of the ABL-MYC-infected mice were transplanted into approximately 20 naive mice. Ascites fluid developed in the transplanted mice were isolated and screened for cells producing monoclonal antibodies (mAbs) that bind to the target peptide antigen.

A cell line (i.e., plasmacytoma) producing a mAb designated 788C was isolated. Binding affinity and isotype specificity of the mAb 788C was confirmed using ELISA. The mAb designated 788C was provided in 1 ml aliquots (approximately) in ascites, together with the associated cell line. This monoclonal antibody preparation against Rv1265 had high titer when assayed using standard procedures (data not shown) and further diagnostic testing herein was conducted using the monoclonal sera as both a capture and a detection reagent, but preferably as a capture reagent due to the lower background of the antibody in this orientation. The mAb designated 788C can be further purified from ascites using protein G or protein A columns.

### c) Polyclonal Antibodies Prepared Against Recombinant M. Tuberculosis Rv1265 Protein

Two additional antibody preparations were prepared against the full length recombinant M. tuberculosis protein Rv1265 (SEQ ID NO: 21), by immunization of chickens using standard procedures. Two separate batches of chicken polyclonal antisera, designated “Ch10” and “Ch11” were raised against the Rv1265 protein, and these were then pooled to produce the antibody designated herein as “Ch10/11”. The polyclonal antibody preparations against Rv1265 had high titers when assayed using standard procedures (data not shown) and further diagnostic testing herein was conducted using the polyclonal sera designated Ch10/11.

### 3. Validation of Rv1265 and Antibodies Thereto as Diagnostic Reagents

To further assess the suitability of Rv1265 as a diagnostic marker for the presence of M. tuberculosis in biological samples when detected using the antibodies designated Ch10/11 and Mo788C, the inventors compared antibody reactivities between cellular extracts of the laboratory isolate H37Rv and clinical M. tuberculosis strains CSU93 and HN878, and the laboratory M. tuberculosis strain H37Rv by western blotting and immune precipitation.

### a) Western Blotting

Briefly, Western blotting was performed on proteins separated by electrophoresis on 10% (w/v) Bis-Tri Nu-PAGE (Invitrogen, Carlsbad Calif., USA) and transferred to PVDF activated membrane (Immobilon-P, Millipore Inc, USA). Following transfer, membranes were incubated in 0.008% DB-71 (Sigma Chemical Co. USA) in 40% (v/v) ethanol/10% (v/v) acetic acid for 7 min, rinsed briefly in 40% (v/v) ethanol/10% (v/v) acetic acid, scanned to visually confirm protein transfer, and rinsed in Tris-buffered saline containing Triton-X100 (TBS-T). Dried membranes were re-activated in methanol, and transferred to blocking buffer (TBS-T containing 1% (w/v) bovine serum albumin) overnight at 4° C. Primary antibody Ch10/11 was diluted to a concentration of 0.5 μg/ml in blocking buffer and incubated with the membranes for 90 min at room temperature, after which time the membranes were washed in TBS-T, incubated with HRP-conjugated secondary antibody i.e., a sheep anti-chicken IgG-HRP conjugate diluted 1:100,000 (v/v), for 60 min at room temperature, and washed as before. Antibody Mo788c was diluted to a concentration of 5 μg/ml in blocking buffer and incubated with the membranes for 90 min at room temperature, after which time the membranes were washed in TBS-T, incubated with HRP-conjugated secondary antibody i.e., a donkey anti-mouse IgG-HRP conjugate diluted 1:50,000 (v/v), for 60 min at room temperature, and washed as before. Binding of HRP-secondary antibody conjugates was detected by incubating membranes in SuperSignal™ West “Femto” Maximum Sensitivity Substrate (Pierce, Inc. USA), and visualizing chemiluminescence using the LAS-3000 multi-imager (FujiFilm Inc., Japan). This experimental protocol was generally applied for Western blotting of other antigens described herein.

Immune-reactive bands of about 25 kDa, consistent with the expected molecular masses of M. tuberculosis Rv1265 protein, were detected in both clinical M. tuberculosis isolates CSU93 and HN878, and in the laboratory strain H37Rv (data not shown) employing

Ch10/11 polyclonal sera. In a control, recombinant Rv1265 protein comprising a hexahistidine tag having an estimated molecular mass of about 28 kDa was also detected at the correct position (not shown). There was little or no detectable background.

Competition Western blot analysis performed essentially as described in Example 1 indicated that binding of the polyclonal sera Ch10/11 to recombinant Rv1265 protein and endogenous Rv1265 protein could be ablated by pre-incubation of antibodies with excess concentration of unlabelled recombinant Rv1265 protein (data not shown).

The available data indicate the specificity of the polyclonal sera Ch10/11 for detecting Rv1265 protein in whole cell lysates.

### b) Immune Precipitation

Further validation of antibody specificity is achieved by immune-precipitation from whole cell extracts of strain H37RV using the Ch10/11 polyclonal antibody, and determining the amino acid sequence of the immune-precipitated protein by LC-MS.

4. Sandwich ELISA Using Antibodies Prepared Against M. tuberculosis Rv1265

This example demonstrates effective detection of Rv1265 protein by sandwich ELISA using the monoclonal antibody Mo788C as a capture reagent and the polyclonal antibody pool designated Ch10/11 as a detection reagent. This antibody pair was selected because, for example, titration of the pooled antibody preparation Ch10/11 and monoclonal antibody Mo788C indicated that Ch10/11 and Mo788C were capable of detecting less protein at 5 μg/ml antibody concentration.

### a) Preferred Antibody Orientation

In a first set of diagnostic tests, a standard sandwich ELISA was performed to determine optimum capture and detection antibodies, and appropriate antibody concentrations for use. Briefly, the wells of an ELISA plate were coated overnight with 50 μl of a 5 μg/ml concentration or 10 μg/ml concentration of Mo788C antibody, or a 2.5 μg/ml concentration or 5 μg/ml concentration of Ch10/11 antibody. Following blocking and washing to remove unbound antibody, recombinant Rv1265 protein was diluted serially 1:3 (v/v) from 500 ng/ml starting concentration to 22.86 pg/ml, and 50 μl aliquots of each dilution were added the wells of the antibody-coated ELISA plates. Following incubation for 1 hour and washing to remove unbound antigen, 50 μl of the alternate detection antibody (i.e., Ch10/11 at 2.5 μg/ml or 5 μg/ml or 10 μg/ml for detecting Rv1265-Mo788C complexes, or Mo788C at 5 μg/ml or 10 μg/ml or 20 μg/ml for detecting Rv1265—Ch10/11 complexes), was contacted with the bound antigen-body complexes. Following incubation at room temperature for 1 hour, plates were washed, incubated with 50 μl of a 1:5000 (v/v) dilution of secondary antibody (i.e., sheep anti-chicken IgG for detecting Ch10/11 or sheep anti-mouse IgG for detecting Mo788C) conjugated to horseradish peroxidase (HRP), washed, incubated with TMB for 30 mins, and absorbance at 450-620 nm was determined after subtraction of background.

These titration experiments (data not shown) indicated that, irrespective of the capture reagent, the signal was not dependent on the concentration of detection antibody. Both systems with Mo788C and Ch10/11 as capture regardless of the concentrations tested showed a similar sensitivity of 9 ng/ml. Proceeding on this basis, we employed Mo788C at 5 μg/ml for capture and Ch10/11 at 2 μg/ml for detection in sandwich ELISA.

In a further experiment (FIG. 71), wells of an ELISA plate were coated overnight with 50 μl of a 5 μg/ml concentration of Ch10/11 or Mo788C antibody. Following blocking and washing to remove unbound antibody, recombinant Rv1265 protein was diluted serially 1:3 (v/v) from 500 ng/ml starting concentration to 22.86 pg/ml, and 50 μl aliquots of each dilution were added the wells of the antibody-coated ELISA plates (x-axis). Following incubation for 1 hour and washing to remove unbound antigen, the alternate detection antibody i.e., Ch10/11 for detecting Rv1265-Mo788C complexes and Mo788C for detecting Rv1265—Ch10/11 complexes, was contacted with the bound antigen-body complexes at a concentration of 2 μg/ml. Following incubation at room temperature for 1 hour, plates were washed, incubated with 50 μl of a 1:5000 (v/v) dilution of secondary antibody (i.e., sheep anti-chicken IgG for detecting Ch10/11 or sheep anti-mouse IgG for detecting Mo788C) conjugated to horseradish peroxidase (HRP), washed, incubated with TMB for 30 mins, and absorbance at 450-620 nm was determined after subtraction of background (y-axis).

Without limiting the invention, data presented in FIG. 71 suggest that lower background effects are observed when the monoclonal antibody M0788C is used as a capture reagent and the polyclonal antibody pool Ch10/11 is used as a detection reagent in sandwich ELISA. The LOD in standard ELISA is about 522 pg/mL.

### 5. Amplified Sandwich ELISA for Detection of M. Tuberculosis Rv1265 Protein

Amplified ELISA was performed essentially as described in this example and in Example 1, using Mo788C antibody as a capture reagent and polyclonal Ch10/11 antibody as a detector antibody, and a biotinylated secondary antibody with HRP-conjugated streptavidin to detect the bound detector antibody.

An ELISA plate was coated overnight with capture antibody Mo788C at 5 μg/ml concentration. Following washing to remove unbound antibody, recombinant Rv1265 protein was diluted serially 1:10 (v/v) from 10 μg/ml starting concentration to 1.0 pg/ml, and 50 μl aliquots of each dilution were added the wells of the antibody-coated ELISA plates (x-axis). Following incubation for 1 hour and washing to remove unbound antigen, antibody Ch10/11 was contacted with the bound antigen-body complexes at 2.0 μg/ml concentration. Following incubation at room temperature for 1 hour, plates were washed, and incubated with 50 μl of a 1:5,000 (v/v) dilution of a secondary antibody consisting of HRP-conjugated sheep anti-chicken IgG (standard sandwich ELISA) or 50 μl of a 1:50,000 (v/v) dilution of biotinylated donkey anti-chicken IgG (amplified sandwich ELISA). Following incubation at room temperature for a further one hour, the plates washed as before. For samples undergoing amplified ELISA, HRP80-streptavidin was then added to the plates which were incubated for a further one hour at room temperature, and washed as before. Finally, all samples were incubated with TMB for 30 mins. Absorbance was determined at 450-620 nm.

As shown in FIG. 72, better results were obtained using of a streptavidin poly-80 horseradish peroxidase (HRP) with biotinylated donkey anti-chicken IgG to detect bound Ch10/11 antibody in ELISA, compared to the standard sandwich ELISA described herein above. More particularly, data indicate significant enhancement of detection using the amplified sandwich ELISA. The limit of detection of this amplified sandwich ELISA is about 60 pg/ml Rv1265 protein, with half-maximum detection of about 5 ng/ml Rv1265 protein. This compares favourably to the observed limit of detection of the standard sandwich ELISA of about 2.6 ng/ml Rv1265 protein, with half-maximum detection of about 100 ng/ml Rv1265 protein. These data also suggested that the Ch10/11 antibody would be superior to Mo788C as a detector antibody in an amplified sandwich ELISA format.

Thus, under the assay conditions tested and with this preferred, albeit not essential, orientation of antibodies, there is low background noise and a LOD of about 60 pg/mL. Such sensitivity of detection coupled with low background in sandwich ELISA is considered by the inventors to be within useful limits.

### 6. Cross-Reactivity Between Anti-Rv1265 Antibodies and Different M. Tuberculosis Isolates

To further assess the suitability of Rv1265 as a diagnostic marker for the presence of M. tuberculosis in biological samples, and to assess the specificities of antibodies prepared against Rv1265 protein, the inventors compared antibody reactivities in amplified sandwich ELISA performed as described herein above between cellular extracts of the clinical M. tuberculosis strains CSU93 and H878 and the laboratory M. tuberculosis strain H37Rv.

Whole cell lysates (1.8 μg/ml, 5.6 μg/ml, 16.7 μg/ml, and 50 μg/ml diluted in blocking buffer) were assayed for the presence of Rv1265 in duplicate by amplified sandwich ELISA, essentially as described herein. The concentration of endogenous Rv1265 protein in the whole cell lysates was calculated by interpolation from the standard curve and corrected for the dilution factor. Levels of endogenous M. tuberculosis Rv1265 protein present in these strains, as determined from two independent experiments is indicated in FIG. 73. Data indicate average Rv1265 levels of about 1 ng/μg cell extract for H37Rv, and about 300 pg/μg in whole cell extracts of the clinical isolates.

In summary, the data obtained to date thus indicate that the Ch10/11 antibody is capable of detecting endogenous Rv1265 protein in whole cell extracts of clinically-relevant and laboratory strains of M. tuberculosis.

### 7. Low Cross-Reactivity Between M. Tuberculosis and Non-Mycobacteria Pathogens

To further assess the suitability of Rv1265 as a diagnostic marker for the presence of M. tuberculosis in biological samples, the inventors compared antibody cross-reactivities in sandwich ELISA performed between cellular extracts of M. tuberculosis strain H37Rv (a laboratory strain), Escherichia coli, Bacillus subtilis or Pseudomonas aeruginosa.

Amplified sandwich ELISA was performed essentially as described herein between different concentrations of recombinant Rv1265 protein and 100 ng/ml and 100 μg/ml cellular extracts of yeast, Escherichia coli, Bacillus subtilis or Pseudomonas aeruginosa.

Data presented in FIG. 74 show low cross-reactivity of antibodies against M. tuberculosis Rv1265 with yeast, Escherichia coli, Bacillus subtilis or Pseudomonas aeruginosa cellular extracts under the conditions tested. In particular there was little or no signal differential between the concentrations of cellular extracts tested, and the signal obtained were not significantly above background. In contrast, the assay detected less than 1 ng/ml recombinant Rv1265 protein.

### 8. Evaluation of Signal Inhibition by Sample

To assess whether or not factors are present in biological samples that are to be tested using the sandwich ELISA of the present invention, e.g., in a point-of-care or field test format, different concentrations of recombinant M. tuberculosis Rv1265 protein (i.e., 0-8 ng/ml) were mixed with serial dilutions of plasma (FIG. 75) or sputum (FIG. 76) and tested in the amplified sandwich ELISA assay format described above without replacement amplification, i.e., utilizing Mo788C as capture antibody, Ch10/11 detector antibody, and a detection system comprising a biotinylated donkey anti-chicken IgG and streptavidin poly-80HRP (“HRP80-streptavidin”).

Under the conditions tested, significant suppression of signal was observed for undiluted or diluted plasma-containing samples (FIG. 78).

In contrast, no suppression of signal was observed for undiluted sputum-containing samples in this set of experiments (FIG. 79). However, in a further set of experiments, sputa samples were spiked with 10 ng/mL recombinant M. tuberculosis Rv1265 protein and the resultant samples serially diluted 1:27 (v/v) over three steps. Samples were incubated overnight and assayed by amplified ELISA as described herein above, or assayed immediately. Data presented in FIGS. 87 and 88 (upper right panel marked “Rv1265”) suggest that some sputa contain factors that inhibit Rv1265 protein signal detection, since signal strength in this case was reduced following addition of undiluted sputum, irrespective of whether or not the assay is performed immediately or following overnight incubation. However, loss of signal strength in this case was inhibited by diluting the sputa by about 1:3 (v/v) and signal was enhanced over expected values if diluted further. Such dilution also abrogated any loss of signal strength due to prolonged incubation of sputa.

Taken together, these data suggest that detection of Rv1265 in sputa is not adversely affected to a substantial degree by sputa-derived factors.

### 9. Cross-Reactivity Between Different Mycobacteria Species

To further assess the suitability of Rv1265 as a diagnostic marker for the presence of M. tuberculosis in biological samples, and to assess the specificities of antibodies prepared against Rv1265 protein, the inventors compared antibody reactivities in amplified sandwich

ELISA performed as described herein above between cellular extracts of the Mycobacteria species M. tuberculosis, M. avium and M. intracellulaire.

Briefly, an ELISA plate was coated overnight with capture antibody Mo788C. Following washing to remove unbound antibody, a cellular extract from each Mycobacteria species was added to the wells of the antibody-coated ELISA plates. As a negative control for each assay, buffer without cellular extract was used. Following incubation for 1 hour and washing to remove unbound antigen, detection antibody Ch10/11 was contacted with the bound antigen-body complexes. Following incubation at room temperature for 1 hour, plates were washed, incubated with 50 μl of diluted secondary antibody (e.g., biotinylated donkey anti-chicken IgG) for 1 hour, washed again, incubated with TMB for 10 mins, and the absorbance at 450-620 nm was determined. Samples were assayed in duplicate over three dilutions of whole cell extracts. A calibration standard curve was produced based on standardized levels of Rv1265 protein.

Data presented in FIGS. 77 and 78 show no detectable cross-reactivity between the three Mycobacteria species, indicating that M. tuberculosis Rv1265 protein is specific to M. tuberculosis under these assay conditions and/or using the selected antibody pair. This suggests utility of antibodies against Rv1265 protein in a species-specific single-analyte diagnostic test, or alternatively, as part of a multi-analyte test in conjunction with antibodies against a other markers of Mycobacteria generally or M. tuberculosis such as described herein or known in the art. For example, antibodies against Rv1265 protein may be employed simultaneously with one or more antibodies against M. tuberculosis KARI protein and/or M. tuberculosis BSX protein and/or M. tuberculosis EF-Tu and/or M. tuberculosis S9 protein as described herein.

Antibodies against Rv1265 protein may also be employed in conjunction with subsequent culture of M. tuberculosis from Rv1265-positive clinical specimens.

### 10. Relative Levels of Rv1265 Protein Detectable in Mycobacterium Cells

To further assess the suitability of Rv1265 as a diagnostic marker for Mycobacteria infection, the levels of Rv1265 protein were determined in whole cell lysates of the M. tuberculosis strains H37Rv, CSU93 and HN878, and in M. tuberculosis, M. avium and M. intracellulaire, relative to 10 other M. tuberculosis antigens including BSX, EF-Tu, P5CR, S9, KARI and TetR-like protein described herein.

Amplified sandwich ELISA was performed essentially as described herein and in this example and in Example 1, to identify relative levels of each antigen according to the standard protocol, with calibration standards included to permit quantitation.

Data shown in FIGS. 130-131 indicate that Rv1265 is a moderately-abundant protein in all three M. tuberculosis strains tested when expressed on the basis of total cellular protein. On this basis, M. tuberculosis BSX, Rv1265 and S9 proteins are expressed at similar levels, and at intermediate levels relative to KARI which is expressed at much higher levels, and EF-Tu expressed at lower levels. Data shown in FIGS. 126-131 confirm the highly-specific expression of Rv1265 in M. tuberculosis compared to M. avium and M. intracellulaire in which the protein is not readily detectable by sandwich ELISA under these conditions, when expression is calculated on a per cell basis (FIGS. 126-127) or per microgram of whole cell lysate protein (FIGS. 128-129) or per microlitre of whole cell lysate filtrate (FIGS. 130-131).

These data suggest utility of Rv1265 as a single-analyte marker of mycobacteria infection, or as part of a multi-analyte test for mycobacteria infection or M. tuberculosis infection in combination with KARI and/or S9 and/or BSX proteins. Other combinations are not excluded for multi-analyte testing of M. tuberculosis infection.

### 11. Replacement Amplification

To further enhance sandwich ELISA sensitivity, the inventors will further modify the basic assay by employing iterative antigen binding following coating of the ELISA plate with capture antibody. Essentially, this results in an increased amount of antigen being bound to the capture antibody notwithstanding the 50 μl volume limitations of a 96-well ELISA plate. Briefly, this iterative antigen loading involves repeating the antigen binding step in the sandwich ELISA several times, e.g., 2 or 3 or 4 or 5 times, etc. before washing and adding detection antibody. Naturally, each aliquot of antigen sample is removed following a standard incubation period before the next aliquot is added. The number of iterations can be modified to optimize the assay (e.g., parameters such as signal:noise ratio, detection limit and amount of antigen detected at half-maximum signal), depending upon the nature of the sample being tested (e.g., sample type), without undue experimentation.

For example, up to about 20 iterations of sample loading (i.e., up to a 20× replacement amplification) may provide a low background signal, and reduce the detection limit for Rv1265 protein.

### Example 6

### Antigen-Based Diagnosis of Tuberculosis or Infection by M. Tuberculosis Using Antibodies that Bind to M. Tuberculosis Elongation Factor-Tu (EF-Tu) Protein

### 1. Identification of EF-Tu Protein in TB-Positive Subjects

Protein fragments were recognized in the immune-globulin fraction of plasma from a TB+ sample. The sequence of one peptide from MALDI MS data matched a protein having SwissProt Accession No. P64789 (SEQ ID NO: 29).

The identified hypothetical protein having the amino acid sequence set forth in SEQ ID NO: 29 was designated as “Elongation Factor-Tu” or “EF-Tu”. The estimated molecular weight of EF-Tu protein is about 43.59 kDa, and the estimated isoelectric point is about 5.28.

### 2. Validation of EF-Tu and Antibodies Thereto as Diagnostic Reagents

### a) Validation of EF-Tu by Detection of Antibodies Against EF-Tu is Patient Sera

To validate EF-Tu as a diagnostic marker of M. tuberculosis infection, full-length recombinant protein was screened by one-site ELISA for immune reactivity against sera of TB+ and TB− subjects. Briefly, recombinant EF-Tu was immobilized onto an ELISA plate at 50 μl per well at a concentration of 20 μg/ml. Plasma from TB and non-TB subjects, diluted 1:100 (v/v) was then added to each well of the ELISA plate. Bound human IgG were detected by binding Sheep anti-Human IgG HRP conjugate diluted 1:50,000 (v/v) and by subsequent colour development with TMB substrate at 50 μl per well. As is shown in FIG. 89, a number of subjects suffering from TB (both HIV+ and HIV⁻) carried antibodies against EF-Tu protein in their sera. These data suggest that subjects infected with M. tuberculosis raise antibodies against EF-Tu, independent of whether or not they are additionally infected with HIV. These data indicate that EF-Tu has utility as a biomarker for diagnosis of M. tuberculosis infection or tuberculosis.

### b) Validation of EF-Tu by Linear B Cell Epitope Mapping of EF-Tu Protein

To further validate EF-Tu as a marker of M. tuberculosis infection, linear B-cell epitope mapping of the protein was performed to identify immunogenic epitopes of the EF-Tu protein. A set of 82 synthetic peptides (PEPSET) was produced from the primary amino acid sequence shown in SEQ ID NO: 29. Each synthetic peptide was attached to biotin via a spacer consisting of the amino acid residues Ser-Gly-Ser-Gly to facilitate their use in ELISA assays.

Sera isolated from subjects diagnosed with TB and control sera isolated from control subjects were screened by one-site ELISA with the peptides of the PEPSET to identify those peptides against which subjects infected with M. tuberculosis raise an immune response. The tested sera were from South African (S.A.) Zulu TB positive individuals, S.A. Zulu TB negative individuals, Chinese TB positive individuals, Chinese TB negative individuals, World Health Organization (WHO) TB positive individuals of unknown race, WHO TB negative individuals of unknown race, and Australian Caucasian TB negative control individuals. Briefly, Nunc-Immune-module maxisorp wells were coated overnight at room temperature or at 4° C. over the weekend with 100 μl/well of 5 μg/ml streptavidin diluted in milli-Q water. The streptavidin was flicked out of the wells and each well was blocked with 400 μl phosphate-buffered saline (PBS) containing 1.0% (w/v) casein, 0.1% (v/v) Tween 20 and 0.1% (w/v) Azide (blocker) per well. After 1 hour, the blocker was removed, and each well was coated with 100 μl of biotinylated peptide in blocker for 1 hour, with agitation of the plate. Subsequently, each well was washed using the Elx405 Auto Plate Washer (Bio-Tek Instruments Inc., Winooski, Vt.) with 0.5×PBS/0.05% Tween 20, allowed to dry on absorbent paper, and either stored at 4° C. with desiccant, or used immediately. This was followed by incubation for 1 hour with agitation in 50 μl of patient serum, diluted 1:50 (v/v) in blocker. Following this incubation, all wells were washed using the Elx405 Auto Plate Washer (Bio-Tek Instruments Inc., Winooski, Vt.) using 0.5×PBS/0.05% Tween 20 in a laminar flow, and tapped dry. 80 μl Sheep anti-human IgG Horse Radish Peroxidase (HRP) conjugate was then added to each well. The conjugate was diluted 1:10,000 (v/v) in PBS/0.1% (w/v), casein/0.1% (v/v), Tween 20/0.1% (w/v) thimerosal, and incubated for 1 hour with agitation. Each well was then washed using the Elx405 Auto Plate Washer (Bio-Tek Instruments Inc., Winooski, Vt.) using PBS. Finally, 80 μl liquid TMB substrate based system (Sigma) was added to each well, and the wells incubated at room temperature in the dark for 20 mins. Reactions were stopped by addition of 80 μl 0.5M sulfuric acid. Each peptide was assayed in duplicate and repeated if duplicates did not appear to be reproducible.

Immunogenic peptides represent outliers in the distribution of peptide absorbencies and were detected following log transformation normalisation by calculation of a normal score statistic, with the mean and standard deviation estimated by a robust M-Estimator. The amino acid sequences of immunogenic peptides of EF-Tu identified by this means are shown in Table 2 below.

TABLE 2

| | SEQ ID NO: |
|---|---|
| Peptide structure | (base peptide) |
| Biotin-SGSGGRVERGVINVNEEVE-NH2 | 30 |
| Biotin-SGSGGVINVNEEVEIVGIR-NH2 | 31 |
| Biotin-SGSGNEEVEIVGIRPSTTK-NH2 | 32 |
| Biotin-SGSGIVGIRPSTTKTTVTG-NH2 | 33 |
| Biotin-SGSGPSTTKTTVTGVEMFR-NH2 | 34 |

### c) Validation of EF-Tu and Antibodies Against EF-Tu by Western Blot

The amino acid sequence of EF-Tu protein from M. tuberculosis strain H37Rv is presented as SEQ ID NO: 29. The translation product has an expected molecular mass of about 43.6 kDa. One-dimensional SDS/PAGE analysis of a hexa-histidine-tagged recombinant EF-Tu protein performed essentially as described in Example 1 showed that the EF-Tu protein migrated as a single band of approximately 45 kDa (data not shown), which is the expected mass of the fusion protein, based on the theoretical mass of the translation product and the hexahistidine tag moiety.

Western blot analyses of whole cell extracts from M. tuberculosis strains H37Rv, CSU93 and HN878 were performed using antibody probes consisting of a polyclonal antibody designated “Ch49” that was prepared in chickens against an EF-Tu/NusA fusion protein or with a monoclonal antibody designated “Mo683B” that had been prepared in mice using an immunogenic peptide derived from the amino acid sequence of EF-Tu (SEQ ID NO: 35). The preparation of these antibodies is described below. The binding of antibodies was detected using HRP-conjugated secondary antibody in each case.

Both antibodies recognized a band in whole cell lysates derived from all three M. tuberculosis strains that had the expected molecule mass of native EF-Tu protein (i.e., about 43.6 kDa), as well as detecting the slightly larger recombinant EF-Tu protein of about 45 kDa (data not shown). There was little or no detectable background.

Competition Western blot analysis of Mo683B was performed essentially as described in Example 1. Briefly, a 100-fold molar excess of recombinant EF-Tu was pre-incubated with Mo683B prior to probing of Western blots containing recombinant EF-Tu protein. The data indicated that binding of the monoclonal antibody to recombinant EF-Tu protein could be ablated by pre-incubation of antibodies with excess concentration of unlabelled recombinant EF-Tu protein (data not shown).

In summary, the available data indicate that antibodies Mo683B and Ch49 bind specifically to M. tuberculosis EF-Tu protein in whole cell extracts.

### 3. Antibodies

Antibodies were prepared against recombinant EF-Tu protein and immunogenic peptides of EF-Tu (see below) using procedures described herein, and screened for their suitability as reagents for diagnosing M. tuberculosis infection.

a) Isolation of Monoclonal Antibodies that Bind to M. Tuberculosis EF-Tu

Antigens selected for monoclonal antibody production were as follows:
(i) recombinant full-length EF-Tu protein fused to the NUS protein; and
(ii) a peptide comprising the amino acid sequence VINVNEEVEIVGIRPSTTKC (SEQ ID NO: 35).

Each of these antigens was provided to NeoClone, Madison, Wis., USA for generation of monoclonal antibodies according to their standard protocol. Briefly, BALB/cByJ female mice were immunized with peptide conjugated to carrier according to Neoclone's standard immunization process. Test bleeds of the immunized mice were performed at regular intervals for use in the quality control sera ELISAs using biotinylated peptide. Polyclonal sera having the highest titer were determined using ELISA. Mice having polyclonal antibody titers of at least 1,000 were used for the ABL-MYC infection process. The spleens of mice having the highest titer of polyclonal antibodies cross-reactive with peptide antigen were used for the ABL-MYC infection, according to NeoClone's standard infection procedure. Splenocytes of the ABL-MYC-infected mice were transplanted into naive mice. Ascites fluid that developed in the transplanted mice were isolated and screened for cells producing monoclonal antibodies (mAbs) that bind to the target peptide antigens.

Fifteen distinct plasmacytomas producing monoclonal antibodies were obtained using the synthetic peptide (SEQ ID NO: 35), and these were designated “Mo524A, “Mo524B”, “Mo524C”, “Mo524D”, “Mo524E”, “Mo524F”, “Mo525B”, “Mo525D”, “Mo525F”, “Mo680A”, “Mo681E”, “Mo682A”, “Mo683B”, “Mo684A” and “Mo685B”). An additional six plasmacytomas producing monoclonal antibodies were produced using the recombinant EF-Tu protein fused to NusA, and these were designated “Mo520C, “Mo521C”, “Mo521D”, “Mo521F”, “Mo522A” and “Mo522E”. Cell lines designated Mo683B, Mo682A, Mo685B and Mo684A were split from the cell line 524D. A cell line designated Mo681E was split from the cell line Mo522E. Cell line Mo680A was split from cell line Mo521F.

In one example, plasmacytomas producing the monoclonal antibodies Mo521F, Mo524D and Mo522E were shown to be capable of binding full-length recombinant EF-Tu.

In another example, mAbs designated Mo524D, Mo521F, Mo683B, Mo682A, Mo685B, Mo684A, Mo681E and Mo680A were purified from ascites using protein G or protein A columns.

Monoclonal antibody preparations were titrated by one-site ELISA as follows. Recombinant EF-Tu protein was coated onto the bottom of an ELISA plate at a concentration of about 17 μg/ml. Aliquots of each of the monoclonal antibodies designated Mo524D, Mo521F, Mo683B, Mo682A, Mo685B, Mo684A, Mo681E and Mo680A were added at various final concentrations to 5 pg/ml. Binding of the antibody was then detected using sheep anti-mouse HRP antibody conjugate under standard conditions. Absorbances were determined at 450 nm and 620 nm, and the difference in absorbance at 450 nm and 620 nm determined. Average data were obtained. Results of this assay are shown in FIG. 90.

In further assays to titre the monoclonal antibodies, recombinant EF-Tu was coated onto the bottom of an ELISA plate at a concentration ranging from about 81.4 ng/ml to about 79.5 ng/ml. Aliquots of each of the monoclonal antibodies designated Mo524D, Mo521F, Mo683B, Mo682A, Mo685B, Mo684A, Mo681E and Mo680A were added at 2.5 μg/ml. Binding of the antibody was then detected using sheep anti-mouse HRP antibody conjugate under standard conditions. Average data were obtained. Results of this assay are shown in FIG. 91.

b) Preparation of Polyclonal Antibodies that Bind to M. Tuberculosis EF-Tu

Two separate batches of polyclonal antibodies were produced by immunization of chickens with the full-length recombinant EF-Tu protein, either alone or fused to a NUS protein were produced using standard methods. Immune-globulin fraction was purified from eggs produced by immunized chickens by ammonium sulphate precipitation. Alternatively, antibodies were purified from serum of immunized hens. Two separate batches of chicken polyclonal antisera were prepared against full-length recombinant protein, and these were designated “Ch49” (prepared against EF-Tu-NusA) and “Ch50” (prepared against recombinant EF-Tu).

Polyclonal antibodies were also prepared by immunization of chickens or rabbits according to standard procedures with the immunogenic EF-Tu peptide set forth in SEQ ID NO: 35, optionally fused at the C-terminus to a cysteine residue. Five separate polyclonal antisera were produced, and these were designated “Ch36”, “Ch37”, “Ch38” (prepared in chickens using SEQ ID NO: 35 with C-terminal cysteine residue), “Rb29” (prepared in rabbits using SEQ ID NO: 35 with C-terminal cysteine residue), and “Rb8” (prepared in rabbits using SEQ ID NO: 35).

The polyclonal antibody preparations against full-length recombinant EF-Tu had high titers when assayed using standard procedures (data not shown) and further diagnostic testing herein was conducted using the polyclonal sera designated Ch49 and Ch50.

### 4. Standard ELISA for Detecting M. Tuberculosis EF-Tu Protein

A sandwich ELISA assay was performed using a chicken anti-EF-Tu polyclonal antibody from one of two immunized hens as a capture antibody and one of three different monoclonal antibodies as a detection antibody, namely the antibodies designated Mo683B, Mo524D and Mo521F. Briefly, the ELISA was performed using the following three standard ELISA protocols:

### a) Antibody Titrations

1. An ELISA plate was coated with chicken polyclonal antibodies from hen number 49 (“Ch49”) at a concentration of 10 μg/ml or 54 ml. Titrating amounts of recombinant EF-Tu from dilutions of 11n 2000 to 1 in 2,275,328 from a stock solution at A₂₈₀=0.235 were added. Antigen detection was performed using the monoclonal antibody Mo683B at a concentration of 5 μg/ml or 2.5 μg/ml followed by detection using Sheep anti-mouse Ig HRP conjugate at a 1:5000 (v/v) dilution. Data are presented in FIG. 92.

2. An ELISA plate was coated with chicken polyclonal antibodies from hen number 50 (“Ch50”) at a concentration of 5 μg/ml. Titrating amounts of recombinant EF-Tu were added at concentrations ranging from about 75.7 ng/ml to about 73.9 pg/ml. Antigen detection was performed using one of the monoclonal antibodies designated Mo683B, Mo524D or Mo521F at a concentration of 5 μg/ml followed by detection using Sheep anti-mouse Ig HRP conjugate at a 1:5000 (v/v) dilution. Data are presented in FIG. 93.

3. An ELISA plate was coated with chicken polyclonal antibodies from either hen number 49 or hen number 50 (“Ch50”) at a concentration of 2.5 μg/ml. Titrating amounts of recombinant EF-Tu were added at concentrations ranging from about 122.9 ng/ml to about 120 pg/ml. Antigen detection was performed using the monoclonal antibody designated Mo683B at a concentration of 2.5 m/ml followed by detection using Sheep anti-mouse Ig HRP conjugate at a 1:5000 (v/v) dilution. Data are presented in FIG. 94.

### b) Preferred Antibody Orientation

This protocol provides an example of how the inventors determine optimum capture and detection antibodies, and appropriate antibody concentrations for use. Briefly, the wells of an ELISA plate are coated overnight with 50 μl of a 5 μg/ml concentration or 10 μg/ml concentration of Mo683B antibody, or a 2.5 μg/ml concentration or 5 mg/ml concentration of Ch49 antibody. Following blocking and washing to remove unbound antibody, recombinant EF-Tu protein is diluted serially 1:3 (v/v) from 500 ng/ml starting concentration to 22.86 pg/ml, and 50 μl aliquots of each dilution are added the wells of the antibody-coated ELISA plates. Following incubation for 1 hour and washing to remove unbound antigen, 50 μl of the alternate detection antibody (i.e., Ch49 at 2.5 μg/ml or 5 μg/ml or 10 μg/ml for detecting EF-Tu-Mo683B complexes, or Mo683B at 5 μg/ml or 10 μg/ml or 20 μg/ml for detecting EF-Tu-Ch49 complexes), is contacted with the bound antigen-body complexes. Following incubation at room temperature for 1 hour, plates are washed, incubated with 50 μl of a 1:5000 (v/v) dilution of secondary antibody (i.e., sheep anti-chicken IgG for detecting Ch49 or sheep anti-mouse IgG for detecting Mo683B) conjugated to horseradish peroxidase (HRP), washed, incubated with TMB for 30 mins, and absorbance at 450-620 nm is then determined after subtraction of background.

In a further experiment, wells of an ELISA plate are coated overnight with 50 μl of a 5 μg/ml concentration of Ch49 or Mo683B antibody. Following blocking and washing to remove unbound antibody, recombinant EF-Tu protein is diluted serially 1:3 (v/v) from 500 ng/ml starting concentration to 22.86 pg/ml, and 50 μl aliquots of each dilution are added the wells of the antibody-coated ELISA plates (x-axis). Following incubation for 1 hour and washing to remove unbound antigen, the alternate detection antibody i.e., Ch49 for detecting EF-Tu-Mo683B complexes and Mo683B for detecting EF-Tu-Ch49 complexes, is contacted with the bound antigen-body complexes at a concentration of 2 μg/ml. Following incubation at room temperature for 1 hour, plates are washed, incubated with 50 μl of a 1:5000 (v/v) dilution of secondary antibody (i.e., sheep anti-chicken IgG for detecting Ch49 or sheep anti-mouse IgG for detecting Mo683B) conjugated to horseradish peroxidase (HRP), washed, incubated with TMB for 30 mins, and absorbance at 450-620 nm is then determined after subtraction of background (y-axis).

### 5. Amplified Sandwich ELISA for Detection of M. Tuberculosis EF-Tu Protein

This example demonstrates factors that are optimized for detection of EF-Tu protein by sandwich ELISA using the polyclonal antibody Ch49 as a capture reagent and the monoclonal antibody designated Mo683B as a detection reagent. This antibody pair was selected because, for example, titration data in the preceding example indicated that the antibody combination detected less EF-Tu protein than combinations of other antibodies described herein. It is to be understood that these protocols provided for exemplifications only and can be readily adapted for other antibody combinations not specifically described.

In this example, amplified ELISA was performed essentially as described in this example and in Example 1, using polyclonal sera Ch49 as a capture reagent and biotinylated monoclonal antibody Mo683B (i.e., “Mo683B-Bio) as a detection reagent. An ELISA plate was coated overnight with capture antibody Ch49 at 2 μg/ml concentration. Following washing to remove unbound antibody, recombinant EF-Tu protein was diluted serially 1:10 (v/v) from 100 ng/ml starting concentration to 1.0 pg/ml, and 50 μl aliquots of each dilution were added the wells of the antibody-coated ELISA plates (x-axis). Following incubation for 1 hour and washing to remove unbound antigen, biotinylated antibody Mo683B-Bio was contacted with the bound antigen-body complexes at 2.0 μg/ml concentration. Following incubation at room temperature for 1 hour, plates were washed, and incubated with 50 μl of a 1:5,000 (v/v) dilution of a secondary antibody consisting of HRP-conjugated sheep anti-mouse IgG (standard sandwich ELISA) or with 50 μl of a 1:2,500 (v/v) dilution of HRP80-streptavidin. Poly-40 streptavidin-HRP conjugate may also be employed in place of HRP80-streptavidin. Plates were then incubated for a further one hour at room temperature, and washed as before. Finally, all samples were incubated with TMB for 30 mins (standard ELISA) or 10 mins (amplified ELISA). Absorbance was determined at 450-620 nm.

As shown in FIG. 95, better results were obtained using a streptavidin poly-80 horseradish peroxidase (HRP) with biotinylated Mo683B, compared to the standard sandwich ELISA described herein above. More particularly, data indicate significant enhancement of detection using the amplified sandwich ELISA. The limit of detection of this amplified sandwich ELISA is about 154 pg/ml EF-Tu protein. This compares favourably to the observed limit of detection of the standard sandwich ELISA of about 2.172 ng/ml EF-Tu protein.

In a separate experiment under similar conditions, the LOD for amplified sandwich ELISA under these conditions was only 46 pg/mL, compared to 296 pg/mL for standard ELISA (data not shown). Such sensitivity of detection coupled with low background in sandwich ELISA is considered by the inventors to be within useful limits.

### 6. Cross-Reactivity Between Anti-EF-Tu Antibodies and Different M. Tuberculosis Isolates

To further assess the suitability of EF-Tu as a diagnostic marker for the presence of M. tuberculosis in biological samples, and to assess the specificities of antibodies prepared against EF-Tu protein, the inventors compared antibody reactivities in sandwich ELISA performed as described herein above between cellular extracts of the clinical M. tuberculosis strains CSU93 and H878 and the laboratory M. tuberculosis strain H37Rv.

The amplified ELISA described herein was also used to detect recombinant EF-Tu protein in whole cell extracts of the laboratory strain H37Rv and the clinical isolates CSU93 and HN878. Recombinant protein (1.8 μg/ml, 5.6 μg/ml, 16.7 μg/ml, and 50 μg/ml diluted in blocking buffer) was added to whole cell lysates, and the samples assayed in duplicate by amplified sandwich ELISA, essentially as described herein. The concentration of endogenous EF-Tu protein in the whole cell lysates was calculated by interpolation from the standard curve and subtraction of the signal corresponding to the recombinant EF-Tu protein spike.

Levels of endogenous M. tuberculosis EF-Tu protein present in these strains, as determined from two independent experiments are indicated in FIG. 96. Data indicate average EF-Tu levels of about 65 pg/μg cell extract for H37Rv, and only about 10 pg/μg in whole cell extracts of the clinical isolates.

In summary, the data obtained to date thus indicate that the Ch49/Mo683B antibody pair is capable of detecting endogenous EF-Tu protein in whole cell extracts of clinically-relevant and laboratory strains of M. tuberculosis.

### 7. Low Cross-Reactivity Between M. Tuberculosis and Non-Mycobacteria Pathogens

To further assess the suitability of EF-Tu as a diagnostic marker for the presence of M. tuberculosis in biological samples, the inventors compared antibody cross-reactivities in amplified sandwich ELISA performed between cellular extracts of M. tuberculosis strain H37Rv (a laboratory strain), Escherichia coli, Bacillus subtilis or Pseudomonas aeruginosa.

Briefly, amplified sandwich ELISA was performed as described herein using different concentrations of recombinant EF-Tu protein and 2 mg/ml and 500 μg/ml cellular extracts of Escherichia coli, Bacillus subtilis or Pseudomonas aeruginosa.

Data presented in FIG. 97 show low cross-reactivity of antibodies against M. tuberculosis EF-Tu with Escherichia coli, Bacillus subtilis or Pseudomonas aeruginosa cellular extracts under the conditions tested. In particular there was little or no signal differential between the concentrations of cellular extracts tested, and the signal obtained were not significantly above background. In contrast, the assay detected less than 1 ng/ml recombinant EF-Tu protein.

### 8. Cross-Reactivity Between Different Mycobacteria Species

To further assess the suitability of EF-Tu as a diagnostic marker for the presence of M. tuberculosis in biological samples, and to assess the specificities of antibodies prepared against EF-Tu protein, the inventors compared antibody reactivities in amplified sandwich ELISA performed as described herein above between cellular extracts of the Mycobacteria species M. tuberculosis, M. avium and M. intracellulaire.

Briefly, an ELISA plate was coated overnight with capture antibody Ch49. Following washing to remove unbound antibody, a cellular extract from each Mycobacteria species was added to the wells of the antibody-coated ELISA plates. As a negative control for each assay, buffer without cellular extract was used. Following incubation for 1 hour and washing to remove unbound antigen, detection antibody Mo683B-Bio was contacted with the bound antigen-body complexes. Following incubation at room temperature for 1 hour, plates were washed, incubated with 50 μl of 1:2,500 (v/v) dilution of HRP80-streptavidin. Poly-40 streptavidin-HRP conjugate may also be employed in place of HRP80-streptavidin. Plates were washed again, incubated with TMB for 10 mins, and the absorbance at 450-620 nm was determined. Samples were assayed in duplicate over three dilutions of whole cell extracts. A calibration standard curve was produced based on standardized levels of EF-Tu protein.

Data presented in FIG. 100 show low-level cross-reactivity to M. avium and M. intracellulaire, however much stronger reactivity to M. tuberculosis was clearly evident. For example, antibodies against EF-Tu protein may be employed in conjunction with subsequent culture of M. tuberculosis from EF-Tu-positive clinical specimens.

The data suggest that EF-Tu is a suitable marker for detection of Mycobacteria generally or for specific detection of M. tuberculosis depending upon the sensitivity of the assay conditions employed. Antibodies against EF-Tu may be employed solus or simultaneously with one or more antibodies against M. tuberculosis Rv1265 and/or M. tuberculosis BSX protein and/or M. tuberculosis KARI and/or M. tuberculosis S9 protein as described herein, and preferably with antibodies selected from antibodies to Rv1265, antibodies to BSX and antibodies to S9, which have low cross-reactivity to the other Mycobacteria tested.

### 9. Detection of EF-Tu Protein in Clinical Samples

To further assess the suitability of EF-Tu as a diagnostic marker for the presence of M. tuberculosis in biological samples, the inventors determined the ability of antibodies to detect endogenous EF-Tu protein in clinical samples obtained from TB-positive subjects who had been diagnosed previously on the basis of smear test and M. tuberculosis culture assay results.

In one example, recombinant M. tuberculosis EF-Tu protein (3.3 ng/ml) was mixed with serial dilutions of sputum (FIG. 96) or plasma (FIG. 97) and tested in an amplified sandwich ELISA assay format described above without replacement amplification, i.e., utilizing Ch49 as capture antibody and a detection system comprising biotinylated Mo683B-Bi monoclonal antibody followed by streptavidin poly-80HRP (“HRP80-streptavidin”).

Under the conditions tested, recombinant EF-Tu could be detected in diluted samples, demonstrating feasibility (FIGS. 98 and 99).

In another example, the ability of antibodies to detect endogenous EF-Tu protein in clinical samples obtained from TB-positive subjects who have been diagnosed previously on the basis of smear test and M. tuberculosis culture assay results is determined essentially as described herein for other markers. Patients are categorized on the basis of both smear and culture test results, and HIV status. Subjects are both smear-negative and culture-negative or alternatively, both smear-positive and culture-positive.

Briefly, standard or amplified sandwich ELISA is performed as described herein above on the sputum samples, which are prepared by a method as described herein, preferably assayed as up to about 20×150 microlitre aliquots under a replacement amplification protocol (see below). ELISA plate is coated overnight with capture antibody Ch49. Following washing to remove unbound antibody, treated sputa are added to the wells of the antibody-coated ELISA plates. As a negative control for each assay, buffer is used. Following incubation for 1 hour and washing to remove unbound antigen, detection antibody Mo683B or Mo683B-Bio is contacted with the bound antigen-body complexes. Following incubation at room temperature for 1 hour, plates were washed, incubated with 50 μl of a secondary antibody (i.e., biotinylated donkey anti-mouse IgG) for Mo683B is employed. Following incubation with Mo683B-Bio detection antibody or biotinylated donkey anti-mouse IgG as appropriate, poly-40 streptavidin-HRP conjugate is added and the samples incubated for 1 hour, washed again, incubated with TMB for 10 mins, and the absorbance at 450-620 nm is determined.

### 10. Evaluation of Signal Inhibition by Sample

To assess whether or not factors are present in biological samples that are to be tested using the sandwich ELISA of the present invention, e.g., in a point-of-care or field test format, recombinant M. tuberculosis EF-Tu protein (3.3 ng/ml) was mixed with serial dilutions of sputum (FIG. 96) or plasma (FIG. 97) and tested in the amplified sandwich ELISA assay format described above i.e., utilizing Ch49 as capture antibody and a detection system comprising biotinylated Mo683B monoclonal antibody followed by streptavidin poly-80HRP (“HRP80-streptavidin”).

Under the conditions tested, significant suppression of signal was observed for undiluted or diluted sputum or plasma-containing samples (FIGS. 98 and 99). In both cases, suppression of signal or quenching could be ablated by diluting sputum or plasma in blocking buffer.

### 11. Relative Levels of EF-Tu Protein Detectable in Mycobacterium Cells

To further assess the suitability of EF-Tu as a diagnostic marker for Mycobacteria infection, the levels of EF-Tu protein were determined in whole cell lysates of the M. tuberculosis strains H37Rv, CSU93 and HN878, and in M. tuberculosis, M. avium and M. intracellulaire, relative to 10 other M. tuberculosis antigens including BSX, KARI, S9, Rv1265, P5CR, and TetR-like protein described herein.

Amplified sandwich ELISA was performed essentially as described in this example and in Example 1, to identify relative levels of each antigen according to the standard protocol, with calibration standards included to permit quantitation.

Data shown in FIGS. 124-125 indicate that EF-Tu is expressed at low levels in M. tuberculosis cells compared to BSX, KARI, Rv1265 and S9 proteins. The EF-Tu protein is also expressed at much higher levels in H37Rv than the clinical isolates tested.

On the other hand, data shown in FIGS. 126-131 indicate that EF-Tu protein is expressed at much higher levels in M. tuberculosis compared to other Mycobacteria tested.

Overall, these data suggest utility of EF-Tu as a generic single-analyte marker of mycobacteria infection, or as part of a multi-analyte test for mycobacteria infection or M. tuberculosis infection e.g., in combination with BSX and/or KARI and/or S9 and/or Rv1265 proteins. Other combinations are not excluded for multi-analyte testing of M. tuberculosis infection.

### 11. Optimizing the Limits of Detection

To further enhance sandwich ELISA sensitivity, a replacement amplification procedure is employed to employ iterative antigen binding following coating of the ELISA plate with capture antibody. Essentially, this will result in an increased amount of antigen being bound to the capture antibody notwithstanding the 50 μl volume limitations of a 96-well ELISA plate. Briefly, this iterative antigen loading involves repeating the antigen binding step in the sandwich ELISA several times, e.g., 2 or 3 or 4 or 5 times, etc. before washing and adding detection antibody. Naturally, each aliquot of antigen sample is removed following a standard incubation period before the next aliquot is added. The number of iterations can be modified to optimize the assay (e.g., parameters such as signal:noise ratio, detection limit and amount of antigen detected at half-maximum signal), depending upon the nature of the sample being tested (e.g., sample type), without undue experimentation.

In one example, five iterations of sample loading (i.e., a 5× replacement amplification) can be employed to provide a low background signal, and a reduced detection limit of M. tuberculosis EF-Tu protein.

In another example, up to about 20 iterations of sample loading (i.e., up to a 20× replacement amplification) may provide a low background signal, and reduce the detection limit for EF-Tu protein.

### Example 7

### Antigen-Based Diagnosis of Tuberculosis or Infection by M. Tuberculosis Using Antibodies that Bind to M. Tuberculosis P5CR Protein

### 1. Identification of P5CR Protein in TB-Positive Subjects

A protein fragment having a molecular weight of about 15 kDa was recognized in the immune-globulin fraction of plasma from TB⁺ samples. The sequences of five peptides from MALDI-TOF data (SEQ ID Nos: 37-41 inclusive) matched a protein having SwissProt Accession No. Q11141 (SEQ ID NO: 36). The percent coverage of Q11141 by these 5 peptides (SEQ ID NOs: 37-41) was about 19%, suggesting that the peptide fragments were derived from this same protein marker.

The identified protein having the amino acid sequence set forth in SEQ ID NO: 36 was designated as “P5CR”. The estimated molecular weight of the P5CR protein is only about 30.2 kDa, and the estimated isoelectric point is about 4.7.

### 2. Antibodies

### a) Antibodies Prepared Against Peptide Fragments of M. Tuberculosis P5CR Protein

Synthetic peptides comprising amino acid residues 43-61 (SEQ ID NO: 42) or residues 238-255 (SEQ ID NO: 43) of full length P5CR were synthesized according to standard procedures. The peptides can be coupled to keyhole limpet Hemocyanin (KHL) via a maleimidocaproyl-N-hydroxysuccinimide linker. To facilitate detection of antibodies raised against these epitopic peptides, they can also be synthesized with a GSGL spacer and attached to biotin. Rabbits were immunized with synthetic peptides comprising the amino acid sequences set forth in SEQ ID NO: 42 or SEQ ID NO: 43 according to standard procedures. Animal bleeds were obtained. All bloods were was collected in sterile containers and sera collected after clot removal.

Four rabbit antibody preparations were obtained: Antibodies Rb33 and Rb34 having specificity against SEQ ID NO: 43; and antibodies Rb37 and Rb38 having specificity against SEQ ID NO: 42.

Polyclonal antibodies were also produced in chickens using a synthetic peptide comprising the sequence set forth in SEQ ID NO: 42.

To titrate polyclonal antisera produced in chickens, rP5CR protein was immobilized at a concentration of 5 μg/ml onto Nunc immune-plates. The solution was removed and wells blocked using blocking buffer (1% (w/v) casein, 0.1% (v/v) Tween 20, 0.1% (w/v) sodium azide in PBS). Blocking buffer was removed and serum diluted in PBS added and incubated for a sufficient time for antibodies to complex with bound P5CR protein, generally for about 1 hour at room temperature. Plates were washed and binding of the antibody to P5CR protein was detected using HRP-conjugated sheep anti-chicken IgG diluted 1:5000 (v/v) in conjugate diluent buffer. Fifty millilitres (50 ml) of TMB (3,3′,5′,5-Tetramethylbenzidine; Sigma) were added to each well and the plate incubated in the dark for 30 minutes. Development was stopped by addition of 50 μL per well of 0.5M sulphuric acid. The optical density of each well was read with a microtitre plate reader (PowerWaveₓ 340 plate reader, Bio-Tek Instruments Inc., Winooski, Vt.) using a wavelength of 450 nm and an extinction at 620 nm. The titration results are shown in FIG. 101.

For testing rabbit antisera, streptavidin (Sigma Aldrich) was diluted to 5 μg/ml in double-distilled water (ddH₂O) and incubated in a Nunc plate overnight at 4° C. The solution was then flicked out of the plate and 250 μL of blocking buffer (1% (w/v) casein, 0.1% (v/v) Tween 20, 0.1% (w/v) sodium azide in PBS) added to each well and incubated at room temperature for 1 hour. The blocking buffer was flicked out and biotinylated peptide (SEQ ID NO: 42) was added in blocking buffer at 3 μg/ml to 50 μl/well and incubated for one hour at room temperature on a shaker. The plate was washed in an Elx405 Auto Plate Washer (Bio-Tek Instruments Inc., Winooski, Vt.), with 0.5×PBS/0.05% (v/v) Tween 20 solution and excess solution tapped out of the plate onto a paper towel. Rabbit sera were diluted in blocking buffer from 1:500 (v/v) to 1:1,024,000 (v/v) and incubated for 1 hour at 50 μl/well at room temperature on a shaker. Plates were washed with the plate washer using 0.5×PBS/0.05% (v/v) Tween 20 solution, and the excess solution tapped out on a paper towel. Binding of the rabbit antibodies to SEQ ID NO: 42 was detected using HRP-conjugated Sheep anti-rabbit IgG (Chemicon) diluted 1:5000 (v/v) in conjugate diluent buffer. Fifty millilitres (50 ml) were added to each well and incubated for one hour at room temperature on a shaker. Plates were washed with the plate washer using 0.5×PBS and excess solution tapped out on a paper towel. Fifty millilitres (50 ml) of TMB (3,3′,5′,5-Tetramethylbenzidine; Sigma) were added to each well and the plates incubated in the dark for 30 minutes. Development was stopped by addition of 50 μL per well of 0.5M sulphuric acid. The optical density of each well was read with a microtitre plate reader (PowerWaveₓ 340 plate reader, Bio-Tek Instruments Inc., Winooski, Vt.) using a wavelength of 450 nm and an extinction at 620 nm. The sera titration data for SEQ ID NO: 42 are shown in FIG. 102.

Although titration data are not shown for antibodies Rb33 and/or Rb34, similar protocols are used to titrate those polyclonal antibodies against a peptide comprising the sequence of SEQ ID NO: 43.

To titrate peptide i.e., SEQ ID NO: 42, a protocol essentially as described supra was used. However, the biotinylated peptide was titrated from 20,480 pg/ml to 100 pg/ml and the rabbit sera were added to the ELISA at dilutions of 1:500 (v/v) and 1:2000 (v/v). Results of this analysis are shown in FIG. 103. Although titration data are not shown for peptide comprising the sequence of SEQ ID NO: 43, similar protocols are used to titrate that peptide against antibodies Rb33 and/or Rb34.

### c) Recombinant Antibodies Prepared Against M. Tuberculosis P5CR

Recombinant antibodies against P5CR were obtained from AbD Serotec, a division of MorphoSys AG, Germany, who produced the antibodies under contract with the applicant/assignee using standard procedures for recombinant antibody production as described herein.

Several recombinant antibodies preparations were obtained from AbD Serotec, designated Ph4549, Ph4464-4470 and Ph4550.2. In standard ELISA, the antibody designated Ph4550.2 was considered to be a high titer antibody and further diagnostic testing herein was thus conducted using the Ph4550.2 antibody.

### d) Antibodies Prepared Against Recombinant M. Tuberculosis P5CR Protein

Two additional antibody preparations were prepared against the full length recombinant M. tuberculosis protein (SEQ ID NO: 36), by immunization of chickens using standard procedures. Two separate batches of chicken polyclonal antisera, designated “Ch6” and “Ch7” were raised against the P5CR protein, and these were then pooled to produce the antibody designated herein as “Ch6/7”.

Three monoclonal antibodies were also produced against recombinant P5CR protein using standard procedures, and these were designated Mo1027D, Mo1028C and Mo1028D.

The polyclonal antibody preparations against P5CR had the highest titers when assayed using standard procedures (data not shown) and further diagnostic testing herein was conducted using the polyclonal sera designated Ch6/7.

### 3. Validation of P5CR and Antibodies

To further assess the suitability of P5CR as a diagnostic marker for the presence of M. tuberculosis in biological samples, the inventors compared antibody reactivities between cellular extracts of the clinical M. tuberculosis strains CSU93 and HN878, and the laboratory M. tuberculosis strain H37Rv by western blotting using Ch6/7 polyclonal sera or, in a separate set of experiments, the recombinant antibody Ph4550.2.

Briefly, Western blotting was performed on proteins separated by electrophoresis on 10% (w/v) Bis-Tri Nu-PAGE (Invitrogen, Carlsbad Calif., USA) and transferred to PVDF activated membrane (Immobilon-P, Millipore Inc, USA). Following transfer, membranes were incubated in 0.008% DB-71 (Sigma Chemical Co. USA) in 40% (v/v) ethanol/10% (v/v) acetic acid for 7 min, rinsed briefly in 40% (v/v) ethanol/10% (v/v) acetic acid, scanned to visually confirm protein transfer, and rinsed in Tris-buffered saline containing Triton-X100 (TBS-T). Dried membranes were re-activated in methanol, and transferred to blocking buffer (TBS-T containing 1% (w/v) bovine serum albumin) overnight at 4° C. Polyclonal sera Ch6/7 or recombinant antibody Ph4550.2 was diluted to a concentration of 0.5 μg/ml in blocking buffer and incubated separately with membranes for 90 min at room temperature, after which time the membranes were washed in TBS-T, incubated with HRP-conjugated secondary antibody i.e., a sheep anti-chicken IgG-HRP conjugate diluted 1:100,000 (v/v), for 60 min at room temperature, and washed as before. Binding of HRP-secondary antibody conjugate was detected by incubating membranes in SuperSignal™ West “Femto” Maximum Sensitivity Substrate (Pierce, Inc. USA), and visualizing chemiluminescence using the LAS-3000 multi-imager (FujiFilm Inc., Japan).

Immune-reactive bands of about 31 kDa, consistent with the expected molecular masses of M. tuberculosis P5CR protein, were detected in both clinical M. tuberculosis isolates CSU93 and HN878, and in the laboratory strain H37Rv (data not shown), using polyclonal sera. The recombinant antibody also detected M. tuberculosis P5CR protein in the clinical M. tuberculosis isolate CSU93 and in the laboratory strain H37Rv, however binding to the P5CR protein in HN878 was much weaker. In control experiments, recombinant P5CR protein comprising a hexahistidine tag having an estimated molecular mass of about 35 kDa was also detected at the correct position (not shown).

Competition Western blot analysis performed essentially as described in Example 1 indicated that binding of both antibodies to recombinant P5CR protein and endogenous EF-Tu protein could be ablated by pre-incubation of antibodies with excess concentration of unlabelled recombinant P5CR protein (data not shown).

In summary, the available data indicate that recombinant antibody Ph4550.2 and polyclonal sera Ch6/7 bind to M. tuberculosis P5CR protein specifically.

### 4. Sandwich ELISA Using Recombinant Ph4550.2 Antibodies and Polyclonal Ch6/7 Sera

This example demonstrates effective detection of P5CR protein by sandwich ELISA using the recombinant antibody Ph4550.2 as a capture reagent and the polyclonal antibody pool designated Ch6/7 as a detection reagent. This antibody pair was selected because, for example, titration of the pooled antibody preparation Ch6/7, recombinant antibody Ph4550.2 and monoclonal antibody Mo1027D against P5CR protein indicated that Ch6/7 and Ph4550.2 were capable of detecting less protein at 5 μg/ml antibody concentration relative to Mo1027D (FIG. 104).

### a) Preferred Antibody Orientation

In a first set of diagnostic tests, a standard sandwich ELISA is performed to determine optimum capture and detection antibodies, and appropriate antibody concentrations for use. Briefly, two ELISA plates are coated with either Ch6/7 or Ph4550.2 antibodies at 2.5 μg/ml and 5 μg/ml concentrations in blocking buffer. Following washing to remove unbound antibody, 50 μl aliquots of recombinant P5CR protein, diluted serially in blocking buffer 1:2 (v/v) from 500 ng/ml starting concentration to 7.8 ng/ml, are added the wells of the antibody-coated ELISA plates. Following incubation for 1 hour and washing to remove unbound antigen, the alternate detection antibody (i.e., Ph4550.2 for detection of Ch6/7-P5CR complexes and Ch6/7 for detection of Ph4550.2-P5CR complexes) is contacted with the plates at concentrations in the range of 1.25 μg/ml to 5 μg/ml. Following incubation at room temperature for 1 hour, plates are washed as before, incubated with 50W of a 1:5000 (v/v) dilution of donkey anti-mouse IgG conjugated to horseradish peroxidase (HRP), washed as before, incubated with TMB for 30 mins, and the absorbances at 450-620 nm are determined.

A preferred, albeit not essential, orientation of antibodies is selected so as to achieve higher signal per unit of recombinant P5CR protein in sandwich ELISA. It is also preferred to select an antibody orientation that provides minimal cross-reactivity between antibodies, as indicated by a low baseline value in the absence of recombinant protein in the assay.

### b) Limit of Detection

To determine the limits of detection of sandwich ELISA for recombinant M. tuberculosis P5CR protein, the assay was performed using a serial dilution of P5CR protein, in the concentration range from 22.86 pg/ml to 50 ng/ml. Antibody concentrations were also varied, such that concentrations of 2 μg/ml, 5 μg/ml and 10 μg/ml of Ph4550.2 capture antibody was employed, and either 5 μg/ml or 10 μg/ml of Ch6/7 detection antibody was employed.

Data presented in FIG. 105 indicate that, under the assay conditions tested, concentrations as low as about 360 pg/ml M. tuberculosis P5CR protein could be detected using 5 μg/ml Ph4550.2 capture antibody and 5 μg/ml Ch6/7 detection antibody. Lower background signal at 22.86 pg/ml protein was also observed under these conditions, compared to assays performed at higher capture antibody concentrations. However, acceptable signal: noise ratios over a broad range of protein concentrations was also observed using 10 μg/ml Ph4550.2 capture antibody and 5 μg/ml Ch6/7 detection antibody.

### 5. Amplified Sandwich ELISA for Detection of M. Tuberculosis P5CR Protein

The inventors also investigated whether or not a biotinylated antibody and streptavidin poly-HRP conjugate could improve ELISA sensitivity compared to conventional biotin-streptavidin-HRP systems or HRP-conjugated secondary antibodies.

As shown in FIG. 106, better results were obtained using of a streptavidin poly-40 horseradish peroxidase (HRP) with biotinylated donkey anti-chicken IgG to detect bound Ch6/7 antibody in ELISA, compared to these other systems, including HRP-conjugated sheep anti-chicken IgG for detecting the polyclonal detection antibody. Similarly, these results were superior to any conjugates capable of detecting recombinant Ph4550.2 antibody when used as a detection antibody. These data suggested that the Ch6/7 antibody would be superior to Ph4550.2 as a detector antibody in an amplified sandwich ELISA format.

Accordingly, further optimization was performed by varying the concentrations of capture antibody Ph4550.2, detector antibody Ch6/7 and amplified streptavidin poly-80 HRP in an amplified sandwich ELISA within the limits shown in FIG. 107.

Data shown in FIG. 107 indicate optimum detection limits for P5CR protein in amplified sandwich ELISA using 10 μg/ml Ph4550.2 capture antibody and 2.5 μg/ml Ch6/7 detection antibody with a 1:10,000 dilution (v/v) amplified streptavidin poly-80 HRP.

In a synthesis of the tests described in the preceding experiments, an amplified sandwich ELISA for P5CR protein was produced. Briefly, an ELISA plate was coated with Ph4550.2 antibody at 5 μg/ml concentration in blocking buffer. Following washing to remove unbound antibody, 50 μl aliquots of recombinant P5CR protein, diluted serially in blocking buffer 1:2 (v/v) from 100 ng/ml starting concentration to 1 pg/ml, was added the wells of the antibody-coated ELISA plates. Following incubation for 1 hour and washing to remove unbound antigen, 2 μg/ml concentration of Ch6/7 antibody in blocking buffer was contacted with the plate. Following incubation at room temperature for 1 hour, the plate was washed as before, incubated with 50 μl of a 1:200,000 (v/v) dilution of biotinylated donkey anti-chicken IgG and a 1:20,000 (v/v) dilution of streptavidin poly-80 HRP, washed as before, incubated with TMB for 30 mins, and the absorbances at 450-620 nm determined.

Data presented in FIG. 108 compare the limits of detection of this amplified sandwich ELISA to a standard sandwich ELISA employing HRP-conjugated sheep anti-chicken IgG as a detection reagent for the Ch6/7 antibodies. The limit of detection of this amplified sandwich ELISA is about 48 pg/ml P5CR protein, with half-maximum detection of about 1 ng/ml P5CR protein. This compares favourably with the standard assay's sensitivity.

### 6. Evaluation of Signal Inhibition by Sample

To assess whether or not factors are present in biological samples that are to be tested using the sandwich ELISA of the present invention, e.g., in a point-of-care or field test format, different concentrations of recombinant M. tuberculosis P5CR protein (i.e., 0-30 pg/ml) were mixed with serial dilutions of plasma (FIG. 109) or sputum (FIG. 110) and tested in the amplified sandwich ELISA assay format described above without replacement amplification, i.e., utilizing Ph4550.2 as capture antibody, Ch6/7 detector antibody, and a detection system comprising a biotinylated donkey anti-chicken IgG and streptavidin poly-80HRP (“HRP80-streptavidin”).

Under the conditions tested, significant suppression of signal was observed for undiluted plasma-containing samples (FIG. 110), however the loss in signal was reduced by diluting plasma samples at least about 1:1 (v/v). A 1:8 (v/v) dilution of plasma sample in blocking buffer resulted in a signal recovery of about 88% compared to the signal obtained using peptide mixed with blocking solution alone i.e., no plasma.

Suppression of signal was also observed for undiluted sputum-containing samples (FIG. 111), however this was proportionately much less than for plasma, and a 1:1 (v/v) dilution of sputum in blocking buffer resulted in a signal enhancement compared to the signal obtained using peptide mixed with blocking solution alone i.e., no sputum.

In summary, the available data suggest that the amplified sandwich ELISA can detect less than about 3.3 pg/ml P5CR protein in biological samples, including diluted plasma or sputum.

### 7. Low Cross-Reactivity Between M. Tuberculosis and Non-Mycobacteria Pathogens

To further assess the suitability of P5CR as a diagnostic marker for the presence of M. tuberculosis in biological samples, the inventors compared antibody cross-reactivities in the amplified sandwich ELISA performed as described in this example, between different concentrations of recombinant P5CR protein and 100 ng/ml and 100 μg/ml cellular extracts of yeast, Escherichia coli, Bacillus subtilis or Pseudomonas aeruginosa.

Data presented in FIG. 112 show low cross-reactivity of antibodies against M. tuberculosis P5CR with yeast, Escherichia coli, Bacillus subtilis or Pseudomonas aeruginosa cellular extracts under the conditions tested. In particular there was little or no signal differential between the concentrations of cellular extracts tested, and the signal obtained were not significantly above background. In contrast, the assay detected less than 1 ng/ml recombinant P5CR protein.

### 8. Cross-Reactivity Between Anti-EF-Tu Antibodies and Different M. Tuberculosis Isolates

The amplified ELISA described herein was also used to detect recombinant P5CR protein in whole cell extracts of the laboratory strain H37Rv and the clinical isolates CSU93 and HN878. Recombinant protein (1.8 μg/ml, 5.6 μg/ml, 16.7 μg/ml, and 50 μg/ml diluted in blocking buffer) was added to whole cell lysates, and the samples assayed in duplicate by amplified sandwich ELISA, essentially as described herein. The concentration of endogenous P5CR protein in the whole cell lysates was calculated by interpolation from the standard curve and subtraction of the signal corresponding to the recombinant P5CR protein spike.

Levels of endogenous M. tuberculosis P5CR protein present in these strains, as determined from two independent experiments is indicated in FIG. 113. Data indicate average P5CR levels in the range of about 5-9 pg/ml of whole cell extract.

In summary, the data obtained to date thus indicate that the Ch6/7 polyclonal sera and recombinant antibody Ph4550.2 are capable of detecting endogenous P5CR protein in whole cell extracts of clinically-relevant and laboratory strains of M. tuberculosis.

### 9. Low Cross-Reactivity Between Different Mycobacteria Species

To further assess the suitability of P5CR as a diagnostic marker for the presence of M. tuberculosis in biological samples, and to assess the specificities of antibodies prepared against P5CR protein, the inventors compared antibody reactivities in amplified sandwich ELISA performed as described herein above between cellular extracts of the Mycobacteria species M. tuberculosis, M. avium and M. intracellulaire.

Briefly, an ELISA plate was coated overnight with capture antibody Ph4550.2. Following washing to remove unbound antibody, a cellular extract from each Mycobacteria species was added to the wells of the antibody-coated ELISA plates. As a negative control for each assay, buffer without cellular extract was used. Following incubation for 1 hour and washing to remove unbound antigen, detection antibody Ch6/7 was contacted with the bound antigen-body complexes. Following incubation at room temperature for 1 hour, plates were washed, incubated with 50 μl of diluted secondary antibody (e.g., biotinylated donkey anti-chicken IgG) for 1 hour, followed by 1:2,500 (v/v) dilution of HRP80-streptavidin (poly-40 streptavidin-HRP conjugate may also be employed in place of HRP80-streptavidin), and then washed again, incubated with TMB for 10 mins, and the absorbance at 450-620 nm was determined. Samples were assayed in duplicate over three dilutions of whole cell extracts. A calibration standard curve was produced based on standardized levels of P5CR protein.

Data presented in FIG. 113 show detectable cross-reactivity between two Mycobacteria species, M. tuberculosis and M. intracellulaire, however no detection of M. avium. These data indicate that M. tuberculosis P5CR protein may be less suited for species-specific detection of M. tuberculosis under these assay conditions or using the selected antibody pair. This does not abrogate utility of antibodies against P5CR protein in a general single-analyte diagnostic test, or alternatively, as part of a multi-analyte test in conjunction with antibodies against KARI protein, and/or a species-specific marker of M. tuberculosis such as described herein or known in the art.

For example, antibodies against P5CR protein may be employed in conjunction with subsequent culture of M. tuberculosis from P5CR-positive clinical specimens.

Alternatively, antibodies against KARI and antibodies against P5CR may be used as a generic Mycobacteria test, wherein the presence of KARI cross-reactivity and the absence of P5CR cross-reactivity is indicative of Mycobacteria other than M. tuberculosis or M. intracellulaire e.g., because it indicates M. avium or wherein cross-reactivity to both KARI and P5CR indicates M. tuberculosis or M. intracellulaire.

Alternatively, or in addition, antibodies against KARI protein and/or P5CR protein may be employed simultaneously with one or more antibodies against M. tuberculosis BSX and/or M. tuberculosis S9 protein and/or M. tuberculosis Rv1265 protein as described herein which have low cross-reactivity to the other Mycobacteria tested. In interpreting such a multi-analyte test, binding of antibodies against both KARI and P5CR proteins indicates the presence M. tuberculosis or M. intracellulaire in the clinical sample and the additional binding of antibodies against M. tuberculosis BSX and/or M. tuberculosis S9 protein and/or M. tuberculosis Rv1265 protein indicates a greater likelihood of M. tuberculosis infection.

### 10. Relative Levels of P5CR Protein Detectable in Mycobacterium Cells

To further assess the suitability of P5CR as a diagnostic marker for Mycobacteria infection, the levels of P5CR protein were determined in whole cell lysates of the M. tuberculosis strains H37Rv, CSU93 and HN878, and in M. tuberculosis, M. avium and M. intracellulaire, relative to 10 other M. tuberculosis antigens including BSX, EF-Tu, KARI, Rv1265, S9 and TetR-like protein described herein.

Amplified sandwich ELISA was performed essentially as described herein and in Example 1, to identify relative levels of each antigen according to the standard protocol, with calibration standards included to permit quantitation.

Data shown in FIGS. 124-125 indicate that P5CR is expressed at low levels in all three M. tuberculosis strains tested when compared to BSX, KARI, Rv1265 and S9, and even when compared to EF-Tu, however is expressed in laboratory and clinically-relevant strains.

Data shown in FIGS. 126-131 indicate that P5CR protein is also expressed at low levels in Mycobacteria generally, as indicated by the low expression in M. tuberculosis, M. avium and M. intracellulaire compared to e.g., BSX, KARI, Rv1265 and S9. Notwithstanding the low expression of P5CR, in the experiment shown in FIG. 127, there is a greater differential in expression in M. tuberculosis and M. intracellulaire than observed from FIG. 113.

Taken together, these data suggest utility of P5CR as a generic single-analyte marker of mycobacteria infection, or as part of a multi-analyte test for mycobacteria infection e.g., in combination with KARI, or as part of a multi-analyte test for M. tuberculosis infection e.g., in combination e.g., with BSX and/or S9 and/or Rv1265. Other combinations are not excluded for multi-analyte testing of M. tuberculosis infection.

### 11. Optimizing the Limits of Detection

To further enhance sandwich ELISA sensitivity, a replacement amplification procedure is employed to employ iterative antigen binding following coating of the ELISA plate with capture antibody. Essentially, this will result in an increased amount of antigen being bound to the capture antibody notwithstanding the 50 μl volume limitations of a 96-well ELISA plate. Briefly, this iterative antigen loading involves repeating the antigen binding step in the sandwich ELISA several times, e.g., 2 or 3 or 4 or 5 times, etc. before washing and adding detection antibody. Naturally, each aliquot of antigen sample is removed following a standard incubation period before the next aliquot is added. The number of iterations can be modified to optimize the assay (e.g., parameters such as signal:noise ratio, detection limit and amount of antigen detected at half-maximum signal), depending upon the nature of the sample being tested (e.g., sample type), without undue experimentation.

For example, up to about 20 iterations of sample loading (i.e., up to a 20× replacement amplification) may provide a low background signal, and reduce the detection limit for P5CR protein.

### Example 8

### Antigen-Based Diagnosis of Tuberculosis or Infection by M. Tuberculosis Using Antibodies that Bind to M. Tuberculosis TetR-like Protein

### 1. Identification of TetR-Like Protein as a Diagnostic Marker of M. Tuberculosis Infection

Protein fragments were recognized in the immune-globulin fraction of plasma and sputum from TB⁺ samples. The sequences of six peptides from MALDI MS data of plasma samples (SEQ ID Nos: 45-56 inclusive) and the sequences of a further four peptides from MALDI MS data of sputum (SEQ ID NOs: 51-54) matched a protein having SwissProt Accession No. 053310 (SEQ ID NO: 44). The percent coverage of 053310 by the 6 plasma-derived peptides (SEQ ID NOs: 45-56) was about 22%, and the percentage coverage of 053310 by the 4 sputum-derived peptides (SEQ ID NOs: 51-54) was 33% suggesting that the peptide fragments in both cases were derived from this same protein marker.

The identified protein having the amino acid sequence set forth in SEQ ID NO: 44 was designated as “TetR” or simply “TetR”. The estimated molecular weight of TetR is about 23.1 kDa, and the estimated isoelectric point is about 4.9.

2. Antibodies that Bind to TetR-like Protein of M. Tuberculosis or TetR-Derived Peptides

### a) Polyclonal Antibodies Prepared Against Synthetic Peptides

Synthetic peptides comprising amino acid residues 147-174 (SEQ ID NO: 55) or residues 113-127 (SEQ ID NO: 56) of full length TetR were synthesized according to standard procedures. These peptides can be coupled separately to keyhole limpet Hemocyanin (KHL) via a maleimidocaproyl-N-hydroxysuccinimide linker.

To facilitate detection of antibodies raised against an epitope of SEQ ID NO: 44, the peptides can also be synthesized separately, each with a GSGS spacer and attached to biotin.

Chickens and rabbits were immunized respectively with recombinant protein comprising the sequence set forth in SEQ ID NO: 44, and with a synthetic peptide comprising the amino acid sequence set forth in SEQ ID NO: 55 according to standard procedures. Animal bleeds were obtained. All bloods were collected in sterile containers and serum collected after clot removal.

To titrate antisera against recombinant protein (SEQ ID NO: 44) produced in chickens, the recombinant protein immunogen was immobilized at a concentration of 5 μg/ml onto Nunc immune-plates. The solution was removed and wells blocked using blocking buffer (1% (w/v) casein, 0.1% (v/v) Tween 20, 0.1% (w/v) sodium azide in PBS). Blocking buffer was removed and serum diluted in the range 1:500 (v/v) to 1:1,024,000 (v/v) in PBS added and incubated for a sufficient time for antibodies to complex with bound putative transcriptional regulatory protein TetR, or TetR-derived peptide, generally for about 1 hour at room temperature. Plates were washed and binding of the antibody to TetR was detected using HRP-conjugated sheep anti-chicken IgG diluted 1:5000 (v/v) in conjugate diluent buffer. Fifty millilitres (50 ml) of TMB (3,3′,5′,5-Tetramethylbenzidine; Sigma) were added to each well and the plate incubated in the dark for 30 minutes. Development was stopped by addition of 50 μL per well of 0.5M sulphuric acid. The optical density of each well was read with a microtitre plate reader (PowerWaveₓ 340 plate reader, Bio-Tek Instruments Inc., Winooski, Vt.) using a wavelength of 450 nm and an extinction at 620 nm. The titration results are shown in FIG. 114.

For testing rabbit antisera against a synthetic peptide comprising SEQ ID NO: 55, streptavidin (Sigma Aldrich) was diluted to 5 μg/ml in double-distilled water (ddH₂O) and incubated in a Nunc plate overnight at 4° C. The solution was then flicked out of the plate and 250 μL of blocking buffer (1% (w/v) casein, 0.1% (v/v) Tween 20, 0.1% (w/v) sodium azide in PBS) added to each well and incubated at room temperature for 1 hour. The blocking buffer was flicked out and biotinylated peptide (SEQ ID NO: 55) diluted to a concentration range of 204.8 ng/ml to 100 pg/ml was added in blocking buffer at 3 μg/ml to 50 μl/well and incubated for one hour at room temperature on a shaker. The plate was washed in an Elx405 Auto Plate Washer (Bio-Tek Instruments Inc., Winooski, Vt.), with 0.5×PBS/0.05% (v/v) Tween 20 solution and excess solution tapped out of the plate onto a paper towel. Rabbit sera and preimmune sera were diluted in blocking buffer 1:500 (v/v) or 1:2000 (v/v) and incubated for 1 hour at 50 μl/well at room temperature on a shaker. Plates were washed with the plate washer using 0.5×PBS/0.05% (v/v) Tween 20 solution, and the excess solution tapped out on a paper towel. Binding of the rabbit antibodies to SEQ ID NO: 55 was detected using HRP-conjugated Sheep anti-rabbit IgG (Chemicon) diluted 1:5000 (v/v) in conjugate diluent buffer. Fifty millilitres (50 ml) were added to each well and incubated for one hour at room temperature on a shaker. Plates were washed with the plate washer using 0.5×PBS and excess solution tapped out on a paper towel. Fifty millilitres (50 ml) of TMB (3,3′,5′,5-Tetramethylbenzidine; Sigma) were added to each well and the plates incubated in the dark for 30 minutes. Development was stopped by addition of 50 μL per well of 0.5M sulphuric acid. The optical density of each well was read with a microtitre plate reader (PowerWaveₓ 340 plate reader, Bio-Tek Instruments Inc., Winooski, Vt.) using a wavelength of 450 nm and an extinction at 620 nm. The data are shown in FIG. 115.

### b) Monoclonal Antibodies Prepared Against Recombinant Protein

The full length recombinant TetR-like protein (SEQ ID NO: 44) was used as an antigen for monoclonal antibody production, according to standard procedures. Approximately 2 mg of protein was provided to NeoClone, Madison, Wis., USA for generation of monoclonal antibodies according to their standard protocol. About 1 mg of the protein was provided as biotinylated peptide for quality control. Five BALB/cByJ female mice were immunized with protein according to Neoclone's standard immunization process. Test bleeds of the immunized mice were performed at regular intervals for use in the quality control sera ELISAs using biotinylated peptide. Polyclonal sera having the highest titer were determined using ELISA. Mice having polyclonal antibody titers of at least 1,000 were used for the ABL-MYC infection process. For each monoclonal antibody to be produced, the spleens of 3 mice having the highest titers of polyclonal antibodies cross-reactive with peptide antigen were used for the ABL-MYC infection, according to NeoClone's standard infection procedure. For each monoclonal antibody to be produced, the splenocytes of the ABL-MYC-infected mice were transplanted into approximately 20 naive mice. Ascites fluid developed in the transplanted mice were isolated and screened for cells producing monoclonal antibodies (mAbs) that bind to the target peptide antigen.

Six cell lines (i.e., plasmacytomas) producing distinct mAbs designated Mo784A, Mo784D, Mo784F, Mo785E, Mo785F and Mo785C were isolated. Binding affinities and isotype specificities of the mAbs were confirmed using ELISA. The mAbs were provided in 1 ml aliquots (approximately) in ascites, together with the associated cell line.

### 3. Standard Sandwich ELISA

This example demonstrates effective detection of TetR-like protein by sandwich ELISA using the polyclonal and monoclonal antibodies described in Example 4, and optimization of standard ELISA using a pool of chicken polyclonal antibodies Ch4 (=Pink 4) and Ch5 (=Pink 5) i.e., Ch4/5 as a capture reagent and the monoclonal antibodies 784F and Mo785E as a detection reagent. The antibody pair Ch4/5 and Mo785E was eventually selected due to, for example, of their higher signal:noise ratio in sandwich ELISA than other antibody combinations described herein.

### a) Preferred Antibody Combination

In a first set of diagnostic tests, a standard sandwich ELISA was performed to determine optimum capture and detection antibodies, and appropriate antibody concentrations for use.

In one example, a standard sandwich ELISA was performed using the polyclonal antisera RCP 18 (=Rb18) as a capture antibody and a pool of polyclonal antibodies designated “Ch4/5” which comprises the polyclonal antibodies Ch4 (=antibody “Pink 4” referred to herein) and Ch5 (=antibody “Pink 5” referred to herein) as detector antibody. Briefly, the wells of an ELISA plate were coated overnight with 50 μl of RCP18 (Rb18) antibody at 5 μg/ml or 10 μg/ml concentration. Following blocking and washing to remove unbound antibody, recombinant TetR-like protein was diluted from 50 ng/ml starting concentration to 80 pg/ml, and 50 μl aliquots of each dilution were added the wells of the antibody-coated ELISA plates. Following incubation for 1 hour and washing to remove unbound antigen, the detection antibody i.e., Ch4/5 for detecting TetR-RCP18 complexes was contacted with the bound antigen-body complexes at a concentration of 5 μg/ml or 10 μg/ml or 20 μg/ml. Following incubation at room temperature for 1 hour, plates were washed, incubated with 50 μl of a 1:5000 (v/v) dilution of secondary antibody (i.e., sheep anti-chicken IgG for detecting Ch4/5) conjugated to horseradish peroxidase (HRP), washed, incubated with TMB for 30 mins, and absorbance at 450-620 nm was determined after subtraction of background.

Data presented in FIG. 116 suggest that the combination of 5 μg/ml RCP18 as capture antibody and 5 μg/ml Ch4/5 as detector antibody is preferred in this sandwich ELISA format.

In a second example, a standard sandwich ELISA was performed using a pool of polyclonal antibodies designated “Ch4/5” which comprises the polyclonal antibodies Ch4 (=antibody “Pink 4” referred to herein) and Ch5 (=antibody “Pink 5” referred to herein) as capture antibody, and the polyclonal antisera RCP18 (=Rb18) as a detector antibody. Briefly, the wells of an ELISA plate were coated overnight with 50 μl of Ch4/5 antibody at 5 μg/ml or 10 μg/ml concentration. Following blocking and washing to remove unbound antibody, recombinant TetR-like protein was diluted from 50 ng/ml starting concentration to 80 pg/ml, and 50 μl aliquots of each dilution were added the wells of the antibody-coated ELISA plate. Following incubation for 1 hour and washing to remove unbound antigen, the detection antibody i.e., RCP18 for detecting TetR-Ch4/5 complexes was contacted with the bound antigen-body complexes at a concentration of 5 μg/ml or 10 μg/ml or 20 μg/ml. Following incubation at room temperature for 1 hour, plates were washed, incubated with 50 μl of a 1:5000 (v/v) dilution of secondary antibody (i.e., sheep anti-rabbit IgG for detecting Ch4/5) conjugated to horseradish peroxidase (HRP), washed, incubated with TMB for 30 mins, and absorbance at 450-620 nm was determined after subtraction of background.

Data presented in FIG. 117 suggest that the combination of 5 μg/ml Ch4/5 as capture antibody and 5 μg/ml RCP 18 as detector antibody is preferred in this sandwich ELISA format, and marginally improved compared to the reverse orientation of antibodies shown in FIG. 116.

In a further diagnostic test, a standard sandwich ELISA was performed using a pool of polyclonal antibodies designated “Ch4/5” which comprises the polyclonal antibodies Ch4 (=antibody “Pink 4” referred to herein) and Ch5 (=antibody “Pink 5” referred to herein) as capture antibody, and one of two monoclonal antibody preparations designated 784F and Mo785E as detector antibody. The wells of an ELISA plate were coated overnight with 50 of Ch4/5 antibody at 500 ng/ml or 1 μg/ml or 2 μg/ml or 4 μg/ml or 8 μg/ml concentration. Following blocking and washing to remove unbound antibody, recombinant TetR-like protein was diluted from 5 ng/ml starting concentration to 2.29 μg/ml, and 50 μl aliquots of each dilution were added the wells of the antibody-coated ELISA plates. Following incubation for 1 hour and washing to remove unbound antigen, the detection antibody i.e., 784F or Mo785E for detecting TetR-Ch4/5 complexes was contacted with the bound antigen-body complexes at a concentration of 2 μg/ml. Following incubation at room temperature for 1 hour, plates were washed, incubated with 50 μl of a 1:5000 (v/v) dilution of secondary antibody (i.e., sheep anti-mouse IgG for detecting the mouse monoclonal antibodies) conjugated to horseradish peroxidase (HRP), washed, incubated with TMB for 30 mins, and absorbance at 450-620 nm was determined after subtraction of background.

Data presented in FIG. 118 suggest that the Mo785E monoclonal antibody provides the lowest background signal and, when combined with the Ch4/5 capture antibody, provides higher signals than the rabbit polyclonal RCP18. The combination of 500 ng/ml Ch4/5 as capture antibody and 2 μg/ml Mo785E as detector antibody provided the lowest background signal, however the combination of 2 μg/ml Ch4/5 as capture antibody and 2 μg/ml Mo785E as detector antibody provided the highest signal:noise ratio in this sandwich ELISA format.

### 3. Validation of Tet-R-like Protein and Antibodies

Briefly, Western blotting was performed on proteins separated by electrophoresis on 10% (w/v) Bis-Tri Nu-PAGE (Invitrogen, Carlsbad Calif., USA) and transferred to PVDF activated membrane (Immobilon-P, Millipore Inc, USA), and probed separately with monoclonal antibody Mo785E or polyclonal antibody Ch4/5. Following transfer, membranes were incubated in 0.008% DB-71 (Sigma Chemical Co. USA) in 40% (v/v) ethanol/10% (v/v) acetic acid for 7 min, rinsed briefly in 40% (v/v) ethanol/10% (v/v) acetic acid, scanned to visually confirm protein transfer, and rinsed in Tris-buffered saline containing Triton-X100 (TBS-T). Dried membranes were re-activated in methanol, and transferred to blocking buffer (TBS-T containing 1% (w/v) bovine serum albumin) overnight at 4° C. Monoclonal antibody Mo785E or polyclonal antibody Ch4/5 was diluted to a concentration of 0.5 μg/ml in blocking buffer and incubated with the membranes for 90 min at room temperature, after which time the membranes were washed in TBS-T, incubated with HRP-conjugated secondary antibody i.e., a sheep anti-mouse IgG-HRP conjugate for Mo785E or donkey anti-chicken IgG-HRP conjugate for Ch4/5, diluted 1:100,000 (v/v), for 60 min at room temperature, and washed as before. Binding of HRP-secondary antibody conjugate was detected by incubating membranes in SuperSignal™ West “Femto” Maximum Sensitivity Substrate (Pierce, Inc. USA), and visualizing chemiluminescence using the LAS-3000 multi-imager (FujiFilm Inc., Japan).

Immune-reactive bands of about 24 kDa, consistent with the expected molecular masses of M. tuberculosis TetR-like protein, were detected by both antibodies in both clinical M. tuberculosis isolates CSU93 and HN878, and in the laboratory strain H37Rv (data not shown). In a control, recombinant TetR-like protein comprising a hexahistidine tag having an estimated molecular mass of about 25 kDa was also detected at the correct position (not shown). In a further experiment, the detection of these bands by Mo785E was prevented by pre-incubation of primary antibody in a 1000-fold molar excess of unlabelled recombinant TetR-like protein (data not shown).

### 5. Amplified Sandwich ELISA Using Polyclonal Ch4/5 Antibody and mAb Mo785E

The inventors also investigated whether or not a biotinylated antibody and streptavidin poly-HRP conjugate could improve ELISA sensitivity compared to conventional biotin-streptavidin-HRP systems or HRP-conjugated secondary antibodies.

An ELISA plate was coated overnight with capture antibody Ch4/5 at 2 μg/ml concentration. Following washing to remove unbound antibody, recombinant TetR-like protein was diluted from 100 ng/ml starting concentration to 490 fg/ml, and 50 μl aliquots of each dilution were added the wells of the antibody-coated ELISA plates. Following incubation for 1 hour, plates were washed to remove unbound antigen. Unlabelled monoclonal antibody Mo785E was contacted with the bound antigen-body complexes at 2.5 μg/ml concentration for standard sandwich ELISA.

For amplified sandwich ELISA, monoclonal antibody Mo785E was biotinylated and the biotinylated antibody contacted with the bound antigen-body complexes at 2.5 μg/ml concentration. Following incubation at room temperature for 1 hour, plates were washed, and incubated with 50 μl of a 1:5,000 (v/v) dilution of a secondary antibody consisting of HRP-conjugated sheep anti-mouse IgG (standard sandwich ELISA) or 50 μl of a 1:2,500 (v/v) dilution of HRP80-streptavidin. Plates were then incubated for a further one hour at room temperature, and washed as before. Finally, all samples were incubated with TMB for 30 mins (standard ELISA) or 10 mins (amplified ELISA). Absorbance was determined at 450-620 nm.

As shown in FIG. 119, there was significant enhancement of detection using the amplified sandwich ELISA under these conditions: The limit of detection of this amplified sandwich ELISA is about 18 pg/ml TetR-like protein, with half-maximum detection of about 1 ng/ml TetR-like protein. This compares favourably to the observed limit of detection of the standard sandwich ELISA of about 79-176 pg/ml TetR-like protein. Such sensitivity of detection coupled with low background in sandwich ELISA is considered by the inventors to be within useful limits.

### 6. Cross-Reactivity Between Antibodies Against TetR-like Protein of Different M. Tuberculosis Isolates

To further assess the suitability of TetR as a diagnostic marker for the presence of M. tuberculosis in biological samples, the inventors compared antibody reactivities between cellular extracts of the clinical M. tuberculosis strains CSU93 and HN878, and the laboratory M. tuberculosis strain H37Rv by amplified sandwich ELISA.

The amplified ELISA described herein was also used to detect recombinant TetR-like protein in whole cell extracts of the laboratory strain H37Rv and the clinical isolates CSU93 and HN878. Recombinant protein (1.8 μg/ml, 5.6 μg/ml, 16.7 μg/ml, and 50 μg/ml diluted in blocking buffer) was added to whole cell lysates, and the samples assayed in duplicate by amplified sandwich ELISA, performed essentially as described herein. The concentration of endogenous TetR-like protein in the whole cell lysates was calculated by interpolation from the standard curve and subtraction of the signal corresponding to the recombinant TetR-like protein spike. Levels of endogenous M. tuberculosis TetR-like protein present in these strains, as determined from two independent experiments is indicated in FIG. 120. Data indicate average TetR levels of about 3-5 μg/μg cell extract for H37Rv and CSU93, and about 9 pg/μg in whole cell extracts of the clinical isolate HN878.

In summary, the data obtained to date thus indicate that the amplified sandwich ELISA assay format is capable of detecting endogenous TetR-like protein in whole cell extracts of clinically-relevant and laboratory strains of M. tuberculosis.

### 7. Low Cross-Reactivity Between M. Tuberculosis and Non-Mycobacteria Pathogens

To further assess the suitability of TetR as a diagnostic marker for the presence of M. tuberculosis in biological samples, the inventors compared antibody cross-reactivities in the amplified sandwich ELISA performed essentially as described herein between different concentrations of recombinant TetR-like protein and cellular extracts of yeast, Escherichia coli, Bacillus subtilis or Pseudomonas aeruginosa. Assay conditions were varied slightly, employing HRP40-streptavidin at 1:2500 (v/v) dilution as opposed to HRP80-streptavidin, and developing TMB for 15 min for signal detection. Buffer without protein or cellular extract served as a negative control. No replacement amplification or iterative sample loading was performed in this diagnostic test.

Data presented in FIG. 121 show no detectable cross-reactivity of antibodies against M. tuberculosis TetR with yeast, Escherichia coli, Bacillus subtilis or Pseudomonas aeruginosa cellular extracts under the conditions tested. In particular there was little or no signal differential between the concentrations of cellular extracts tested, and the signal obtained were not significantly above background. In contrast, the assay detected as little as about 10 pg/ml recombinant TetR-like protein.

### 8. Cross-Reactivity Between Different Mycobacteria Species

To further assess the suitability of TetR-like as a diagnostic marker for the presence of M. tuberculosis in biological samples, and to assess the specificities of antibodies prepared against TetR-like protein, the inventors compared antibody reactivities in amplified sandwich ELISA performed as described herein above between cellular extracts of the Mycobacteria species M. tuberculosis, M. avium and M. intracellulaire.

Briefly, an ELISA plate was coated overnight with capture antibody Ch4/5. Following washing to remove unbound antibody, a cellular extract from each Mycobacteria species was added to the wells of the antibody-coated ELISA plates. As a negative control for each assay, buffer without cellular extract was used. Following incubation for 1 hour and washing to remove unbound antigen, biotinylated detection antibody Mo785E-Bio was contacted with the bound antigen-body complexes. Following incubation at room temperature for 1 hour, plates were washed, incubated with 1:2,500 (v/v) dilution of HRP80-streptavidin (poly-40 streptavidin-HRP conjugate may also be employed in place of HRP80-streptavidin), and then washed again, incubated with TMB for 10 mins, and the absorbance at 450-620 nm was determined. Samples were assayed in duplicate over three dilutions of whole cell extracts. A calibration standard curve was produced based on standardized levels of TetR-like protein.

Data presented in FIG. 122 show strong cross-reactivity between anti-TetR antibodies and whole cell extracts from M. tuberculosis, with almost undetectable cross-reactivity in whole cell extracts of M. avium and M. intracellulaire. In contrast, filtrates of whole cell extracts resulted in significant and comparable cross-reactivity in both M. tuberculosis and M. intracellulaire, suggesting that, similar to P5CR, M. tuberculosis TetR-like protein may be may be less suited for species-specific detection of M. tuberculosis under these assay conditions or using the selected antibody pair. This does not abrogate utility of antibodies against TetR-like protein in a general single-analyte diagnostic test, or alternatively, as part of a multi-analyte test in conjunction with antibodies against KARI protein, and/or a species-specific marker of M. tuberculosis such as described herein or known in the art. For example, antibodies against TetR-like protein may be employed in conjunction with subsequent culture of M. tuberculosis from P5CR-positive clinical specimens.

Alternatively, antibodies against KARI and antibodies against TetR-like may be used as a generic Mycobacteria test, wherein the presence of KARI cross-reactivity and the absence of TetR-like cross-reactivity is indicative of Mycobacteria other than M. tuberculosis or M. intracellulaire e.g., because it indicates M. avium or wherein cross-reactivity to both KARI and TetR-like indicates M. tuberculosis or M. intracellulaire.

Alternatively, or in addition, antibodies against KARI protein and/or TetR-like protein may be employed simultaneously with one or more antibodies against M. tuberculosis BSX and/or M. tuberculosis S9 protein and/or M. tuberculosis Rv1265 protein as described herein which have low cross-reactivity to the other Mycobacteria tested. In interpreting such a multi-analyte test, binding of antibodies against both KARI and TetR-like proteins indicates the presence M. tuberculosis or M. intracellulaire in the clinical sample and the additional binding of antibodies against M. tuberculosis BSX and/or M. tuberculosis S9 protein and/or M. tuberculosis Rv1265 protein indicates a greater likelihood of M. tuberculosis infection.

### 9. Relative Levels of TetR-like Protein Detectable in Mycobacterium Cells

To further assess the suitability of TetR-like as a diagnostic marker for Mycobacteria infection, the levels of TetR-like protein were determined in whole cell lysates of the M. tuberculosis strains H37Rv, CSU93 and HN878, and in M. tuberculosis, M. avium and M. intracellulaire, relative to 10 other M. tuberculosis antigens including TetR-like, TetR-like, P5CR, TetR-like, TetR-like and TetR-like protein described herein.

Amplified sandwich ELISA was performed as described in this example, to identify relative levels of each antigen according to the standard protocol, with calibration standards included to permit quantitation.

Data shown in FIGS. 124-131 indicate that TetR-like is expressed at low levels in all three M. tuberculosis strains tested, comparable to P5CR protein expression.

On the other hand, data shown in FIGS. 126-131 indicate that TetR-like protein is also relatively specific to M. tuberculosis and not detectable under the assay conditions employed in either M. avium or M. intracellulaire.

These data suggest utility of TetR-like as a generic single-analyte marker of mycobacteria or M. tuberculosis infection, or as part of a multi-analyte test for mycobacteria infection or M. tuberculosis infection in combination with BSX and/or KARIT and/or S9 and/or Rv1265 proteins. Other combinations are not excluded for multi-analyte testing of M. tuberculosis infection.

### 11. Optimizing the Limits of Detection

To further enhance sandwich ELISA sensitivity, a replacement amplification procedure is employed to employ iterative antigen binding following coating of the ELISA plate with capture antibody. Essentially, this will result in an increased amount of antigen being bound to the capture antibody notwithstanding the 50 μl volume limitations of a 96-well ELISA plate. Briefly, this iterative antigen loading involves repeating the antigen binding step in the sandwich ELISA several times, e.g., 2 or 3 or 4 or 5 times, etc. before washing and adding detection antibody. Naturally, each aliquot of antigen sample is removed following a standard incubation period before the next aliquot is added. The number of iterations can be modified to optimize the assay (e.g., parameters such as signal:noise ratio, detection limit and amount of antigen detected at half-maximum signal), depending upon the nature of the sample being tested (e.g., sample type), without undue experimentation.

For example, up to about 20 iterations of sample loading (i.e., up to a 20× replacement amplification) may provide a low background signal, and reduce the detection limit for RV1265 protein.

### Example 9

### Microorganism Deposits of Exemplary Plasmacytomas Expressing Monoclonal Antibodies for Detection of M. Tuberculosis

Without conceding that the deposit of any biological material is required to perform the invention as broadly described herein, or any example hereof, the following plasmacytomas have been deposited under the provisions of the Budapest treaty on the International Recognition of the Deposit of Microorganisms for the Purposes of Patent Procedure with the American Type Culture Collection (ATCC), as follows:

1. The mouse cell line designated 2B1C11 producing monoclonal antibody Mo2B1 (syn. 2B1) that binds that binds to KARI protein from Mycobacterium complex, especially M. tuberculosis, was deposited with the ATCC on May 21, 2009 and assigned ATCC Accession No. ______;
2. The mouse plasmacytoma cell line designated PRO 0107-639F producing monoclonal antibody Mo639F that binds to BSX protein rom Mycobacterium complex, especially M. tuberculosis, was deposited with the ATCC on Mar. 6, 2008 and assigned ATCC Accession No. PTA-9013;
3. The mouse plasmacytoma cell line designated PRO 0126-1025F producing monoclonal antibody Mo1025F that binds to S9 protein from Mycobacterium complex, especially M. tuberculosis, was deposited with the ATCC on Mar. 6, 2008 and assigned ATCC Accession No. PTA-9011;
4. The mouse plasmacytoma cell line designated PRO 0122-785E producing monoclonal antibody Mo785E that binds to Rv1265 protein from Mycobacterium complex, especially M. tuberculosis, was deposited with the ATCC on May 16, 2007 and assigned ATCC Accession No. PTA-8441;
5. The mouse plasmacytoma cell line designated PRO 0123-788C producing monoclonal antibody Mo788C that binds to TetR-like protein from Mycobacterium complex, especially M. tuberculosis, was deposited with the ATCC on May 16, 2007 and assigned ATCC Accession No. PTA-8440; and
6. The mouse plasmacytoma cell line designated PRO 0107-524D producing monoclonal antibody Mo524D that binds to EF-Tu protein from Mycobacterium complex, especially M. tuberculosis, was deposited with the ATCC on Mar. 6, 2008 and assigned ATCC Accession No. PTA-9012.

1-8. (canceled)

9. An isolated or recombinant antibody that binds specifically to an immunogenic protein of a mycobacterium of the Mycobacterium tuberculosis complex that is a putative Ketol-acid reductoisomerase (KARI) or an immunogenic peptide or immunogenic fragment or epitope thereof or to a fusion protein or protein aggregate comprising said immunogenic KARI protein, peptide, fragment or epitope.

10-14. (canceled)

15. An isolated antibody-producing cell or antibody-producing cell population that produces an antibody according to claim 9.

16-18. (canceled)

19. A composition comprising the isolated or recombinant antibody according to claim 9 and a pharmaceutically acceptable carrier, diluent or excipient.

20. A method of diagnosing tuberculosis or an infection by one or more mycobacteria of the M. tuberculosis complex in a subject comprising detecting in a biological sample from said subject antibodies against an immunogenic KARI protein of a mycobacterium of the M. tuberculosis complex or an immunogenic KARI peptide or immunogenic KARI fragment or epitope thereof, wherein the presence of said antibodies in the sample is indicative of infection.

21. The method of claim 20 comprising contacting a biological sample derived from the subject with the isolated or recombinant immunogenic KARI protein or an immunogenic KARI peptide or immunogenic KARI fragment or epitope thereof for a time and under conditions sufficient for an antigen-antibody complex to form and then detecting the formation of an antigen-antibody complex.

22-26. (canceled)

27. The method of claim 21 further comprising contacting a biological sample derived from the subject with an immunogenic protein or peptide from one or more mycobacteria of the Mycobacterium tuberculosis complex other than isolated or recombinant immunogenic KARI protein or an immunogenic KARI peptide or immunogenic KARI fragment or epitope thereof.

28. The method according to claim 27 wherein the immunogenic protein or peptide of other than isolated or recombinant immunogenic KARI protein or an immunogenic KARI peptide or immunogenic KARI fragment or epitope thereof is selected from the group consisting of BSX protein (UnitProtKB/TrEMBL Accession No. A5TZK2; SEQ ID NO: 2) and/or ribosomal protein S9 (UniProtKB/Swiss-Prot Accession No. A5U8B8; SEQ ID NO: 14) and/or protein Rv1265 (UniProtKB/Swiss-Prot Accession No. P64789; SEQ ID NO: 21) and/or elongation factor-Tu (EF-Tu) protein (UniProtKB/Swiss-Prot Accession No. A5U071; SEQ ID NO: 28-29) and/or P5CR protein (UniProtKB/Swiss-Prot Accession No. Q11141; SEQ ID NO: 36) and/or TetR-like protein (UnitProtKB/TrEMBL Accession No. A1QW92; SEQ ID NO: 44) and/or glutamine synthase (GS) protein (UnitProtKB/TrEMBL Accession No. O33342), an immunogenic peptide derived from said BSX protein, an immunogenic peptide derived from said S9, an immunogenic peptide derived from said Rv1265, an immunogenic peptide derived from said EF-Tu protein, an immunogenic peptide derived from said P5CR protein, an immunogenic peptide derived from said TetR-like protein and an immunogenic peptide derived from GS protein, and combinations thereof.

29. The method according to claim 27 wherein the immunogenic protein or peptide other than isolated or recombinant immunogenic KARI protein or an immunogenic KARI peptide or immunogenic KARI fragment or epitope thereof is selected from the group consisting of BSX protein (UnitProtKB/TrEMBL Accession No. A5TZK2; SEQ ID NO: 2), ribosomal protein S9 (UniProtKB/Swiss-Prot Accession No. A5U8B8; SEQ ID NO: 14), protein Rv1265 (UniProtKB/Swiss-Prot Accession No. P64789; SEQ ID NO: 21), an immunogenic peptide derived from said BSX protein, an immunogenic peptide derived from said S9 and an immunogenic peptide derived from said Rv1265, and combinations thereof.

30. The method according to claim 29 wherein the immunogenic protein or peptide other than isolated or recombinant immunogenic KARI protein or an immunogenic KARI peptide or immunogenic KARI fragment or epitope thereof is selected from the group consisting of BSX protein (UnitProtKB/TrEMBL Accession No. A5TZK2; SEQ ID NO: 2), protein Rv1265 (UniProtKB/Swiss-Prot Accession No. P64789; SEQ ID NO: 21), an immunogenic peptide derived from said BSX protein and an immunogenic peptide derived from said Rv1265, and combinations thereof.

31. A method of diagnosing tuberculosis or infection by one or more mycobacteria of the M. tuberculosis complex in a subject comprising detecting in a biological sample from said subject an immunogenic KARI protein or an immunogenic KARI peptide or immunogenic KARI fragment or epitope thereof using an isolated or recombinant antibody that binds specifically to an immunogenic KARI protein of the M. tuberculosis complex or an immunogenic peptide or immunogenic fragment or epitope thereof or to a fusion protein or protein aggregate comprising said immunogenic KARI protein, peptide, fragment or epitope, wherein the presence of said protein or immunogenic fragment or epitope in the sample is indicative of disease, disease progression or infection.

32. The method of claim 31 comprising contacting a biological sample derived from the subject with the isolated or recombinant antibody for a time and under conditions sufficient for an antigen-antibody complex to form and then detecting the formation of an antigen-antibody complex.

33. The method of claim 32 comprising performing an enzyme-linked immune-sorbent assay (ELISA).

34. The method of claim 33 wherein the ELISA is a sandwich ELISA using a capture antibody and a detection antibody.

35. The method according to claim 31 wherein the sample comprises an extract from brain, breast, ovary, lung, colon, pancreas, testes, liver, muscle, bone or mixtures thereof.

36. The method of claim 31 wherein the sample comprises a body fluid.

37. The method of claim 36 wherein the body fluid is sputum, serum, plasma, whole blood, saliva, urine, pleural fluid or mixtures thereof or a derivative thereof.

38. The method according to claim 31 comprising contacting a sample with antibodies that bind to KARI or immunogenic KARI peptide or fragment or epitope and with antibodies that bind to one or more proteins from one or more mycobacteria of the M. tuberculosis complex wherein said one or more proteins is(are) selected from the group consisting of BSX protein (UnitProtKB/TrEMBL Accession No. A5TZK2; SEQ ID NO: 2) and/or ribosomal protein S9 (UniProtKB/Swiss-Prot Accession No. A5U8B8; SEQ ID NO: 14) and/or protein Rv1265 (UniProtKB/Swiss-Prot Accession No. P64789; SEQ ID NO: 21) and/or elongation factor-Tu (EF-Tu) protein (UniProtKB/Swiss-Prot Accession No. A5U071; SEQ ID NO: 28-29) and/or P5CR protein (UniProtKB/Swiss-Prot Accession No. Q11141; SEQ ID NO: 36) and/or TetR-like protein (UnitProtKB/TrEMBL Accession No. A1QW92; SEQ ID NO: 44) and/or glutamine synthase (GS) protein (UnitProtKB/TrEMBL Accession No. O33342), an immunogenic peptide derived from said BSX protein, an immunogenic peptide derived from said S9, an immunogenic peptide derived from said Rv1265, an immunogenic peptide derived from said EF-Tu protein, an immunogenic peptide derived from said P5CR protein, an immunogenic peptide derived from said TetR-like protein and an immunogenic peptide derived from GS protein, and combinations thereof.

39-40. (canceled)

41. The method according to claim 31 wherein the subject is an immune-compromised or immune-deficient subject.

42. The method of claim 41 wherein the immune-compromised or immune deficient subject is infected with human immune-deficiency virus (HIV).

43. A method for determining the response of a subject having tuberculosis or an infection by one or more mycobacteria of the M. tuberculosis complex to treatment with a therapeutic compound for said tuberculosis or infection, said method comprising detecting a KARI protein or an immunogenic fragment or epitope thereof in a biological sample from said subject using the isolated or recombinant antibody according to claim 9, wherein a level of the protein or fragment or epitope that is enhanced, or not decreased or decreasing, compared to the level of that protein or fragment or epitope detectable in a normal or healthy subject indicates that the subject is not responding to said treatment or has not been rendered free of disease or infection.

44-54. (canceled)

55. A method for determining the response of a subject having tuberculosis or an infection by one or more mycobacteria of the M. tuberculosis complex to treatment with a therapeutic compound for said tuberculosis or infection, said method comprising detecting a KARI protein or an immunogenic fragment or epitope thereof in a biological sample from said subject using the isolated or recombinant antibody according to claim 9, wherein a level of the protein or fragment or epitope that is lower than the level of the protein or fragment or epitope detectable in a subject suffering from tuberculosis or infection by said one or more mycobacteria indicates that the subject is responding to said treatment or has been rendered free of disease or infection.

56-66. (canceled)

67. A method of monitoring disease progression, responsiveness to therapy or infection status by one or more mycobacteria of the M. tuberculosis complex in a subject comprising determining the level of M. tuberculosis KARI protein or an immunogenic fragment or epitope thereof in a biological sample from said subject at different times using the isolated or recombinant antibody according to claim 9, wherein a change in the level of the KARI protein, fragment or epitope indicates a change in disease progression, responsiveness to therapy or infection status of the subject.

68-79. (canceled)

80. A method of treatment of tuberculosis or infection by one or more mycobacteria of the M. tuberculosis complex comprising:
(i) performing a method according to claim 31 thereby detecting the presence of one or more of said mycobacteria in a biological sample from a subject; and
(ii) administering a therapeutically effective amount of a pharmaceutical composition to reduce the number of pathogenic bacilli in the lung, blood or lymph system of the subject.

81. A method of treatment of tuberculosis or infection by one or more mycobacteria of the M. tuberculosis complex comprising:
(i) performing a method according to claim 43 thereby detecting the presence of one or more of said mycobacteria in a biological sample from a subject being treated with a first pharmaceutical composition; and
(ii) administering a therapeutically effective amount of a second pharmaceutical composition to reduce the number of pathogenic bacilli in the lung, blood or lymph system of the subject.

82. A kit for diagnosing tuberculosis and/or detecting one or more mycobacteria of the M. tuberculosis complex in a biological sample, said kit comprising:
(i) one or more isolated or recombinant antibodies according to claim 9 or an immune reactive fragment thereof that bind specifically to the isolated or recombinant immunogenic KARI protein or an immunogenic KARI peptide or immunogenic KARI fragment or epitope thereof or to a fusion protein or protein aggregate comprising said immunogenic KARI protein, peptide, fragment or epitope; and
(ii) means for detecting the formation of an antigen-antibody complex, packaged with instructions for use.

83-86. (canceled)

87. A solid matrix comprising an isolated or recombinant antibody according to claim 9 adsorbed thereto.

88-96. (canceled)


SEQUENCE LISTING (60 sequences)

SEQ ID NO: 1 - protein, 333 aa, Mycobacterium tuberculosis
MFYDDDADLSIIQGRKVGVIGYGSQGHAHSLSLRDSGVQVRVGLKQGSRSRPKVEEQGLDVDTPAEVAKWADVVMVLAPDTAQAEIFAGDIEPNLKPGDALFFGHGLNVHFGLIKPPADVAVAMVAPKGPGHLVRRQFVDGKGVPCLVAVEQDPRGDGLALALSYAKAIGGTRAGVIKTTFKDETETDLFGEQTVLCGGTEELVKAGFEVMVEAGYPAELAYFEVLHELKLIVDLMYEGGLARMYYSVSDTAEFGGYLSGPRVIDAGTKERMRDILREIQDGSFVHKLVADVEGGNKQLEELRRQNAEHPIEVVGKKLRDLMSWVDRPITETA

SEQ ID NO: 2 - protein, 140 aa, Mycobacterium tuberculosis
MSSEEKLAAKVSTKASDVASDIGSFIRSQRETAHVSMRQLAERSGVSNPYLSQVERGLRKPSADVLSQIAKALRVSAEVLYVRAGILEPSETSQVRDAIITDTAITERQKQILLDIYASFTHQNEATREECPSDPTPTDD

SEQ ID NO: 3 - protein, 15 aa, artificial sequence
  Immunogenic peptide antigen derived from M. tuberculosis BSX protein
MRQLAERSGVSNPYL

SEQ ID NO: 4 - protein, 15 aa, artificial sequence
  Immunogenic peptide antigen derived from M. tuberculosis BSX protein
ERGLRKPSADVLSQI

SEQ ID NO: 5 - protein, 15 aa, artificial sequence
  Immunogenic peptide antigen derived from M. tuberculosis BSX protein
LRKPSADVLSQIAKA

SEQ ID NO: 6 - protein, 15 aa, artificial sequence
  Immunogenic peptide antigen derived from M. tuberculosis BSX protein
PSADVLSQIAKALRV

SEQ ID NO: 7 - protein, 15 aa, artificial sequence
  Immunogenic peptide antigen derived from M. tuberculosis BSX protein
SQIAKALRVSAEVLY

SEQ ID NO: 8 - protein, 15 aa, artificial sequence
  Immunogenic peptide antigen derived from M. tuberculosis BSX protein
AKALRVSAEVLYVRA

SEQ ID NO: 9 - protein, 15 aa, artificial sequence
  Immunogenic peptide antigen derived from M. tuberculosis BSX protein
VRAGILEPSETSQVR

SEQ ID NO: 10 - protein, 15 aa, artificial sequence
  Immunogenic peptide antigen derived from M. tuberculosis BSX protein
TAITERQKQILLDIY

SEQ ID NO: 11 - protein, 16 aa, artificial sequence
  Immunogenic peptide antigen derived from M. tuberculosis BSX protein
SQIAKALRVSAEVLYV

SEQ ID NO: 12 - protein, 15 aa, artificial sequence
  Immunogenic peptide antigen derived from M. tuberculosis BSX protein
MSSEEKLCDPTPTDD

SEQ ID NO: 13 - protein, 16 aa, artificial sequence
  Immunogenic peptide antigen derived from M. tuberculosis BSX protein
VRAGILEPSETSQVRC

SEQ ID NO: 14 - protein, 151 aa, Mycobacterium tuberculosis
MTETTPAPQTPAAPAGPAQSFVLERPIQTVGRRKEAVVRVRLVPGTGKFDLNGRSLEDYFPNKVHQQLIKAPLVTVDRVESFDIFAHLGGGGPSGQAGALRLGIARALILVSPEDRPALKKAGFLTRDPRATERKKYGLKKARKAPQYSKR

SEQ ID NO: 15 - protein, 8 aa, artificial sequence
  M. tuberculosis S9 peptide fragment
APLVTVDR

SEQ ID NO: 16 - protein, 7 aa, artificial sequence
  M. tuberculosis S9 peptide fragment
VHQQLIK

SEQ ID NO: 17 - protein, 6 aa, artificial sequence
  M. tuberculosis S9 peptide fragment
FDLNGR

SEQ ID NO: 18 - protein, 15 aa, artificial sequence
  M. tuberculosis S9 peptide fragment
VHQQLIKAPLVTVDR

SEQ ID NO: 19 - protein, 22 aa, artificial sequence
  M. tuberculosis S9 N-terminal peptide fragment
MTETTPAPQTPAAPAGPAQSFC

SEQ ID NO: 20 - protein, 25 aa, artificial sequence
  M. tuberculosis S9 N-terminal peptide fragment
MTETTPAPQTPAAPAGPAQSFGSGL

SEQ ID NO: 21 - protein, 226 aa, Mycobacterium tuberculosis
MVLARPDAVFAPARNRCHVSLPVNAMSLKMKVCNHVIMRHHHMHGRRYGRPGGWQQAQQPDASGAAEWFAGRLPEDWFDGDPTVIVDREEITVIGKLPGLESPEEESAARASGRVSRFRDETRPERMTIADEAQNRYGRKVSWGVEVGGERILFTHIAVPVMTRLKQPERQVLDTLVDAGVARSRSDALAWSVKLVGEHTEEWLAKLRTAMSAVDDLRAQGPDLPA

SEQ ID NO: 22 - protein, 12 aa, artificial sequence
  M. tuberculosis hypothetical protein Rv1265/MT1303 peptide fragment
KVSWGVEVGGER

SEQ ID NO: 23 - protein, 13 aa, artificial sequence
  M. tuberculosis hypothetical protein Rv1265/MT1303 peptide fragment
ILFTHIAVPVMTR

SEQ ID NO: 24 - protein, 10 aa, artificial sequence
  M. tuberculosis hypothetical protein Rv1265/MT1303 peptide fragment
TAMSAVDDLR

SEQ ID NO: 25 - protein, 18 aa, artificial sequence
  M. tuberculosis hypothetical protein Rv1265/MT1303 peptide fragment
TAMSAVDDLRAQGPDLPA

SEQ ID NO: 26 - protein, 14 aa, artificial sequence
  M. tuberculosis hypothetical protein Rv1265/MT1303 peptide fragment
ARNRSHVSLPVNAC

SEQ ID NO: 27 - protein, 11 aa, artificial sequence
  M. tuberculosis hypothetical protein Rv1265/MT1303 peptide fragment
EITVIGKLPGC

SEQ ID NO: 28 - protein, 396 aa, Mycobacterium tuberculosis
VAKAKFQRTKPHVNIGTIGHVDHGKTTLTAAITKVLHDKFPDLNETKAFDQIDNAPEERQRGITINIAHVEYQTDKRHYAHVDAPGHADYIKNMITGAAQMDGAILVVAATDGPMPQTREHVLLARQVGVPYILVALNKADAVDDEELLELVEMEVRELLAAQEFDEDAPVVRVSALKALEGDAKWVASVEELMNAVDESIPDPVRETDKPFLMPVEDVFTITGRGTVVTGRVERGVINVNEEVEIVGIRPSTTKTTVTGVEMFRKLLDQGQAGDNVGLLLRGVKREDVERGQVVTKPGTTTPHTEFEGQVYILSKDEGGRHTPFFNNYRPQFYFRTTDVTGVVTLPEGTEMVMPGDNTNISVKLIQPVAMDEGLRFAIREGGRTVGAGRVTKIIK

SEQ ID NO: 29 - protein, 396 aa, Mycobacterium tuberculosis
MAKAKFQRTKPHVNIGTIGHVDHGKTTLTAAITKVLHDKFPDLNETKAFDQIDNAPEERQRGITINIAHVEYQTDKRHYAHVDAPGHADYIKNMITGAAQMDGAILVVAATDGPMPQTREHVLLARQVGVPYILVALNKADAVDDEELLELVEMEVRELLAAQEFDEDAPVVRVSALKALEGDAKWVASVEELMNAVDESIPDPVRETDKPFLMPVEDVFTITGRGTVVTGRVERGVINVNEEVEIVGIRPSTTKTTVTGVEMFRKLLDQGQAGDNVGLLLRGVKREDVERGQVVTKPGTTTPHTEFEGQVYILSKDEGGRHTPFFNNYRPQFYFRTTDVTGVVTLPEGTEMVMPGDNTNISVKLIQPVAMDEGLRFAIREGGRTVGAGRVTKIIK

SEQ ID NO: 30 - protein, 19 aa, artificial sequence
  Peptide derived from M. tuberculosis Elongation factor-Tu comprising N-terminal biotin and N-terminal Ser-Gly-Ser-Gly spacer
SGSGGRVERGVINVNEEVE

SEQ ID NO: 31 - protein, 19 aa, artificial sequence
  Peptide derived from M. tuberculosis Elongation factor-Tu comprising N-terminal biotin and N-terminal Ser-Gly-Ser-Gly spacer
SGSGGVINVNEEVEIVGIR

SEQ ID NO: 32 - protein, 19 aa, artificial sequence
  Peptide derived from M. tuberculosis Elongation factor-Tu comprising N-terminal biotin and N-terminal Ser-Gly-Ser-Gly spacer
SGSGNEEVEIVGIRPSTTK

SEQ ID NO: 33 - protein, 19 aa, artificial sequence
  Peptide derived from M. tuberculosis Elongation factor-Tu comprising N-terminal biotin and N-terminal Ser-Gly-Ser-Gly spacer
SGSGIVGIRPSTTKTTVTG

SEQ ID NO: 34 - protein, 19 aa, artificial sequence
  Peptide derived from M. tuberculosis Elongation factor-Tu comprising N-terminal biotin and N-terminal Ser-Gly-Ser-Gly spacer
SGSGPSTTKTTVTGVEMFR

SEQ ID NO: 35 - protein, 20 aa, artificial sequence
  Immunogenic peptide antigen derived from M. tuberculosis elongation factor tu protein
VINVNEEVEIVGIRPSTTKC

SEQ ID NO: 36 - protein, 295 aa, Mycobacterium tuberculosis
MLFGMARIAIIGGGSIGEALLSGLLRAGRQVKDLVVAERMPDRANYLAQTYSVLVTSAADAVENATFVVVAVKPADVEPVIADLANATAAAENDSAEQVFVTVVAGITIAYFESKLPAGTPVVRAMPNAAALVGAGVTALAKGRFVTPQQLEEVSALFDAVGGVLTVPESQLDAVTAVSGSGPAYFFLLVEALVDAGVGVGLSRQVATDLAAQTMAGSAAMLLERMEQDQGGANGELMGLRVDLTASRLRAAVTSPGGTTAAALRELERGGFRMAVDAAVQAAKSRSEQLRITPE

SEQ ID NO: 37 - protein, 18 aa, artificial sequence
  immunogenic peptide from M. tuberculosis P5CR protein
AMPNAAALVGAGVTALAK

SEQ ID NO: 38 - protein, 20 aa, artificial sequence
  immunogenic peptide from M. tuberculosis P5CR protein
AMPNAAALVGAGVTALAKGR

SEQ ID NO: 39 - protein, 16 aa, artificial sequence
  immunogenic peptide from M. tuberculosis P5CR protein
MDQDQGGANGELMGLR

SEQ ID NO: 40 - protein, 8 aa, artificial sequence
  immunogenic peptide from M. tuberculosis P5CR protein
ELERGGFR

SEQ ID NO: 41 - protein, 12 aa, artificial sequence
  immunogenic peptide from M. tuberculosis P5CR protein
MAVDAAVQAAKS

SEQ ID NO: 42 - protein, 19 aa, artificial sequence
  immunogenic peptide from M. tuberculosis P5CR protein
RANYLAQTYSVLVTSAADA

SEQ ID NO: 43 - protein, 18 aa, artificial sequence
  immunogenic peptide from M. tuberculosis P5CR protein
MGLRVDLTASRLRAAVTS

SEQ ID NO: 44 - protein, 213 aa, Mycobacterium tuberculosis
MPRQAGRWSPTALRILGAAAELIALRGYSSTSTRDIAAAVGVEQPAIYKHFSAKRDILAALVRLAVEWPLELFGHITAMPVPAVVKLHRWLTESLDHLHASPYVLVSILITPDLHQESFVAERELVAEMERALVGLIETGQGEGDVRAMHPLSAARLVQALFDALALPEFAVSPDEIVEFAMTALLSDPDRLAEIRAAADALEIQTAPPDRGL

SEQ ID NO: 45 - protein, 19 aa, artificial sequence
  Peptide fragment from M. tuberculosis putative transcriptional regulatory protein of TetR family of proteins
MATTQVPASLAGMPRQAGR

SEQ ID NO: 46 - protein, 14 aa, artificial sequence
  Peptide fragment from M. tuberculosis putative transcriptional regulatory protein of TetR family of proteins
ATTQVPASLAGMPR

SEQ ID NO: 47 - protein, 18 aa, artificial sequence
  Peptide fragment from M. tuberculosis putative transcriptional regulatory protein of TetR family of proteins
ATTQVPASLAGMPRQAGR

SEQ ID NO: 48 - protein, 7 aa, artificial sequence
  Peptide fragment from M. tuberculosis putative transcriptional regulatory protein of TetR family of proteins
WSPTALR

SEQ ID NO: 49 - protein, 8 aa, artificial sequence
  Peptide fragment from M. tuberculosis putative transcriptional regulatory protein of TetR family of proteins
GYSSTSTR

SEQ ID NO: 50 - protein, 17 aa, artificial sequence
  Peptide fragment from M. tuberculosis putative transcriptional regulatory protein of TetR family of proteins
AAADALEIQTAPPDRGL

SEQ ID NO: 51 - protein, 22 aa, artificial sequence
  Peptide fragment from M. tuberculosis putative transcriptional regulatory protein of TetR family of proteins
PVRWPQAPPWCGNYATLGSVMK

SEQ ID NO: 52 - protein, 19 aa, artificial sequence
  Peptide fragment from M. tuberculosis putative transcriptional regulatory protein of TetR family of proteins
AALPGLVADMTADAELNAR

SEQ ID NO: 53 - protein, 12 aa, artificial sequence
  Peptide fragment from M. tuberculosis putative transcriptional regulatory protein of TetR family of proteins
LIELIGGATMLR

SEQ ID NO: 54 - protein, 24 aa, artificial sequence
  Peptide fragment from M. tuberculosis putative transcriptional regulatory protein of TetR family of proteins
MLLYPDDMLDDAWVDQTTAIVVRG

SEQ ID NO: 55 - protein, 29 aa, artificial sequence
  Synthetic immunogenic peptide fragment from M. tuberculosis putative transcriptional regulatory protein of TetR family of proteins
RAMHPLSAARLVQALFDALALPEFAVSPC

SEQ ID NO: 56 - protein, 16 aa, artificial sequence
  Synthetic immunogenic peptide fragment from M. tuberculosis putative transcriptional regulatory protein of TetR family of proteins
DLHQESFVAERELVAC

SEQ ID NO: 57 - protein, 25 aa, artificial sequence
  Immunogenic peptide derived from M. tuberculosis glutamine synthase protein
RGTDGSAVFADSNGPHGMSSMFRSF

SEQ ID NO: 58 - protein, 20 aa, artificial sequence
  Immunogenic peptide derived from M. tuberculosis glutamine synthase protein
WASGYRGLTPASDYNIDYAI

SEQ ID NO: 59 - protein, 26 aa, artificial sequence
  Immunogenic peptide derived from M. tuberculosis glutamine synthase protein
RGTDGSAVFADSNGPHGMSSMFRSFC

SEQ ID NO: 60 - protein, 21 aa, artificial sequence
  Immunogenic peptide derived from M. tuberculosis glutamine synthase protein
WASGYRGLTPASDYNIDYAIC